(19) United States
(12) Patent Application Publication
(10) Pub. No.: US 2006/0147946 A1
(43) Pub. Date: Jul. 6, 2006

(54) Novel calcium channel variants and methods of use thereof

(76) Inventors: Pinchas Akiva, Ramat-Gan (IL); Alexander Dlber, Rishon-LeZion (IL); Sarah Pollock, Tel-Aviv (IL); Zurit Levine, Herzlia (IL); Sergey Nemzer, RaAnana (IL); Vladimir Grebinskiy, Highland Park, NJ (US); Brian Meloon, Baltimore, MD (US); Andrew Olson, Northport, NJ (US); Avi Rosenberg, Kfar Saba (IL); Ami Haviv, Hod-HaSharon (IL); Shaul Zevin, Mevaseret Zion (IL); Tomer Zekhari, Givataim (IL); Zipi Shaged, Tel-Aviv (IL); Moshe Olshansky, Haifa (IL); Arial Farkash, Haifa (IL); Eyal Privman, Tel-Aviv (IL); Amit Novik, Beit-HaSharon (IL); Naomi Keren, Givat Shmuel (IL); Gad S. Cojocaru, Ramat-HaSharon (IL); Yossi Cohen, Banstead (GB); Ronen Shemesh, Modiln (IL); Osnat Sella-Tavor, Kfar Kish (IL); Liat Mintz, East Brunswick, NJ (US); Hanquing Xie, Lambertville, NJ (US); Dvir Dahary, Tel-Aviv (IL); Erez Levanon, Petach Tikva (IL); Shiri Freilich, Haifa (IL); Nili Beck, Kfar Saba (IL); Wei-Yong Zhu, Plainsboro, NJ (US); Alon Wasserman, New York, NY (US); Chen Chermesh, Mishmar HaShiva (IL); Idit Azar, Tel-Aviv (IL); Rotem Sorek, Rechovot (IL); Jeanne Bernstein, Kfar Yona (IL)

Correspondence Address: STAAS & HALSEY LLP, SUITE 700, 1201 NEW YORK AVENUE, N.W., WASHINGTON, DC, 20005, US

(21) Appl. No.: 11/051,725

(22) Filed: Jan. 27, 2005

Related U.S. Application Data
(60) Provisional application No. 60/539,129, filed on Jan. 27, 2004.

(51) Int. Cl. C12Q 1/68; C07H 21/04; C12P 21/06; C07K 14/705

(52) U.S. Cl. 435/6; 435/69.1; 435/320.1; 435/325; 530/350; 536/23.5

(57) ABSTRACT

Novel alpha 1 subunit splice variants, including nucleic and amino acid sequences. Methods of use thereof are also described.

### CROSS-REFERENCE TO RELATED APPLICATION(S)

This application is related to Novel Calcium Channel Variants and Methods of use thereof, and claims priority to the below U.S. provisional applications which are incorporated by reference herein:

Application No. 60/539,129 filed Jan. 27, 2004—Methods and Systems for Annotating Biomolecular Sequences

### SEQUENCE LISTING

The instant application contains a “lengthy” Sequence Listing which has been submitted via CD-R in lieu of a printed paper copy, and is hereby incorporated by reference in its entirety. Said CD-R, recorded on Apr. 16, 2005, are labeled CRF, “Copy 1” and “Copy 2”, respectively, and each contains only one identical 1.061 Mb file (18471010.APP).

### FIELD OF THE INVENTION

The present invention is of novel calcium channel splice variants, including nucleic acid sequences and amino acid sequences, and methods of use thereof.

### BACKGROUND OF THE INVENTION

Voltage-sensitive calcium channels, also termed voltage-gated calcium channels, have many important physiological functions, for example for regulating cardiac function. Cardiac and vascular smooth muscle cells have calcium channels within the cell membrane. Calcium influx through these channels initiates a process of electromechanical coupling which ultimately leads to muscle contraction.

Different voltage-gated calcium channels have different structures and are blocked by different drugs. For example, the L-type (or long lasting) calcium channel is blocked by dihydropyridines, phenylalkylamines, and benzothiazepines. The channel structure features four transmembrane domains that form the calcium channel as a pore through the membrane. L-type calcium channels are characterized by large sustained conductance and slow inactivation. These channels appear at high levels in the heart and smooth muscle, and are responsible for the plateau phase (slow inward current) of the action potential.

T-type calcium channels are structurally similar to L-type channels, but have different behavior. They inactivate rapidly, and are involved in cardiac pacemaker activity and triggering contraction in vascular smooth muscle. They have high abundance in SA (sinoatrial) nodal tissue but low abundance in adult ventricular myocardium (see below for a description of the effect of calcium channel function on heart function). These channels are typically less sensitive to calcium channel blockers that affect L-type calcium channels with the exception of mibefradil.

N-type calcium channels are found only in neuronal cells, and are generally not sensitive to the cardiac specific calcium channel blockers. N (neuronal), P (Purkinje cell), Q (granular cell) and R (toxin-resistant) channels can be distinguished depending on their tissue expression pattern and toxin sensitivity, respectively.

Various disorders are associated with calcium channel activity. A “disorder associated with calcium channel activity” as used herein is a physiological malfunction arising from inappropriate calcium channel behavior. Such disorders include, for example, but are not limited to cardiovascular disease, pulmonary hypertension, peripheral vascular disorder, migraine disorder, mania, epilepsy, depression, hyperuricemia, and asthma (achalasia asthma and bronchial asthma).

Part of the importance of classifying calcium channels according to their sensitivity to calcium channel blockers is that the above diseases may be treated through the use of calcium channel blockers, which reduce calcium influx through the channel.

Calcium channel blockers are a chemically diverse class of compounds having important therapeutic value in the control of a variety of diseases including several cardiovascular disorders, such as hypertension, angina, and cardiac arrhythmias (Fleckenstein, Cir. Res. v. 52, (suppl. 1), p. 13-16 (1983); Fleckenstein, Experimental Facts and Therapeutic Prospects, John Wiley, New York (1983); McCall, D., Curr Pract Cardiol, v. 10, p. 1-11 (1985)).

These drugs prevent or slow the entry of calcium into cells by regulating cellular calcium channels. (Remington, The Science and Practice of pharmacy, Nineteenth Edition, Mack Publishing Company, Eaton, Pa., p. 963 (1995)). The regulation of calcium entry into the cells of the cardiovascular system is highly important for proper cardiac activity. The ability to regulate the entry of calcium into cardiac and vascular smooth muscle cells is a powerful therapeutic approach in the treatment of angina and hypertension respectively. Likewise, blocking calcium influx into cardiac tissues and conduction systems provides a useful approach to control certain types of arrhythmia.

Calcium channel blockers are also believed to be useful in the treatment of other disorders in which the regulation of calcium plays a role in normal hemostasis. Such disorders include, for example, pulmonary hypertension, peripheral vascular disease, mild congestive heart failure, hypertrophic subaortic stenosis, protection against ischemic injury, stroke, migraine, tumor resistance to anti-neoplastic drugs, achalasia, esophageal spasms, bronchial asthma, premature labor, dysmenorrhea, and enhancement of success in renal transplantation. (Remington, The Science and Practice of Pharmacy, Nineteenth Edition, Mack Publishing Company, Eaton, Pa., p. 963 (1995)).

Most of the currently available calcium channel blockers belong to one of three major chemical groups of drugs, the dihydropyridines, such as nifedipine, the phenyl alkyl amines, such as verapamil, and the benzothiazepines such as diltiazem.

Negative inotropic effects are seen with some of the L-type channel blockers (a direct effect on myocardial L-type channels). These effects result in reduced force of contraction of the myocardium. Therefore, these blockers are avoided in individuals with cardiomyopathy, or weakened heart muscle.

The negative inotropic effect is due to reduced inward movement of Ca++ during the action potential plateau phase (due to inhibition of slow (L-type) channel). Some calcium channel blockers, such as dihydropyridines, have very modest negative inotropic effects. Mibefradil (a T-type channel blocker) has no negative inotropic effects because there appear to be few T-type channels in adult ventricular muscle.

Negative chronotropic/dromotropic effects (pacemaker activity/conduction velocity) are also seen with some of the Ca++ channel blockers. Negative chronotropic effects result in slowing of the rate of myocardial contraction (time between p waves). Dromotropic effects relate to the time that the heart requires complete a single cycle of contraction (the p-t interval).

Verapamil (and to a lesser extent diltiazem) decreases the rate of recovery of the slow channel in AV conduction system and SA node, and therefore acts directly to depress SA node pacemaker activity and slow conduction. Contraction starts with depolarization of the SA (sinoatrial) node, followed by a wave of depolarization of the atrial tissue and contraction of the atria, causing blood to pour into the ventricles. The AV (atrioventricular) node depolarizes and causes the ventricles to contract, thereby pumping blood out of the heart and into the circulatory system.

Ca++-channel block by verapamil and diltiazem is frequency- and voltage-dependent, making them more effective in cells that are rapidly depolarizing. Mibefradil has negative chronotropic and dromotropic effects.

T-type channels are important for regulating Ca++ influx in pacemaker cells and cells of the conduction system. Nifedipine and related dihydropyridines do not have significant direct effects on the atrioventricular conduction system or sinoatrial node at normal doses, and therefore do not have direct effects on conduction or automaticity. Dihydropyridines can cause reflex increases in heart rate because of their potent vasodilating effects.

Hemodynamic effects of calcium channel blockers used as drugs tend to include decreasing coronary vascular resistance and increasing coronary blood flow, as well as decreasing peripheral resistance via vasodilatation of arterioles. However, these drugs typically do not affect venous tone.

The structure of calcium channels is important for their sensitivity to different calcium channel blockers. Certain calcium channel splice variants are known, but their function is not always understood. Furthermore, splice variants may have important physiological and regulatory effects on tissues for which calcium channel function is important, such as for cardiac tissue for example. However, these effects have not been explored and their overall importance on cardiac physiology and function, and the physiology and function of other such tissues that are affected by calcium channel function, is not known.

Therefore, exploration of calcium channel splice variants would clearly be useful to develop new drugs and to understand the physiological importance of such splice variants.

### SUMMARY OF THE INVENTION

The present invention provides a plurality of different splice variants of the voltage-gated calcium channel alpha subunit, which is a very important subunit in terms of the regulation of calcium channel function.

According to preferred embodiments of the present invention, there is provided an isolated polynucleotide comprising a nucleic acid sequence according to any of SEQ ID NO 23-44, or the complement thereof. Preferably, the nucleic acid comprises a polynucleotide encoding for the amino acid sequence of any of SEQ ID NOs 1-22. Optionally and preferably, the nucleotide sequence encodes for a polypeptide consisting of the amino acid sequence of any of SEQ ID NOs 1-22.

According to other preferred embodiments of the present invention, there is provided an isolated polypeptide comprising a polypeptide according to any of SEQ ID NOs 1-22. Preferably, the polypeptide consists of the amino acid sequence according to any of SEQ ID NOs 1-22.

According to preferred embodiments of the present invention, there is provided an expression vector comprising a nucleotide sequence encoding for any of SEQ ID NOs 1-22, wherein the nucleotide sequence is transcriptionally coupled to an exogenous promoter. Preferably, the nucleotide sequence encodes for a polypeptide consisting of the amino acid sequence according to any of SEQ ID NOs 1-22. More preferably, the nucleotide sequence comprises any of SEQ ID NO 23-44. Optionally and more preferably, the nucleotide sequence consists of the sequence of any of SEQ ID NO 23-44.

According to preferred embodiments of the present invention, there is provided a recombinant cell comprising the expression vector of claim 6, wherein the cell comprises an RNA polymerase recognized by the promoter. Optionally, the recombinant cell is made by a process comprising the step of introducing the expression vector described herein into the cell.

According to preferred embodiments of the present invention, there is provided a method of preparing a splice variant polypeptide comprising growing the recombinant cell as described herein under conditions wherein the polypeptide is expressed from the expression vector.

According to preferred embodiments of the present invention, there is provided a method of screening for compounds able to bind selectively to a splice variant according to the present invention comprising the steps of: (a) providing a splice variant polypeptide according to any of SEQ ID NOs 1-22; (b) providing a WT protein polypeptide that is not the splice variant polypeptide, (c) contacting the splice variant polypeptide and the WT polypeptide with a test preparation comprising one or more compounds; and (d) determining the binding of the test preparation to the splice variant polypeptide and the WT polypeptide, wherein a test preparation which binds the splice variant polypeptide but does not bind the WT polypeptide contains a compound that selectively binds the splice variant polypeptide.

Preferably, the splice variant polypeptide is obtained by expression of the polypeptide from an expression vector comprising a polynucleotide encoding the amino acid sequence according to any of SEQ ID NOs 1-22. More preferably, the polypeptide consists of the amino acid sequence according to any of SEQ ID NOs 1-22.

According to preferred embodiments of the present invention, there is provided a method of screening for a compound able to bind to or interact with a splice variant protein or a fragment thereof comprising the steps of: (a) expressing a polypeptide comprising the amino acid sequence according to any of SEQ ID NOs 1-22 or fragment thereof from a recombinant nucleic acid; (b) providing to the polypeptide a labeled ligand that specifically binds to the polypeptide and a test preparation comprising one or more compounds; and (c) measuring the effect of the test preparation on binding of the labeled ligand to the polypeptide, wherein a test preparation that alters the binding of the labeled ligand to the polypeptide contains a compound that binds to or interacts with the polypeptide. Optionally, steps (b) and (c) are performed in vitro. Also optionally, steps (a), (b) and (c) are performed using a whole cell.

Preferably, the polypeptide is expressed from an expression vector. Also preferably, the ligand is a calcium channel-binder. More preferably, the polypeptide consists of an amino acid sequence according to any of SEQ ID NOs 1-22 or a fragment thereof. Most preferably, the test preparation contains one compound.

According to preferred embodiments of the present invention, preferably any of the nucleic acid and/or amino acid sequences described herein further comprises any sequence having at least about 70%, preferably at least about 80%, more preferably at least about 90%, most preferably at least about 95% homology thereto.

All nucleic acid sequences and/or amino acid sequences shown herein as embodiments of the present invention relate to their isolated form, as isolated polynucleotides (including for all transcripts), oligonucleotides (including for all segments, amplicons and primers), peptides (including for all tails, bridges, insertions or heads, optionally including other antibody epitopes as described herein) and/or polypeptides (including for all proteins). It should be noted that oligonucleotide and polynucleotide, or peptide and polypeptide, may optionally be used interchangeably.

It should be noted that for the sequence listing, the amino acid sequences are listed first. The nucleic acid sequences are listed next according to the following nomenclature for the sequence names: SEQ ID NO:23

>T80376_T2 (1 T80376_P2)

For this name, the variant protein name appears in parentheses, proceeded by the relevant amino acid SEQ ID NO.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1: Mutually exclusive behavior gives two different transcripts; each of them contains one of the mutually exclusive exons (marked as green and yellow boxes).

FIG. 2 illustrates some aspects of an embodiment of the present invention with regard to mutually exclusive exons in the CACNA1C gene (SEQ ID NOS 189-190, respectively).

FIG. 3 shows an example for one possibly novel mutually exclusive exon in the SORCS3 gene (from UCSC genome browser, genome.ucsc.edu). The common exon is marked by an orange arrow and the putative exon is marked by red arrow. Only one human mRNA supports this gene. The two hills marked by orange and red circles, depict areas of human-mouse high conservation.

### DESCRIPTION OF PREFERRED EMBODIMENTS

Voltage gated calcium channels usually (although not necessarily) feature five subunits arranged in a complex that includes a pore through which the calcium ion may enter the cell. The alpha 1 subunit forms the pore itself and is encoded by CACNA1 genes. In order to form a functional calcium channel complex, the alpha 1 subunit coassembles with at least three accessory subunits encoded by two gene families: an intracellular beta subunit encoded by a CACNB gene, and an extracellular alpha 2 subunit linked by a di-sulphide bond to the membrane-anchoring delta subunit, both of which are encoded by a CACNA2D gene. In skeletal muscle, an additional accessory transmembranal gamma subunit is part of the channel complex. For at least some variants, such as the LVA channel (T-type), only the alpha subunit is required for functionality.

The alpha 1 subunit of the channel has a typical structural motif which involves four domains, each containing a series of six transmembrane alpha-helical segments, numbered S1-S6, which are connected by both intracellular and extracellular loops. The alpha 1 subunit itself determines the main characteristics of the cation channel complex such as its ion selectivity, voltage sensitivity, pharmacology (particularly sensitivity to calcium channel blockers) and binding characteristics for endogenous and exogenous ligands. The voltage sensitivity of cation channels is determined by the S4 segments, which are thought to move outward upon depolarization causing the channels to open. Calcium flows through the ion conducting pore, which is thought to be lined by the S5-S6 loops of all four domains. Although the activation gate is believed to lie within the pore formed by the alpha 1 subunit, the location of the inactivation gate is not clear.

The alpha 1 subunits are encoded by only ten different genes that are known to date, yet (in combination with the accessory subunits) these alpha 1 subunits must mediate a wide variety of functions. These functions are specific to particular types of calcium channels as described above. Furthermore, malfunction of calcium channels can result in markedly different disease patterns and characteristics, which further demonstrates the wide variety of functions achieved with such a small number of different genes. Structural diversity of alpha 1 subunits, and hence functional diversity, is increased through splice variants. Different splice variants of alpha 1 subunits may have strikingly different structures and hence different functions.

Alternative splicing is a process by which several mRNA isoforms can be generated from a single gene. It occurs by using different 5′-3′ pairs of splice sites while processing different molecules of the same pre-mRNA. Alternative splicing involving coding exons results in the production of several proteins from the same mRNA. Some alternative splicing events are obligatory, causing the production of two or more mRNA variants at constant ratios in all cells in which the gene is expressed. Other events are facultative and depend on such factors as sex, cell type, developmental stage, or physiological signals. Alternative splicing increases the diversity of the protein inventory within and between cells, and adds an additional regulatory dimension to the expression pattern of the organism.

In order to obtain mature mRNA which can be directly translated into a protein sequence, the non-coding regions corresponding to introns of the DNA must be removed from the precursor pre-mRNA by the splicing process. The borders of these introns are determined according to patterns of typical nucleotide sequences within the intron and adjacent exons.

Voltage-gated calcium channels are thought to have evolved by multiple gene duplication from a common ancestral channel gene encoding a one-domain potassium channel. The intron-exon boundaries have been shown to be conserved between different CACNA1 genes and also between species. The gene structure within the CACNA1 gene family is generally well conserved at coding regions for segments S1-S5 of all four domains. The remaining regions, especially those coding for the domain interlinkers and the S6 segments of all four domains, show less conserved gene structure suggesting that these portions of the protein vary in order to provide the required functional diversity. These less conserved coding regions coincide with the regions in which alternative splicing to result in the production of different transcripts, and hence different proteins, with similar function and sequence but different expression patterns, also known as splice isoforms.

Specific examples of splice variants of alpha 1 subunits of calcium channels are described in greater detail below. The effect of the changed amino acid sequence, and hence structure of the alpha 1 subunit protein, on calcium channel function is also described in greater detail below. These functional differences may optionally be exploited in order to better design small molecules and/or other drugs for regulating calcium channel function, for example in order to design a drug that either specifically binds or alternatively specifically does not bind to a particular variant calcium channel, determined by a type of alpha 1 subunit splice variant that is present. Various preferred embodiments of the present invention related to these calcium channel splice variants are described in greater detail below.

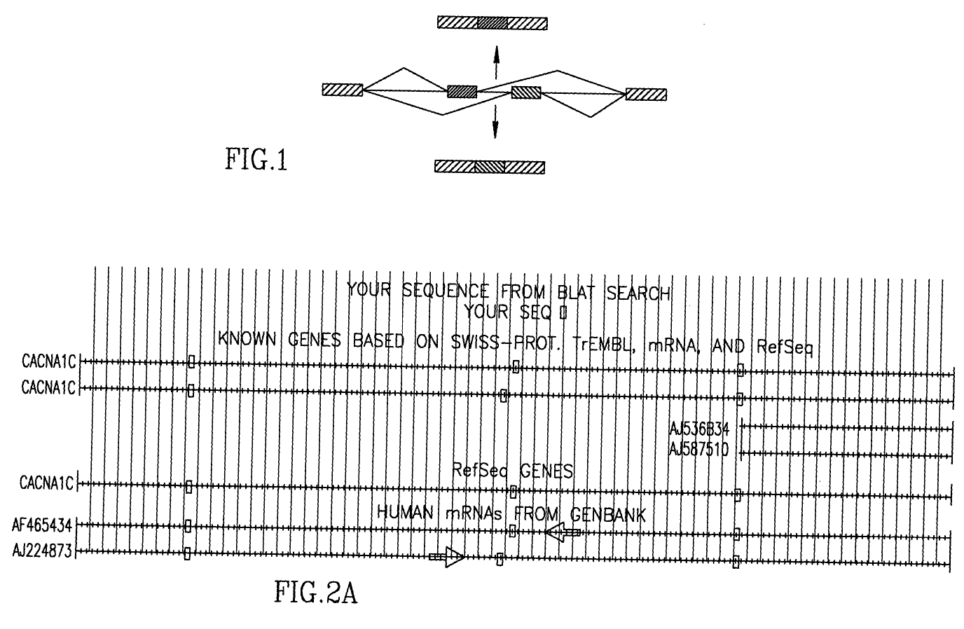

Some of the splice variants of the present invention feature mutually exclusive exons with regard to the known protein (WT or wild type protein). Mutually exclusive exons are two exons that are alternatively spliced in a unique way: if one of them is included in the transcript, the other one is not, and vice versa (FIG. 1). Of the known alternative splicing events, 9% are of the mutually exclusive exon type. Mutually exclusive exons are located in tandem and are usually flanked by introns of different phases (the relative position of the intron within or between codons). Thus, if both exons were incorporated into the same product it would result in frameshift that will lead to nonsense transcript. Examples 2, 4, 5, 15 and 19 below feature mutually exclusive exons.

According to preferred embodiments of the present invention, the present invention optionally and preferably encompasses any amino acid sequence or fragment thereof encoded by a nucleic acid sequence corresponding to a splice variant protein as described herein, including any oligopeptide or peptide relating to such an amino acid sequence or fragment, including but not limited to the unique amino acid sequences of these proteins that are depicted as tails, heads, insertions, edges or bridges. The present invention also optionally encompasses antibodies capable of recognizing, and/or being elicited by, such oligopeptides or peptides.

The present invention also optionally and preferably encompasses any nucleic acid sequence or fragment thereof, or amino acid sequence or fragment thereof, corresponding to a splice variant of the present invention as described above, optionally for any application.

In another embodiment, the present invention relates to bridges, tails, heads and/or insertions, and/or analogs, homologs and derivatives of such peptides. Such bridges, tails, heads and/or insertions are described in greater detail below with regard to the Examples.

As used herein a “tail” refers to a peptide sequence at the end of an amino acid sequence that is unique to a splice variant according to the present invention. Therefore, a splice variant having such a tail may optionally be considered as a chimera, in that at least a first portion of the splice variant is typically highly homologous (often 100% identical) to a portion of the corresponding known protein, while at least a second portion of the variant comprises the tail.

As used herein a “head” refers to a peptide sequence at the beginning of an amino acid sequence that is unique to a splice variant according to the present invention. Therefore, a splice variant having such a head may optionally be considered as a chimera, in that at least a first portion of the splice variant comprises the head, while at least a second portion is typically highly homologous (often 100% identical) to a portion of the corresponding known protein.

As used herein “an edge portion” refers to a connection between two portions of a splice variant according to the present invention that were not joined in the wild type or known protein. An edge may optionally arise due to a join between the above “known protein” portion of a variant and the tail, for example, and/or may occur if an internal portion of the wild type sequence is no longer present, such that two portions of the sequence are now contiguous in the splice variant that were not contiguous in the known protein. A “bridge” may optionally be an edge portion as described above, but may also include a join between a head and a “known protein” portion of a variant, or a join between a tail and a “known protein” portion of a variant, or a join between an insertion and a “known protein” portion of a variant.

Optionally and preferably, a bridge between a tail or a head or a unique insertion, and a “known protein” portion of a variant, comprises at least about 10 amino acids, more preferably at least about 20 amino acids, most preferably at least about 30 amino acids, and even more preferably at least about 40 amino acids, in which at least one amino acid is from the tail/head/insertion and at least one amino acid is from the “known protein” portion of a variant. Also optionally, the bridge may comprise any number of amino acids from about 10 to about 40 amino acids (for example, 10, 11, 12, 13 . . . 37, 38, 39, 40 amino acids in length, or any number in between).

It should be noted that a bridge cannot be extended beyond the length of the sequence in either direction, and it should be assumed that every bridge description is to be read in such manner that the bridge length does not extend beyond the sequence itself.

Furthermore, bridges are described with regard to a sliding window in certain contexts below. For example, certain descriptions of the bridges feature the following format: a bridge between two edges (in which a portion of the known protein is not present in the variant) may optionally be described as follows: a bridge portion of CONTIG-NAME_P1 (representing the name of the protein), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise XX (2 amino acids in the center of the bridge, one from each end of the edge), having a structure as follows (numbering according to the sequence of CONTIG-NAME_P1): a sequence starting from any of amino acid numbers 49−x to 49 (for example); and ending at any of amino acid numbers 50+((n−2)−x) (for example), in which x varies from 0 to n−2. In this example, it should also be read as including bridges in which n is any number of amino acids between 10-50 amino acids in length. Furthermore, the bridge polypeptide cannot extend beyond the sequence, so it should be read such that 49−x (for example) is not less than 1, nor 50+((n−2)−x) (for example) greater than the total sequence length.

In another embodiment, this invention provides antibodies specifically recognizing the splice variants and polypeptide fragments thereof of this invention. Preferably such antibodies differentially recognize splice variants of the present invention but do not recognize a corresponding known protein (such known proteins are discussed with regard to their splice variants in the Examples below).

In another embodiment, this invention provides an isolated nucleic acid molecule encoding for a splice variant according to the present invention, having a nucleotide sequence as set forth in any one of the sequences listed herein, or a sequence complementary thereto. In another embodiment, this invention provides an isolated nucleic acid molecule, having a nucleotide sequence as set forth in any one of the sequences listed herein, or a sequence complementary thereto. In another embodiment, this invention provides an oligonucleotide of at least about 12 nucleotides, specifically hybridizable with the nucleic acid molecules of this invention. In another embodiment, this invention provides vectors, cells, liposomes and compositions comprising the isolated nucleic acids of this invention.

Nucleic Acid Sequences and Oligonucleotides

Various embodiments of the present invention encompass nucleic acid sequences described hereinabove; fragments thereof, sequences hybridizable therewith, sequences homologous thereto, sequences encoding similar polypeptides with different codon usage, altered sequences characterized by mutations, such as deletion, insertion or substitution of one or more nucleotides, either naturally occurring or artificially induced, either randomly or in a targeted fashion.

The present invention encompasses nucleic acid sequences described herein; fragments thereof, sequences hybridizable therewith, sequences homologous thereto [e.g., at least 50%, at least 55%, at least 60%, at least 65%, at least 70%, at least 75%, at least 80%, at least 85%, at least 95% or more say 100% identical to the nucleic acid sequences set forth below], sequences encoding similar polypeptides with different codon usage, altered sequences characterized by mutations, such as deletion, insertion or substitution of one or more nucleotides, either naturally occurring or man induced, either randomly or in a targeted fashion. The present invention also encompasses homologous nucleic acid sequences (i.e., which form a part of a polynucleotide sequence of the present invention) which include sequence regions unique to the polynucleotides of the present invention.

In cases where the polynucleotide sequences of the present invention encode previously unidentified polypeptides, the present invention also encompasses novel polypeptides or portions thereof, which are encoded by the isolated polynucleotide and respective nucleic acid fragments thereof described hereinabove.

Thus, the present invention provides isolated polynucleotides each encoding a polypeptide which is at least 50%, at least 55%, at least 60%, at least 65%, at least 70%, at least 75%, at least 80%, %, at least 85%, %, at least 90%, at least 95% or more, say 100% identical to a polypeptide sequence listed in the Examples section or sequence listing, as determined using the LALIGN software of EMBnet switzerland (http://www.ch.embnet.org/index.html) using default parameters.

A “nucleic acid fragment” or an “oligonucleotide” or a “polynucleotide” are used herein interchangeably to refer to a polymer of nucleic acids. A polynucleotide sequence of the present invention refers to a single or double stranded nucleic acid sequences which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is composed of genomic and cDNA sequences. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

Preferred embodiments of the present invention encompass oligonucleotide probes.

An example of an oligonucleotide probe which can be utilized by the present invention is a single stranded polynucleotide which includes a sequence complementary to the unique sequence region of any variant according to the present invention, including but not limited to a nucleotide sequence coding for an amino sequence of a bridge, tail, head and/or insertion according to the present invention, and/or the equivalent portions of any nucleotide sequence given herein (including but not limited to a nucleotide sequence of a node, segment or amplicon described herein).

Alternatively, an oligonucleotide probe of the present invention can be designed to hybridize with a nucleic acid sequence encompassed by any of the above nucleic acid sequences, particularly the portions specified above, including but not limited to a nucleotide sequence coding for an amino sequence of a bridge, tail, head and/or insertion according to the present invention, and/or the equivalent portions of any nucleotide sequence given herein (including but not limited to a nucleotide sequence of a node, segment or amplicon described herein).

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

Oligonucleotides used according to this aspect of the present invention are those having a length selected from a range of about 10 to about 200 bases preferably about 15 to about 150 bases, more preferably about 20 to about 100 bases, most preferably about 20 to about 50 bases. Preferably, the oligonucleotide of the present invention features at least 17, at least 18, at least 19, at least 20, at least 22, at least 25, at least 30 or at least 40, bases specifically hybridizable with the biomarkers of the present invention.

The oligonucleotides of the present invention may comprise heterocylic nucleosides consisting of purines and the pyrimidines bases, bonded in a 3′ to 5′ phosphodiester linkage.

Preferably used oligonucleotides are those modified at one or more of the backbone, internucleoside linkages or bases, as is broadly described hereinunder.

Specific examples of preferred oligonucleotides useful according to this aspect of the present invention include oligonucleotides containing modified backbones or non-natural internucleoside linkages. Oligonucleotides having modified backbones include those that retain a phosphorus atom in the backbone, as disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466, 677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides which can be used according to the present invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic, includes peptide nucleic acid (PNA). United States patents that teach the preparation of PNA compounds include, but are not limited to, U.S. Pat. Nos. 5,539,082; 5,714,331; and 5,719,262, each of which is herein incorporated by reference. Other backbone modifications, which can be used in the present invention are disclosed in U.S. Pat. No. 6,303,374.

Oligonucleotides of the present invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases particularly useful for increasing the binding affinity of the oligomeric compounds of the invention include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications.

Another modification of the oligonucleotides of the invention involves chemically linking to the oligonucleotide one or more moieties or conjugates, which enhance the activity, cellular distribution or cellular uptake of the oligonucleotide. Such moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety, as disclosed in U.S. Pat. No. 6,303,374.

It is not necessary for all positions in a given oligonucleotide molecule to be uniformly modified, and in fact more than one of the aforementioned modifications may be incorporated in a single compound or even at a single nucleoside within an oligonucleotide.

It will be appreciated that oligonucleotides of the present invention may include further modifications for more efficient use as diagnostic agents and/or to increase bioavailability, therapeutic efficacy and reduce cytotoxicity.

Expression of the Polynucleotide Sequence of the Present Invention

To enable cellular expression of the polynucleotides of the present invention, a nucleic acid construct (or an “expression vector”) according to the present invention may be used, which includes at least a coding region of one of the above nucleic acid sequences, and further includes at least one cis acting regulatory element. As used herein, the phrase “cis acting regulatory element” refers to a polynucleotide sequence, preferably a promoter, which binds a trans acting regulator and regulates the transcription of a coding sequence located downstream thereto.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of the present invention is active in the specific cell population transformed. Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166). The nucleic acid construct of the present invention can further include an enhancer, which can be adjacent or distant to the promoter sequence and can function in up regulating the transcription therefrom.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for the present invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for the present invention include those derived from SV40.

In addition to the elements already described, the expression vector of the present invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of the present invention can further include additional polynucleotide sequences that allow, for example, the translation of several proteins from a single mRNA such as an internal ribosome entry site (IRES) and sequences for genomic integration of the promoter-chimeric polypeptide.

The nucleic acid construct of the present invention preferably further includes an appropriate selectable marker and/or an origin of replication. Preferably, the nucleic acid construct utilized is a shuttle vector, which can propagate both in E. coli (wherein the construct comprises an appropriate selectable marker and origin of replication) and be compatible for propagation in cells, or integration in a gene and a tissue of choice. The construct according to the present invention can be, for example, a plasmid, a bacmid, a phagemid, a cosmid, a phage, a virus or an artificial chromosome.

Examples of suitable constructs include, but are not limited to, pcDNA3, pcDNA3.1 (+/−), pGL3, PzeoSV2 (+/−), pDisplay, pEF/myc/cyto, pCMV/myc/cyto each of which is commercially available from Invitrogen Co. (www.invitrogen.com). Examples of retroviral vector and packaging systems are those sold by Clontech, San Diego, Calif., including Retro-X vectors pLNCX and pLXSN, which permit cloning into multiple cloning sites and the trasgene is transcribed from CMV promoter. Vectors derived from Mo-MuLV are also included such as pBabe, where the transgene will be transcribed from the 5′LTR promoter.

Viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by the present invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I) and kidney cells may be targeted using the heterologous promoter present in the baculovirus Autographa californica nucleopolyhedrovirus (AcMNPV) as described in Liang C Y et al., 2004 (Arch Virol. 149: 51-60).

Recombinant viral vectors are useful for in vivo expression of the polynucleotide sequence of the present invention since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Various methods can be used to introduce the expression vector of the present invention into stem cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of the present invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Other than containing the necessary elements for the transcription and translation of the inserted coding sequence, the expression construct of the present invention can also include sequences engineered to enhance stability, production, purification, yield or toxicity of the expressed peptide. For example, the expression of a fusion protein or a cleavable fusion protein comprising Met variant of the present invention and a heterologous protein can be engineered. Such a fusion protein can be designed so that the fusion protein can be readily isolated by affinity chromatography; e.g., by immobilization on a column specific for the heterologous protein. Where a cleavage site is engineered between the Met moiety and the heterologous protein, the Met moiety can be released from the chromatographic column by treatment with an appropriate enzyme or agent that disrupts the cleavage site [e.g., see Booth et al. (1988) Immunol. Lett. 19:65-70; and Gardella et al., (1990) J. Biol. Chem. 265:15854-15859].

As mentioned hereinabove, a variety of prokaryotic or eukaryotic cells can be used as host-expression systems to express the polypeptides of the present invention. These include, but are not limited to, microorganisms, such as bacteria transformed with a recombinant bacteriophage DNA, plasmid DNA or cosmid DNA expression vector containing the coding sequence; yeast transformed with recombinant yeast expression vectors containing the coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence. Mammalian expression systems can also be used to express the polypeptides of the present invention.

Examples of bacterial constructs include the pET series of E. coli expression vectors [Studier et al. (1990) Methods in Enzymol. 185:60-89).

In yeast, a number of vectors containing constitutive or inducible promoters can be used, as disclosed in U.S. Pat. No. 5,932,447. Alternatively, vectors can be used which promote integration of foreign DNA sequences into the yeast chromosome.

In cases where plant expression vectors are used, the expression of the coding sequence can be driven by a number of promoters. For example, viral promoters such as the 35S RNA and 19S RNA promoters of CaMV [Brisson et al. (1984) Nature 310:511-514], or the coat protein promoter to TMV [Takamatsu et al. (1987) EMBO J. 6:307-311] can be used. Alternatively, plant promoters such as the small subunit of RUBISCO [Coruzzi et al. (1984) EMBO J. 3:1671-1680 and Brogli et al., (1984) Science 224:838-843] or heat shock promoters, e.g., soybean hsp 17.5-E or hsp17.3-B [Gurley et al. (1986) Mol. Cell. Biol. 6:559-565] can be used. These constructs can be introduced into plant cells using Ti plasmid, R1 plasmid, plant viral vectors, direct DNA transformation, microinjection, electroporation and other techniques well known to the skilled artisan. See, for example, Weissbach & Weissbach, 1988, Methods for Plant Molecular Biology, Academic Press, NY, Section VIII, pp 421-463.

Other expression systems such as insects and mammalian host cell systems which are well known in the art and are further described hereinbelow can also be used by the present invention.

Recovery of the recombinant polypeptide is effected following an appropriate time in culture. The phrase “recovering the recombinant polypeptide” refers to collecting the whole fermentation medium containing the polypeptide and need not imply additional steps of separation or purification. Not withstanding the above, polypeptides of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

Hybridization Assays

Detection of a nucleic acid of interest in a biological sample may optionally be effected by hybridization-based assays using an oligonucleotide probe (non-limiting examples of probes according to the present invention were previously described).

Traditional hybridization assays include PCR, RT-PCR, Real-time PCR, RNase protection, in-situ hybridization, primer extension, Southern blots (DNA detection), dot or slot blots (DNA, RNA), and Northern blots (RNA detection) (NAT type assays are described in greater detail below). More recently, PNAs have been described (Nielsen et al. 1999, Current Opin. Biotechnol. 10:71-75). Other detection methods include kits containing probes on a dipstick setup and the like.

Hybridization based assays which allow the detection of a variant of interest (i.e., DNA or RNA) in a biological sample rely on the use of oligonucleotides which can be 10, 15, 20, or 30 to 100 nucleotides long preferably from 10 to 50, more preferably from 40 to 50 nucleotides long.

Thus, the isolated polynucleotides (oligonucleotides) of the present invention are preferably hybridizable with any of the herein described nucleic acid sequences under moderate to stringent hybridization conditions.

Moderate to stringent hybridization conditions are characterized by a hybridization solution such as containing 10% dextrane sulfate, 1 M NaCl, 1% SDS and 5×106 cpm 32P labeled probe, at 65° C., with a final wash solution of 0.2×SSC and 0.1% SDS and final wash at 65° C. and whereas moderate hybridization is effected using a hybridization solution containing 10% dextrane sulfate, 1 M NaCl, 1% SDS and 5×106 cpm 32P labeled probe, at 65° C., with a final wash solution of 1×SSC and 0.1% SDS and final wash at 50° C.

More generally, hybridization of short nucleic acids (below 200 bp in length, e.g. 17-40 bp in length) can be effected using the following exemplary hybridization protocols which can be modified according to the desired stringency; (i) hybridization solution of 6×SSC and 1% SDS or 3 M TMACI, 0.01 M sodium phosphate (pH 6.8), 1 mM EDTA (pH 7.6), 0.5% SDS, 100 mg/ml denatured salmon sperm DNA and 0.1% nonfat dried milk, hybridization temperature of 1-1.5° C. below the Tm, final wash solution of 3 M TMACI, 0.01 M sodium phosphate (pH 6.8), 1 mM EDTA (pH 7.6), 0.5% SDS at 1-1.5° C. below the Tm; (ii) hybridization solution of 6×SSC and 0.1% SDS or 3 M TMACI, 0.01 M sodium phosphate (pH 6.8), 1 mM EDTA (pH 7.6), 0.5% SDS, 100 mg/ml denatured salmon sperm DNA and 0.1% nonfat dried milk, hybridization temperature of 2-2.5° C. below the Tm, final wash solution of 3 M TMACI, 0.01 M sodium phosphate (pH 6.8), 1 mM EDTA (pH 7.6), 0.5% SDS at 1-1.5° C. below the Tm, final wash solution of 6×SSC, and final wash at 22° C.; (iii) hybridization solution of 6×SSC and 1% SDS or 3 M TMACI, 0.01 M sodium phosphate (pH 6.8), 1 mM EDTA (pH 7.6), 0.5% SDS, 100 mg/ml denatured salmon sperm DNA and 0.1% nonfat dried milk, hybridization temperature.

The detection of hybrid duplexes can be carried out by a number of methods. Typically, hybridization duplexes are separated from unhybridized nucleic acids and the labels bound to the duplexes are then detected. Such labels refer to radioactive, fluorescent, biological or enzymatic tags or labels of standard use in the art. A label can be conjugated to either the oligonucleotide probes or the nucleic acids derived from the biological sample.

Probes can be labeled according to numerous well known methods. Non-limiting examples of radioactive labels include 3H, 14C, 32P, and 35S. Non-limiting examples of detectable markers include ligands, fluorophores, chemiluminescent agents, enzymes, and antibodies. Other detectable markers for use with probes, which can enable an increase in sensitivity of the method of the invention, include biotin and radio-nucleotides. It will become evident to the person of ordinary skill that the choice of a particular label dictates the manner in which it is bound to the probe.

For example, oligonucleotides of the present invention can be labeled subsequent to synthesis, by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent. Alternatively, when fluorescently-labeled oligonucleotide probes are used, fluorescein, lissamine, phycoerythrin, rhodamine (Perkin Elmer Cetus), Cy2, Cy3, Cy3.5, Cy5, Cy5.5, Cy7, FluorX (Amersham) and others [e.g., Kricka et al. (1992), Academic Press San Diego, Calif] can be attached to the oligonucleotides.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays. For instance, samples may be hybridized to an irrelevant probe and treated with RNAse A prior to hybridization, to assess false hybridization.

Although the present invention is not specifically dependent on the use of a label for the detection of a particular nucleic acid sequence, such a label might be beneficial, by increasing the sensitivity of the detection. Furthermore, it enables automation. Probes can be labeled according to numerous well known methods.

As commonly known, radioactive nucleotides can be incorporated into probes of the invention by several methods. Non-limiting examples of radioactive labels include 3H, 14C, 32P, and 35S.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays.

Probes of the invention can be utilized with naturally occurring sugar-phosphate backbones as well as modified backbones including phosphorothioates, dithionates, alkyl phosphonates and a-nucleotides and the like. Probes of the invention can be constructed of either ribonucleic acid (RNA) or deoxyribonucleic acid (DNA), and preferably of DNA.

Amino Acid Sequences and Peptides

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

Polypeptide products can be biochemically synthesized such as by employing standard solid phase techniques. Such methods include but are not limited to exclusive solid phase synthesis, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Solid phase polypeptide synthesis procedures are well known in the art and further described by John Morrow Stewart and Janis Dillaha Young, Solid Phase Peptide Syntheses (2nd Ed., Pierce Chemical Company, 1984).

Synthetic polypeptides can optionally be purified by preparative high performance liquid chromatography [Creighton T. (1983) Proteins, structures and molecular principles. WH Freeman and Co. N.Y.], after which their composition can be confirmed via amino acid sequencing.

In cases where large amounts of a polypeptide are desired, it can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

The present invention also encompasses polypeptides encoded by the polynucleotide sequences of the present invention, as well as polypeptides according to the amino acid sequences described herein. The present invention also encompasses homologues of these polypeptides, such homologues can be at least 50%, at least 55%, at least 60%, at least 65%, at least 70%, at least 75%, at least 80%, at least 85%, at least 95% or more say 100% homologous to the amino acid sequences set forth below, as can be determined using BlastP software of the National Center of Biotechnology Information (NCBI) using default parameters, optionally and preferably including the following: filtering on (this option filters repetitive or low-complexity sequences from the query using the Seg (protein) program), scoring matrix is BLOSUM62 for proteins, word size is 3, E value is 10, gap costs are 11, 1 (initialization and extension), and number of alignments shown is 50. Finally, the present invention also encompasses fragments of the above described polypeptides and polypeptides having mutations, such as deletions, insertions or substitutions of one or more amino acids, either naturally occurring or artificially induced, either randomly or in a targeted fashion.

It will be appreciated that peptides identified according the present invention may be degradation products, synthetic peptides or recombinant peptides as well as peptidomimetics, typically, synthetic peptides and peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2—NH, CH2—S, CH2—S═O, O═C—NH, CH2—O, CH2—CH2, S═C—NH, CH═CH or CF═CH, backbone modifications, and residue modification. Methods for preparing peptidomimetic compounds are well known in the art and are specified. Further details in this respect are provided hereinunder.

Peptide bonds (—CO—NH—) within the peptide may be substituted, for example, by N-methylated bonds (—N(CH3)-CO—), ester bonds (—C(R)H—C—O—O—C(R)—N—), ketomethylen bonds (—CO—CH2-), *-aza bonds (—NH—N(R)—CO—), wherein R is any alkyl, e.g., methyl, carba bonds (—CH2—NH—), hydroxyethylene bonds (—CH(OH)—CH2-), thioamide bonds (—CS—NH—), olefinic double bonds (—CH═CH—), retro amide bonds (—NH—CO—), peptide derivatives (—N(R)—CH2-CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as Phenylglycine, TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Table 1 non-conventional or modified amino acids which can be used with the present invention.

TABLE 1

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| Carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| Carboxylate | | L-N-methylglutamic acid | Nmglu |
| Cyclohexylalanine | Chexa | L-N-methylhistidine | Nmhis |
| Cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3- | Nbhe |
| | | diphenylpropyl)glycine | |
| D-N-methylleucine | Dnmleu | N-(3-indolylyethyl) glycine | Nhtrp |
| D-N-methyllysine | Dnmlys | N-methyl-γ-aminobutyrate | Nmgabu |
| N- | Nmchexa | D-N-methylmethionine | Dnmmet |
| methylcyclohexylalanine | | | |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomo | Mhphe |
| | | phenylalanine | |
| L-α-methylisoleucine | Mile | N-(2-methylthioethyl)glycine | Nmet |
| D-N-methylglutamine | Dnmgln | N-(3- | Narg |
| | | guanidinopropyl)glycine | |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N- | Nmchexa | D-N-methylmethionine | Dnmmet |
| methylcyclohexylalanine | | | |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α- | Mhphe |
| | | methylhomophenylalanine | |
| L-α-methylisoleucine | Mile | N-2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α-methylvaline | Mtrp | L-α-methyltyrosine | Mtyr |
| L-α-methylleucine | Mval | L-N- | Nmhphe |
| | Nnbhm | methylhomophenylalanine | |
| N-(N-(2,2-diphenylethyl) | | N-(N-(3,3-diphenylpropyl) | |
| carbamylmethyl-glycine | Nnbhm | carbamylmethyl(1)glycine | Nnbhe |
| 1-carboxy-1-(2,2-diphenyl | Nmbc | | |
| ethylamino)cyclopropane | | | |

Since the peptides of the present invention are preferably utilized in therapeutics which require the peptides to be in soluble form, the peptides of the present invention preferably include one or more non-natural or natural polar amino acids, including but not limited to serine and threonine which are capable of increasing peptide solubility due to their hydroxyl-containing side chain.

The peptides of the present invention are preferably utilized in a linear form, although it will be appreciated that in cases where cyclicization does not severely interfere with peptide characteristics, cyclic forms of the peptide can also be utilized.

The peptides of present invention can be biochemically synthesized such as by using standard solid phase techniques. These methods include exclusive solid phase synthesis well known in the art, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Synthetic peptides can be purified by preparative high performance liquid chromatography and the composition of which can be confirmed via amino acid sequencing.

In cases where large amounts of the peptides of the present invention are desired, the peptides of the present invention can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463 and also as described above.

Peptide sequences which exhibit high therapeutic activity, such as by competing with wild type signaling proteins of the same signaling pathway, can be also uncovered using computational biology. Software programs useful for displaying three-dimensional structural models, such as RIBBONS (Carson, M., 1997. Methods in Enzymology 277, 25), O (Jones, T A. et al., 1991. Acta Crystallogr. A47, 110), DINO (DINO: Visualizing Structural Biology (2001) http://www.dino3d.org); and QUANTA, INSIGHT, SYBYL, MACROMODE, ICM, MOLMOL, RASMOL and GRASP (reviewed in Kraulis, J., 1991. Appl Crystallogr. 24, 946) can be utilized to model interactions between the polypeptides of the present invention and prospective peptide sequences to thereby identify peptides which display the highest probability of binding for example to a respective ligand (e.g., IL-10). Computational modeling of protein-peptide interactions has been successfully used in rational drug design, for further details, see Lam et al., 1994. Science 263, 380; Wlodawer et al., 1993. Ann Rev Biochem. 62, 543; Appelt, 1993. Perspectives in Drug Discovery and Design 1, 23; Erickson, 1993. Perspectives in Drug Discovery and Design 1, 109, and Mauro M J. et al., 2002. J Clin Oncol. 20, 325-34.

Antibodies

“Antibody” refers to a polypeptide ligand that is preferably substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad-immunoglobulin variable region genes. Antibodies exist, e.g., as intact immunoglobulins or as a number of well characterized fragments produced by digestion with various peptidases. This includes, e.g., Fab′ and F(ab)′2 fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy chain variable region.

The functional fragments of antibodies, such as Fab, F(ab′)2, and Fv that are capable of binding to macrophages, are described as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)₂, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression, vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′) or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Preferably, the antibody of this aspect of the present invention specifically binds at least one epitope of the polypeptide variants of the present invention. As used herein, the term “epitope” refers to any antigenic determinant on an antigen to which the paratope of an antibody binds.

Epitopic determinants usually consist of chemically active surface groupings of molecules such as amino acids or carbohydrate side chains and usually have specific three dimensional structural characteristics, as well as specific charge characteristics.

Optionally, a unique epitope may be created in a variant due to a change in one or more post-translational modifications, including but not limited to glycosylation and/or phosphorylation, as described below. Such a change may also cause a new epitope to be created, for example through removal of glycosylation at a particular site.

An epitope according to the present invention may also optionally comprise part or all of a unique sequence portion of a variant according to the present invention in combination with at least one other portion of the variant which is not contiguous to the unique sequence portion in the linear polypeptide itself, yet which are able to form an epitope in combination. One or more unique sequence portions may optionally combine with one or more other non-contiguous portions of the variant (including a portion which may have high homology to a portion of the known protein) to form an epitope.

Display Libraries

According to still another aspect of the present invention there is provided a display library comprising a plurality of display vehicles (such as phages, viruses or bacteria) each displaying at least 6, at least 7, at least 8, at least 9, at least 10, 10-15, 12-17, 15-20, 15-30 or 20-50 consecutive amino acids derived from the polypeptide sequences of the present invention.

Since in therapeutic applications it is highly desirable to employ the minimal and most efficacious polypeptide regions, which still exert therapeutic function, identification of such peptide regions can be effected using various approaches, including, for example, display techniques as described herein.

Methods of constructing such display libraries are well known in the art. Such methods are described in, for example, Young A C, et al., “The three-dimensional structures of a polysaccharide binding antibody to Cryptococcus neoformans and its complex with a peptide from a phage display library: implications for the identification of peptide mimotopes” J Mol Biol 1997 Dec. 12; 274(4):622-34; Giebel L B et al. “Screening of cyclic peptide phage libraries identifies ligands that bind streptavidin with high affinities” Biochemistry 1995 Nov. 28; 34(47):15430-5; Davies E L et al., “Selection of specific phage-display antibodies using libraries derived from chicken immunoglobulin genes” J Immunol Methods 1995 Oct. 12; 186(1):125-35; Jones C R T al. “Current trends in molecular recognition and bioseparation” J Chromatogr A 1995 Jul. 14; 707(1):3-22; Deng S J et al. “Basis for selection of improved carbohydrate-binding single-chain antibodies from synthetic gene libraries” Proc Natl Acad Sci USA 1995 May 23; 92(11):4992-6; and Deng S J et al. “Selection of antibody single-chain variable fragments with improved carbohydrate binding by phage display” J Biol Chem 1994 Apr. 1; 269(13):9533-8, which are incorporated herein by reference.

A brief description of different alpha 1 subunit types is now provided. These subunits are named as follows: alpha IX, in which the letter “X” is replaced by A, B, C, D, E, F, G, H, I or S. These different subunits cause the assembled calcium channel to have different functions. Furthermore, these subunits are expressed in particular tissues, which also affects their resultant function. The resultant channels are named as follows: CavX.Y, in which “X.Y” is a number indicating their function (1.1, 1.2, 1.3 and 1.4 are all L-type channels, 3.1, 3.2 and 3.3 are all T-type channels and the rest 2.1, 2.2, 2.3 are P/Q, N, R-type channels).

### EXAMPLE 1

This Example relates to the variant Z39724_P4 (SEQ ID NO:16; nucleic acid sequence is given by SEQ ID NO:38), which is a variant alpha 1 subunit according to the present invention, and more specifically, is a splice variant of the known protein CCAE_HUMAN (all known proteins are referred to herein according to their SwissProt accession number), which is the voltage-dependent R-type calcium channel alpha-1E subunit, also known as the voltage-gated calcium channel alpha subunit Cav2.3. This protein is encoded by the gene CACNA1E. The alpha-1E form of the alpha 1 subunit results in formation of R-type calcium channels, which belong to the “high-voltage activated” (HVA) group and are blocked completely by nickel, and partially by omega-agatoxin-IIIA (omega-Aga-IIIA). These channels are however insensitive to dihydropyridines, for example, as well as to various other toxins. Calcium channels containing the alpha-1E subunit could be involved in the modulation of firing patterns of neurons, which is clearly important for thought processes. The tissue specificity is believed to be in neuronal tissues and in kidney.

The structure of this alpha1 subunit is as follows. Each of the four internal repeats contains five hydrophobic transmembrane segments (S1, S2, S3, S5, S6) and one positively charged transmembrane segment (S4). S4 segments probably represent the voltage-sensor and are characterized by a series of positively charged amino acids at every third position.

The structure of the splice variant according to the present invention features a unique head and a unique tail, as well as an internal skipped exon, as compared to the known protein sequence. An alignment is provided at the end of this section, while the comparison between the two sequences is described below.

According to preferred embodiments of the present invention, there is provided an isolated chimeric polypeptide encoding for Z39724_P4, comprising a first amino acid sequence being at least 70%, optionally at least 80%, preferably at least 85%, more preferably at least 90% and most preferably at least 95% homologous to a polypeptide having the sequence (SEQ ID NO: 45) MRGLSPDRRLPEPAPCPAAGSPEPRSAGRRQRHVLALPEEEAWRPQQCALLEADASDEVGMLPASPRAASGFDNFFQVQEGEGQGWEGAMALEAGSSPFLPVSPEVMKRRRGGLIEQRDIIKAHEAHKMQSTPQARRKEWE corresponding to amino acids 1-141 of Z39724_P4, a second amino acid sequence being at least 90% homologous to (SEQ ID NO: 46) MARFGEAVVARPGSGDGDSDQSRNRQGTPVPASGQAAAYKQTKAQRARTMALYNPIPVRQNCFTVNRSLFIFGEDNIVRKYAKKLIDWPPFEYMILATIIANCIVLALEQHLPEDDKTPMSRRLEKTEPYFIGIFCFEAGIKIVALGFIFHKGSYLRNGWNVMDFIVVLSGILATAGTHFNTHVDLRTLRAVRVLRPLKLVSGIPSLQIVLKSIMKAMVPLLQIGLLLFFAILMFAIIGLEFYSGKLHRACFMNNSGILEGFDPPHPCGVQGCPAGYECKDWIGPNDGITQFDNILFAVLTVFQCITMEGWTTVLYNTNDALGATWNWLYFIPLIIIGSFFVLNLVLGVLSGEFAKERERVENRRAFMKLRRQQQIERELNGYRAWIDKAEEVMLAEENKNAGTSALEVLRRATIKRSRTEAMTRDSSDEHCVDISSVGTPLARASIKSAKVDGVSYFRHKERLLRISIRHMVKSQVFYWIVLSLVALNTACVAIVHHNQPQWLTHLLYYAEFLFLGLFLLEMSLKMYGMGPRLYFHSSFNCFDFGVTVGSIFEVVWAIFRPGTSFGISVLRALRLLRIFKITKYWASLRNLVVSLMSSMKSIISLLFLLFLFIVVFALLGMQLFGGRFNFNDGTPSANFDTFPAAI corresponding to amino acids 1-647 of CCAE_HUMAN, which also corresponds to amino acids 142-788 of Z39724_P4, a bridging amino acid M corresponding to amino acid 789 of Z39724_P4, a third amino acid sequence being at least 90% homologous to (SEQ ID NO: 47) TVFQILTGEDWNEVMYNGIRSQGGVSSGMWSAIYFIVLTLFGNYTLLNVFLAIAVDNLANAQELTKDEQEEEEAFNQKHALQKAKEVSPMSAPNMPSIE corresponding to amino acids 649-747 of CCAE_HUMAN, which also corresponds to amino acids 790-888 of Z39724_P4, a fourth amino acid sequence being at least 90% homologous to (SEQ ID NO: 48) RERRRRHHMSVWEQRTSQLRKHMQMSSQEALNREEAPTMNPLNPLNPLSSLNPLNAHPSLYRRPRAIEG corresponding to amino acids 767-835 of CCAE_HUMAN, which also corresponds to amino acids 889-957 of Z39724_P4, a unique insertion (fifth amino acid sequence) being at least 70%, optionally at least 80%, preferably at least 85%, more preferably at least 90% and most preferably at least 95% homologous to a polypeptide having the sequence LAL corresponding to amino acids 958-960 of Z39724_P4, a sixth amino acid sequence being at least 90% homologous to (SEQ ID NO: 49) GLALEKFEEERISRGGSLKGDGGDRSSALDNQRTPLSLGQREPPWLARPCHGNCDPTQQEAGGGEAVVTFEDRARHRQSQRRSRHRRVRTEGKESSSASRSRSASQERSLDEAMPTEGEKDHELRGNHGAKEPTIQEERAQDLRRTNSLMVSRGSGLAGGLDEADTPLVLPHPELEVGKHVVLTEQEPEGSSEQALLGNVQLDMGRVISQSEPDLSCITANTDKATTESTSVTVAIPDVDPLVDSTVVHISNKTDGEASPLKEAEIREDEEEVEKKKQKKEKRETGKAMVPHSSMFIFSTTNPIRRACHYIVNLRYFEMCILLVIAASSIALAAEDPVLTNSERNKVLRYFDYVFTGVFTFEMVIKMIDQGLILQDGSYFRDLWNILDFVVVVGALVAFALANALGTNKGRDIKTIKSLRVLRVLRPLKTIKRLPKLKAVFDCVVTSLKNVFNILIVYKLFMFIFAVIAVQLFKGKFFYCTDSSKDTEKECIGNYVDHEKNKMEVKGREWKRHEFHYDNIIWALLTLFTVSTGEGWPQVLQHSVDVTEEDRGPSRSNRMEMSIFYVVYFVVFPFFFVNIFVALIIITFQEQGDKMMEECSLEKNERACIDFAISAKPLTRYMPQNRHTFQYRVWHFVVSPSFEYTIMAMIALNTVVLMMKYYSAPCTYELALKYLNIAFTMVFSLECVLKVIAFGFLNYFRDTWNIFDFITVIGSITEIILTDSKLVNTSGFNMSFLKLFRAARLIKLLRQGYTIRILLWTFVQSFKALPYVCLLIAMLFFIYAIIGMQVFGNIKLDEESHINRHNNFRSFFGSLMLLFRSATGEAWQEIMLSCLGEKGCEPDTTAPSGQNENERCGTDLAYVYFVSFIFFCSFLMLNLFVAVIMDNFEYLTRDSSILGPHHLDEFVRVWAEYDRAA corresponding to amino acids 838-1754 of CCAE_HUMAN, which also corresponds to amino acids 961-1877 of Z39724_P4, and a seventh amino acid sequence being at least 70%, optionally at least 80%, preferably at least 85%, more preferably at least 90% and most preferably at least 95% homologous to a polypeptide having the sequence (SEQ ID NO: 50) WCVGPSAAPAGPRAKVSGVPREEAGIGATQMPACYRRKGDSS corresponding to amino acids 1878-1919 of Z39724_P4, wherein said first amino acid sequence, second amino acid sequence, bridging amino acid, third amino acid sequence, fourth amino acid sequence, fifth amino acid sequence, sixth amino acid sequence and seventh amino acid sequence are contiguous and in a sequential order.

According to other preferred embodiments of the present invention, there is provided an isolated polypeptide encoding for a head of Z39724_P4, comprising a polypeptide being at least 70%, optionally at least about 80%, preferably at least about 85%, more preferably at least about 90% and most preferably at least about 95% homologous to the sequence (SEQ ID NO: 51) MRGLSPDRRLPEPAPCPAAGSPEPRSAGRRQRHVLALPEEEAWRPQQCALLEADASDEVGMLPASPRAASGFDNFFQVQEGEGQGWEGAMALEAGSSPFLPVSPEVMKRRRGGLIEQRDIIKAHEAHKMQSTPQARRKEWE of Z39724_P4.

According to other preferred embodiments of the present invention, there is provided an isolated chimeric polypeptide encoding for an edge portion of Z39724_P4, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise ER, having a structure as follows: a sequence starting from any of amino acid numbers 888−x to 889; and ending at any of amino acid numbers 889+((n−2)−x), in which x varies from 0 to n−2.

According to other preferred embodiments of the present invention, there is provided an isolated polypeptide encoding for an edge portion of Z39724_P4, comprising an amino acid sequence being at least 70%, optionally at least about 80%, preferably at least about 85%, more preferably at least about 90% and most preferably at least about 95% homologous to the sequence encoding for LAL, corresponding to Z39724_P4.

According to other preferred embodiments of the present invention, there is provided an isolated polypeptide encoding for a tail of Z39724_P4, comprising a polypeptide being at least 70%, optionally at least about 80%, preferably at least about 85%, more preferably at least about 90% and most preferably at least about 95% homologous to the sequence (SEQ ID NO: 52) WCVGPSAAPAGPRAKVSGVPREEAGIGATQMPACYRRKGDSS in Z39724_P4.

Domains affected by alternative splicing of this variant, as can be seen from the comparison above, include the cytoplasmic loop between domain II and domain III, and the cytoplasmic C-terminus region of the protein.

Without wishing to be limited by a single hypothesis, there are a number of possible changes in the function of the channel resulting from the alternative splicing of the variant. The intracellular loop connecting domains II and III mediates interaction with effector proteins such as the calcium release channel for skeletal muscle excitation-contraction coupling, synaptic proteins such as syntaxin and SNAP25 (soluble attachment proteins of NSF-N-ethylmaleimide-sensitive fusion protein) or proteins for neuronal excitation-exocytosis coupling. Therefore changes to (or the absence of) these regions would be expected to affect interactions with such effector proteins and hence overall calcium channel function.

Changes in the loop between domains II and III are known to result in functional effects on R— type alpha-1E proteins. For example, in R-type Cav2.3 channels, exon 19 can be spliced out, which results in the absence of calcium-dependent slowing of inactivation and acceleration of recovery from inactivation. The resultant calcium channel has decreased inactivation by calcium influx, and has been found to be abundant in murine cerebellum, the islets of Langerhans and kidney.

The C-terminus forms a large portion (about one third) of the alpha 1 subunit and varies significantly in the CACNA1 gene family, indicating that it may be important to differential function of calcium channels. It is encoded by 3-14 exons, and the protein contains several regulatory elements such as binding sites for calcium, calmodulin and G-proteins. In Cav2.2 channels, the C-terminus is important for targeting the channels to synapses. Mutations in this region have been shown to be responsible for diseases such as spinocerebellar atxia type 6 (described in greater detail above). At least two different C-terminal motifs, having different lengths, have been found in different alpha 1 subunits. These motifs may be generated through the formation of different splice variants. For example, the P/Q Cav2.1 isoforms are generated by alternative splice donor sites at the 5′ end of exon 47, causing a stop codon to form and the protein to be truncated. An alternative donor site results in insertion of five bases leading to a frame shift and an elongation of the C-terminus by 244 amino acids.

In all three types of Cav2 channels, there are one to two exons coding for distal parts of the C-terminus that may potentially be spliced out (exons 43 and 44 in P/Q-type Cav2.1, exon 46 in the N-type Cav2.2, and exon 45 in R-type Cav2.3). Generally, the shorter the C-terminus becomes, the greater the current amplitude and the stronger the calcium dependence of inactivation, such that calcium influx causes the channel to deactivate more quickly. The reduction of current amplitude seen with these splice variants may be due to regulatory effects, such as mRNA destabilization or reduction of targeting to the membrane. On the other hand, it has been hypothesized that because the C-terminus mobility may contribute to removing the calcium-calmodulin complex from the inner mouth of the pore, a shorter C-terminus may contribute to this mobility and accelerate inactivation (Kobrinsky et al. 2003).

From the above description of the effect of changes to the C-terminus domain of this variant, it is expected that the variant will have greater current amplitude from the WT and a stronger calcium dependence of inactivation from the WT. In addition, the changes in the cytoplasmic loop between domain II and domain III might result in the absence of calcium-dependent slowing of inactivation and acceleration of recovery from inactivation.

Sequence name: CCAE_HUMAN

documentation:

of: Z39724_P4 (resides 142-1877 of SEQ ID NO: 16)_x

CCAE_HUMAN (SEQ ID NO: 53). . .

segment 1/1:

Quality: 16713.00

Escore: 0

Matching length: 1735 Total

length: 1755

Matching Percent Similarity: 99.88 Matching Percent

Identity: 99.83

Total Percent Similarity: 98.75 Total Percent

Identity: 98.69

Gaps: 2

| | . . . . . | |
|---|---|---|
| 142 | MARFGEAVVARPGSGDGDSDQSRNRQGTPVPASGQAAAYKQTKAQRARTM | 191 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MARFGEAVVARPGSGDGDSDQSRNRQGTPVPASGQAAAYKQTKAQRARTM | 50 |
| | . . . . . | |
| 192 | ALYNPIPVRQNCFTVNRSLFIFGEDNIVRKYAKKLIDWPPFEYMILATII | 241 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | ALYNPIPVRQNCFTVNRSLFIFGEDNIVRKYAKKLIDWPPFEYMILATII | 100 |
| | . . . . . | |
| 242 | ANCIVLALEQHLPEDDKTPMSRRLEKTEPYFIGIFCFEAGIKIVALGFIF | 291 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | ANCIVLALEQHLPEDDKTPMSRRLEKTEPYFIGIFCFEAGIKIVALGFIF | 150 |
| | . . . . . | |
| 292 | HKGSYLRNGWNVMDFIVVLSGILATAGTHFNTHVDLRTLRAVRVLRPLKL | 341 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | HKGSYLRNGWNVMDFIVVLSGILATAGTHFNTHVDLRTLRAVRVLRPLKL | 200 |
| | . . . . . | |
| 342 | VSGIPSLQIVLKSIMKAMVPLLQIGLLLFFAILMFAIIGLEFYSGKLHRA | 391 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | VSGIPSLQIVLKSIMKAMVPLLQIGLLLFFAILMFAIIGLEFYSGKLHRA | 250 |
| | . . . . . | |
| 392 | CFMNNSGILEGFDPPHPCGVQGCPAGYECKDWIGPNDGITQFDNILFAVL | 441 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | CFMNNSGILEGFDPPHPCGVQGCPAGYECKDWIGPNDGITQFDNILFAVL | 300 |
| | . . . . . | |
| 442 | TVFQCITMEGWTTVLYNTNDALGATWNWLYFIPLIIIGSFFVLNLVLGVL | 491 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | TVFQCITMEGWTTVLYNTNDALGATWNWLYFIPLIIIGSFFVLNLVLGVL | 350 |
| | . . . . . | |
| 492 | SGEFAKERERVENRRAFMKLRRQQQIERELNGYRAWIDKAEEVMLAEENK | 541 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 351 | SGEFAKERERVENRRAFMKLRRQQQIERELNGYRAWIDKAEEVMLAEENK | 400 |
| | . . . . . | |
| 542 | NAGTSALEVLRRATIKRSRTEAMTRDSSDEHCVDISSVGTPLARASIKSA | 591 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 401 | NAGTSALEVLRRATIKRSRTEAMTRDSSDEHCVDISSVGTPLARASIKSA | 450 |
| | . . . . . | |
| 592 | KVDGVSYFRHKERLLRISIRHMVKSQVFYWIVLSLVALNTACVAIVHHNQ | 641 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 451 | KVDGVSYFRHKERLLRISIRHMVKSQVFYWIVLSLVALNTACVAIVHHNQ | 500 |
| | . . . . . | |
| 642 | PQWLTHLLYYAEFLFLGLFLLEMSLKMYGMGPRLYFHSSFNCFDFGVTVG | 691 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 501 | PQWLTHLLYYAEFLFLGLFLLEMSLKMYGMGPRLYFHSSFNCFDFGVTVG | 550 |
| | . . . . . | |
| 692 | SIFEVVWAIFRPGTSFGISVLRALRLLRIFKITKYWASLRNLVVSLMSSM | 741 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 551 | SIFEVVWAIFRPGTSFGISVLRALRLLRIFKITKYWASLRNLVVSLMSSM | 600 |
| | . . . . . | |
| 742 | KSIISLLFLLFLFIVVFALLGMQLFGGRFNFNDGTPSANFDTFPAAIMTV | 791 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 601 | KSIISLLFLLFLFIVVFALLGMQLFGGRFNFNDGTPSANFDTFPAAIMTV | 650 |
| | . . . . . | |
| 792 | FQILTGEDWNEVMYNGIRSQGGVSSGMWSAIYFIVLTLFGNYTLLNVFLA | 841 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 651 | FQILTGEDWNEVMYNGIRSQGGVSSGMWSAIYFIVLTLFGNYTLLNVFLA | 700 |
| | . . . . . | |
| 842 | IAVDNLANAQELTKDEQEEEEAFNQKHALQKAKEVSPMSAPNMPSIE... | 888 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 701 | IAVDNLANAQELTKDEQEEEEAFNQKHALQKAKEVSPMSAPNMPSIERDR | 750 |
| | . . . . . | |
| 889 | ................RERRRRHHMSVWEQRTSQLRKHMQMSSQEALNRE | 922 |
| | \|\|\|\|\|\|\|\|\|\|\|\|\|\|\|\|\|\|\|\|\|\|\|\|\|\|\|\|\|\|\|\|\|\| | |
| 751 | RRRHHMSMWEPRSSHLRERRRRHHMSVWEQRTSQLRKHMQMSSQEALNRE | 800 |
| | . . . . . | |
| 923 | EAPTMNPLNPLNPLSSLNPLNAHPSLYRRPRAIEGLALGLALEKFEEERI | 972 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 801 | EAPTMNPLNPLNPLSSLNPLNAHPSLYRRPRAIEGLALGLALEKFEEERI | 849 |
| | . . . . . | |
| 973 | SRGGSLKGDGGDRSSALDNQRTPLSLGQREPPWLARPCHGNCDPTQQEAG | 1022 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 850 | SRGGSLKGDGGDRSSALDNQRTPLSLGQREPPWLARPCHGNCDPTQQEAG | 899 |
| | . . . . . | |
| 1023 | GGEAVVTFEDRARHRQSQRRSRHRRVRTEGKESSSASRSRSASQERSLDE | 1072 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 900 | GGEAVVTFEDRARHRQSQRRSRHRRVRTEGKESSSASRSRSASQERSLDE | 949 |
| | . . . . . | |
| 1073 | AMPTEGEKDHELRGNHGAKEPTIQEERAQDLRRTNSLMVSRGSGLAGGLD | 1122 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 950 | AMPTEGEKDHELRGNHGAKEPTIQEERAQDLRRTNSLMVSRGSGLAGGLD | 999 |
| | . . . . . | |
| 1123 | EADTPLVLPHPELEVGKHVVLTEQEPEGSSEQALLGNVQLDMGRVISQSE | 1172 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1000 | EADTPLVLPHPELEVGKHVVLTEQEPEGSSEQALLGNVQLDMGRVISQSE | 1049 |
| | . . . . . | |
| 1173 | PDLSCITANTDKATTESTSVTVAIPDVDPLVDSTVVHISNKTDGEASPLK | 1222 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1050 | PDLSCITANTDKATTESTSVTVAIPDVDPLVDSTVVHISNKTDGEASPLK | 1099 |
| | . . . . . | |
| 1223 | EAEIREDEEEVEKKKQKKEKRETGKAMVPHSSMFIFSTTNPIRRACHYIV | 1272 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1100 | EAEIREDEEEVEKKKQKKEKRETGKAMVPHSSMFIFSTTNPIRRACHYIV | 1149 |
| | . . . . . | |
| 1273 | NLRYFEMCILLVIAASSIALAAEDPVLTNSERNKVLRYFDYVFTGVFTFE | 1322 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1150 | NLRYFEMCILLVIAASSIALAAEDPVLTNSERNKVLRYFDYVFTGVFTFE | 1199 |
| | . . . . . | |
| 1323 | MVIKMIDQGLILQDGSYFRDLWNILDFVVVVGALVAFALANALGTNKGRD | 1372 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1200 | MVIKMIDQGLILQDGSYFRDLWNILDFVVVVGALVAFALANALGTNKGRD | 1249 |
| | . . . . . | |
| 1373 | IKTIKSLRVLRVLRPLKTIKRLPKLKAVFDCVVTSLKNVFNILIVYKLFM | 1422 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1250 | IKTIKSLRVLRVLRPLKTIKRLPKLKAVFDCVVTSLKNVFNILIVYKLFM | 1299 |
| | . . . . . | |
| 1423 | FIFAVIAVQLFKGKFFYCTDSSKDTEKECIGNYVDHEKNKMEVKGREWKR | 1472 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1300 | FIFAVIAVQLFKGKFFYCTDSSKDTEKECIGNYVDHEKNKMEVKGREWKR | 1349 |
| | . . . . . | |
| 1473 | HEFHYDNIIWALLTLFTVSTGEGWPQVLQHSVDVTEEDRGPSRSNRMEMS | 1522 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1350 | HEFHYDNIIWALLTLFTVSTGEGWPQVLQHSVDVTEEDRGPSRSNRMEMS | 1399 |
| | . . . . . | |
| 1523 | IFYVVYFVVFPFFFVNIFVALIIITFQEQGDKMMEECSLEKNERACIDFA | 1572 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1400 | IFYVVYFVVFPFFFVNIFVALIIITFQEQGDKMMEECSLEKNERACIDFA | 1449 |
| | . . . . . | |
| 1573 | ISAKPLTRYMPQNRHTFQYRVWHFVVSPSFEYTIMAMIALNTVVLMMKYY | 1622 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1450 | ISAKPLTRYMPQNRHTFQYRVWHFVVSPSFEYTIMAMIALNTVVLMMKYY | 1499 |
| | . . . . . | |
| 1623 | SAPCTYELALKYLNIAFTMVFSLECVLKVIAFGFLNYFRDTWNIFDFITV | 1672 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1500 | SAPCTYELALKYLNIAFTMVFSLECVLKVIAFGFLNYFRDTWNIFDFITV | 1549 |
| | . . . . . | |
| 1673 | IGSITEIILTDSKLVNTSGFNMSFLKLFRAARLIKLLRQGYTIRILLWTF | 1722 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1550 | IGSITEIILTDSKLVNTSGFNMSFLKLFRAARLIKLLRQGYTIRILLWTF | 1599 |
| | . . . . . | |
| 1723 | VQSFKALPYVCLLIAMLFFIYAIIGMQVFGNIKLDEESHINRHNNFRSFF | 1772 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1600 | VQSFKALPYVCLLIAMLFFIYAIIGMQVFGNIKLDEESHINRHNNFRSFF | 1649 |
| | . . . . . | |
| 1773 | GSLMLLFRSATGEAWQEIMLSCLGEKGCEPDTTAPSGQNENERCGTDLAY | 1822 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1650 | GSLMLLFRSATGEAWQEIMLSCLGEKGCEPDTTAPSGQNENERCGTDLAY | 1699 |
| | . . . . . | |
| 1823 | VYFVSFIFFCSFLMLNLFVAVIMDNFEYLTRDSSILGPHHLDEPVRVWAE | 1872 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1700 | VYFVSFIFFCSFLMLNLFVAVIMDNFEYLTRDSSILGPHHLDEFVRVWAE | 1749 |
| 1873 | YDRAA | 1877 |
| | \|\|\|\|\| | |
| 1750 | YDRAA | 1754 |

### EXAMPLE 2

This Example relates to the variant T59742_P2 (SEQ ID NO:11; the nucleic acid sequence is given by SEQ ID NO:33), which is a variant alpha 1 subunit according to the present invention, and more specifically, is a splice variant of the known protein CCAA_HUMAN. This protein is the voltage-dependent P/Q-type calcium channel alpha-1A subunit, also referred to as the alpha subunit Cav2.1 or as the alpha 1A subunit. This subunit form is found in L type calcium channels, particularly in neural tissues. These calcium channels belong to the “high-voltage activated” (HVA) group and are blocked by the funnel toxin (Ftx) and by the omega-agatoxin-IVA (omega-Aga-IVA). They are however insensitive to dihydropyridines and certain other toxins.

Tissue specificity of the known protein is believed to be brain specific, as the alpha 1A subunit is mainly found in cerebellum, cerebral cortex, thalamus and hypothalamus. No expression is seen in heart, kidney, liver or muscle. Each of the four internal repeats contains five hydrophobic transmembrane segments (S1, S2, S3, S5, S6) and one positively charged transmembrane segment (S4). S4 segments probably represent the voltage-sensor and are characterized by a series of positively charged amino acids at every third position.

Defects in this subunit result in a number of different diseases, including spinocerebellar atxia type 6. This is an autosomal dominant disorder characterized by slowly progressive cerebellar ataxia of the limbs and gait, dysarthria, nystagmus, and mild vibratory and proprioceptive sensory loss. These symptoms are probably explained by severe loss of cerebellar Purkinje cells. Other defects in the CACNA1A gene are the cause of familial hemiplegic migraine (FHM), also known as migraine familial hemiplegic 1 (MHP1). FHM, a rare autosomal dominant subtype of migraine with aura, is associated with ictal hemiparesis and, in some families, progressive cerebellar atrophy.

Yet other such defects cause episodic ataxia type 2 (EA-2), also known as acetazolamide-responsive hereditary paroxysmal cerebellar ataxia (APCA). This autosomal dominant disorder is characterized by acetozolamide-responsive attacks of cerebellar ataxia and migraine-like symptoms, interictal nystagmus, and cerebellar atrophy.

The structure of the splice variant according to the present invention features a unique head, a unique insertion and a unique tail, as compared to the known protein sequence. An alignment is provided at the end of this section, while the comparison between the two sequences is described below.

According to preferred embodiments of the present invention, there is provided an isolated chimeric polypeptide encoding for T59742_P2, comprising a first amino acid sequence being at least 70%, optionally at least 80%, preferably at least 85%, more preferably at least 90% and most preferably at least 95% homologous to a polypeptide having the sequence (SEQ ID NO: 54) MSSWPTRPTKSTAPPRPGGKNGT corresponding to amino acids 1-23 of T59742_P2, a second amino acid sequence being at least 90% homologous to (SEQ ID NO: 55) TIIANCIVLALEQHLPDDDKTPMSERLDDTEPYFIGIFCFEAGIKIIALGFAFHKGSYLRNGWNVMDFVVVLTGILATVGTEFDLRTLRAVRVLRPLKLVSGIPSLQVVLKSIMKAMIPLLQIGLLLFFAILIFAIIGLEFYMGKFHTTCFEEGTDDIQGESPAPCGTEEPARTCPNGTKCQPYWEGPNNGITQFDNILFAVLTVFQCITMEGWTDLLYNSNDASGNTWNWLYFIPLIIIGSFFMLNLVLGVLSGEFAKERERVENRRAFLKLRRQQQIERELNGYMEWISKAEEVILAEDETDGEQRHPFDGALRRTTIKKSKTDLLNPEEAEDQLADIASVGSPFARASIKSAKLENSTFFHKKERRMRFYIRRMVKTQAFYWTVLSLVALNTLCVAIVHYNQPEWLSDFLYYAEFIFLGLFMSEMFIKMYGLGTRPYFHSSFNCFDCGVIIGSIFEVIWAVIKPGTSFGISVLRALRLLRIFKVTKYWASLRNLVVSLLNSMKSIISLLFLLFLFIVVFALLGMQLFGGQFNFDEGTPPTNFDTFPAAIMTVFQILTGEDWNEVMYDGIKSQGGVQGGMVFSIYFIVLTLFGNYTLLNVFLAIAVDNLANAQELTKDEQEEEEAANQKLALQKAKEVAEVSPLSAANMSIAVKEQQKNQKPAKSVWEQRTSEMRKQNLLASREALYNEMDPDERWKAAYTRHLRPDMKTHLDRPLVVDPQENRNNNTNKSRAAEPTVDQRLGQQRAEDFLRKQARYHDRARDPSGSAGLDARRPWAGSQEAELSREGPYGRESDHHAREGSLEQPGFWEGEAERGKAGDPHRRHVHRQGGSRESRSGSPRTGADGEHRRHRAHRRPGEEGPEDKAERRARHREGSRPARGGEGEGEGPDGGERRRRHRHGAPATYEGDARREDKERRHRRRKENQGSGVPVSGPNLSTTRPIQQDLGRQDPPLAEDIDNMKNNKLATAESAAPHGSLGHAGLPQSPAKMGNSTDPGPMLAIPAMATNPQNAASRRTPNNPGNPSNPGPPKTPENSLIVTNPSGTQTNSAKTARKPDHTTVDIPPACPPPLNHTVVQVNKNANPDPLPKKEEEKKEEEEDDRGEDGPKPMPPYSSMFILSTTNPLRRLCHYILNLRYFEMCILMVIAMSSIALAAEDPVQPNAPRNNVLRYFDYVFTGVFTFEMVIKMIDLGLVLHQGAYFRDLWNILDFIVVSGALVAFAFTGNSKGKDINTIKSLRVLRVLRPLKTIKRLPKLKAVFDCVVNSLKNVFNILIVYMLFMFIFAVVAVQLFKGKFFHCTDESKEFEKDCRGKYLLYEKNEVKARDREWKKYEFHYDNVLWALLTLFTVSTGEGWPQVLKHSVDATFENQGPSPGYRMEMSIFYVVYFVVFPFFFVNIFVALIIITFQEQGDKMMEEYSLEKNERACIDFAISAKPLTRHMPQNKQSFQYRMWQFVVSPPFEYTIMAMIALNTIVLMMKFYGASVAYENALRVFNIVFTSLFSLECVLKVMAFGILNYFRDAWNIFDFVTVLGSITDILVTEFGNNFINLSFLRLFRAARLIKLLRQGYTIRILLWTFVQSFKALPYVCLLIAMLFFIYAIIGMQVFGNIGIDVEDEDSDEDEFQITEHNNFRTFFQALMLLFRSATGEAWHNIMLSCLSGKPCDKNSGILTRECGNEFAYFYFVSFIFLCSFLMLNLFVAVIMDNFEYLTRDSSILGPHHLDEYVRVWAEYDPAA corresponding to amino acids 107-1843 of CCAA_HUMAN, which also corresponds to amino acids 24-1760 of T59742_P2, a third amino acid sequence being at least 70%, optionally at least 80%, preferably at least 85%, more preferably at least 90% and most preferably at least 95% homologous to a polypeptide having the sequence (SEQ ID NO: 56) CGRIHYKDMYSLLRVISPPLGLGKKCPHRVAC corresponding to amino acids 1761-1792 of T59742_P2, a fourth amino acid sequence being at least 90% homologous to (SEQ ID NO: 57) KRLLRMDLPVADDNTVHFNSTLMALIRTALDIKIAKGGADKQQMDAELRKEMMAIWPNLSQKTLDLLVTPHKSTDLTVGKIYAAMMIMEYYRQSKAKKLQAMREEQDRTPLMFQRMEPPSPTQEGGPGQNALPSTQLDPGGALMAHESGLKESPSWVTQRAQEMFQKTGTWSPEQGPPTDMPNSQPNSQSVEMREMGRDGYSDSEHYLPMEGQGRAASMPRLPAENQRRRGRPRGNNLSTISDTSPMKRSASVLGPKARRLDDYSLERVPPEENQRHHQRRRDRSHRASERSLGRYTDVDTGLGTDLSMTTQSGDLPSKERDQERGRPKDRKHRQHHHHHHHHHHPPPPDKDRYAQERPDHGRARARDQRWSRSPSEGREHMAHRQGSSSVSGSPAPSTSGTSTPRRGRRQLPQTPSTPRPHVSYSPVIRKAGGSGP corresponding to amino acids 1876-2312 of CCAA_HUMAN, which also corresponds to amino acids 1793-2229 of T59742_P2 and a fifth amino acid sequence being at least 70%, optionally at least 80%, preferably at least 85%, more preferably at least 90% and most preferably at least 95% homologous to a polypeptide having the sequence (SEQ ID NO: 58) RSSSSSSSSSSSRRWPGRAGRPPAALGGTQAPRPSLWPEIGRPRGATAAAARPGWRGGSQARPGASPPGPVDTAGPGGRHLARTCPRGPRVPGTMATTGAPTTTRPMARAAGAARRPWPGPTTRHPPYDTRPRAPPGARPGLPGPRARPAPRLLGTAGDSPTATTRRTDWPGPAGRAPGRACTNPTARVTMIGAKPGRGGARPAPHAPHAHTPPEEPRRGRGGPAQRARERASRETPDSGEARAGPQGCPAETLGQKRPSWAATAPPNQPRSPHPRQGLSGGRQGADKPHSQGI corresponding to amino acids 2230-2523 of T59742_P2, wherein said first amino acid sequence, second amino acid sequence, third amino acid sequence, fourth amino acid sequence, and fifth amino acid sequence are contiguous and in a sequential order.

According to preferred embodiments of the present invention, there is provided an isolated polypeptide encoding for a head of T59742_P2, comprising a polypeptide being at least 70%, optionally at least about 80%, preferably at least about 85%, more preferably at least about 90% and most preferably at least about 95% homologous to the sequence (SEQ ID NO: 59) MSSWPTRPTKSTAPPRPGGKNGT of T59742_P2.

According to preferred embodiments of the present invention, there is provided an isolated polypeptide encoding for a tail of T59742_P2, comprising a polypeptide being at least 70%, optionally at least about 80%, preferably at least about 85%, more preferably at least about 90% and most preferably at least about 95% homologous to the sequence (SEQ ID NO: 60) RSSSSSSSSSSSRRWPGRAGRPPAALGGTQAPRPSLWPEIGRPRGATAAAARPGWRGGSQARPGASPPGPVDTAGPGGRHLARTCPRGPRVPGTMATTGAPTTTRPMARAAGAARRPWPGPTTRHPPYDTRPRAPPGARPGLPGPRARPAPRLLGTAGDSPTATTRRTDWPGPAGRAPGRACTNPTARVTMIGAKPGRGGARPAPHAPHAHTPPEEPRRGRGGPAQRARERASRETPDSGEARAGPQGCPAETLGQKRPSWAATAPPNQPRSPHPRQGLSGGRQGADKPHSQGI in T59742_P2.

According to preferred embodiments of the present invention, there is provided an isolated polypeptide encoding for a unique insertion of T59742_P2, comprising a polypeptide being at least 70%, optionally at least about 80%, preferably at least about 85%, more preferably at least about 90% and most preferably at least about 95% homologous to the sequence (SEQ ID NO: 61) CGRIHYKDMYSLLRVISPPLGLGKKCPHRVAC in T59742_P2.

Domains affected by alternative splicing in this variant include the cytoplasmic N-terminus region of the protein, the S1 transmembrane domain of domain I and the cytoplasmic C-terminus region of the protein.

Without wishing to be limited by a single hypothesis, it should be noted that these affected domains may affect the function of the resulting calcium channel. In particular, as described above, at least two different C-terminal motifs, having different lengths, have been found in different alpha 1 subunits. These motifs may be generated through the formation of different splice variants. For example, the P/Q Cav2.1 isoforms are generated by alternative splice donor sites at the 5′ end of exon 47, causing a stop codon to form and the protein to be truncated. An alternative donor site results in insertion of five bases leading to a frame shift and an elongation of the C-terminus by 244 amino acids.

For these neuronal channels, as the C-terminus becomes shorter, the current amplitude increases and calcium-dependent inactivation also increases.

Furthermore, with regard to L-type channels specifically, particular domains in the C-terminus region (L, K and an IA motif) have been found to bind calmodulin (CaM) involved in Ca⁺²-induced inactivation. In addition, these channels contain a highly specific Ca⁺² sensor composed of motifs which are important for Ca+2-dependent inactivation of the channel. Ca⁺² loading of this sensor was shown to modulate the CaM affinity of CaM-binding site in the domain.

From the above description of the effect of changes to the C-terminus domain of this variant, it is expected that the variant has a greater current amplitude as compared to the known protein and a stronger calcium dependence of inactivation (ie greater sensitivity to calcium influx).

Sequence name: CCAA_HUMAN

documentation:

of: T59742_P2 (SEQ ID NO: 11)×CCAA_HUMAN (SEQ ID NO: 62) . . .

Alignment segment 1/1:

Quality: 2130.00

Escore: 0

Matching length: 2170 Total

length: 2559

Matching Percent Similarity: 100.00 Matching Percent

Identity: 100.00

Total Percent Similarity: 84.80 Total Percent

Identity: 84.80

Gaps: 4

Alignment:

| | . . . . . | |
|---|---|---|
| 1 | MSSWPTRPTKSTAPPRPGGKNGTTIIANCIVLALEQHLPDDDKTPMSERL | 50 |
| | \|\|\|\|\|\|\|\|\|\|\|\|\|\|\|\|\|\|\|\|\|\|\|\|\|\|\| | |
| 107 | .......................TIIANCIVLALEQHLPDDDKTPMSERL | 133 |
| | . . . . . | |
| 51 | DDTEPYFIGIFCFEAGIKIIALGFAFHKGSYLRNGWNVMDFVVVLTGILA | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 134 | DDTEPYFIGIFCFEAGIKIIALGFAFHKGSYLRNGWNVMDFVVVLTGILA | 183 |
| | . . . . . | |
| 101 | TVGTEFDLRTLRAVRVLRPLKLVSGIPSLQVVLKSIMKAMIPLLQIGLLL | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 184 | TVGTEFDLRTLRAVRVLRPLKLVSGIPSLQVVLKSIMKAMIPLLQIGLLL | 233 |
| | . . . . . | |
| 151 | FFAILIFAIIGLEFYMGKFHTTCFEEGTDDIQGESPAPCGTEEPARTCPN | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 234 | FFAILIFAIIGLEFYMGKFHTTCFEEGTDDIQGESPAPCGTEEPARTCPN | 283 |
| | . . . . . | |
| 201 | GTKCQPYWEGPNNGITQFDNILFAVLTVFQCITMEGWTDLLYNSNDASGN | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 284 | GTKCQPYWEGPNNGITQFDNILFAVLTVFQCITMEGWTDLLYNSNDASGN | 333 |
| | . . . . . | |
| 251 | TWNWLYFIPLIIIGSFFMLNLVLGVLSGEFAKERERVENRRAFLKLRRQQ | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 334 | TWNWLYFIPLIIIGSFFMLNLVLGVLSGEFAKERERVENRRAFLKLRRQQ | 383 |
| | . . . . . | |
| 301 | QIERELNGYMEWISKAEEVILAEDETDGEQRHPFDGALRRTTIKKSKTDL | 350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 384 | QIERELNGYMEWISKAEEVILAEDETDGEQRHPFDGALRRTTIKKSKTDL | 433 |
| | . . . . . | |
| 351 | LNPEEAEDQLADIASVGSPFARASIKSAKLENSTFFHKKERRMRFYIRRM | 400 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 434 | LNPEEAEDQLADIASVGSPFARASIKSAKLENSTFFHKKERRMRFYIRRM | 483 |
| | . . . . . | |
| 401 | VKTQAFYWTVLSLVALNTLCVAIVHYNQPEWLSDFLYYAEFIFLGLFMSE | 450 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 484 | VKTQAFYWTVLSLVALNTLCVAIVHYNQPEWLSDFLYYAEFIFLGLGMSE | 533 |
| | . . . . . | |
| 451 | MFIKMYGLGTRPYFHSSFNCFDCGVIIGSIFEVIWAVIKPGTSFGISVLR | 500 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 534 | MFIKMYGLGTRPYFHSSFNCFDCGVIIGSIFEVIWAVIKPGTSFGISVLR | 583 |
| | . . . . . | |
| 501 | ALRLLRIFKVTKYWASLRNLVVSLLNSMKSIISLLFLLFLFIVVFALLGM | 550 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 584 | ALRLLRIFKVTKYWASLRNLVVSLLNSMKSIISLLFLLFLFIVVFALLGM | 633 |
| | . . . . . | |
| 551 | QLFGGQFNFDEGTPPTNFDTFPAAIMTVFQILTGEDWNEVMYDGIKSQGG | 600 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 634 | QLFGGQFNFDEGTPPTNFDTFPAAIMTVFQILTGEDWNEVMYDGIKSQGG | 683 |
| | . . . . . | |
| 601 | VQGGMVFSIYFIVLTLFGNYTLLNVFLAIAVDNLANAQELTKDEQEEEEA | 650 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 684 | VQGGMVFSIYFIVLTLFGNYTLLNVFLAIAVDNLANAQELTKDEQEEEEA | 733 |
| | . . . . . | |
| 651 | ANQKLALQKAKEVAEVSPLSAANMSIAVKEQQKNQKPAKSVWEQRTSEMR | 700 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 734 | ANQKLALQKAKEVAEVSPLSAANMSIAVKEQQKNQKPAKSVWEQRTSEMR | 783 |
| | . . . . . | |
| 701 | KQNLLASREALYNEMDPDERWKAAYTRHLRPDMKTHLDRPLVVDPQENRN | 750 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 784 | KQNLLASREALYNEMDPDERWKAAYTRHLRPDMKTHLDRPLVVDPQENRN | 833 |
| | . . . . . | |
| 751 | NNTNKSRAAEPTVDQRLGQQRAEDFLRKQARYHDRARDPSGSAGLDARRP | 800 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 834 | NNTNKSRAAEPTVDQRLGQQRAEDFLRKQARYHDRARDPSGSAGLDARRP | 883 |
| | . . . . . | |
| 801 | WAGSQEAELSREGPYGRESDHHAREGSLEQPGFWEGEAERGKAGDPHRRH | 850 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 884 | WAGSQEAELSREGPYGRESDHHAREGSLEQPGFWEGEAERGKAGDPHRRH | 933 |
| | . . . . . | |
| 851 | VHRQGGSRESRSGSPRTGADGEHRRHRAHRRPGEEGPEDKAERRARHREG | 900 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 934 | VHRQGGSRESRSGSPRTGADGEHRRHRAHRRPGEEGPEDKAERRARHREG | 983 |
| | . . . . . | |
| 901 | SRPARGGEGEGEGPDGGERRRRHRHGAPATYEGDARREDKERRHRRRKEN | 950 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 984 | SRPARGGEGEGEGPDGGERRRRHRHGAPATYEGDARREDKERRHRRRKEN | 1033 |
| | . . . . . | |
| 951 | QGSGVPVSGPNLSTTRPIQQDLGRQDPPLAEDIDNMKNNKLATAESAAPH | 1000 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1034 | QGSGVPVSGPNLSTTRPIQQDLGRQDPPLAEDIDNMKNNKLATAESAAPH | 1083 |
| | . . . . . | |
| 1001 | GSLGHAGLPQSPAKMGNSTDPGPMLAIPAMATNPQNAASRRTPNNPGNPS | 1050 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1084 | GSLGHAGLPQSPAKMGNSTDPGPMLAIPAMATNPQNAASRRTPNNPGNPS | 1133 |
| | . . . . . | |
| 1051 | NPGPPKTPENSLIVTNPSGTQTNSAKTARKPDHTTVDIPPACPPPLNHTV | 1100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1134 | NPGPPKTPENSLIVTNPSGTQTNSAKTARKPDHTTVDIPPACPPPLNHTV | 1183 |
| | . . . . . | |
| 1101 | VQVNKNANPDPLPKKEEEKKEEEEDDRGEDGPKPMPPYSSMFILSTTNPL | 1150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1184 | VQVNKNANPDPLPKKEEEKKEEEEDDRGEDGPKPMPPYSSMFILSTTNPL | 1233 |
| | . . . . . | |
| 1151 | RRLCHYILNLRYFEMCILMVIAMSSIALAAEDPVQPNAPRNNVLRYFDYV | 1200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1234 | RRLCHYILNLRYFEMCILMVIAMSSIALAAEDPVQPNAPRNNVLRYFDYV | 1283 |
| | . . . . . | |
| 1201 | FTGVFTFEMVIKMIDLGLVLHQGAYFRDLWNILDFIVVSGALVAFAFTGN | 1250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1284 | FTGVFTFEMVIKMIDLGLVLHQGAYFRDLWNILDFIVVSGALVAFAFTGN | 1333 |
| | . . . . . | |
| 1251 | SKGKDINTIKSLRVLRVLRPLKTIKRLPKLKAVFDCVVNSLKNVFNILIV | 1300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1334 | SKGKDINTIKSLRVLRVLRPLKTIKRLPKLKAVFDCVVNSLKNVFNILIV | 1383 |
| | . . . . . | |
| 1301 | YMLFMFIFAVVAVQLFKGKFFHCTDESKEFEKDCRGKYLLYEKNEVKARD | 1350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1384 | YMLFMFIFAVVAVQLFKGKFFHCTDESKEFEKDCRGKYLLYEKNEVKARD | 1433 |
| | . . . . . | |
| 1351 | REWKKYEFHYDNVLWALLTLFTVSTGEGWPQVLKHSVDATFENQGPSPGY | 1400 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1434 | REWKKYEFHYDNVLWALLTLFTVSTGEGWPQVLKHSVDATFENQGPSPGY | 1483 |
| | . . . . . | |
| 1401 | RMEMSIFYVVYFVVFPFFFVNIFVALIIITFQEQGDKMMEEYSLEKNERA | 1450 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1484 | RMEMSIFYVVYFVVFPFFFVNIFVALIIITFQEQGDKMMEEYSLEKNERA | 1533 |
| | . . . . . | |
| 1451 | CIDFAISAKPLTRHMPQNKQSFQYRMWQFVVSPPFEYTIMAMIALNTIVL | 1500 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1534 | CIDFAISAKPLTRHMPQNKQSFQYRMWQFVVSPPFEYTIMAMIALNTIVL | 1583 |
| | . . . . . | |
| 1501 | MMKFYGASVAYENALRVFNIVFTSLFSLECVLKVMAFGILNYFRDAWNIF | 1550 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1584 | MMKFYGASVAYENALRVFNIVFTSLFSLECVLKVMAFGILNYFRDAWNIF | 1633 |
| | . . . . . | |
| 1551 | DFVTVLGSITDILVTEFGNNFINLSFLRLFRAARLIKLLRQGYTIRILLW | 1600 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1634 | DFVTVLGSITDILVTEFGNNFINLSFLRLFRAARLIKLLRQGYTIRILLW | 1683 |
| | . . . . . | |
| 1601 | TFVQSFKALPYVCLLIAMLFFIYAIIGMQVFGNIGIDVEDEDSDEDEFQI | 1650 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1684 | TFVQSFKALPYVCLLIAMLFFIYAIIGMQVFGNIGIDVEDEDSDEDEFQI | 1733 |
| | . . . . . | |
| 1651 | TEHNNFRTFFQALMLLFRSATGEAWHNIMLSCLSGKPCDKNSGILTRECG | 1700 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1734 | TEHNNFRTFFQALMLLFRSATGEAWHNIMLSCLSGKPCDKNSGILTRECG | 1783 |
| | . . . . . | |
| 1701 | NEFAYFYFVSFIFLCSFLMLNLFVAVIMDNFEYLTRDSSILGPHHLDEYV | 1750 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1784 | NEFAYFYFVSFIFLCSFLMLNLFVAVIMDNFEYLTRDSSILGPHHLDEYV | 1833 |
| | . . . . . | |
| 1751 | RVWAEYDPAA...............................CGRI | 1764 |
| | \|\|\|\|\|\|\|\|\|\| | |
| 1834 | RVWAEYDPAAWGRMPYLDMYQMLRHMSPPLGLGKKCPARVA.... | 1875 |
| | . . . . . | |
| 1765 | HYKDMYSLLRVISPPLGLGKKCPHRVACKRLLRMDLPVADDNTVHFNSTL | 1814 |
| | \|\|\|\|\|\|\|\|\|\|\|\|\|\|\|\|\|\|\|\|\|\| | |
| 1876 | ............................KRLLRMDLPVADDNTVHFNSTL | 1897 |
| | . . . . . | |
| 1815 | MALIRTALDIKIAKGGADKQQMDAELRKEMMAIWPNLSQKTLDLLVTPHK | 1864 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1898 | MALIRTALDIKIAKGGADKQQMDAELRKEMMAIWPNLSQKTLDLLVTPHK | 1947 |
| | . . . . . | |
| 1865 | STDLTVGKIYAAMMIMEYYRQSKAKKLQAMREEQDRTPLMFQRMEPPSPT | 1914 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1948 | STDLTVGKIYAAMMIMEYYRQSKAKKLQAMREEQDRTPLMFQRMEPPSPT | 1997 |
| | . . . . . | |
| 1915 | QEGGPGQNALPSTQLDPGGALMAHESGLKESPSWVTQRAQEMFQKTGTWS | 1964 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1998 | QEGGPGQNALPSTQLDPGGALMAHESGLKESPSWVTQRAQEMFQKTGTWS | 2047 |
| | . . . . . | |
| 1965 | PEQGPPTDMPNSQPNSQSVEMREMGRDGYSDSEHYLPMEGQGRAASMPRL | 2014 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2048 | PEQGPPTDMPNSQPNSQSVEMREMGRDGYSDSEHYLPMEGQGRAASMPRL | 2097 |
| | . . . . . | |
| 2015 | PAENQRRRGRPRGNNLSTISDTSPMKRSASVLGPKARRLDDYSLERVPPE | 2064 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2098 | PAENQRRRGRPRGNNLSTISDTSPMKRSASVLGPKARRLDDYSLERVPPE | 2147 |
| | . . . . . | |
| 2065 | ENQRHHQRRRDRSHRASERSLGRYTDVDTGLGTDLSMTTQSGDLPSKERD | 2114 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2148 | ENQRHHQRRRDRSHRASERSLGRYTDVDTGLGTDLSMTTQSGDLPSKERD | 2197 |
| | . . . . . | |
| 2115 | QERGRPKDRKHRQHHHHHHHHHHPPPPDKDRYAQERPDHGRARARDQRWS | 2164 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2198 | QERGRPKDRKHRQHHHHHHHHHHPPPPDKDRYAQERPDHGRARARDQRWS | 2247 |
| | . . . . . | |
| 2165 | RSPSEGREHMAHRQGSSSVSGSPAPSTSGTSTPRRGRRQLPQTPSTPRPH | 2214 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2248 | RSPSEGREHMAHRQGSSSVSGSPAPSTSGTSTPRRGRRQLPQTPSTPRPH | 2297 |
| | . . . . . | |
| 2215 | VSYSPVIRKAGGSGPRSSSSSSSSSSSRRWPGRAGRPPAALGGTQAPRPS | 2264 |
| | \|\|\|\|\|\|\|\|\|\|\|\|\|\|\| | |
| 2298 | VSYSPVIRKAGGSGP................................... | 2312 |
| | . . . . . | |
| 2265 | LWPEIGRPRGATAAAARPGWRGGSQARPGASPPGPVDTAGPGGRHLARTC | 2314 |
| 2312 | .................................................. | 2312 |
| | . . . . . | |
| 2315 | PRGPRVPGTMATTGAPTTTRPMARAAGAARRPWPGPTTRHPPYDTRPRAP | 2364 |
| 2312 | .................................................. | 2312 |
| | . . . . . | |
| 2365 | PGARPGLPGPRARPAPRLLGTAGDSPTATTRRTDWPGPAGRAPGRACTNP | 2414 |
| 2312 | .................................................. | 2312 |
| | . . . . . | |
| 2415 | TARVTMIGAKPGRGGARPAPHAPHAHTPPEEPRRGRGGPAQRARERASRE | 2464 |
| 2312 | .................................................. | 2312 |
| | . . . . . | |
| 2465 | TPDSGEARAGPQGCPAETLGQKRPSWAATAPPNQPRSPHPRQGLSGGRQG | 2514 |
| 2312 | .................................................. | 2312 |
| 2515 | ADKPHSQGI | 2523 |
| 2312 | ......... | 2312 |

### EXAMPLE 3

This Example relates to the variant T59742_P7 (SEQ ID NO:10; the nucleic acid sequence is given by SEQ ID NO:32), which is a variant alpha 1 subunit according to the present invention, and more specifically, is a splice variant of the known protein CCAA_HUMAN. The known protein structure and function is described above in Example 2.

The structure of the splice variant according to the present invention features a unique head and a unique tail, as compared to the known protein sequence. An alignment is provided at the end of this section, while the comparison between the two sequences is described below.

According to preferred embodiments of the present invention, there is provided an isolated chimeric polypeptide encoding for T59742_P7, comprising a first amino acid sequence being at least 70%, optionally at least 80%, preferably at least 85%, more preferably at least 90% and most preferably at least 95% homologous to a polypeptide having the sequence (SEQ ID NO: 63) MSSWPTRPTKSTAPPRPGGKNGT corresponding to amino acids 1-23 of T59742_P7, a second amino acid sequence being at least 90% homologous to (SEQ ID NO: 64) TIIANCIVLALEQHLPDDDKTPMSERLDDTEPYFIGIFCFEAGIKIIALGFAFHKGSYLRNGWNVMDFVVVLTGILATVGTEFDLRTLRAVRVLRPLKLVSGIPSLQVVLKSIMKAMIPLLQIGLLLFFAILIFAIIGLEFYMGKFHTTCFEEGTDDIQGESPAPCGTEEPARTCPNGTKCQPYWEGPNNGITQFDNILFAVLTVFQCITMEGWTDLLYNSNDASGNTWNWLYFIPLIIIGSFFMLNLVLGVLSGEFAKERERVENRRAFLKLRRQQQIERELNGYMEWISKAEEVILAEDETDGEQRHPFDGALRRTTIKKSKTDLLNPEEAEDQLADIASVGSPFARASIKSAKLENSTFFHKKERRMRFYIRRMVKTQAFYWTVLSLVALNTLCVAIVHYNQPEWLSDFLYYAEFIFLGLFMSEMFIKMYGLGTRPYFHSSFNCFDCGVIIGSIFEVIWAVIKPGTSFGISVLRALRLLRIFKVTKYWASLRNLVVSLLNSMKSIISLLFLLFLFIVVFALLGMQLFGGQFNFDEGTPPTNFDTFPAAIMTVFQILTGEDWNEVMYDGIKSQGGVQGGMVFSIYFIVLTLFGNYTLLNVFLAIAVDNLANAQELTKDEQEEEEAANQKLALQKAKEVAEVSPLSAANMSIAVKEQQKNQKPAKSVWEQRTSEMRKQNLLASREALYNEMDPDERWKAAYTRHLRPDMKTHLDRPLVVDPQENRNNNTNKSRAAEPTVDQRLGQQRAEDFLRKQARYHDRARDPSGSAGLDARRPWAGSQEAELSREGPYGRESDHHAREGSLEQPGFWEGEAERGKAGDPHRRHVHRQGGSRESRSGSPRTGADGEHRRHRAHRRPGEEGPEDKAERRARHREGSRPARGGEGEGEGPDGGERRRRHRHGAPATYEGDARREDKERRHRRRKENQGSGVPVSGPNLSTTRPIQQDLGRQDPPLAEDIDNMKNNKLATAESAAPHGSLGHAGLPQSPAKMGNSTDPGPMLAIPAMATNPQNAASRRTPNNPGNPSNPGPPKTPENSLIVTNPSGTQTNSAKTARKPDHTTVDIPPACPPPLNHTVVQVNKNANPDPLPKKEEEKKEEEEDDRGEDGPKPMPPYSSMFILSTTNPLRRLCHYILNLRYFEMCILMVIAMSSIALAAEDPVQPNAPRNNVLRYFDYVFTGVFTFEMVIKMIDLGLVLHQGAYFRDLWNILDFIVVSGALVAFAFTGNSKGKDINTIKSLRVLRVLRPLKTIKRLPKLKAVFDCVVNSLKNVFNILIVYMLFMFIFAVVAVQLFKGKFFHCTDESKEFEKDCRGKYLLYEKNEVKARDREWKKYEFHYDNVLWALLTLFTVSTGEGWPQVLKHSVDATFENQGPSPGYRMEMSIFYVVYFVVFPFFFVNIFVALIIITFQEQGDKMMEEYSLEKNERACIDFAISAKPLTRHMPQNKQSFQYRMWQFVVSPPFEYTIMAMIALNTIVLMMKFYGASVAYENALRVFNIVFTSLFSLECVLKVMAFGILNYFRDAWNIFDFVTVLGSITDILVTEFG corresponding to amino acids 107-1651 of CCAA_HUMAN, which also corresponds to amino acids 24-1568 of T59742_P7, and a third amino acid sequence being at least 70%, optionally at least 80%, preferably at least 85%, more preferably at least 90% and most preferably at least 95% homologous to a polypeptide having the sequence (SEQ ID NO: 66) AWSRATPPSPRS corresponding to amino acids 1569-1580 of T59742_P7, wherein said first amino acid sequence, second amino acid sequence and third amino acid sequence are contiguous and in a sequential order.

According to preferred embodiments of the present invention, there is provided an isolated polypeptide encoding for a head of T59742_P7, comprising a polypeptide being at least 70%, optionally at least about 80%, preferably at least about 85%, more preferably at least about 90% and most preferably at least about 95% homologous to the sequence (SEQ ID NO: 65) MSSWPTRPTKSTAPPRPGGKNGT of T59742_P7.

According to preferred embodiments of the present invention, there is provided an isolated polypeptide encoding for a tail of T59742_P7, comprising a polypeptide being at least 70%, optionally at least about 80%, preferably at least about 85%, more preferably at least about 90% and most preferably at least about 95% homologous to the sequence (SEQ ID NO: 66) AWSRATPPSPRS in T59742_P7.

Domains affected by alternative splicing of this variant are as follows: the cytoplasmic N-terminus region of the protein; the S1 transmembrane domain of domain I; the extracellular loop between S3 and S4 transmembrane domains of domain IV; the S4 transmembrane domain of domain IV; the extracellular loop between S4 and S5 transmembrane domains of domain IV; the S5 transmembrane domain of domain IV; the pore loop region between S5 and S6 transmembrane domains of domain IV; the S6 transmembrane domain of domain IV; and the cytoplasmic C-terminus region of the protein.

The effects on the C-terminal regions of this alpha 1 subunit type are described above with regard to Example 2.

The extracellular loops between S3 and S4 may influence S4 voltage sensor function because of their close proximity to the S4 segments, which must move upon depolarization, as previously described. It is possible that alternative splicing in these areas could affect the voltage sensor function of S4, even if the S4 region itself were not altered.

With regard to effects in domain IV, as this splice variant is a variant of alpha subunit Cav2.1, even introducing only two amino acids (NP) by insertion of exon 31a can have a significant functional effect. In P/Q-type Cav2.1 channels, the presence of NP slows activation and inactivation and decreases affinity to ω-agatoxin IVA. The NP variant is found in Q-type (low ω-agatoxin IVA affinity) channels, while the variant lacking NP is found in P-type (high affinity) calcium channels. The more rapidly gating P-type calcium channel has been found in cerebellar Purkinje cells, as well as pancreatic beta cells.

From the above description of the effect of changes to the C-terminus domain of this variant, it is expected that the variant has a greater current amplitude as compared to the known protein, as well as stronger calcium dependence of inactivation (greater sensitivity to calcium influx).

From the above description of changes in the extracellular loop between S3 and S4 transmembrane domains of domain IV, it is expected that the variant has a different voltage sensor function as compared to the known protein.

Sequence name: CCAA_HUMAN

documentation:

of: T59742_P7 (residues 24-1568 or SEQ ID NO: 10)×CCAA_HUMAN (SEQ ID NO: 67). . .

segment 1/1:

Quality: 15207.00

Escore: 0

Matching length: 1545 Total

length: 1545

Matching Percent Similarity: 100.00 Matching Percent

Identity: 100.00

Total Percent Similarity: 100.00 Total Percent

Identity: 100.00

Gaps: 0

| | . . . . . | |
|---|---|---|
| 24 | TIIANCIVLALEQHLPDDDKTPMSERLDDTEPYFIGIFCFEAGIKIIALG | 73 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 107 | TIIANCIVLALEQHLPDDDKTPMSERLDDTEPYFIGIFCFEAGIKIIALG | 156 |
| | . . . . . | |
| 74 | FAFHKGSYLRNGWNVMDFVVVLTGILATVGTEFDLRTLRAVRVLRPLKLV | 123 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 157 | FAFHKGSYLRNGWNVMDFVVVLTGILATVGTEFDLRTLRAVRVLRPLKLV | 206 |
| | . . . . . | |
| 124 | SGIPSLQVVLKSIMKAMIPLLQIGLLLFFAILIFAIIGLEFYMGKFHTTC | 173 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 207 | SGIPSLQVVLKSIMKAMIPLLQIGLLLFFAILIFAIIGLEFYMGKFHTTC | 256 |
| | . . . . . | |
| 174 | FEEGTDDIQGESPAPCGTEEPARTCPNGTKCQPYWEGPNNGITQFDNILF | 223 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 257 | FEEGTDDIQGESPAPCGTEEPARTCPNGTKCQPYWEGPNNGITQFDNILF | 306 |
| | . . . . . | |
| 224 | AVLTVFQCITMEGWTDLLYNSNDASGNTWNWLYFIPLIIIGSFFMLNLVL | 273 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 307 | AVLTVFQCITMEGWTDLLYNSNDASGNTWNWLYFIPLIIIGSFFMLNLVL | 356 |
| | . . . . . | |
| 274 | GVLSGEFAKERERVENRRAFLKLRRQQQIERELNGYMEWISKAEEVILAE | 323 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 357 | GVLSGEFAKERERVENRRAFLKLRRQQQIERELNGYMEWISKAEEVILAE | 406 |
| | . . . . . | |
| 324 | DETDGEQRHPFDGALRRTTIKKSKTDLLNPEEAEDQLADIASVGSPFARA | 373 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 407 | DETDGEQRHPFDGALRRTTIKKSKTDLLNPEEAEDQLADIASVGSPFARA | 456 |
| | . . . . . | |
| 374 | SIKSAKLENSTFFHKKERRMRFYIRRMVKTQAFYWTVLSLVALNTLCVAI | 423 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 457 | SIKSAKLENSTFFHKKERRMRFYIRRMVKTQAFYWTVLSLVALNTLCVAI | 506 |
| | . . . . . | |
| 424 | VHYNQPEWLSDFLYYAEFIFLGLFMSEMFIKMYGLGTRPYFHSSFNCFDC | 473 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 507 | VHYNQPEWLSDFLYYAEFIFLGLFMSEMFIKMYGLGTRPYFHSSFNCFDC | 556 |
| | . . . . . | |
| 474 | GVIIGSIFEVIWAVIKPGTSFGISVLRALRLLRIFKVTKYWASLRNLVVS | 523 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 557 | GVIIGSIFEVIWAVIKPGTSFGISVLRALRLLRIFKVTKYWASLRNLVVS | 606 |
| | . . . . . | |
| 524 | LLNSMKSIISLLFLLFLFIVVFALLGMQLFGGQFNFDEGTPPTNFDTFPA | 573 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 607 | LLNSMKSIISLLFLLFLFIVVFALLGMQLFGGQFNFDEGTPPTNFDTFPA | 656 |
| | . . . . . | |
| 574 | AIMTVFQILTGEDWNEVMYDGIKSQGGVQGGMVFSIYFIVLTLFGNYTLL | 623 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 657 | AIMTVFQILTGEDWNEVMYDGIKSQGGVQGGMVFSIYFIVLTLFGNYTLL | 706 |
| | . . . . . | |
| 624 | NVFLAIAVDNLANAQELTKDEQEEEEAANQKLALQKAKEVAEVSPLSAAN | 673 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 707 | NVFLAIAVDNLANAQELTKDEQEEEEAANQKLALQKAKEVAEVSPLSAAN | 756 |
| | . . . . . | |
| 674 | MSIAVKEQQKNQKPAKSVWEQRTSEMRKQNLLASREALYNEMDPDERWKA | 723 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 757 | MSIAVKEQQKNQKPAKSVWEQRTSEMRKQNLLASREALYNEMDPDERWKA | 806 |
| | . . . . . | |
| 724 | AYTRHLRPDMKTHLDRPLVVDPQENRNNNTNKSRAAEPTVDQRLGQQRAE | 773 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 807 | AYTRHLRPDMKTHLDRPLVVDPQENRNNNTNKSRAAEPTVDQRLGQQRAE | 856 |
| | . . . . . | |
| 774 | DFLRKQARYHDRARDPSGSAGLDARRPWAGSQEAELSREGPYGRESDHHA | 823 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 857 | DFLRKQARYHDRARDPSGSAGLDARRPWAGSQEAELSREGPYGRESDHHA | 906 |
| | . . . . . | |
| 824 | REGSLEQPGFWEGEAERGKAGDPHRRHVHRQGGSRESRSGSPRTGADGEH | 873 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 907 | REGSLEQPGFWEGEAERGKAGDPHRRHVHRQGGSRESRSGSPRTGADGEH | 956 |
| | . . . . . | |
| 874 | RRHRAHRRPGEEGPEDKAERRARHREGSRPARGGEGEGEGPDGGERRRRH | 923 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 957 | RRHRAHRRPGEEGPEDKAERRARHREGSRPARGGEGEGEGPDGGERRRRH | 1006 |
| | . . . . . | |
| 924 | RHGAPATYEGDARREDKERRHRRRKENQGSGVPVSGPNLSTTRPIQQDLG | 973 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1007 | RHGAPATYEGDARREDKERRHRRRKENQGSGVPVSGPNLSTTRPIQQDLG | 1056 |
| | . . . . . | |
| 974 | RQDPPLAEDIDNMKNNKLATAESAAPHGSLGHAGLPQSPAKMGNSTDPGP | 1023 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1057 | RQDPPLAEDIDNMKNNKLATAESAAPHGSLGHAGLPQSPAKMGNSTDPGP | 1106 |
| | . . . . . | |
| 1024 | MLAIPAMATNPQNAASRRTPNNPGNPSNPGPPKTPENSLIVTNPSGTQTN | 1073 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1107 | MLAIPAMATNPQNAASRRTPNNPGNPSNPGPPKTPENSLIVTNPSGTQTN | 1156 |
| | . . . . . | |
| 1074 | SAKTARKPDHTTVDIPPACPPPLNHTVVQVNKNANPDPLPKKEEEKKEEE | 1123 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1157 | SAKTARKPDHTTVDIPPACPPPLNHTVVQVNKNANPDPLPKKEEEKKEEE | 1206 |
| | . . . . . | |
| 1124 | EDDRGEDGPKPMPPYSSMFILSTTNPLRRLCHYILNLRYFEMCILMVIAM | 1173 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1207 | EDDRGEDGPKPMPPYSSMFILSTTNPLRRLCHYILNLRYFEMCILMVIAM | 1256 |
| | . . . . . | |
| 1174 | SSIALAAEDPVQPNAPRNNVLRYFDYVFTGVFTFEMVIKMIDLGLVLHQG | 1223 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1257 | SSIALAAEDPVQPNAPRNNVLRYFDYVFTGVFTFEMVIKMIDLGLVLHQG | 1306 |
| | . . . . . | |
| 1224 | AYFRDLWNILDFIVVSGALVAFAFTGNSKGKDINTIKSLRVLRVLRPLKT | 1273 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1307 | AYFRDLWNILDFIVVSGALVAFAFTGNSKGKDINTIKSLRVLRVLRPLKT | 1356 |
| | . . . . . | |
| 1274 | IKRLPKLKAVFDCVVNSLKNVFNILIVYMLFMFIFAVVAVQLFKGKFFHC | 1323 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1357 | IKRLPKLKAVFDCVVNSLKNVFNILIVYMLFMFIFAVVAVQLFKGKFFHC | 1406 |
| | . . . . . | |
| 1324 | TDESKEFEKDCRGKYLLYEKNEVKARDREWKKYEFHYDNVLWALLTLFTV | 1373 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1407 | TDESKEFEKDCRGKYLLYEKNEVKARDREWKKYEFHYDNVLWALLTLFTV | 1456 |
| | . . . . . | |
| 1374 | STGEGWPQVLKHSVDATFENQGPSPGYRMEMSIFYVVYFVVFPFFFVNIF | 1423 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1457 | STGEGWPQVLKHSVDATFENQGPSPGYRMEMSIFYVVYFVVFPFFFVNIF | 1506 |
| | . . . . . | |
| 1424 | VALIIITFQEQGDKMMEEYSLEKNERACIDFAISAKPLTRHMPQNKQSFQ | 1473 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1507 | VALIIITFQEQGDKMMEEYSLEKNERACIDFAISAKPLTRHMPQNKQSFQ | 1556 |
| | . . . . . | |
| 1474 | YRMWQFVVSPPFEYTIMAMIALNTIVLMMKFYGASVAYENALRVFNIVFT | 1523 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1557 | YRMWQFVVSPPFEYTIMAMIALNTIVLMMKFYGASVAYENALRVFNIVFT | 1606 |
| | . . . . | |
| 1524 | SLFSLECVLKVMAFGILNYFRDAWNIFDFVTVLGSITDILVTEFG | 1568 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1607 | SLFSLECVLKVMAFGILNYFRDAWNIFDFVTVLGSITDILVTEFG | 1651 |

### EXAMPLE 4

This Example relates to the variant T59742_P3 (SEQ ID NO:14; the nucleic acid sequence is given by SEQ ID NO:36), which is a variant alpha 1 subunit according to the present invention, and more specifically, is a splice variant of the known protein CCAA_HUMAN. The known protein structure and function is described above in Example 2.

The structure of the splice variant according to the present invention features a unique insertion and a unique tail, as compared to the known protein sequence. An alignment is provided at the end of this section, while the comparison between the two sequences is described below.

According to preferred embodiments of the present invention, there is provided an isolated chimeric polypeptide encoding for T59742_P3, comprising a first amino acid sequence being at least 90% homologous to (SEQ ID NO: 68) MARFGDEMPARYGGGGSGAAAGVVVGSGGGRGAGGSRQGGQPGAQRMYKQSMAQRARTMALYNPIPVRQNCLTVNRSLFLFSEDNVVRKYAKKITEWPPFEYMILATIIANCIVLALEQHLPDDDKTPMSERLDDTEPYFIGIFCFEAGIKIIALGFAFHKGSYLRNGWNVMDFVVVLTGILATVGTEFDLRTLRAVRVLRPLKLVSGIPSLQVVLKSIMKAMIPLLQIGLLLFFAILIFAIIGLEFYMGKFHTTCFEEGTDDIQGESPAPCGTEEPARTCPNGTKCQPYWEGPNNGITQFDNILFAVLTVFQCITMEGWTDLLYNSNDASGNTWNWLYFIPLIIIGSFFMLNLVLGVLSGEFAKERERVENRRAFLKLRRQQQIERELNGYMEWISKAEEVILAEDETDGEQRHPFDGALRRTTIKKSKTDLLNPEEAEDQLADIASVGSPFARASIKSAKLENSTFFHKKERRMRFYIRRMVKTQAFYWTVLSLVALNTLCVAIVHYNQPEWLSDFLYYAEFIFLGLFMSEMFIKMYGLGTRPYFHSSFNCFDCGVIIGSIFEVIWAVIKPGTSFGISVLRALRLLRIFKVTKYWASLRNLVVSLLNSMKSIISLLFLLFLFIVVFALLGMQLFGGQFNFDEGTPPTNFDTFPAAIMTVFQILTGEDWNEVMYDGIKSQGGVQGGMVFSIYFIVLTLFGNYTLLNVFLAIAVDNLANAQELTKDEQEEEEAANQKLALQKAKEVAEVSPLSAANMSIAVKEQQKNQKPAKSVWEQRTSEMRKQNLLASREALYNEMDPDERWKAAYTRHLRPDMKTHLDRPLVVDPQENRNNNTNKSRAAEPTVDQRLGQQRAEDFLRKQARYHDRARDPSGSAGLDARRPWAGSQEAELSREGPYGRESDHHAREGSLEQPGFWEGEAERGKAGDPHRRHVHRQGGSRESRSGSPRTGADGEHRRHRAHRRPGEEGPEDKAERRARHREGSRPARGGEGEGEGPDGGERRRRHRHGAPATYEGDARREDKERRHRRRKENQGSGVPVSGPNLSTTRPIQQDLGRQDPPLAEDIDNMKNNKLATAESAAPHGSLGHAGLPQSPAKMGNSTDPGPMLAIPAMATNPQNAASRRTPNNPGNPSNPGPPKTPENSLIVTNPSGTQTNSAKTARKPDHTTVDIPPACPPPLNHTVVQVNKNANPDPLPKKEEEKKEEEEDDRGEDGPKPMPPYSSMFILSTTNPLRRLCHYILNLRYFEMCILMVIAMSSIALAAEDPVQPNAPRNNVLRYFDYVFTGVFTFEMVIKMIDLGLVLHQGAYFRDLWNILDFIVVSGALVAFAFTGNSKGKDINTIKSLRVLRVLRPLKTIKRLPKLKAVFDCVVNSLKNVFNILIVYMLFMFIFAVVAVQLFKGKFFHCTDESKEFEKDCRGKYLLYEKNEVKARDREWKKYEFHYDNVLWALLTLFTVSTGEGWPQVLKHSVDATFENQGPSPGYRMEMSIFYVVYFVVFPFFFVNIFVALIIITFQEQGDKMMEEYSLEKNERACIDFAISAKPLTRHMPQNKQSFQYRMWQFVVSPPFEYTIMAMIALNTIVLMMKFYGASVAYENALRVFNIVFTSLFSLECVLKVMAFGILNYFRDAWNIFDFVTVLGSITDILVTEFGNNFINLSFLRLFRAARLIKLLRQGYTIRILLWTFVQSFKALPYVCLLIAMLFFIYAIIGMQVFGNIGIDVEDEDSDEDEFQITEHNNFRTFFQALMLLFRSATGEAWHNIMLSCLSGKPCDKNSGILTRECGNEFAYFYFVSFIFLCSFLMLNLFVAVIMDNFEYLTRDSSILGPHHLDEYVRVWAEYDPAA corresponding to amino acids 1-1843 of CCAA_HUMAN, which also corresponds to amino acids 1-1843 of T59742_P3, a second amino acid sequence being at least 70%, optionally at least 80%, preferably at least 85%, more preferably at least 90% and most preferably at least 95% homologous to a polypeptide having the sequence (SEQ ID NO: 73) CGRIHYKDMYSLLRVISPPLGLGKKCPHRVAC corresponding to amino acids 1844-1875 of T59742_P3, a third amino acid sequence being at least 90% homologous to (SEQ ID NO: 69) KRLLRMDLPVADDNTVHFNSTLMALIRTALDIKIAKGGADKQQMDAELRKEMMAIWPNLSQKTLDLLVTPHKSTDLTVGKIYAAMMIMEYYRQSKAKKLQAMREEQDRTPLMFQRMEPPSPTQEGGPGQNALPSTQLDPGGALMAHESGLKESPSWVTQRAQEMFQKTGTWSPEQGPPTDMPNSQPNSQSVEMREMGRDGYSDSEHYLPMEGQGRAASMPRLPAENQRRRGRPRGNNLSTISDTSPMKRSASVLGPKARRLDDYSLERVPPEENQRHHQRRRDRSHRASERSLGRYTDVDTGLGTDLSMTTQSGDLPSKERDQERGRPKDRKHRQHHHHHHHHHHPPPPDKDRYAQERPDHGRARARDQRWSRSPSEGREHMAHRQGSSSVSGSPAPSTSGTSTPRRGRRQLPQTPSTPRPHVSYSPVIRKAGGSGP corresponding to amino acids 1876-2312 of CCAA_HUMAN, which also corresponds to amino acids 1876-2312 of T59742_P3, a bridging amino acid R corresponding to amino acid 2313 of T59742_P3, a fourth amino acid sequence being at least 90% homologous to (SEQ ID NO: 70) QQQQQQQQQQ corresponding to amino acids 2314-2323 of CCAA_HUMAN, which also corresponds to amino acids 2314-2323 of T59742_P3 and a fifth amino acid sequence being at least 70%, optionally at least 80%, preferably at least 85%, more preferably at least 90% and most preferably at least 95% homologous to a polypeptide having the sequence (SEQ ID NO: 71) HSSSSSSSSSSSRRWPGRAGRPPAALGGTQAPRPSLWPEIGRPRGATAAAARPGWRGGSQARPGASPPGPVDTAGPGGRHLARTCPRGPRVPGTMATTGAPTTTRPMARAAGAARRPWPGPTTRHPPYDTRPRAPPGARPGLPGPRARPAPRLLGTAGDSPTATTRRTDWPGPAGRAPGRACTNPTARVTMIGAKPGRGGARPAPHAPHAHTPPEEPRRGRGGPAQRARERASRETPDSGEARAGPQGCPAETLGQKRPSWAATAPPNQPRSPHPRQGLSGGRQGADKPHSQGI corresponding to amino acids 2324-2617 of T59742_P3, wherein said first amino acid sequence, second amino acid sequence, third amino acid sequence, bridging amino acid, fourth amino acid sequence, and fifth amino acid sequence are contiguous and in a sequential order.

According to preferred embodiments of the present invention, there is provided an isolated polypeptide encoding for a tail of T59742_P3, comprising a polypeptide being at least 70%, optionally at least about 80%, preferably at least about 85%, more preferably at least about 90% and most preferably at least about 95% homologous to the sequence (SEQ ID NO: 72) HSSSSSSSSSSSRRWPGRAGRPPAALGGTQAPRPSLWPEIGRPRGATAAAARPGWRGGSQARPGASPPGPVDTAGPGGRHLARTCPRGPRVPGTMATTGAPTTTRPMARAAGAARRPWPGPTTRHPPYDTRPRAPPGARPGLPGPRARPAPRLLGTAGDSPTATTRRTDWPGPAGRAPGRACTNPTARVTMIGAKPGRGGARPAPHAPHAHTPPEEPRRGRGGPAQRARERASRETPDSGEARAGPQGCPAETLGQKRPSWAATAPPNQPRSPHPRQGLSGGRQGADKPHSQGI in T59742_P3.

According to preferred embodiments of the present invention, there is provided an isolated polypeptide encoding for a unique of T59742_P3, comprising a polypeptide being at least 70%, optionally at least about 80%, preferably at least about 85%, more preferably at least about 90% and most preferably at least about 95% homologous to the sequence (SEQ ID NO: 73) CGRIHYKDMYSLLRVISPPLGLGKKCPHRVAC in T59742_P3.

Domains affected by alternative splicing include the cytoplasmic C-terminus region of the protein.

The effects on the C-terminal regions of this alpha 1 subunit type are described above with regard to Example 2.

From the above description of the effect of changes to the C-terminus domain of this variant, it is expected that the variant has greater current amplitude as compared to the known protein, and a stronger calcium dependence of inactivation.

In addition, since there is a change in the calcium-binding region, it is expected that the calcium binding characteristics of the channel will differ from those of the known protein and will be modulated in some manner (increased or decreased).

Sequence name: CCAA_HUMAN

documentation:

of: T59742_P3 (SEQ ID NO: 14)×CCAA_HUMAN (SEQ ID NO: 74) . . .

Alignment segment 1/1:

Quality: 2246.00

Escore: 0

Matching length: 2276

length: 2653

Matching Percent Similarity: 100.00 Matching Percent

Identity: 100.00

Total Percent Similarity: 85.79 Total Percent

Identity: 85.79

Gaps: 3

Alignment:

| | . . . . . | |
|---|---|---|
| 1 | MARFGDEMPARYGGGGSGAAAGVVVGSGGGRGAGGSRQGGQPGAQRMYKQ | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MARFGDEMPARYGGGGSGAAAGVVVGSGGGRGAGGSRQGGQPGAQRMYKQ | 50 |
| | . . . . . | |
| 51 | SMAQRARTMALYNPIPVRQNCLTVNRSLFLFSEDNVVRKYAKKITEWPPF | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | SMAQRARTMALYNPIPVRQNCLTVNRSLFLFSEDNVVRKYAKKITEWPPF | 100 |
| | . . . . . | |
| 101 | EYMILATIIANCIVLALEQHLPDDDKTPMSERLDDTEPYFIGIFCFEAGI | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | EYMILATIIANCIVLALEQHLPDDDKTPMSERLDDTEPYFIGIFCFEAGI | 150 |
| | . . . . . | |
| 151 | KIIALGFAFHKGSYLRNGWNVMDFVVVLTGILATVGTEFDLRTLRAVRVL | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | KIIALGFAFHKGSYLRNGWNVMDFVVVLTGILATVGTEFDLRTLRAVRVL | 200 |
| | . . . . . | |
| 201 | RPLKLVSGIPSLQVVLKSIMKAMIPLLQIGLLLFFAILIFAIIGLEFYMG | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | RPLKLVSGIPSLQVVLKSIMKAMIPLLQIGLLLFFAILIFAIIGLEFYMG | 250 |
| | . . . . . | |
| 251 | KFHTTCFEEGTDDIQGESPAPCGTEEPARTCPNGTKCQPYWEGPNNGITQ | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | KFHTTCFEEGTDDIQGESPAPCGTEEPARTCPNGTKCQPYWEGPNNGITQ | 300 |
| | . . . . . | |
| 301 | FDNILFAVLTVFQCITMEGWTDLLYNSNDASGNTWNWLYFIPLIIIGSFF | 350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | FDNILFAVLTVFQCITMEGWTDLLYNSNDASGNTWNWLYFIPLIIIGSFF | 350 |
| | . . . . . | |
| 351 | MLNLVLGVLSGEFAKERERVENRRAFLKLRRQQQIERELNGYMEWISKAE | 400 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 351 | MLNLVLGVLSGEFAKERERVENRRAFLKLRRQQQIERELNGYMEWISKAE | 400 |
| | . . . . . | |
| 401 | EVILAEDETDGEQRHPFDGALRRTTIKKSKTDLLNPEEAEDQLADIASVG | 450 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 401 | EVILAEDETDGEQRHPFDGALRRTTIKKSKTDLLNPEEAEDQLADIASVG | 450 |
| | . . . . . | |
| 451 | SPFARASIKSAKLENSTFFHKKERRMRFYIRRMVKTQAFYWTVLSLVALN | 500 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 451 | SPFARASIKSAKLENSTFFHKKERRMRFYIRRMVKTQAFYWTVLSLVALN | 500 |
| | . . . . . | |
| 501 | TLCVAIVHYNQPEWLSDFLYYAEFIFLGLFMSEMFIKMYGLGTRPYFHSS | 550 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 501 | TLCVAIVHYNQPEWLSDFLYYAEFIFLGLFMSEMFIKMYGLGTRPYFHSS 550 | |
| | . . . . . | |
| 551 | FNCFDCGVIIGSIFEVIWAVIKPGTSFGISVLRALRLLRIFKVTKYWASL | 600 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 551 | FNCFDCGVIIGSIFEVIWAVIKPGTSFGISVLRALRLLRIFKVTKYWASL | 600 |
| | . . . . . | |
| 601 | RNLVVSLLNSMKSIISLLFLLFLFIVVFALLGMQLFGGQFNFDEGTPPTN | 650 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 601 | RNLVVSLLNSMKSIISLLFLLFLFIVVFALLGMQLFGGQFNFDEGTPPTN | 650 |
| | . . . . . | |
| 651 | FDTFPAAIMTVFQILTGEDWNEVMYDGIKSQGGVQGGMVFSIYFIVLTLF | 700 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 651 | FDTFPAAIMTVFQILTGEDWNEVMYDGIKSQGGVQGGMVFSIYFIVLTLF | 700 |
| | . . . . . | |
| 701 | GNYTLLNVFLAIAVDNLANAQELTKDEQEEEEAANQKLALQKAKEVAEVS | 750 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 701 | GNYTLLNVFLAIAVDNLANAQELTKDEQEEEEAANQKLALQKAKEVAEVS | 750 |
| | . . . . . | |
| 751 | PLSAANMSIAVKEQQKNQKPAKSVWEQRTSEMRKQNLLASREALYNEMDP | 800 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 751 | PLSAANMSIAVKEQQKNQKPAKSVWEQRTSEMRKQNLLASREALYNEMDP | 800 |
| | . . . . . | |
| 801 | DERWKAAYTRHLRPDMKTHLDRPLVVDPQENRNNNTNKSRAAEPTVDQRL | 850 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 801 | DERWKAAYTRHLRPDMKTHLDRPLVVDPQENRNNNTNKSRAAEPTVDQRL | 850 |
| | . . . . . | |
| 851 | GQQRAEDFLRKQARYHDRARDPSGSAGLDARRPWAGSQEAELSREGPYGR | 900 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 851 | GQQRAEDFLRKQARYHDRARDPSGSAGLDARRPWAGSQEAELSREGPYGR | 900 |
| | . . . . . | |
| 901 | ESDHHAREGSLEQPGFWEGEAERGKAGDPHRRHVHRQGGSRESRSGSPRT | 950 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 901 | ESDHHAREGSLEQPGFWEGEAERGKAGDPHRRHVHRQGGSRESRSGSPRT | 950 |
| | . . . . . | |
| 951 | GADGEHRRHRAHRRPGEEGPEDKAERRARHREGSRPARGGEGEGEGPDGG | 1000 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 951 | GADGEHRRHRAHRRPGEEGPEDKAERRARHREGSRPARGGEGEGEGPDGG | 1000 |
| | . . . . . | |
| 1001 | ERRRRHRHGAPATYEGDARREDKERRHRRRKENQGSGVPVSGPNLSTTRP | 1050 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1001 | ERRRRHRHGAPATYEGDARREDKERRHRRRKENQGSGVPVSGPNLSTTRP | 1050 |
| | . . . . . | |
| 1051 | IQQDLGRQDPPLAEDIDNMKNNKLATAESAAPHGSLGHAGLPQSPAKMGN | 1100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1051 | IQQDLGRQDPPLAEDIDNMKNNKLATAESAAPHGSLGHAGLPQSPAKMGN | 1100 |
| | . . . . . | |
| 1101 | STDPGPMLAIPAMATNPQNAASRRTPNNPGNPSNPGPPKTPENSLIVTNP | 1150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1101 | STDPGPMLAIPAMATNPQNAASRRTPNNPGNPSNPGPPKTPENSLIVTNP | 1150 |
| | . . . . . | |
| 1151 | SGTQTNSAKTARKPDHTTVDIPPACPPPLNHTVVQVNKNANPDPLPKKEE | 1200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1151 | SGTQTNSAKTARKPDHTTVDIPPACPPPLNHTVVQVNKNANPDPLPKKEE | 1200 |
| | . . . . . | |
| 1201 | EKKEEEEDDRGEDGPKPMPPYSSMFILSTTNPLRRLCHYILNLRYFEMCI | 1250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1201 | EKKEEEEDDRGEDGPKPMPPYSSMFILSTTNPLRRLCHYILNLRYFEMCI | 1250 |
| | . . . . . | |
| 1251 | LMVIAMSSIALAAEDPVQPNAPRNNVLRYFDYVFTGVFTFEMVIKMIDLG | 1300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1251 | LMVIAMSSIALAAEDPVQPNAPRNNVLRYFDYVFTGVFTFEMVIKMIDLG | 1300 |
| | . . . . . | |
| 1301 | LVLHQGAYFRDLWNILDFIVVSGALVAFAFTGNSKGKDINTIKSLRVLRV | 1350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1301 | LVLHQGAYFRDLWNILDFIVVSGALVAFAFTGNSKGKDINTIKSLRVLRV | 1350 |
| | . . . . . | |
| 1351 | LRPLKTIKRLPKLKAVFDCVVNSLKNVFNILIVYMLFMFIFAVVAVQLFK | 1400 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1351 | LRPLKTIKRLPKLKAVFDCVVNSLKNVFNILIVYMLFMFIFAVVAVQLFK | 1400 |
| | . . . . . | |
| 1401 | GKFFHCTDESKEFEKDCRGKYLLYEKNEVKARDREWKKYEFHYDNVLWAL | 1450 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1401 | GKFFHCTDESKEFEKDCRGKYLLYEKNEVKARDREWKKYEFHYDNVLWAL | 1450 |
| | . . . . . | |
| 1451 | LTLFTVSTGEGWPQVLKHSVDATFENQGPSPGYRMEMSIFYVVYFVVFPF | 1500 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1451 | LTLFTVSTGEGWPQVLKHSVDATFENQGPSPGYRMEMSIFYVVYFVVFPF | 1500 |
| | . . . . . | |
| 1501 | FFVNIFVALIIITFQEQGDKMMEEYSLEKNERACIDFAISAKPLTRHMPQ | 1550 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1501 | FFVNIFVALIIITFQEQGDKMMEEYSLEKNERACIDFAISAKPLTRHMPQ | 1550 |
| | . . . . . | |
| 1551 | NKQSFQYRMWQFVVSPPFEYTIMAMIALNTIVLMMKFYGASVAYENALRV | 1600 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1551 | NKQSFQYRMWQFVVSPPFEYTIMAMIALNTIVLMMKFYGASVAYENALRV | 1600 |
| | . . . . . | |
| 1601 | FNIVFTSLFSLECVLKVMAFGILNYFRDAWNIFDFVTVLGSITDILVTEF | 1650 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1601 | FNIVFTSLFSLECVLKVMAFGILNYFRDAWNIFDFVTVLGSITDILVTEF | 1650 |
| | . . . . . | |
| 1651 | GNNFINLSFLRLFRAARLIKLLRQGYTIRILLWTFVQSFKALPYVCLLIA | 1700 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1651 | GNNFINLSFLRLFRAARLIKLLRQGYTIRILLWTFVQSFKALPYVCLLIA | 1700 |
| | . . . . . | |
| 1701 | MLFFIYAIIGMQVFGNIGIDVEDEDSDEDEFQITEHNNFRTFFQALMLLF | 1750 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1701 | MLFFIYAIIGMQVFGNIGIDVEDEDSDEDEFQITEHNNFRTFFQALMLLF | 1750 |
| | . . . . . | |
| 1751 | RSATGEAWHNIMLSCLSGKPCDKNSGILTRECGNEFAYFYFVSFIFLCSF | 1800 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1751 | RSATGEAWHNIMLSCLSGKPCDKNSGILTRECGNEFAYFYFVSFIFLCSF | 1800 |
| | . . . . . | |
| 1801 | LMLNLFVAVIMDNFEYLTRDSSILGPHHLDEYVRVWAEYDPAA....... | 1843 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1801 | LMLNLFVAVIMDNFEYLTRDSSILGPHHLDEYVRVWAEYDPAAWGRMPYL | 1850 |
| | . . . . . | |
| 1844 | .........................CGRIHYKDMYSLLRVISPPLG | 1864 |
| 1851 | DMYQMLRHMSPPLGLGKKCPARVAY..................... | 1875 |
| | . . . . . | |
| 1865 | LGKKCPHRVACKRLLRMDLPVADDNTVHFNSTLMALIRTALDIKIAKGGA | 1914 |
| | \|\|\|\|\|\|\|\|\|\|\|\|\|\|\|\|\|\|\|\|\|\|\|\|\|\|\|\|\|\|\|\|\|\|\|\|\|\|\| | |
| 1876 | ...........KRLLRMDLPVADDNTVHFNSTLMALIRTALDIKIAKGGA | 1914 |
| | . . . . . | |
| 1915 | DKQQMDAELRKEMMAIWPNLSQKTLDLLVTPHKSTDLTVGKIYAAMMIME | 1964 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1915 | DKQQMDAELRKEMMAIWPNLSQKTLDLLVTPHKSTDLTVGKIYAAMMIME | 1964 |
| | . . . . . | |
| 1965 | YYRQSKAKKLQAMREEQDRTPLMFQRMEPPSPTQEGGPGQNALPSTQLDP | 2014 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1965 | YYRQSKAKKLQAMREEQDRTPLMFQRMEPPSPTQEGGPGQNALPSTQLDP | 2014 |
| | . . . . . | |
| 2015 | GGALMAHESGLKESPSWVTQRAQEMFQKTGTWSPEQGPPTDMPNSQPNSQ | 2064 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2015 | GGALMAHESGLKESPSWVTQRAQEMFQKTGTWSPEQGPPTDMPNSQPNSQ | 2064 |
| | . . . . . | |
| 2065 | SVEMREMGRDGYSDSEHYLPMEGQGRAASMPRLPAENQRRRGRPRGNNLS | 2114 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2065 | SVEMREMGRDGYSDSEHYLPMEGQGRAASMPRLPAENQRRRGRPRGNNLS | 2114 |
| | . . . . . | |
| 2115 | TISDTSPMKRSASVLGPKARRLDDYSLERVPPEENQRHHQRRRDRSHRAS | 2164 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2115 | TISDTSPMKRSASVLGPKARRLDDYSLERVPPEENQRHHQRRRDRSHRAS | 2164 |
| | . . . . . | |
| 2165 | ERSLGRYTDVDTGLGTDLSMTTQSGDLPSKERDQERGRPKDRKHRQHHHH | 2214 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2165 | ERSLGRYTDVDTGLGTDLSMTTQSGDLPSKERDQERGRPKDRKHRQHHHH | 2214 |
| | . . . . . | |
| 2215 | HHHHHHPPPPDKDRYAQERPDHGRARARDQRWSRSPSEGREHMAHRQGSS | 2264 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2215 | HHHHHHPPPPDKDRYAQERPDHGRARARDQRWSRSPSEGREHMAHRQGSS | 2264 |
| | . . . . . | |
| 2265 | SVSGSPAPSTSGTSTPRRGRRQLPQTPSTPRPHVSYSPVIRKAGGSGPRQ | 2314 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2265 | SVSGSPAPSTSGTSTPRRGRRQLPQTPSTPRPHVSYSPVIRKAGGSGP.. | 2312 |
| | . . . . . | |
| 2315 | QQQQQQQQQHSSSSSSSSSSSRRWPGRAGRPPAALGGTQAPRPSLWPEIG | 2364 |
| 2312 | .................................................. | 2312 |
| | . . . . . | |
| 2365 | RPRGATAAAARPGWRGGSQARPGASPPGPVDTAGPGGRHLARTCPRGPRV | 2414 |
| 2312 | .................................................. | 2312 |
| | . . . . . | |
| 2415 | PGTMATTGAPTTTRPMARAAGAARRPWPGPTTRHPPYDTRPRAPPGARPG | 2464 |
| 2312 | .................................................. | 2312 |
| | . . . . . | |
| 2465 | LPGPRARPAPRLLGTAGDSPTATTRRTDWPGPAGRAPGRACTNPTARVTM | 2514 |
| 2312 | .................................................. | 2312 |
| | . . . . . | |
| 2515 | IGAKPGRGGARPAPHAPHAHTPPEEPRRGRGGPAQRARERASRETPDSGE | 2564 |
| 2312 | .................................................. | 2312 |
| | . . . . . | |
| 2565 | ARAGPQGCPAETLGQKRPSWAATAPPNQPRSPHPRQGLSGGRQGADKPHS | 2614 |
| 2312 | .................................................. | 2312 |
| 2615 | QGI | 2617 |
| 2312 | ... | 2312 |

### EXAMPLE 5

This Example relates to the variant T59742_P4 (SEQ ID NO:12; the nucleic acid sequence is given by SEQ ID NO:34), which is a variant alpha 1 subunit according to the present invention, and more specifically, is a splice variant of the known protein CCAA_HUMAN. The known protein structure and function is described above in Example 2.

The structure of the splice variant according to the present invention features a unique head, a unique insertion, a skipped exon and a unique tail, as compared to the known protein sequence. An alignment is provided at the end of this section, while the comparison between the two sequences is described below.

According to preferred embodiments of the present invention, there is provided an isolated chimeric polypeptide encoding for T59742_P4, comprising a first amino acid sequence being at least 70%, optionally at least 80%, preferably at least 85%, more preferably at least 90% and most preferably at least 95% homologous to a polypeptide having the sequence (SEQ ID NO: 75) MSSWPTRPTKSTAPPRPGGKNGT corresponding to amino acids 1-23 of T59742_P4, a second amino acid sequence being at least 90% homologous to (SEQ ID NO: 76) TIIANCIVLALEQHLPDDDKTPMSERLDDTEPYFIGIFCFEAGIKIIALGFAFHKGSYLRNGWNVMDFVVVLTGILATVGTEFDLRTLRAVRVLRPLKLVSGIPSLQVVLKSIMKAMIPLLQIGLLLFFAILIFAIIGLEFYMGKFHTTCFEEGTDDIQGESPAPCGTEEPARTCPNGTKCQPYWEGPNNGITQFDNILFAVLTVFQCITMEGWTDLLYNSNDASGNTWNWLYFIPLIIIGSFFMLNLVLGVLSGEFAKERERVENRRAFLKLRRQQQIERELNGYMEWISKAEEVILAEDETDGEQRHPFDGALRRTTIKKSKTDLLNPEEAEDQLADIASVGSPFARASIKSAKLENSTFFHKKERRMRFYIRRMVKTQAFYWTVLSLVALNTLCVAIVHYNQPEWLSDFLYYAEFIFLGLFMSEMFIKMYGLGTRPYFHSSFNCFDCGVIIGSIFEVIWAVIKPGTSFGISVLRALRLLRIFKVTKYWASLRNLVVSLLNSMKSIISLLFLLFLFIVVFALLGMQLFGGQFNFDEGTPPTNFDTFPAAIMTVFQILTGEDWNEVMYDGIKSQGGVQGGMVFSIYFIVLTLFGNYTLLNVFLAIAVDNLANAQELTKDEQEEEEAANQKLALQKAKEVAEVSPLSAANMSIAVKEQQKNQKPAKSVWEQRTSEMRKQNLLASREALYNEMDPDERWKAAYTRHLRPDMKTHLDRPLVVDPQENRNNNTNKSRAAEPTVDQRLGQQRAEDFLRKQARYHDRARDPSGSAGLDARRPWAGSQEAELSREGPYGRESDHHAREGSLEQPGFWEGEAERGKAGDPHRRHVHRQGGSRESRSGSPRTGADGEHRRHRAHRRPGEEGPEDKAERRARHREGSRPARGGEGEGEGPDGGERRRRHRHGAPATYEGDARREDKERRHRRRKENQGSGVPVSGPNLSTTRPIQQDLGRQDPPLAEDIDNMKNNKLATAESAAPHGSLGHAGLPQSPAKMGNSTDPGPMLAIPAMATNPQNAASRRTPNNPGNPSNPGPPKTPENSLIVTNPSGTQTNSAKTARKPDHTTVDIPPACPPPLNHTVVQVNKNANPDPLPKKEEEKKEEEEDDRGEDGPKPMPPYSSMFILSTTNPLRRLCHYILNLRYFEMCILMVIAMSSIALAAEDPVQPNAPRNNVLRYFDYVFTGVFTFEMVIKMIDLGLVLHQGAYFRDLWNILDFIVVSGALVAFAFTGNSKGKDINTIKSLRVLRVLRPLKTIKRLPKLKAVFDCVVNSLKNVFNILIVYMLFMFIFAVVAVQLFKGKFFHCTDESKEFEKDCRGKYLLYEKNEVKARDREWKKYEFHYDNVLWALLTLFTVSTGEGWPQVLKHSVDATFENQGPSPGYRMEMSIFYVVYFVVFPFFFVNIFVALIIITFQEQGDKMMEEYSLEKNERACIDFAISAKPLTRHMPQNKQSFQYRMWQFVVSPPFEYTIMAMIALNTIVLMMKFYGASVAYENALRVFNIVFTSLFSLECVLKVMAFGILNYFRDAWNIFDFVTVLGSITDILVTEFGNNFINLSFLRLFRAARLIKLLRQGYTIRILLWTFVQSFKALPYVCLLIAMLFFIYAIIGMQVFGNIGIDVEDEDSDEDEFQITEHNNFRTFFQALMLLFRSATGEAWHNIMLSCLSGKPCDKNSGILTRECGNEFAYFYFVSFIFLCSFLMLNLFVAVIMDNFEYLTRDSSILGPHHLDEYVRVWAEYDPAA corresponding to amino acids 107-1843 of CCAA_HUMAN, which also corresponds to amino acids 24-1760 of T59742_P4, a third amino acid sequence being at least 70%, optionally at least 80%, preferably at least 85%, more preferably at least 90% and most preferably at least 95% homologous to a polypeptide having the sequence (SEQ ID NO: 77) CGRIHYKDMYSLLRVISPPLGLGKKCPHRVAC corresponding to amino acids 1761-1792 of T59742_P4, a fourth amino acid sequence being at least 90% homologous to (SEQ ID NO: 78) KRLLRMDLPVADDNTVHFNSTLMALIRTALDIKIAKGGADKQQMDAELRKEMMAIWPNLSQKTLDLLVTPHKSTDLTVGKIYAAMMIMEYYRQSKAKKLQAMREEQDRTPLMFQRMEPPSPTQEGGPGQNALPSTQLDPGGALMAHESGLKESPSWVTQRAQEMFQKTGTWSPEQGPPTDMPNSQPNSQSVEMREMGRDGYSDSEHYLPMEGQGRAASMPRLPAENQ corresponding to amino acids 1876-2102 of CCAA_HUMAN, which also corresponds to amino acids 1793-2019 of T59742_P4, 10th amino acid sequence being at least 90% homologous to (SEQ ID NO: 79) TISDTSPMKRSASVLGPKARRLDDYSLERVPPEENQRHHQRRRDRSHRASERSLGRYTDVDTGLGTDLSMTTQSGDLPSKERDQERGRPKDRKHRQHHHHHHHHHHPPPPDKDRYAQERPDHGRARARDQRWSRSPSEGREHMAHRQGSSSVSGSPAPSTSGTSTPRRGRRQLPQTPSTPRPHVSYSPVIRKAGGSGP corresponding to amino acids 2115-2312 of CCAA_HUMAN, which also corresponds to amino acids 2020-2217 of T59742_P4 and a fifth amino acid sequence being at least 70%, optionally at least 80%, preferably at least 85%, more preferably at least 90% and most preferably at least 95% homologous to a polypeptide having the sequence (SEQ ID NO: 80) RSSSSSSSSSSSRRWPGRAGRPPAALGGTQAPRPSLWPEIGRPRGATAAAARPGWRGGSQARPGASPPGPVDTAGPGGRHLARTCPRGPRVPGTMATTGAPTTTRPMARAAGAARRPWPGPTTRHPPYDTRPRAPPGARPGLPGPRARPAPRLLGTAGDSPTATTRRTDWPGPAGRAPGRACTNPTARVTMIGAKPGRGGARPAPHAPHAHTPPEEPRRGRGGPAQRARERASRETPDSGEARAGPQGCPAETLGQKRPSWAATAPPNQPRSPHPRQGLSGGRQGADKPHSQGI corresponding to amino acids 2218-2511 of T59742_P4, wherein said first amino acid sequence, second amino acid sequence, third amino acid sequence, fourth amino acid sequence and fifth amino acid sequence are contiguous and in a sequential order.

According to preferred embodiments of the present invention, there is provided an isolated polypeptide encoding for a head of T59742_P4, comprising a polypeptide being at least 70%, optionally at least about 80%, preferably at least about 85%, more preferably at least about 90% and most preferably at least about 95% homologous to the sequence (SEQ ID NO: 81) MSSWPTRPTKSTAPPRPGGKNGT of T59742_P4.

According to preferred embodiments of the present invention, there is provided an isolated chimeric polypeptide encoding for an edge portion of T59742_P4, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise QT, having a structure as follows: a sequence starting from any of amino acid numbers 2019−x to 2019; and ending at any of amino acid numbers 2020+((n−2)−x), in which x varies from 0 to n−2.

According to preferred embodiments of the present invention, there is provided an isolated polypeptide encoding for a tail of T59742_P4, comprising a polypeptide being at least 70%, optionally at least about 80%, preferably at least about 85%, more preferably at least about 90% and most preferably at least about 95% homologous to the sequence (SEQ ID NO: 82) RSSSSSSSSSSSRRWPGRAGRPPAALGGTQAPRPSLWPEIGRPRGATAAAARPGWRGGSQARPGASPPGPVDTAGPGGRHLARTCPRGPRVPGTMATTGAPTTTRPMARAAGAARRPWPGPTTRHPPYDTRPRAPPGARPGLPGPRARPAPRLLGTAGDSPTATTRRTDWPGPAGRAPGRACTNPTARVTMIGAKPGRGGARPAPHAPHAHTPPEEPRRGRGGPAQRARERASRETPDSGEARAGPQGCPAETLGQKRPSWAATAPPNQPRSPHPRQGLSGGRQGADKPHSQGI in T59742_P4.

According to preferred embodiments of the present invention, there is provided an isolated polypeptide encoding for a unique insertion of T59742_P4, comprising a polypeptide being at least 70%, optionally at least about 80%, preferably at least about 85%, more preferably at least about 90% and most preferably at least about 95% homologous to the sequence (SEQ ID NO: 83) CGRIHYKDMYSLLRVISPPLGLGKKCPHRVAC in T59742_P4.

Domains affected by alternative splicing include the cytoplasmic N-terminus region of the protein; the S1 transmembrane domain of domain I and the cytoplasmic C-terminus region of the protein.

The effects on the C-terminal regions of this alpha 1 subunit type are described above with regard to Example 2.

From the above description of the effect of changes to the C-terminus domain of this variant, it is expected that the variant has a greater current amplitude as compared to the known protein, and a stronger calcium dependence of inactivation.

In addition, since there is a change in the calcium-binding region (comparing to swissprot annotation), it is expected that the calcium binding characteristics of the resultant channel will differ from those of the known protein.

Sequence name: CCAA_HUMAN

documentation:

of: T59742_P4 (SEQ ID NO: 12)×CCAA_HUMAN (SEQ ID NO: 84). . .

segment 1/1:

Quality: 2108.00

Escore: 0

Matching lenth: 2158 Total

length: 2559

Matching Percent Similarity: 100.00 Matching Percent

Identity: 100.00

Total Percent Similarity: 84.33 Total Percent

Identity: 84.33

Gaps: 5

Alignment:

| | . . . . . | |
|---|---|---|
| 1 | MSSWPTRPTKSTAPPRPGGKNGTTIIANCIVLALEQHLPDDDKTPMSERL | 50 |
| | \|\|\|\|\|\|\|\|\|\|\|\|\|\|\|\|\|\|\|\|\|\|\|\|\|\|\| | |
| 107 | .......................TIIANCIVLALEQHLPDDDKTPMSERL | 133 |
| | . . . . . | |
| 51 | DDTEPYFIGIFCFEAGIKIIALGFAFHKGSYLRNGWNVMDFVVVLTGILA | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 134 | DDTEPYFIGIFCFEAGIKIIALGFAFHKGSYLRNGWNVMDFVVVLTGILA | 183 |
| | . . . . . | |
| 101 | TVGTEFDLRTLRAVRVLRPLKLVSGIPSLQVVLKSIMKAMIPLLQIGLLL | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 184 | TVGTEFDLRTLRAVRVLRPLKLVSGIPSLQVVLKSIMKAMIPLLQIGLLL | 233 |
| | . . . . . | |
| 151 | FFAILIFAIIGLEFYMGKFHTTCFEEGTDDIQGESPAPCGTEEPARTCPN | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 234 | FFAILIFAIIGLEFYMGKFHTTCFEEGTDDIQGESPAPCGTEEPARTCPN | 283 |
| | . . . . . | |
| 201 | GTKCQPYWEGPNNGITQEDNILFAVLTVFQCITMEGWTDLLYNSNDASGN | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 284 | GTKCQPYWEGPNNGITQFDNILFAVLTVFQCITMEGWTDLLYNSNDASGN | 333 |
| | . . . . . | |
| 251 | TWNWLYFIPLIIIGSFFMLNLVLGVLSGEFAKERERVENRRAFLKLRRQQ | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 334 | TWNWLYFIPLIIIGSFFMLNLVLGVLSGEFAKERERVENRRAFLKLRRQQ | 383 |
| | . . . . . | |
| 301 | QIERELNGYMEWISKAEEVILAEDETDGEQRHPFDGALRRTTIKKSKTDL | 350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 384 | QIERELNGYMEWISKAEEVILAEDETDGEQRHPFDGALRRTTIKKSKTDL | 433 |
| | . . . . . | |
| 351 | LNPEEAEDQLADIASVGSPFARASIKSAKLENSTFFHKKERRMRFYIRRM | 400 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 434 | LNPEEAEDQLADIASVGSPFARASIKSAKLENSTFFHKKERRMRFYIRRM | 483 |
| | . . . . . | |
| 401 | VKTQAFYWTVLSLVALNTLCVAIVHYNQPEWLSDFLYYAEFIFLGLFMSE | 450 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 484 | VKTQAFYWTVLSLVALNTLCVAIVHYNQPEWLSDFLYYAEFIFLGLFMSE | 533 |
| | . . . . . | |
| 451 | MFIKMYGLGTRPYFHSSFNCFDCGVIIGSIFEVIWAVIKPGTSFGISVLR | 500 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 534 | MFIKMYGLGTRPYFHSSFNCFDCGVIIGSIFEVIWAVIKPGTSFGISVLR | 583 |
| | . . . . . | |
| 501 | ALRLLRIFKVTKYWASLRNLVVSLLNSMKSIISLLFLLFLFIVVFALLGM | 550 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 584 | ALRLLRIFKVTKYWASLRNLVVSLLNSMKSIISLLFLLFLFIVVFALLGM | 633 |
| | . . . . . | |
| 551 | QLFGGQFNFDEGTPPTNFDTFPAAIMTVFQILTGEDWNEVMYDGIKSQGG | 600 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 634 | QLFGGQFNFDEGTPPTNFDTFPAAIMTVFQILTGEDWNEVMYDGIKSQGG | 683 |
| | . . . . . | |
| 601 | VQGGMVFSIYFIVLTLFGNYTLLNVFLAIAVDNLANAQELTKDEQEEEEA | 650 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 684 | VQGGMVFSIYFIVLTLFGNYTLLNVFLAIAVDNLANAQELTKDEQEEEEA | 733 |
| | . . . . . | |
| 651 | ANQKLALQKAKEVAEVSPLSAANMSIAVKEQQKNQKPAKSVWEQRTSEMR | 700 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 734 | ANQKLALQKAKEVAEVSPLSAANMSIAVKEQQKNQKPAKSVWEQRTSEMR | 783 |
| | . . . . . | |
| 701 | KQNLLASREALYNEMDPDERWKAAYTRHLRPDMKTHLDRPLVVDPQENRN | 750 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 784 | KQNLLASREALYNEMDPDERWKAAYTRHLRPDMKTHLDRPLVVDPQENRN | 833 |
| | . . . . . | |
| 751 | NNTNKSRAAEPTVDQRLGQQRAEDFLRKQARYHDRARDPSGSAGLDARRP | 800 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 834 | NNTNKSRAAEPTVDQRLGQQRAEDFLRKQARYHDRARDPSGSAGLDARRP | 883 |
| | . . . . . | |
| 801 | WAGSQEAELSREGPYGRESDHHAREGSLEQPGFWEGEAERGKAGDPHRRH | 850 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 884 | WAGSQEAELSREGPYGRESDHHAREGSLEQPGFWEGEAERGKAGDPHRRH | 933 |
| | . . . . . | |
| 851 | VHRQGGSRESRSGSPRTGADGEHRRHRAHRRPGEEGPEDKAERRARHREG | 900 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 934 | VHRQGGSRESRSGSPRTGADGEHRRHRAHRRPGEEGPEDKAERRARHREG | 983 |
| | . . . . . | |
| 901 | SRPARGGEGEGEGPDGGERRRRHRHGAPATYEGDARREDKERRHRRRKEN | 950 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 984 | SRPARGGEGEGEGPDGGERRRRHRHGAPATYEGDARREDKERRHRRRKEN | 1033 |
| | . . . . . | |
| 951 | QGSGVPVSGPNLSTTRPIQQDLGRQDPPLAEDIDNMKNNKLATAESAAPH | 1000 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1034 | QGSGVPVSGPNLSTTRPIQQDLGRQDPPLAEDIDNMKNNKLATAESAAPH | 1083 |
| | . . . . . | |
| 1001 | GSLGHAGLPQSPAKMGNSTDPGPMLAIPAMATNPQNAASRRTPNNPGNPS | 1050 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1084 | GSLGHAGLPQSPAKMGNSTDPGPMLAIPAMATNPQNAASRRTPNNPGNPS | 1133 |
| | . . . . . | |
| 1051 | NPGPPKTPENSLIVTNPSGTQTNSAKTARKPDHTTVDIPPACPPPLNHTV | 1100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1134 | NPGPPKTPENSLIVTNPSGTQTNSAKTARKPDHTTVDIPPACPPPLNHTV | 1183 |
| | . . . . . | |
| 1101 | VQVNKNANPDPLPKKEEEKKEEEEDDRGEDGPKPMPPYSSMFILSTTNPL | 1150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1184 | VQVNKNANPDPLPKKEEEKKEEEEDDRGEDGPKPMPPYSSMFILSTTNPL | 1233 |
| | . . . . . | |
| 1151 | RRLCHYILNLRYFEMCILMVIAMSSIALAAEDPVQPNAPRNNVLRYFDYV | 1200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1234 | RRLCHYILNLRYFEMCILMVIAMSSIALAAEDPVQPNAPRNNVLRYFDYV | 1283 |
| | . . . . . | |
| 1201 | FTGVFTFEMVIKMIDLGLVLHQGAYFRDLWNILDFIVVSGALVAFAFTGN | 1250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1284 | FTGVFTFEMVIKMIDLGLVLHQGAYFRDLWNILDFIVVSGALVAFAFTGN | 1333 |
| | . . . . . | |
| 1251 | SKGKDINTIKSLRVLRVLRPLKTIKRLPKLKAVFDCVVNSLKNVFNILIV | 1300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1334 | SKGKDINTIKSLRVLRVLRPLKTIKRLPKLKAVFDCVVNSLKNVFNILIV | 1383 |
| | . . . . . | |
| 1301 | YMLFMFIFAVVAVQLFKGKFFHCTDESKEFEKDCRGKYLLYEKNEVKARD | 1350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1384 | YMLFMFIFAVVAVQLFKGKFFHCTDESKEFEKDCRGKYLLYEKNEVKARD | 1433 |
| | . . . . . | |
| 1351 | REWKKYEFHYDNVLWALLTLFTVSTGEGWPQVLKHSVDATFENQGPSPGY | 1400 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1434 | REWKKYEFHYDNVLWALLTLFTVSTGEGWPQVLKHSVDATFENQGPSPGY | 1483 |
| | . . . . . | |
| 1401 | RMEMSIFYVVYFVVFPFFFVNIFVALIIITFQEQGDKMMEEYSLEKNERA | 1450 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1484 | RMEMSIFYVVYFVVFPFFFVNIFVALIIITFQEQGDKMMEEYSLEKNERA | 1533 |
| | . . . . . | |
| 1451 | CIDFAISAKPLTRHMPQNKQSFQYRMWQFVVSPPFEYTIMAMIALNTIVL | 1500 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1534 | CIDFAISAKPLTRHMPQNKQSFQYRMWQFVVSPPFEYTIMAMIALNTIVL | 1583 |
| | . . . . . | |
| 1501 | MMKFYGASVAYENALRVFNIVFTSLFSLECVLKVMAFGILNYFRDAWNIF | 1550 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1584 | MMKFYGASVAYENALRVFNIVFTSLFSLECVLKVMAFGILNYFRDAWNIF | 1633 |
| | . . . . . | |
| 1551 | DFVTVLGSITDILVTEFGNNFINLSFLRLFRAARLIKLLRQGYTIRILLW | 1600 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1634 | DFVTVLGSITDILVTEFGNNFINLSFLRLFRAARLIKLLRQGYTIRILLW | 1683 |
| | . . . . . | |
| 1601 | TFVQSFKALPYVCLLIAMLFFIYAIIGMQVFGNIGIDVEDEDSDEDEFQI | 1650 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1684 | TFVQSFKALPYVCLLIAMLFFIYAIIGMQVFGNIGIDVEDEDSDEDEFQI | 1733 |
| | . . . . . | |
| 1651 | TEHNNFRTFFQALMLLFRSATGEAWHNIMLSCLSGKPCDKNSGILTRECG | 1700 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1734 | TEHNNFRTFFQALMLLFRSATGEAWHNIMLSCLSGKPCDKNSGILTRECG | 1783 |
| | . . . . . | |
| 1701 | NEFAYFYFVSFIFLCSFLMLNLFVAVIMDNFEYLTRDSSILGPHHLDEYV | 1750 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1784 | NEFAYFYFVSFIFLCSFLMLNLFVAVIMDNFEYLTRDSSILGPHHLDEYV | 1833 |
| | . . . . . | |
| 1751 | RVWAEYDPAA................................CGRI | 1764 |
| | \|\|\|\|\|\|\|\|\|\| | |
| 1834 | RVWAEYDPAAWGRMPYLDMYQMLRHMSPPLGLGKKCPARVAY.... | 1875 |
| | . . . . . | |
| 1765 | HYKDMYSLLRVISPPLGLGKKCPHRVACKRLLRMDLPVADDNTVHFNSTL | 1814 |
| | \|\|\|\|\|\|\|\|\|\|\|\|\|\|\|\|\|\|\|\|\|\| | |
| 1876 | ............................KRLLRMDLPVADDNTVHFNSTL | 1897 |
| | . . . . . | |
| 1815 | MALIRTALDIKIAKGGADKQQMDAELRKEMMAIWPNLSQKTLDLLVTPHK | 1864 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1898 | MALIRTALDIKIAKGGADKQQMDAELRKEMMAIWPNLSQKTLDLLVTPHK | 1947 |
| | . . . . . | |
| 1865 | STDLTVGKIYAAMMIMEYYRQSKAKKLQAMREEQDRTPLMFQRMEPPSPT | 1914 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1948 | STDLTVGKIYAAMMIMEYYRQSKAKKLQAMREEQDRTPLMFQRMEPPSPT | 1997 |
| | . . . . . | |
| 1915 | QEGGPGQNALPSTQLDPGGALMAHESGLKESPSWVTQRAQEMFQKTGTWS | 1964 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1998 | QEGGPGQNALPSTQLDPGGALMAHESGLKESPSWVTQRAQEMFQKTGTWS | 2047 |
| | . . . . . | |
| 1965 | PEQGPPTDMPNSQPNSQSVEMREMGRDGYSDSEHYLPMEGQGRAASMPRL | 2014 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2048 | PEQGPPTDMPNSQPNSQSVEMREMGRDGYSDSEHYLPMEGQGRAASMPRL | 2097 |
| | . . . . . | |
| 2015 | PAENQ............TISDTSPMKRSASVLGPKARRLDDYSLERVPPE | 2052 |
| | \|\|\|\|\| \|\|\|\|\|\|\|\|\|\|\|\|\|\|\|\|\|\|\|\|\|\|\|\|\|\|\|\|\|\|\|\|\| | |
| 2098 | PAENQRRRGRPRGNNLSTISDTSPMKRSASVLGPKARRLDDYSLERVPPE | 2147 |
| | . . . . . | |
| 2053 | ENQRHHQRRRDRSHRASERSLGRYTDVDTGLGTDLSMTTQSGDLPSKERD | 2102 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2148 | ENQRHHQRRRDRSHRASERSLGRYTDVDTGLGTDLSMTTQSGDLPSKERD | 2197 |
| | . . . . . | |
| 2103 | QERGRPKDRKHRQHHHHHHHHHHPPPPDKDRYAQERPDHGRARARDQRWS | 2152 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2198 | QERGRPKDRKHRQHHHHHHHHHHPPPPDKDRYAQERPDHGRARARDQRWS | 2247 |
| | . . . . . | |
| 2153 | RSPSEGREHMAHRQGSSSVSGSPAPSTSGTSTPRRGRRQLPQTPSTPRPH | 2202 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2248 | RSPSEGREHMAHRQGSSSVSGSPAPSTSGTSTPRRGRRQLPQTPSTPRPH | 2297 |
| | . . . . . | |
| 2203 | VSYSPVIRKAGGSGPRSSSSSSSSSSSRRWPGRAGRPPAALGGTQAPRPS | 2252 |
| | \|\|\|\|\|\|\|\|\|\|\|\|\|\|\| | |
| 2298 | VSYSPVIRKAGGSGP................................... | 2312 |
| | . . . . . | |
| 2253 | LWPEIGRPRGATAAAARPGWRGGSQARPGASPPGPVDTAGPGGRHLARTC | 2302 |
| 2312 | .................................................. | 2312 |
| | . . . . . | |
| 2303 | PRGPRVPGTMATTGAPTTTRPMARAAGAARRPWPGPTTRHPPYDTRPRAP | 2352 |
| 2312 | .................................................. | 2312 |
| | . . . . . | |
| 2353 | PGARPGLPGPRARPAPRLLGTAGDSPTATTRRTDWPGPAGRAPGRACTNP | 2402 |
| 2312 | .................................................. | 2312 |
| | . . . . . | |
| 2403 | TARVTMIGAKPGRGGARPAPHAPHAHTPPEEPRRGRGGPAQRARERASRE | 2452 |
| 2312 | .................................................. | 2312 |
| | . . . . . | |
| 2453 | TPDSGEARAGPQGCPAETLGQKRPSWAATAPPNQPRSPHPRQGLSGGRQG | 2502 |
| 2312 | .................................................. | 2312 |
| 2503 | ADKPHSQGI | 2511 |
| 2312 | ......... | 2312 |

### EXAMPLE 6

This Example relates to the variant T59742_P5 (SEQ ID NO:13; the nucleic acid sequence is given by SEQ ID NO:35), which is a variant alpha 1 subunit according to the present invention, and more specifically, is a splice variant of the known protein CCAA_HUMAN. The known protein structure and function is described above in Example 2.

The structure of the splice variant according to the present invention features a unique head, a skipped exon and a unique tail, as compared to the known protein sequence. An alignment is provided at the end of this section, while the comparison between the two sequences is described below.

According to preferred embodiments of the present invention, there is provided an isolated chimeric polypeptide encoding for T59742_P5, comprising a first amino acid sequence being at least 70%, optionally at least 80%, preferably at least 85%, more preferably at least 90% and most preferably at least 95% homologous to a polypeptide having the sequence (SEQ ID NO: 85) MSSWPTRPTKSTAPPRPGGKNGT corresponding to amino acids 1-23 of T59742_P5, a second amino acid sequence being at least 90% homologous to (SEQ ID NO: 86) TIIANCIVLALEQHLPDDDKTPMSERLDDTEPYFIGIFCFEAGIKIIALGFAFHKGSYLRNGWNVMDFVVVLTGILATVGTEFDLRTLRAVRVLRPLKLVSGIPSLQVVLKSIMKAMIPLLQIGLLLFFAILIFAIIGLEFYMGKFHTTCFEEGTDDIQGESPAPCGTEEPARTCPNGTKCQPYWEGPNNGITQFDNILFAVLTVFQCITMEGWTDLLYNSNDASGNTWNWLYFIPLIIIGSFFMLNLVLGVLSGEFAKERERVENRRAFLKLRRQQQIERELNGYMEWISKAEEVILAEDETDGEQRHPFDGALRRTTIKKSKTDLLNPEEAEDQLADIASVGSPFARASIKSAKLENSTFFHKKERRMRFYIRRMVKTQAFYWTVLSLVALNTLCVAIVHYNQPEWLSDFLYYAEFIFLGLFMSEMFIKMYGLGTRPYFHSSFNCFDCGVIIGSIFEVIWAVIKPGTSFGISVLRALRLLRIFKVTKYWASLRNLVVSLLNSMKSIISLLFLLFLFIVVFALLGMQLFGGQFNFDEGTPPTNFDTFPAAIMTVFQILTGEDWNEVMYDGIKSQGGVQGGMVFSIYFIVLTLFGNYTLLNVFLAIAVDNLANAQELTKDEQEEEEAANQKLALQKAKEVAEVSPLSAANMSIAVKEQQKNQKPAKSVWEQRTSEMRKQNLLASREALYNEMDPDERWKAAYTRHLRPDMKTHLDRPLVVDPQENRNNNTNKSRAAEPTVDQRLGQQRAEDFLRKQARYHDRARDPSGSAGLDARRPWAGSQEAELSREGPYGRESDHHAREGSLEQPGFWEGEAERGKAGDPHRRHVHRQGGSRESRSGSPRTGADGEHRRHRAHRRPGEEGPEDKAERRARHREGSRPARGGEGEGEGPDGGERRRRHRHGAPATYEGDARREDKERRHRRRKENQGSGVPVSGPNLSTTRPIQQDLGRQDPPLAEDIDNMKNNKLATAESAAPHGSLGHAGLPQSPAKMGNSTDPGPMLAIPAMATNPQNAASRRTPNNPGNPSNPGPPKTPENSLIVTNPSGTQTNSAKTARKPDHTTVDIPPACPPPLNHTVVQVNKNANPDPLPKKEEEKKEEEEDDRGEDGPKPMPPYSSMFILSTTNPLRRLCHYILNLRYFEMCILMVIAMSSIALAAEDPVQPNAPRNNVLRYFDYVFTGVFTFEMVIKMIDLGLVLHQGAYFRDLWNILDFIVVSGALVAFAFTGNSKGKDINTIKSLRVLRVLRPLKTIKRLPKLKAVFDCVVNSLKNVFNILIVYMLFMFIFAVVAVQLFKGKFFHCTDESKEFEKDCRGKYLLYEKNEVKARDREWKKYEFHYDNVLWALLTLFTVSTGEGWPQVLKHSVDATFENQGPSPGYRMEMSIFYVVYFVVFPFFFVNIFVALIIITFQEQGDKMMEEYSLEKNERACIDFAISAKPLTRHMPQNKQSFQYRMWQFVVSPPFEYTIMAMIALNTIVLMMKFYGASVAYENALRVFNIVFTSLFSLECVLKVMAFGILNYFRDAWNIFDFVTVLGSITDILVTEFGNNFINLSFLRLFRAARLIKLLRQGYTIRILLWTFVQSFKALPYVCLLIAMLFFIYAIIGMQVFGNIGIDVEDEDSDEDEFQITEHNNFRTFFQALMLLFRSATGEAWHNIMLSCLSGKPCDKNSGILTRECGNEFAYFYFVSFIFLCSFLMLNLFVAVIMDNFEYLTRDSSILGPHHLDEYVRVWAEYDPAAWGRMPYLDMYQMLRHMSPPLGLGKKCPARVAYKRLLRMDLPVADDNTVHFNSTLMALIRTALDIKIAKGGADKQQMDAELRKEMMAIWPNLSQKTLDLLVTPHKSTDLTVGKIYAAMMIMEYYRQSKAKKLQAMREEQDRTPLMFQRMEPPSPTQEGGPGQNALPSTQLDPGGALMAHESGLKESPSWVTQRAQEMFQKTGTWSPEQGPPTDMPNSQPNSQSVEMREMGRDGYSDSEHYLPMEGQGRAASMPRLPAENQ corresponding to amino acids 107-2102 of CCAA_HUMAN, which also corresponds to amino acids 24-2019 of T59742_P5, a third amino acid sequence being at least 90% homologous to (SEQ ID NO: 87) TISDTSPMKRSASVLGPKARRLDDYSLERVPPEENQRHHQRRRDRSHRASERSLGRYTDVDTGLGTDLSMTTQSGDLPSKERDQERGRPKDRKHRQHHHHHHHHHHPPPPDKDRYAQERPDHGRARARDQRWSRSPSEGREHMAHRQGSSSVSGSPAPSTSGTSTPRRGRRQLPQTPSTPRPHVSYSPVIRKAGGSGP corresponding to amino acids 2115-2312 of CCAA_HUMAN, which also corresponds to amino acids 2020-2217 of T59742_P5, and a fourth amino acid sequence being at least 70%, optionally at least 80%, preferably at least 85%, more preferably at least 90% and most preferably at least 95% homologous to a polypeptide having the sequence (SEQ ID NO: 88) RSSSSSSSSSSSRRWPGRAGRPPAALGGTQAPRPSLWPEIGRPRGATAAAARPGWRGGSQARPGASPPGPVDTAGPGGRHLARTCPRGPRVPGTMATTGAPTTTRPMARAAGAARRPWPGPTTRHPPYDTRPRAPPGARPGLPGPRARPAPRLLGTAGDSPTATTRRTDWPGPAGRAPGRACTNPTARVTMIGAKPGRGGARPAPHAPHAHTPPEEPRRGRGGPAQRARERASRETPDSGEARAGPQGCPAETLGQKRPSWAATAPPNQPRSPHPRQGLSGGRQGADKPHSQGI corresponding to amino acids 2218-2511 of T59742_P5, wherein said first amino acid sequence, second amino acid sequence, third amino acid sequence and fourth amino acid sequence are contiguous and in a sequential order.

According to preferred embodiments of the present invention, there is provided an isolated polypeptide encoding for a head of T59742_P5, comprising a polypeptide being at least 70%, optionally at least about 80%, preferably at least about 85%, more preferably at least about 90% and most preferably at least about 95% homologous to the sequence (SEQ ID NO: 89) MSSWPTRPTKSTAPPRPGGKNGT of T59742_P5.

According to preferred embodiments of the present invention, there is provided an isolated chimeric polypeptide encoding for an edge portion of T59742_P5, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise QT, having a structure as follows: a sequence starting from any of amino acid numbers 2019−x to 2020; and ending at any of amino acid numbers 2020+((n−2)−x), in which x varies from 0 to n−2.

According to preferred embodiments of the present invention, there is provided an isolated polypeptide encoding for a tail of T59742_P5, comprising a polypeptide being at least 70%, optionally at least about 80%, preferably at least about 85%, more preferably at least about 90% and most preferably at least about 95% homologous to the sequence (SEQ ID NO: 90) RSSSSSSSSSSSRRWPGRAGRPPAALGGTQAPRPSLWPEIGRPRGATAAAARPGWRGGSQARPGASPPGPVDTAGPGGRHLARTCPRGPRVPGTMATTGAPTTTRPMARAAGAARRPWPGPTTRHPPYDTRPRAPPGARPGLPGPRARPAPRLLGTAGDSPTATTRRTDWPGPAGRAPGRACTNPTARVTMIGAKPGRGGARPAPHAPHAHTPPEEPRRGRGGPAQRARERASRETPDSGEARAGPQGCPAETLGQKRPSWAATAPPNQPRSPHPRQGLSGGRQGADKPHSQGI in T59742_P5.

Domains affected by alternative splicing include the cytoplasmic N-terminus region of the protein; the S1 transmembrane domain of domain I and the cytoplasmic C-terminus region of the protein.

Effects of these changes are described with regard to Example 2 above.

From the above description of the effect of changes to the C-terminus domain of this variant, it is expected that the variant has a greater current amplitude as compared to the known protein and a stronger calcium dependence of inactivation.

Sequence name: CCAA_HUMAN

documentation:

of: T59742_P5 (resideues 24-2217 of SEQ ID NO: 13)×CCAA_HUMAN (SEQ ID NO: 91) . . .

segment 1/1:

Quality: 21614.00

Escore: 0

Matching length: 2194 Total

length: 2206

Matching Percent Similarity: 100.00 Matching Percent

Identity: 100.00

Total Percent Similarity: 99.46 Total Percent

Identity: 99.46

Gaps: 1

| | . . . . . | |
|---|---|---|
| 24 | TIIANCIVLALEQHLPDDDKTPMSERLDDTEPYFIGIFCFEAGIKIIALG | 73 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 107 | TIIANCIVLALEQHLPDDDKTPMSERLDDTEPYFIGIFCFEAGIKIIALG | 156 |
| | . . . . . | |
| 74 | FAFHKGSYLRNGWNVMDFVVVLTGILATVGTEFDLRTLRAVRVLRPLKLV | 123 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 157 | FAFHKGSYLRNGWNVMDFVVVLTGILATVGTEFDLRTLRAVRVLRPLKLV | 206 |
| | . . . . . | |
| 124 | SGIPSLQVVLKSIMKAMIPLLQIGLLLFFAILIFAIIGLEFYMGKFHTTC | 173 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 207 | SGIPSLQVVLKSIMKAMIPLLQIGLLLFFAILIFAIIGLEFYMGKFHTTC | 256 |
| | . . . . . | |
| 174 | FEEGTDDIQGESPAPCGTEEPARTCPNGTKCQPYWEGPNNGITQFDNILF | 223 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 257 | FEEGTDDIQGESPAPCGTEEPARTCPNGTKCQPYWEGPNNGITQFDNILF | 306 |
| | . . . . . | |
| 224 | AVLTVFQCITMEGWTDLLYNSNDASGNTWNWLYFIPLIIIGSFFMLNLVL | 273 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 307 | AVLTVFQCITMEGWTDLLYNSNDASGNTWNWLYFIPLIIIGSFFMLNLVL | 356 |
| | . . . . . | |
| 274 | GVLSGEFAKERERVENRRAFLKLRRQQQIERELNGYMEWISKAEEVILAE | 323 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 357 | GVLSGEFAKERERVENRRAFLKLRRQQQIERELNGYMEWISKAEEVILAE | 406 |
| | . . . . . | |
| 324 | DETDGEQRHPFDGALRRTTIKKSKTDLLNPEEAEDQLADIASVGSPFARA | 373 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 407 | DETDGEQRHPFDGALRRTTIKKSKTDLLNPEEAEDQLADIASVGSPFARA | 456 |
| | . . . . . | |
| 374 | SIKSAKLENSTFFHKKERRMRFYIRRMVKTQAFYWTVLSLVALNTLCVAI | 423 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 457 | SIKSAKLENSTFFHKKERRMRFYIRRMVKTQAFYWTVLSLVALNTLCVAI | 506 |
| | . . . . . | |
| 424 | VHYNQPEWLSDFLYYAEFIFLGLFMSEMFIKMYGLGTRPYFHSSFNCFDC | 473 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 507 | VHYNQPEWLSDFLYYAEFIFLGLFMSEMFIKMYGLGTRPYFHSSFNCFDC | 556 |
| | . . . . . | |
| 474 | GVIIGSIFEVIWAVIKPGTSFGISVLRALRLLRIFKVTKYWASLRNLVVS | 523 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 557 | GVIIGSIFEVIWAVIKPGTSFGISVLRALRLLRIFKVTKYWASLRNLVVS | 606 |
| | . . . . . | |
| 524 | LLNSMKSIISLLFLLFLFIVVFALLGMQLFGGQFNFDEGTPPTNFDTFPA | 573 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 607 | LLNSMKSIISLLFLLFLFIVVFALLGMQLFGGQFNFDEGTPPTNFDTFPA | 656 |
| | . . . . . | |
| 574 | AIMTVFQILTGEDWNEVMYDGIKSQGGVQGGMVFSIYFIVLTLFGNYTLL | 623 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 657 | AIMTVFQILTGEDWNEVMYDGIKSQGGVQGGMVFSIYFIVLTLFGNYTLL | 706 |
| | . . . . . | |
| 624 | NVFLAIAVDNLANAQELTKDEQEEEEAANQKLALQKAKEVAEVSPLSAAN | 673 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 707 | NVFLAIAVDNLANAQELTKDEQEEEEAANQKLALQKAKEVAEVSPLSAAN | 756 |
| | . . . . . | |
| 674 | MSIAVKEQQKNQKPAKSVWEQRTSEMRKQNLLASREALYNEMDPDERWKA | 723 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 757 | MSIAVKEQQKNQKPAKSVWEQRTSEMRKQNLLASREALYNEMDPDERWKA | 806 |
| | . . . . . | |
| 724 | AYTRHLRPDMKTHLDRPLVVDPQENRNNNTNKSRAAEPTVDQRLGQQRAE | 773 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 807 | AYTRHLRPDMKTHLDRPLVVDPQENRNNNTNKSRAAEPTVDQRLGQQRAE | 856 |
| | . . . . . | |
| 774 | DFLRKQARYHDRARDPSGSAGLDARRPWAGSQEAELSREGPYGRESDHHA | 823 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 857 | DFLRKQARYHDRARDPSGSAGLDARRPWAGSQEAELSREGPYGRESDHHA | 906 |
| | . . . . . | |
| 824 | REGSLEQPGFWEGEAERGKAGDPHRRHVHRQGGSRESRSGSPRTGADGEH | 873 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 907 | REGSLEQPGFWEGEAERGKAGDPHRRHVHRQGGSRESRSGSPRTGADGEH | 956 |
| | . . . . . | |
| 874 | RRHRAHRRPGEEGPEDKAERRARHREGSRPARGGEGEGEGPDGGERRRRH | 923 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 957 | RRHRAHRRPGEEGPEDKAERRARHREGSRPARGGEGEGEGPDGGERRRRH | 1006 |
| | . . . . . | |
| 924 | RHGAPATYEGDARREDKERRHRRRKENQGSGVPVSGPNLSTTRPIQQDLG | 973 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1007 | RHGAPATYEGDARREDKERRHRRRKENQGSGVPVSGPNLSTTRPIQQDLG | 1056 |
| | . . . . . | |
| 974 | RQDPPLAEDIDNMKNNKLATAESAAPHGSLGHAGLPQSPAKMGNSTDPGP | 1023 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1057 | RQDPPLAEDIDNMKNNKLATAESAAPHGSLGHAGLPQSPAKMGNSTDPGP | 1106 |
| | . . . . . | |
| 1024 | MLAIPAMATNPQNAASRRTPNNPGNPSNPGPPKTPENSLIVTNPSGTQTN | 1073 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1107 | MLAIPAMATNPQNAASRRTPNNPGNPSNPGPPKTPENSLIVTNPSGTQTN | 1156 |
| | . . . . . | |
| 1074 | SAKTARKPDHTTVDIPPACPPPLNHTVVQVNKNANPDPLPKKEEEKKEEE | 1123 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1157 | SAKTARKPDHTTVDLPPACPPPLNHTVVQVNKNANPDPLPKKEEEKKEEE | 1206 |
| | . . . . . | |
| 1124 | EDDRGEDGPKPMPPYSSMFILSTTNPLRRLCHYILNLRYFEMCILMVIAM | 1173 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1207 | EDDRGEDGPKPMPPYSSMFILSTTNPLRRLCHYILNLRYFEMCILMVIAM | 1256 |
| | . . . . . | |
| 1174 | SSIALAAEDPVQPNAPRNNVLRYFDYVFTGVFTFEMVIKMIDLGLVLHQG | 1223 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1257 | SSIALAAEDPVQPNAPRNNVLRYFDYVFTGVFTFEMVIKMIDLGLVLHQG | 1306 |
| | . . . . . | |
| 1224 | AYFRDLWNILDFIVVSGALVAFAFTGNSKGKDINTIKSLRVLRVLRPLKT | 1273 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1307 | AYFRDLWNILDFIVVSGALVAFAFTGNSKGKDINTIKSLRVLRVLRPLKT | 1356 |
| | . . . . . | |
| 1274 | IKRLPKLKAVFDCVVNSLKNVFNILIVYMLFMFIFAVVAVQLFKGKFFHC | 1323 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1357 | IKRLPKLKAVFDCVVNSLKNVFNILIVYMLFMFIFAVVAVQLFKGKFFHC | 1406 |
| | . . . . . | |
| 1324 | TDESKEFEKDCRGKYLLYEKNEVKARDREWKKYEFHYDNVLWALLTLFTV | 1373 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1407 | TDESKEFEKDCRGKYLLYEKNEVKARDREWKKYEFHYDNVLWALLTLFTV | 1456 |
| | . . . . . | |
| 1374 | STGEGWPQVLKHSVDATFENQGPSPGYRMEMSIFYVVYFVVFPFFFVNIF | 1423 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1457 | STGEGWPQVLKHSVDATFENQGPSPGYRMEMSIFYVVYFVVFPFFFVNIF | 1506 |
| | . . . . . | |
| 1424 | VALIIITFQEQGDKMMEEYSLEKNERACIDFAISAKPLTRHMPQNKQSFQ | 1473 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1507 | VALIIITFQEQGDKMMEEYSLEKNERACIDFAISAKPLTRHMPQNKQSFQ | 1556 |
| | . . . . . | |
| 1474 | YRMWQFVVSPPFEYTIMAMIALNTIVLMMKFYGASVAYENALRVFNIVFT | 1523 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1557 | YRMWQFVVSPPFEYTIMAMIALNTIVLMMKFYGASVAYENALRVFNIVFT | 1606 |
| | . . . . . | |
| 1524 | SLFSLECVLKVMAFGILNYFRDAWNIFDFVTVLGSITDILVTEFGNNFIN | 1573 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1607 | SLFSLECVLKVMAFGILNYFRDAWNIFDFVTVLGSITDILVTEFGNNFIN | 1656 |
| | . . . . . | |
| 1574 | LSFLRLFRAARLIKLLRQGYTIRILLWTFVQSFKALPYVCLLIAMLFFIY | 1623 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1657 | LSFLRLFRAARLIKLLRQGYTIRILLWTFVQSFKALPYVCLLIAMLFFIY | 1706 |
| | . . . . . | |
| 1624 | AIIGMQVFGNIGIDVEDEDSDEDEFQITEHNNFRTFFQALMLLFRSATGE | 1673 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1707 | AIIGMQVFGNIGIDVEDEDSDEDEFQITEHNNFRTFFQALMLLFRSATGE | 1756 |
| | . . . . . | |
| 1674 | AWHNIMLSCLSGKPCDKNSGILTRECGNEFAYFYFVSFIFLCSFLMLNLF | 1723 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1757 | AWHNIMLSCLSGKPCDKNSGILTRECGNEFAYFYFVSFIFLCSFLMLNLF | 1806 |
| | . . . . . | |
| 1724 | VAVIMDNFEYLTRDSSILGPHHLDEYVRVWAEYDPAAWGRMPYLDMYQML | 1773 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1807 | VAVIMDNFEYLTRDSSILGPHHLDEYVRVWAEYDPAAWGRMPYLDMYQML | 1856 |
| | . . . . . | |
| 1774 | RHMSPPLGLGKKCPARVAYKRLLRMDLPVADDNTVHFNSTLMALIRTALD | 1823 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1857 | RHMSPPLGLGKKCPARVAYKRLLRMDLPVADDNTVHFNSTLMALIRTALD | 1906 |
| | . . . . . | |
| 1824 | IKIAKGGADKQQMDAELRKEMMAIWPNLSQKTLDLLVTPHKSTDLTVGKI | 1873 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1907 | IKIAKGGADKQQMDAELRKEMMAIWPNLSQKTLDLLVTPHKSTDLTVGKI | 1956 |
| | . . . . . | |
| 1874 | YAAMMIMEYYRQSKAKKLQAMREEQDRTPLMFQRMEPPSPTQEGGPGQNA | 1923 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1957 | YAAMMIMEYYRQSKAKKLQAMREEQDRTPLMFQRMEPPSPTQEGGPGQNA | 2006 |
| | . . . . . | |
| 1924 | LPSTQLDPGGALMAHESGLKESPSWVTQRAQEMFQKTGTWSPEQGPPTDM | 1973 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2007 | LPSTQLDPGGALMAHESGLKESPSWVTQRAQEMFQKTGTWSPEQGPPTDM | 2056 |
| | . . . . . | |
| 1974 | PNSQPNSQSVEMREMGRDGYSDSEHYLPMEGQGRAASMPRLPAENQ.... | 2019 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2057 | PNSQPNSQSVEMREMGRDGYSDSEHYLPMEGQGRAASMPRLPAENQRRRG | 2106 |
| | . . . . . | |
| 2020 | ........TISDTSPMKRSASVLGPKARRLDDYSLERVPPEENQRHHQRR | 2061 |
| | \|\|\|\|\|\|\|\|\|\|\|\|\|\|\|\|\|\|\|\|\|\|\|\|\|\|\|\|\|\|\|\|\|\|\|\|\|\|\|\|\|\| | |
| 2107 | RPRGNNLSTISDTSPMKRSASVLGPKARRLDDYSLERVPPEENQRHHQRR | 2156 |
| | . . . . . | |
| 2062 | RDRSHRASERSLGRYTDVDTGLGTDLSMTTQSGDLPSKERDQERGRPKDR | 2111 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2157 | RDRSHRASERSLGRYTDVDTGLGTDLSMTTQSGDLPSKERDQERGRPKDR | 2206 |
| | . . . . . | |
| 2122 | KHRQHHHHHHHHHHPPPPDKDRYAQERPDHGRARARDQRWSRSPSEGREH | 2161 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2207 | KHRQHHHHHHHHHHPPPPDKDRYAQERPDHGRARARDQRWSRSPSEGREH | 2256 |
| | . . . . . | |
| 2162 | MAHRQGSSSVSGSPAPSTSGTSTPRRGRRQLPQTPSTPRPHVSYSPVIRK | 2211 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2257 | MAHRQGSSSVSGSPAPSTSGTSTPRRGRRQLPQTPSTPRPHVSYSPVIRK | 2306 |
| 2212 | AGGSGP | 2217 |
| | \|\|\|\|\|\| | |
| 2307 | AGGSGP | 2312 |

### EXAMPLE 7

This Example relates to the variant T59742_P9 (SEQ ID NO:15; the nucleic acid sequence is given by SEQ ID NO:37), which is a variant alpha 1 subunit according to the present invention, and more specifically, is a splice variant of the known protein CCAA_HUMAN. The known protein structure and function is described above in Example 2.

The structure of the splice variant according to the present invention features a unique head and a unique tail, as compared to the known protein sequence. An alignment is provided at the end of this section, while the comparison between the two sequences is described below.

According to preferred embodiments of the present invention, there is provided an isolated chimeric polypeptide encoding for T59742_P9, comprising a first amino acid sequence being at least 70%, optionally at least 80%, preferably at least 85%, more preferably at least 90% and most preferably at least 95% homologous to a polypeptide having the sequence (SEQ ID NO: 92) MSSWPTRPTKSTAPPRPGGKNGT corresponding to amino acids 1-23 of T59742_P9, a second amino acid sequence being at least 90% homologous to (SEQ ID NO: 93) TIIANCIVLALEQHLPDDDKTPMSERLDDTEPYFIGIFCFEAGIKIIALGFAFHKGSYLRNGWNVMDFVVVLTGILATVGTEFDLRTLRAVRVLRPLKLVSGIPSLQVVLKSIMKAMIPLLQIGLLLFFAILIFAIIGLEFYMGKFHTTCFEEGTDDIQGESPAPCGTEEPARTCPNGTKCQPYWEGPNNGITQFDNILFAVLTVFQCITMEGWTDLLYNSNDASGNTWNWLYFIPLIIIGSFFMLNLVLGVLSGEFAKERERVENRRAFLKLRRQQQIERELNGYMEWISKAEEVILAEDETDGEQRHPFDGALRRTTIKKSKTDLLNPEEAEDQLADIASVGSPFARASIKSAKLENSTFFHKKERRMRFYIRRMVKTQAFYWTVLSLVALNTLCVAIVHYNQPEWLSDFLYYAEFIFLGLFMSEMFIKMYGLGTRPYFHSSFNCFDCGVIIGSIFEVIWAVIKPGTSFGISVLRALRLLRIFKVTKYWASLRNLVVSLLNSMKSIISLLFLLFLFIVVFALLGMQLFGGQFNFDEGTPPTNFDTFPAAIMTVFQILTGEDWNEVMYDGIKSQGGVQGGMVFSIYFIVLTLFGNYTLLNVFLAIAVDNLANAQELTKDEQEEEEAANQKLALQKAKEVAEVSPLSAANMSIAVKEQQKNQKPAKSVWEQRTSEMRKQNLLASREALYNEMDPDERWKAAYTRHLRPDMKTHLDRPLVVDPQENRNNNTNKSRAAEPTVDQRLGQQRAEDFLRKQARYHDRARDPSGSAGLDARRPWAGSQEAELSREGPYGRESDHHAREGSLEQPGFWEGEAERGKAGDPHRRHVHRQGGSRESRSGSPRTGADGEHRRHRAHRRPGEEGPEDKAERRARHREGSRPARGGEGEGEGPDGGERRRRHRHGAPATYEGDARREDKERRHRRRKENQGSGVPVSGPNLSTTRPIQQDLGRQDPPLAEDIDNMKNNKLATAESAAPHGSLGHAGLPQSPAKMGNSTDPGPMLAIPAMATNPQNAASRRTPNNPGNPSNPGPPKTPENSLIVTNPSGTQTNSAKTARKPDHTTVDIPPACPPPLNHTVVQVNKNANPDPLPKKEEEKKEEEEDDRGEDGPKPMPPYSSMFILSTTNPLRRLCHYILNLRYFEMCILMVIAMSSIALAAEDPVQPNAPRNNVLRYFDYVFTGVFTFEMVIKMIDLGLVLHQGAYFRDLWNILDFIVVSGALVAFAFTGNSKGKDINTIKSLRVLRVLRPLKTIKRLPKLKAVFDCVVNSLKNVFNILIVYMLFMFIFAVVAVQLFKGKFFHCTDESKEFEKDCRGKYLLYEKNEVKARDREWKKYEFHYDNVLWALLTLFTVSTGEGWPQVLKHSVDATFENQGPSPGYRMEMSIFYVVYFVVFPFFFVNIFVALIIITFQEQGDKMMEEYSLEKNERACIDFAISAKPLTRHMPQNKQSFQYRMWQFVVSPPFEYTIMAMIALNTIVLMMKFYGASVAYENALRVFNIVFTSLFSLECVLKVMAFGILNYFRDAWNIFDFVTVLGSITDILVTEFGNNFINLSFLRLFRAARLIKLLRQGYTIRILLWTFVQSFK corresponding to amino acids 107-1690 of CCAA_HUMAN, which also corresponds to amino acids 24-1607 of T59742_P9, and a third amino acid sequence being at least 70%, optionally at least 80%, preferably at least 85%, more preferably at least 90% and most preferably at least 95% homologous to a polypeptide having the sequence (SEQ ID NO: 94) CPTPTTRHTHTHTRSQTHTRVLAGYTKPYYYCLQKSI corresponding to amino acids 1608-1644 of T59742_P9, wherein said first amino acid sequence, second amino acid sequence and third amino acid sequence are contiguous and in a sequential order.

According to preferred embodiments of the present invention, there is provided an isolated polypeptide encoding for a head of T59742_P9, comprising a polypeptide being at least 70%, optionally at least about 80%, preferably at least about 85%, more preferably at least about 90% and most preferably at least about 95% homologous to the sequence (SEQ ID NO: 95) MSSWPTRPTKSTAPPRPGGKNGT of T59742_P9.

According to preferred embodiments of the present invention, there is provided an isolated polypeptide encoding for a tail of T59742_P9, comprising a polypeptide being at least 70%, optionally at least about 80%, preferably at least about 85%, more preferably at least about 90% and most preferably at least about 95% homologous to the sequence (SEQ ID NO: 96) CPTPTTRHTHTHTRSQTHTRVLAGYTKPYYYCLQKSI in T59742_P9.

Domains affected by alternative splicing include the cytoplasmic N-terminus region of the protein; the S1 transmembrane domain of domain I; the S5 transmembrane domain of domain IV; the pore loop region between S5 and S6 transmembrane domains of domain IV; the S6 transmembrane domain of domain IV; the cytoplasmic C-terminus region of the protein.

The effect of these changes is described with regard to Examples 2-6 above.

From the above description of the effect of changes to the C-terminus domain of this variant, it is expected that the variant has greater current amplitude as compared to the known protein and a stronger calcium dependence of inactivation.

Sequence name: CCAA_HUMAN

documentation:

of: T59742_P9 (residues 24-1607 of SEQ ID NO: 15)×CCAA_HUMAN (SEQ ID NO: 97). . .

segment 1/1:

Quality: `15581.00

Escore:

Matching length: 1584 Total

length: 1584

Matching Percent Similaritya: 100.00 Matching Percent

Identity: 100.00

Total Percent Similarityu: 100.00 Total Percent

Identity; 100.00

Gaps: 0

| | . . . . . | |
|---|---|---|
| 24 | TIIANCIVLALEQHLPDDDKTPMSERLDDTEPYFIGIFCFEAGIKIIALG | 73 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 107 | TIIANCIVLALEQHLPDDDKTPMSERLDDTEPYFIGIFCFEAGIKIIALG | 156 |
| | . . . . . | |
| 74 | FAFHKGSYLRNGWNVMDFVVVLTGILATVGTEFDLRTLRAVRVLRPLKLV | 123 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 157 | FAFHKGSYLRNGWNVMDFVVVLTGILATVGTEFDLRTLRAVRVLRPLKLV | 206 |
| | . . . . . | |
| 124 | SGIPSLQVVLKSIMKAMIPLLQIGLLLFFAILIFAIIGLEFYMGKFHTTC | 173 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 207 | SGIPSLQVVLKSIMKAMIPLLQIGLLLFFAILIFAIIGLEFYMGKFHTTC | 256 |
| | . . . . . | |
| 174 | FEEGTDDIQGESPAPCGTEEPARTCPNGTKCQPYWEGPNNGITQFDNILF | 223 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 257 | FEEGTDDIQGESPAPCGTEEPARTCPNGTKCQPYWEGPNNGITQFDNILF | 306 |
| | . . . . . | |
| 224 | AVLTVFQCITMEGWTDLLYNSNDASGNTWNWLYFIPLIIIGSFFMLNLVL | 273 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 307 | AVLTVFQCITMEGWTDLLYNSNDASGNTWNWLYFIPLIIIGSFFMLNLVL | 356 |
| | . . . . . | |
| 274 | GVLSGEFAKERERVENRRAFLKLRRQQQIERELNGYMEWISKAEEVILAE | 323 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 357 | GVLSGEFAKERERVENRRAFLKLRRQQQIERELNGYMEWISKAEEVILAE | 406 |
| | . . . . . | |
| 324 | DETDGEQRHPFDGALRRTTIKKSKTDLLNPEEAEDQLADIASVGSPFARA | 373 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 407 | DETDGEQRHPFDGALRRTTIKKSKTDLLNPEEAEDQLADIASVGSPFARA | 456 |
| | . . . . . | |
| 374 | SIKSAKLENSTFFHKKERRMRFYIRRMVKTQAFYWTVLSLVALNTLCVAI | 423 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 457 | SIKSAKLENSTFFHKKERRMRFYIRRMVKTQAFYWTVLSLVALNTLCVAI | 506 |
| | . . . . . | |
| 424 | VHYNQPEWLSDFLYYAEFIFLGLFMSEMFIKMYGLGTRPYFHSSFNCFDC | 473 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 507 | VHYNQPEWLSDFLYYAEFIFLGLFMSEMFIKMYGLGTRPYFHSSFNCFDC | 556 |
| | . . . . . | |
| 474 | GVIIGSIFEVIWAVIKPGTSFGISVLRALRLLRIFKVTKYWASLRNLVVS | 523 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 557 | GVIIGSIFEVIWAVIKPGTSFGISVLRALRLLRIFKVTKYWASLRNLVVS | 606 |
| | . . . . . | |
| 524 | LLNSMKSIISLLFLLFLFIVVFALLGMQLFGGQFNFDEGTPPTNFDTFPA | 573 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 607 | LLNSMKSIISLLFLLFLFIVVFALLGMQLFGGQFNFDEGTPPTNFDTFPA | 656 |
| | . . . . . | |
| 574 | AIMTVFQILTGEDWNEVMYDGIKSQGGVQGGMVFSIYFIVLTLFGNYTLL | 623 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 657 | AIMTVFQILTGEDWNEVMYDGIKSQGGVQGGMVFSIYFIVLTLFGNYTLL | 706 |
| | . . . . . | |
| 624 | NVFLAIAVDNLANAQELTKDEQEEEEAANQKLALQKAKEVAEVSPLSAAN | 673 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 707 | NVFLAIAVDNLANAQELTKDEQEEEEAANQKLALQKAKEVAEVSPLSAAN | 756 |
| | . . . . . | |
| 674 | MSIAVKEQQKNQKPAKSVWEQRTSEMRKQNLLASREALYNEMDPDERWKA | 723 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 757 | MSIAVKEQQKNQKPAKSVWEQRTSEMRKQNLLASREALYNEMDPDERWKA | 806 |
| | . . . . . | |
| 724 | AYTRHLRPDMKTHLDRPLVVDPQENRNNNTNKSRAAEPTVDQRLGQQRAE | 773 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 807 | AYTRHLRPDMKTHLDRPLVVDPQENRNNNTNKSRAAEPTVDQRLGQQRAE | 856 |
| | . . . . . | |
| 774 | DFLRKQARYHDRARDPSGSAGLDARRPWAGSQEAELSREGPYGRESDHHA | 823 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 857 | DFLRKQARYHDRARDPSGSAGLDARRPWAGSQEAELSREGPYGRESDHHA | 906 |
| | . . . . . | |
| 824 | REGSLEQPGFWEGEAERGKAGDPHRRHVHRQGGSRESRSGSPRTGADGEH | 873 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 907 | REGSLEQPGFWEGEAERGKAGDPHRRHVHRQGGSRESRSGSPRTGADGEH | 956 |
| | . . . . . | |
| 874 | RRHRAHRRPGEEGPEDKAERRARHREGSRPARGGEGEGEGPDGGERRRRH | 923 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 957 | RRHRAHRRPGEEGPEDKAERRARHREGSRPARGGEGEGEGPDGGERRRRH | 1006 |
| | . . . . . | |
| 924 | RHGAPATYEGDARREDKERRHRRRKENQGSGVPVSGPNLSTTRPIQQDLG | 973 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1007 | RHGAPATYEGDARREDKERRHRRRKENQGSGVPVSGPNLSTTRPIQQDLG | 1056 |
| | . . . . . | |
| 974 | RQDPPLAEDIDNMKNNKLATAESAAPHGSLGHAGLPQSPAKMGNSTDPGP | 1023 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1057 | RQDPPLAEDIDNMKNNKLATAESAAPHGSLGHAGLPQSPAKMGNSTDPGP | 1106 |
| | . . . . . | |
| 1024 | MLAIPAMATNPQNAASRRTPNNPGNPSNPGPPKTPENSLIVTNPSGTQTN | 1073 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1107 | MLAIPAMATNPQNAASRRTPNNPGNPSNPGPPKTPENSLIVTNPSGTQTN | 1156 |
| | . . . . . | |
| 1074 | SAKTARKPDHTTVDIPPACPPPLNHTVVQVNKNANPDPLPKKEEEKKEEE | 1123 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1157 | SAKTARKPDHTTVDIPPACPPPLNHTVVQVNKNANPDPLPKKEEEKKEEE | 1206 |
| | . . . . . | |
| 1124 | EDDRGEDGPKPMPPYSSMFILSTTNPLRRLCHYILNLRYFEMCILMVIAM | 1173 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1207 | EDDRGEDGPKPMPPYSSMFILSTTNPLRRLCHYILNLRYFEMCILMVIAM | 1256 |
| | . . . . . | |
| 1174 | SSIALAAEDPVQPNAPRNNVLRYFDYVFTGVFTFEMVIKMIDLGLVLHQG | 1223 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1257 | SSIALAAEDPVQPNAPRNNVLRYFDYVFTGVFTFEMVIKMIDLGLVLHQG | 1306 |
| | . . . . . | |
| 1224 | AYFRDLWNILDFIVVSGALVAFAFTGNSKGKDINTIKSLRVLRVLRPLKT | 1273 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1307 | AYFRDLWNILDFIVVSGALVAFAFTGNSKGKDINTIKSLRVLRVLRPLKT | 1356 |
| | . . . . . | |
| 1274 | IKRLPKLKAVFDCVVNSLKNVFNILIVYMLFMFIFAVVAVQLFKGKFFHC | 1323 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1357 | IKRLPKLKAVFDCVVNSLKNVFNILIVYMLFMFIFAVVAVQLFKGKFFHC | 1406 |
| | . . . . . | |
| 1324 | TDESKEFEKDCRGKYLLYEKNEVKARDREWKKYEFHYDNVLWALLTLFTV | 1373 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1407 | TDESKEFEKDCRGKYLLYEKNEVKARDREWKKYEFHYDNVLWALLTLFTV | 1456 |
| | . . . . . | |
| 1374 | STGEGWPQVLKHSVDATFENQGPSPGYRMEMSIFYVVYFVVFPFFFVNIF | 1423 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1457 | STGEGWPQVLKHSVDATFENQGPSPGYRMEMSIFYVVYFVVFPFFFVNIF | 1506 |
| | . . . . . | |
| 1424 | VALIIITFQEQGDKMMEEYSLEKNERACIDFAISAKPLTRHMPQNKQSFQ | 1473 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1507 | VALIIITFQEQGDKMMEEYSLEKNERACIDFAISAKPLTRHMPQNKQSFQ | 1556 |
| | . . . . . | |
| 1474 | YRMWQFVVSPPFEYTIMAMIALNTIVLMMKFYGASVAYENALRVFNIVFT | 1523 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1557 | YRMWQFVVSPPFEYTIMAMIALNTIVLMMKFYGASVAYENALRVFNIVFT | 1606 |
| | . . . . . | |
| 1524 | SLFSLECVLKVMAFGILNYFRDAWNIFDFVTVLGSITDILVTEFGNNFIN | 1573 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1607 | SLFSLECVLKVMAFGILNYFRDAWNIFDFVTVLGSITDILVTEFGNNFIN | 1656 |
| | . . . | |
| 1574 | LSFLRLFRAARLIKLLRQGYTIRILLWTFVQSFK | 1607 |
| | \|\|\|\|\|\|\|\|\|\|\|\|\|\|\|\|\|\|\|\|\|\|\|\|\|\|\|\|\|\|\|\|\|\| | |
| 1657 | LSFLRLFRAARLIKLLRQGYTIRILLWTFVQSFK | 1690 |

### EXAMPLE 8

This Example relates to the variant AA019974_P4 (SEQ ID NO:17; the nucleic acid sequence is given by SEQ ID NO:39), which is a variant alpha 1 subunit according to the present invention, and more specifically, is a splice variant of the known protein CCAF_HUMAN, which is encoded by the CACNA1F gene. This protein is an alpha 1F subunit, which forms a voltage-dependent L-type calcium channel. The subunit is also known as an alpha subunit Cav1.4.

The isoform alpha-1F is found in L-type calcium channels, which belong to the “high-voltage activated” (HVA) group. They are blocked by dihydropyridines (DHP), phenylalkylamines, benzothiazepines, and by omega-agatoxin-IIIA (omega-Aga-IIIA), but are insensitive to certain other toxins.

This particular alpha subunit is typically found in skeletal muscle and retina. It has the following structure: each of the four internal repeats contains five hydrophobic transmembrane segments (S1, S2, S3, S5, S6) and one positively charged transmembrane segment (S4). S4 segments probably represent the voltage-sensor and are characterized by a series of positively charged amino acids at every third position.

Defects in the CACNA1F gene are the cause of incomplete X-linked congenital stationary night blindness type 2 (CSNB2). CSNB2 is a nonprogressive retinal disorder characterized by decreased visual acuity and loss of night vision.

The structure of the splice variant according to the present invention features a unique insertion and a unique tail, as compared to the known protein sequence. An alignment is provided at the end of this section, while the comparison between the two sequences is described below.

According to preferred embodiments of the present invention, there is provided an isolated chimeric polypeptide encoding for AA019974_P4, comprising a first amino acid sequence being at least 90% homologous to (SEQ ID NO: 98) MSESEGGKDTTPEPSPANGAGPGPEWGLCPGPPAVEGESSGASGLGTPKRRNQHSKHKTVAVASAQRSPRALFCLTLANPLRRSCISIVEWKPFDILILLTIFANCVALGVYIPFPEDDSNTANHNLEQVEYVFLVIFTVETVLKIVAYGLVLHPSAYIRNGWNLLDFIIVVVGLFSVLLEQGPGRPGDAPHTGGKPGGFDVKALRAFRVLRPLRLVSGVPSLHIVLNSIMKALVPLLHIALLVLFVIIIYAIIGLELFLGRMHKTCYFLGSDMEAEEDPSPCASSGSGRACTLNQTECRGRWPGPNGGITNFDNFFFAMLTVFQCVTMEGWTDVLYWMQDAMGYELPWVYFVSLVIFGSFFVLNLVLGVLSGEFSKEREKAKARGDFQKQREKQQMEEDLRGYLDWITQAEELDMEDPSADDNLG corresponding to amino acids 1-426 of CCAF_HUMAN, which also corresponds to amino acids 1-426 of AA019974_P4, a second amino acid sequence being at least 70%, optionally at least 80%, preferably at least 85%, more preferably at least 90% and most preferably at least 95% homologous to a polypeptide having the sequence (SEQ ID NO: 99) SMAEEGRAGHR corresponding to amino acids 427-437 of AA019974_P4, a third amino acid sequence being at least 90% homologous to (SEQ ID NO: 100) PQLAELTNRRRGRLRWFSHSTRSTHSTSSHASLPASDTGSMTETQGDEDEEEGALASCTRCLNKIMKTRVCRRLRRANRVLRARCRRAVKSNACYWAVLLLVFLNTLTIASEHHGQPVWLTQIQEYANKVLLCLFTVEMLLKLYGLGPSAYVSSFFNRFDCFVVCGGILETTLVEVGAMQPLGISVLRCVRLLRIFKVTRHWASLSNLVASLLNSMKSIASLLLLLFLFIIIFSLLGMQLFGGKFNFDQTHTKRSTFDTFPQALLTVFQILTGEDWNVVMYDGIMAYGGPFFPGMLVCIYFIILFICGNYILLNVFLAIAVDNLASGDAGTAKDKGGEKSNEKDLPQENEGLVPGVEKEEEEGARREGADMEEEEEEEEEEEEEEEEEGAGGVELLQEVVPKEKVVPIPEGSAFFCLSQTNPLRKGCHTLIHHHVFTNLILVFIILSSVSLAAEDPIRAHSFRNHILGYFDYAFTSIFTVEILLKMTVFGAFLHRGSFCRSWFNMLDLLVVSVSLISFGIHSSAISVVKILRVLRVLRPLRAINRAKGLKHVVQCVFVAIRTIGNIMIVTTLLQFMFACIGVQLFKGKFYTCTDEAKHTPQECKGSFLVYPDGDVSRPLVRERLWVNSDFNFDNVLSAMMALFTVSTFEGWPALLYKAIDAYAEDHGPIYNYRVEISVFFIVYIIIIAFFMMNIFVGFVIITFRAQGEQEYQNCELDKNQRQCVEYALKAQPLRRYIPKNPHQYRVWATVNSAAFEYLMFLLILLNTVALAMQHYEQTAPFNYAMDILNMVFTGLFTIEMVLKIIAFKPKHYFTDAWNTFDALIVVGSIVDIAVTEVNNGGHLGESSEDSSRISITFFRLFRVMRLVKLLSKGEGIRTLLWTFIKSFQALPYVALLIAMIFFIYAVIGMQMFGKVALQDGTQINRNNNFQTFPQAVLLLFRCATGEAWQEIMLASLPGNRCDPESDFGPGEEFTCGSNFAIAYFISFFMLCAFLIINLFVAVIMDNFDYLTRDWSILGPHHLDEFKRIWSEYDPGAK corresponding to amino acids 427-1463 of CCAF_HUMAN, which also corresponds to amino acids 438-1474 of AA019974_P4, and a fourth amino acid sequence being at least 70%, optionally at least 80%, preferably at least 85%, more preferably at least 90% and most preferably at least 95% homologous to a polypeptide having the sequence (SEQ ID NO: 101) YALYTLT corresponding to amino acids 1475-1481 of AA019974_P4, wherein said first amino acid sequence, second amino acid sequence, third amino acid sequence and fourth amino acid sequence are contiguous and in a sequential order.

According to preferred embodiments of the present invention, there is provided an isolated polypeptide encoding for an edge portion (unique insertion) of AA019974_P4, comprising an amino acid sequence being at least 70%, optionally at least about 80%, preferably at least about 85%, more preferably at least about 90% and most preferably at least about 95% homologous to the sequence encoding for (SEQ ID NO: 102) SMAEEGRAGHR, corresponding to AA019974_P4.

According to preferred embodiments of the present invention, there is provided an isolated polypeptide encoding for a tail of AA019974_P4, comprising a polypeptide being at least 70%, optionally at least about 80%, preferably at least about 85%, more preferably at least about 90% and most preferably at least about 95% homologous to the sequence (SEQ ID NO: 103) YALYTLT in AA019974_P4.

Domains affected by alternative splicing include the cytoplasmic loop between domain I and domain II, and the cytoplasmic C-terminus region of the protein.

This loop is an important point of interaction of G-beta-gamma with Ca⁺² channels, and this interaction may determine G protein specificity for modulation. The effect of these changes on the C-terminus region may be found with regard to Example 2.

From the above description of the effect of changes to the C-terminus domain of this variant, it is expected that the variant has an increase of current amplitude without change of voltage dependence of gating. It is also expected to modulate the Ca⁺²-dependent inactivation of the channel.

In addition, the changes in the cytoplasmic loop between domain I and domain II may result in changes in the G protein specificity for modulation.

Sequence name: CCAF_HUMAN

docuemtation:

of: AA019974_P4 (residues 1-1474 of SEQ ID NO: 17)×CCAF_HUMAN (SEQ ID NO: 104) . . .

segment 1/1:

Quality: 14184.00

Escore: 0

Matching length: 1463 Total

length: 1474

Matching Percent Similarity: 100.00 Matching Percent

Identity: 100.00

Total Percent Similarity: 99.25 Total Percent

Identity: 99.25

Gaps: 1

| | . . . . . | |
|---|---|---|
| 1 | MSESEGGKDTTPEPSPANGAGPGPEWGLCPGPPAVEGESSGASGLGTPKR | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MSESEGGKDTTPEPSPANGAGPGPEWGLCPGPPAVEGESSGASGLGTPKR | 50 |
| | . . . . . | |
| 51 | RNQHSKHKTVAVASAQRSPRALFCLTLANPLRRSCISIVEWKPFDILILL | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | RNQHSKHKTVAVASAQRSPRALFCLTLANPLRRSCISIVEWKPFDILILL | 100 |
| | . . . . . | |
| 101 | TIFANCVALGVYIPFPEDDSNTANHNLEQVEYVFLVIFTVETVLKIVAYG | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | TIFANCVALGVYIPFPEDDSNTANHNLEQVEYVFLVIFTVETVLKIVAYG | 150 |
| | . . . . . | |
| 151 | LVLHPSAYIRNGWNLLDFIIVVVGLFSVLLEQGPGRPGDAPHTGGKPGGF | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | LVLHPSAYIRNGWNLLDFIIVVVGLFSVLLEQGPGRPGDAPHTGGKPGGF | 200 |
| | . . . . . | |
| 201 | DVKALRAFRVLRPLRLVSGVPSLHIVLNSIMKALVPLLHIALLVLFVIII | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | DVKALRAFRVLRPLRLVSGVPSLHIVLNSIMKALVPLLHIALLVLFVIII | 250 |
| | . . . . . | |
| 251 | YAIIGLELFLGRMHKTCYFLGSDMEAEEDPSPCASSGSGRACTLNQTECR | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | YAIIGLELFLGRMHKTCYFLGSDMEAEEDPSPCASSGSGRACTLNQTECR | 300 |
| | . . . . . | |
| 301 | GRWPGPNGGITNFDNFFFAMLTVFQCVTMEGWTDVLYWMQDAMGYELPWV | 350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | GRWPGPNGGITNFDNFFFAMLTVFQCVTMEGWTDVLYWMQDAMGYELPWV | 350 |
| | . . . . . | |
| 351 | YFVSLVIFGSFFVLNLVLGVLSGEFSKEREKAKARGDFQKQREKQQMEED | 400 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 351 | YFVSLVIFGSFFVLNLVLGVLSGEFSKEREKAKARGDFQKQREKQQMEED | 400 |
| | . . . . . | |
| 401 | LRGYLDWITQAEELDMEDPSADDNLGSMAEEGRAGHRPQLAELTNRRRGR | 450 |
| | \|\|\|\|\|\|\|\|\|\|\|\|\|\|\|\|\|\|\|\|\|\|\|\|\|\| \|\|\|\|\|\|\|\|\|\|\|\|\| | |
| 401 | LRGYLDWITQAEELDMEDPSADDNLG...........PQLAELTNRRRGR | 439 |
| | . . . . . | |
| 451 | LRWFSHSTRSTHSTSSHASLPASDTGSMTETQGDEDEEEGALASCTRCLN | 500 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 440 | LRWFSHSTRSTHSTSSHASLPASDTGSMTETQGDEDEEEGALASCTRCLN | 489 |
| | . . . . . | |
| 501 | KIMKTRVCRRLRRANRVLRARCRRAVKSNACYWAVLLLVFLNTLTIASEH | 550 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 490 | KIMKTRVCRRLRRANRVLRARCRRAVKSNACYWAVLLLVFLNTLTIASEH | 539 |
| | . . . . . | |
| 551 | HGQPVWLTQIQEYANKVLLCLFTVEMLLKLYGLGPSAYVSSFFNRFDCFV | 600 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 540 | HGQPVWLTQIQEYANKVLLCLFTVEMLLKLYGLGPSAYVSSFFNRFDCFV | 589 |
| | . . . . . | |
| 601 | VCGGILETTLVEVGAMQPLGISVLRCVRLLRIFKVTRHWASLSNLVASLL | 650 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 590 | VCGGILETTLVEVGAMQPLGISVLRCVRLLRIFKVTRHWASLSNLVASLL | 639 |
| | . . . . . | |
| 651 | NSMKSIASLLLLLFLFIIIFSLLGMQLFGGKFNFDQTHTKRSTFDTFPQA | 700 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 640 | NSMKSIASLLLLLFLFIIIFSLLGMQLFGGKFNFDQTHTKRSTFDTFPQA | 689 |
| | . . . . . | |
| 701 | LLTVFQILTGEDWNVVMYDGIMAYGGPFFPGMLVCIYFIILFICGNYILL | 750 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 690 | LLTVFQILTGEDWNVVMYDGIMAYGGPFFPGMLVCIYFIILFICGNYILL | 739 |
| | . . . . . | |
| 751 | NVFLAIAVDNLASGDAGTAKDKGGEKSNEKDLPQENEGLVPGVEKEEEEG | 800 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 740 | NVFLAIAVDNLASGDAGTAKDKGGEKSNEKDLPQENEGLVPGVEKEEEEG | 789 |
| | . . . . . | |
| 801 | ARREGADMEEEEEEEEEEEEEEEEEGAGGVELLQEVVPKEKVVPIPEGSA | 850 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 790 | ARREGADMEEEEEEEEEEEEEEEEEGAGGVELLQEVVPKEKVVPIPEGSA | 839 |
| | . . . . . | |
| 851 | FFCLSQTNPLRKGCHTLIHHHVFTNLILVFIILSSVSLAAEDPIRAHSFR | 900 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 840 | FFCLSQTNPLRKGCHTLIHHHVFTNLILVFIILSSVSLAAEDPIRAHSFR | 889 |
| | . . . . . | |
| 901 | NHILGYFDYAFTSIFTVEILLKMTVFGAFLHRGSFCRSWFNMLDLLVVSV | 950 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 890 | NHILGYFDYAFTSIFTVEILLKMTVFGAFLHRGSFCRSWFNMLDLLVVSV | 939 |
| | . . . . . | |
| 951 | SLISFGIHSSAISVVKILRVLRVLRPLRAINRAKGLKHVVQCVFVAIRTI | 1000 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 940 | SLISFGIHSSAISVVKILRVLRVLRPLRAINRAKGLKHVVQCVFVAIRTI | 989 |
| | . . . . . | |
| 1001 | GNIMIVTTLLQFMFACIGVQLFKGKFYTCTDEAKHTPQECKGSFLVYPDG | 1050 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 990 | GNIMIVTTLLQFMFACIGVQLFKGKFYTCTDEAKHTPQECKGSFLVYPDG | 1039 |
| | . . . . . | |
| 1051 | DVSRPLVRERLWVNSDFNFDNVLSAMMALFTVSTFEGWPALLYKAIDAYA | 1100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1040 | DVSRPLVRERLWVNSDFNFDNVLSAMMALFTVSTFEGWPALLYKAIDAYA | 1089 |
| | . . . . . | |
| 1101 | EDHGPIYNYRVEISVFFIVYIIIIAFFMMNIFVGFVIITFRAQGEQEYQN | 1150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1090 | EDHGPIYNYRVEISVFFIVYIIIIAFFMMNIFVGFVIITFRAQGEQEYQN | 1139 |
| | . . . . . | |
| 1151 | CELDKNQRQCVEYALKAQPLRRYIPKNPHQYRVWATVNSAAFEYLMFLLI | 1200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1140 | CELDKNQRQCVEYALKAQPLRRYIPKNPHQYRVWATVNSAAFEYLMFLLI | 1189 |
| | . . . . . | |
| 1201 | LLNTVALAMQHYEQTAPFNYAMDILNMVFTGLFTIEMVLKIIAFKPKHYF | 1250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1190 | LLNTVALAMQHYEQTAPFNYAMDILNMVFTGLFTIEMVLKIIAFKPKHYF | 1239 |
| | . . . . . | |
| 1251 | TDAWNTFDALIVVGSIVDIAVTEVNNGGHLGESSEDSSRISITFFRLFRV | 1300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1240 | TDAWNTFDALIVVGSIVDIAVTEVNNGGHLGESSEDSSRISITFERLFRV | 1289 |
| | . . . . . | |
| 1301 | MRLVKLLSKGEGIRTLLWTFIKSFQALPYVALLIAMIFFIYAVIGMQMFG | 1350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1290 | MRLVKLLSKGEGIRTLLWTFIKSFQALPYVALLIAMIFFIYAVIGMQMFG | 1339 |
| | . . . . . | |
| 1351 | KVALQDGTQINRNNNFQTFPQAVLLLFRCATGEAWQEIMLASLPGNRCDP | 1400 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1340 | KVALQDGTQINRNNNFQTFPQAVLLLFRCATGEAWQEIMLASLPGNRCDP | 1389 |
| | . . . . . | |
| 1401 | ESDFGPGEEFTCGSNFAIAYFISFFMLCAFLIINLFVAVIMDNFDYLTRD | 1450 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1390 | ESDFGPGEEFTCGSNFAIAYFISFFMLCAFLIINLFVAVIMDNFDYLTRD | 1439 |
| | . . | |
| 1451 | WSILGPHHLDEEKRIWSEYDPGAK | 1474 |
| | \|\|\|\|\|\|\|\|\|\|\|\|\|\|\|\|\|\|\|\|\|\|\|\| | |
| 1440 | WSILGPHHLDEFKRIWSEYDPGAK | 1463 |

### EXAMPLE 9

This Example relates to the variant R12947_P13 (SEQ ID NO:18; the nucleic acid sequence is given by SEQ ID NO:40), which is a variant alpha 1 subunit according to the present invention, and more specifically, is a splice variant of the known protein CCAG_HUMAN, which is encoded by the CACNA1G gene. This protein is the alpha 1G subunit and results in a voltage-dependent T-type calcium channel. The subunit is also known as alpha subunit Cav3.1.

T-type calcium channels belong to the “low-voltage activated (LVA)” group and are strongly blocked by mibefradil, as described above. These channels are characterized by voltage-dependent inactivation. T-type channels serve pacemaking functions in both central neurons and cardiac nodal cells and support calcium signaling in secretory cells and vascular smooth muscle, as previously described.

This alpha subunit is highly expressed in brain, in particular in the amygdala, subthalamic nuclei, cerebellum and thalamus, where it may be involved in neuronal processing. It has moderate but highly localized expression in heart, with low expression in placenta, kidney and lung.

This alpha subunit has the following structure. Each of the four internal repeats contains five hydrophobic transmembrane segments (S1, S2, S3, S5, S6) and one positively charged transmembrane segment (S4). S4 segments probably represent the voltage-sensor and are characterized by a series of positively charged amino acids at every third position. The linker region between repeat III and IV probably play a role in the inactivation of the channel. The C-terminal part may be implicated in the anchoring of the protein to the membrane.

In response to an increase in the level of intracellular calcium, the T-type channels are activated by CaM-kinase II.

The structure of the splice variant according to the present invention features a unique insertion and a skipped exon, as compared to the known protein sequence. An alignment is provided at the end of this section, while the comparison between the two sequences is described below.

According to preferred embodiments of the present invention, there is provided an isolated chimeric polypeptide encoding for R12947_P13, comprising a first amino acid sequence being at least 90% homologous to (SEQ ID NO: 105) MDEEEDGAGAEESGQPRSFMRLNDLSGAGGRPGPGSAEKDPGSADSEAEGLPYPALAPVVFFYLSQDSRPRSWCLRTVCNPWFERISMLVILLNCVTLGMFRPCEDIACDSQRCRILQAFDDFIFAFFAVEMVVKMVALGIFGKKCYLGDTWNRLDFFIVIAGMLEYSLDLQNVSFSAVRTVRVLRPLRAINRVPSMRILVTLLLDTLPMLGNVLLLCFFVFFIFGIVGVQLWAGLLRNRCFLPENFSLPLSVDLERYYQTENEDESPFICSQPRENGMRSCRSVPTLRGDGGGGPPCGLDYEAYNSSSNTTCVNWNQYYTNCSAGEHNPFKGAINFDNIGYAWIAIFQVITLEGWVDIMYFVMDAHSFYNFIYFILLIIVGSFFMINLCLVVIATQFSETKQRESQLMREQRVRFLSNASTLASFSEPGSCYEELLKYLVYILRKAARRLAQVSRAAGVRVGLLSSPAPLGGQETQPSSSCSRSHRRLSVHHLVHHHHHHHHHYHLGNGTLRAPRASPEIQDRDANGSRRLMLPPPSTPALSGAPPGGAESVHSFYHADCHLEPVRCQAPPPRSPSEASGRTVGSGKVYPTVHTSPPPETLKEKALVEVAASSGPPTLTSLNIPPGPYSSMHKLLETQSTGACQSSCKISSPCLKADSGACGPDSCPYCARAGAGEVELADREMPDSDSEAVYEFTQDAQHSDLRDPHSRRQRSLGPDAEPSSVLAFWRLICDTFRKIVDSKYFGRGIMIAILVNTLSMGIEYHEQPEELTNALEISNIVFTSLFALEMLLKLLVYGPFGYIKNPYNIFDGVIVVISVWEIVGQQGGGLSVLRTFRLMRVLKLVRFLPALQRQLVVLMKTMDNVATFCMLLMLFIFIFSILGMHLFGCKFASERDGDTLPDRKNFDSLLWAIVTVFQILTQEDWNKVLYNGMASTSSWAALYFIALMTFGNYVLFNLLVAILVEGFQAE corresponding to amino acids 1-970 of CCAG_HUMAN, which also corresponds to amino acids 1-970 of R12947_P13, a second amino acid sequence being at least 90% homologous to (SEQ ID NO: 106) GDANKSESEPDFFSPSLDGDGDRKKCLALVSLGEHPELRKSLLPPLIIHTAATPMSLPKSTSTGLGEALGPASRRTSSSGSAEPGAAHEMKSPPSARSSPHSPWSAASSWTSRRSSRNSLGRAPSLKRRSPSGERRSLLSGEGQESQDEEESSEEERASPAGSDHRHRGSLEREAKSSFDLPDTLQVPGLHRTASGRGSASEHQDCNGKSASGRLARALRPDDPPLDGDDADDEGNLSKGERVRAWIRARLPACCLERDSWSAYIFPPQSRFRLLCHRIITHKMFDHVVLVIIFLNCITIAMERPKIDPHSAERIFLTLSNYIFTAVFLAEMTVKVVALGWCFGEQAYLRSSWNVLDGLLVLISVIDILVSMVSDSGTKILGMLRVLRLLRTLRPLRVISRAQGLKLVVETLMSSLKPIGNIVVICCAFFIIFGILGVQLFKGKFFVCQGEDTRNITNKSDCAEASYRWVRHKYNFDNLGQALMSLFVLASKDGWVDIMYDGLDAVGVDQQPIMNHNPWMLLYFISFLLIVAFFVLNMFVGVVVENFHKCRQHQEEEEARRREEKRLRRLEKKRR corresponding to amino acids 994-1568 of CCAG_HUMAN, which also corresponds to amino acids 971-1545 of R12947_P13, a third amino acid sequence being at least 70%, optionally at least 80%, preferably at least 85%, more preferably at least 90% and most preferably at least 95% homologous to a polypeptide having the sequence (SEQ ID NO: 109) SKEKQMAD corresponding to amino acids 1546-1553 of R12947_P13, a fourth amino acid sequence being at least 90% homologous to (SEQ ID NO: 107) LMLDDVIASGSSASAASEAQCKPYYSDYSRFRLLVHHLCTSHYLDLFITGVIGLNVVTMAMEHYQQPQILDEALKICNYIFTVIFVLESVFKLVAFGFRRFFQDRWNQLDLAIVLLSIMGITLEEIEVNASLPINPTIIRIMRVLRIARVLKLLKMAVGMRALLDTVMQALPQVGNLGLLFMLLFFIFAALGVELFGDLECDETHPCEGLGRHATFRNFGMAFLTLFRVSTGDNWNGIMKDTLRDCDQESTCYNTVISPIYFVSFVLTAQFVLVNVVIAVLMKHLEESNKEAKEEAEEAELELEMKTLSPQPHSPLGSPFLWPGVEGPDSPDSPKPGALHPAAHARSASHFSLEHPT corresponding to amino acids 1570-1927 of CCAG_HUMAN, which also corresponds to amino acids 1554-1911 of R12947_P13, and a fifth amino acid sequence being at least 90% homologous to (SEQ ID NO: 108) MQPHPTELPGPDLLTVRKSGVSRTHSLPNDSYMCRHGSTAEGPLGHRGWGLPKAQSGSVLSVHSQPADTSYILQLPKDAPHLLQPHSAPTWGTIPKLPPPGRSPLAQRPLRRQAAIRTDSLDVQGLGSREDLLAEVSGPSPPLARAYSFWGQSSTQAQQHSRSHSKISKHMTPPAPCPGPEPNWGKGPPETRSSLELDTELSWISGDLLPPGGQEEPPSPRDLKKCYSVEAQSCQRRPTSWLDEQRRHSIAVSCLDSGSQPHLGTDPSNLGGQPLGGPGSRPKKKLSPPSITIDPPESQGPRTPPSPGICLRRRAPSSDSKDPLASGPPDSMAASPSPKKDVLSLSGLSSDPADLDP corresponding to amino acids 2021-2377 of CCAG_HUMAN, which also corresponds to amino acids 1912-2268 of R12947_P13, wherein said first amino acid sequence, second amino acid sequence, third amino acid sequence, fourth amino acid sequence and fifth amino acid sequence are contiguous and in a sequential order.

According to preferred embodiments of the present invention, there is provided an isolated chimeric polypeptide encoding for an edge portion of R 12947_P13, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EG, having a structure as follows: a sequence starting from any of amino acid numbers 970−x to 971; and ending at any of amino acid numbers 971+((n−2)−x), in which x varies from 0 to n−2.

According to preferred embodiments of the present invention, there is provided an isolated polypeptide encoding for an edge portion of R12947_P13, comprising an amino acid sequence being at least 70%, optionally at least about 80%, preferably at least about 85%, more preferably at least about 90% and most preferably at least about 95% homologous to the sequence encoding for (SEQ ID NO: 109) SKEKQMAD, corresponding to R12947_P13.

According to preferred embodiments of the present invention, there is provided an isolated chimeric polypeptide encoding for an edge portion of R12947_P13, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise TM, having a structure as follows: a sequence starting from any of amino acid numbers 1911−x to 1911; and ending at any of amino acid numbers 1912+((n−2)−x), in which x varies from 0 to n−2.

Domains affected by alternative splicing includes the extracellular loop between S1 and S2 transmembrane domains of domain II; the S2 transmembrane domain of domain II; and the cytoplasmic loop between domain III and domain IV.

An alternative splice donor site of exon 25, which encodes for the cytoplasmic loop between domain III and domain IV of this alpha subunit type, has been described that leads to skipping of seven amino acid residues, (SEQ ID NO: 110) KAKQMA. The effect on the calcium channel is a rightward shift of activation and inactivation kinetics, and slowing of activation kinetics. In the same channel type, the skipping of exon 26 (which encodes for for the cytoplasmic loop between domain III and domain IV) leads to an 18-amino-acid deletion with a left shift of inactivation kinetics and accelerated activation kinetics.

From the above description of the effect of changes to the cytoplasmic loop between domain III and domain IV of this variant, it is expected that the variant will have a right shift of activation and inactivation and slowing of activation kinetics or a left shift of inactivation and accelerated activation kinetics.

Sequence name: CCAG_HUMAN

documentation:

of: R12947_P13 (SEQ ID NO: 18)×CCAG_HUMAN (SEQ ID NO: 111) . . .

segment 1/1:

Quality: 21913.00

Escore: 0

Matching length: 2261 Total

length: 2384

Matching Percent Similarity: 100.00 Matching Percent

Identity: 99.96

Total Percent Similarity: 94.84 Total Percent

Identity: 94.80

Gaps: 3

| | . . . . . | |
|---|---|---|
| 1 | MDEEEDGAGAEESGQPRSFMRLNDLSGAGGRPGPGSAEKDPGSADSEAEG | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MDEEEDGAGAEESGQPRSFMRLNDLSGAGGRPGPGSAEKDPGSADSEAEG | 50 |
| | . . . . . | |
| 51 | LPYPALAPVVFFYLSQDSRPRSWCLRTVCNPWFERISMLVILLNCVTLGM | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | LPYPALAPVVFFYLSQDSRPRSWCLRTVCNPWFERISMLVILLNCVTLGM | 100 |
| | . . . . . | |
| 101 | FRPCEDIACDSQRCRILQAFDDFIFAFFAVEMVVKMVALGIFGKKCYLGD | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | FRPCEDIACDSQRCRILQAFDDFIFAFFAVEMVVKMVALGIFGKKCYLGD | 150 |
| | . . . . . | |
| 151 | TWNRLDFFIVIAGMLEYSLDLQNVSFSAVRTVRVLRPLRAINRVPSMRIL | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | TWNRLDFFIVIAGMLEYSLDLQNVSFSAVRTVRVLRPLRAINRVPSMRIL | 200 |
| | . . . . . | |
| 201 | VTLLLDTLPMLGNVLLLCFFVFFIFGIVGVQLWAGLLRNRCFLPENFSLP | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | VTLLLDTLPMLGNVLLLCFFVFFIFGIVGVQLWAGLLRNRCFLPENFSLP | 250 |
| | . . . . . | |
| 251 | LSVDLERYYQTENEDESPFICSQPRENGMRSCRSVPTLRGDGGGGPPCGL | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | LSVDLERYYQTENEDESPFICSQPRENGMRSCRSVPTLRGDGGGGPPCGL | 300 |
| | . . . . . | |
| 301 | DYEAYNSSSNTTCVNMNQYYTNCSAGEHNPFKGAINFDNIGYAWIAIFQV | 350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | DYEAYNSSSNTTCVNMNQYYTNCSAGEHNPFKGAINFDNIGYAWIAIFQV | 350 |
| | . . . . . | |
| 351 | ITLEGWVDIMYFVMDAHSFYNFIYFILLIIVGSFFMINLCLVVIATQFSE | 400 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 351 | ITLEGWVDIMYFVMDAHSFYNFIYFILLIIVGSFFMINLCLVVIATQFSE | 400 |
| | . . . . . | |
| 401 | TKQRESQLMREQRVRFLSNASTLASFSEPGSCYEELLKYLVYILRKAARR | 450 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 401 | TKQRESQLMREQRVRFLSNASTLASFSEPGSCYEELLKYLVYILRKAARR | 450 |
| | . . . . . | |
| 451 | LAQVSRAAGVRVGLLSSPAPLGGQETQPSSSCSRSHRRLSVHHLVHHHHH | 500 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 451 | LAQVSRAAGVRVGLLSSPAPLGGQETQPSSSCSRSHRRLSVHHLVHHHHH | 500 |
| | . . . . . | |
| 501 | HHHHYHLGNGTLRAPRASPEIQDRDANGSRRLMLPPPSTPALSGAPPGGA | 550 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 501 | HHHHYHLGNGTLRAPRASPEIQDRDANGSRRLMLPPPSTPALSGAPPGGA | 550 |
| | . . . . . | |
| 551 | ESVHSFYHADCHLEPVRCQAPPPRSPSEASGRTVGSGKVYPTVHTSPPPE | 600 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 551 | ESVHSFYHADCHLEPVRCQAPPPRSPSEASGRTVGSGKVYPTVHTSPPPE | 600 |
| | . . . . . | |
| 601 | TLKEKALVEVAASSGPPTLTSLNIPPGPYSSMHKLLETQSTGACQSSCKI | 650 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 601 | TLKEKALVEVAASSGPPTLTSLNIPPGPYSSMHKLLETQSTGACQSSCKI | 650 |
| | . . . . . | |
| 651 | SSPCLKADSGACGPDSCPYCARAGAGEVELADREMPDSDSEAVYEFTQDA | 700 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 651 | SSPCLKADSGACGPDSCPYCARAGAGEVELADREMPDSDSEAVYEFTQDA | 700 |
| | . . . . . | |
| 701 | QHSDLRDPHSRRQRSLGPDAEPSSVLAFWRLICDTFRKIVDSKYFGRGIM | 750 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 701 | QHSDLRDPHSRRQRSLGPDAEPSSVLAFWRLICDTFRKIVDSKYFGRGIM | 750 |
| | . . . . . | |
| 751 | IAILVNTLSMGIEYHEQPEELTNALEISNIVFTSLFALEMLLKLLVYGPF | 800 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 751 | IAILVNTLSMGIEYHEQPEELTNALEISNIVFTSLFALEMLLKLLVYGPF | 800 |
| | . . . . . | |
| 801 | GYIKNPYNIFDGVIVVISVWEIVGQQGGGLSVLRTFRLMRVLKLVRFLPA | 850 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 801 | GYIKNPYNIFDGVIVVISVWEIVGQQGGGLSVLRTFRLMRVLKLVRFLPA | 850 |
| | . . . . . | |
| 851 | LQRQLVVLMKTMDNVATFCMLLMLFIFIFSILGMHLFGCKFASERDGDTL | 900 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 851 | LQRQLVVLMKTMDNVATFCMLLMLFIFIFSILGMHLFGCKFASERDGDTL | 900 |
| | . . . . . | |
| 901 | PDRKNFDSLLQAIVTVFQILTQEDWNKVLYNGMASTSSWAALYFIALMTF | 950 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 901 | PDRKNFDSLLQAIVTVFQILTQEDWNKVLYNGMASTSSWAALYFIALMTF | 950 |
| | . . . . . | |
| 951 | GNYVLFNLLVAILVEGFQAE.......................GDANKSE | 977 |
| | \|\|\|\|\|\|\|\|\|\|\|\|\|\|\|\|\|\|\|\| \|\|\|\|\|\|\| | |
| 951 | GNYVLFNLLVAILVEGFQAEEISKREDASGQLSCIQLPVDSQGGDANKSE | 1000 |
| | . . . . . | |
| 978 | SEPDFFSPSLDGDGDRKKCLALVSLGEHPELRKSLLPPLIIHTAATPMSL | 1027 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1001 | SEPDFFSPSLDGDGDRKKCLALVSLGEHPELRKSLLPPLIIHTAATPMSL | 1050 |
| | . . . . . | |
| 1028 | PKSTSTGLGEALGPASRRTSSSGSAEPGAAHEMKSPPSARSSPHSPWSAA | 1077 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1051 | PKSTSTGLGEALGPASRRTSSSGSAEPGAAHEMKSPPSARSSPHSPWSAA | 1100 |
| | . . . . . | |
| 1078 | SSWTSRRSSRNSLGRAPSLKRRSPSGERRSLLSGEGQESQDEEESSEEER | 1127 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1101 | SSWTSRRSSRNSLGRAPSLKRRSPSGERRSLLSGEGQESQDEEESSEEER | 1150 |
| | . . . . . | |
| 1128 | ASPAGSDHRHRGSLEREAKSSFDLPDTLQVPGLHRTASGRGSASEHQDCN | 1177 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1151 | ASPAGSDHRHRGSLEREAKSSFDLPDTLQVPGLHRTASGRGSASEHQDCN | 1200 |
| | . . . . . | |
| 1178 | GKSASGRLARALRPDDPPLDGDDADDEGNLSKGERVRAWIRARLPACCLE | 1227 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1201 | GKSASGRLARALRPDDPPLDGDDADDEGNLSKGERVRAWIRARLPACCLE | 1250 |
| | . . . . . | |
| 1228 | RDSWSAYIFPPQSRFRLLCHRIITHKMFDHVVLVIIFLNCITIAMERPKI | 1277 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1251 | RDSWSAYIFPPQSRFRLLCHRIITHKMFDHVVLVIIFLNCITIAMERPKI | 1300 |
| | . . . . . | |
| 1278 | DPHSAERIFLTLSNYIFTAVFLAEMTVKVVALGWCFGEQAYLRSSWNVLD | 1327 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1301 | DPHSAERIFLTLSNYIFTAVFLAEMTVKVVALGWCFGEQAYLRSSWNVLD | 1350 |
| | . . . . . | |
| 1328 | GLLVLISVIDILVSMVSDSGTKILGMLRVLRLLRTLRPLRVISRAQGLKL | 1377 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1351 | GLLVLISVIDILVSMVSDSGTKILGMLRVLRLLRTLRPLRVISRAQGLKL | 1400 |
| | . . . . . | |
| 1378 | VVETLMSSLKPIGNIVVICCAFFIIFGILGVQLFKGKFFVCQGEDTRNIT | 1427 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1401 | VVETLMSSLKPIGNIVVICCAFFIIFGILGVQLFKGKFFVCQGEDTRNIT | 1450 |
| | . . . . . | |
| 1428 | NKSDCAEASYRWVRHKYNFDNLGQALMSLFVLASKDGWVDLMYDGLDAVG | 1477 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1451 | NKSDCAEASYRWVRHKYNFDNLGQALMSLFVLASKDGWVDIMYDGLDAVG | 1500 |
| | . . . . . | |
| 1478 | VDQQPIMNHNPWMLLYFISFLLIVAFFVLNMFVGVVVENFHKCRQHQEEE | 1527 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1501 | VDQQPIMNHNPWMLLYFISFLLIVAFFVLNMFVGVVVENFHKCRQHQEEE | 1550 |
| | . . . . . | |
| 1528 | EARRREEKRLRRLEKKRRSKEKQMADLMLDDVIASGSSASAASEAQCKPY | 1577 |
| | \|\|\|\|\|\|\|\|\|\|\|\|\|\|\|\|\|\| :\|\|\|\|\|\|\|\|\|\|\|\|\|\|\|\|\|\|\|\|\|\|\|\| | |
| 1551 | EARRREEKRLRRLEKKRR.......NLMLDDVIASGSSASAASEAQCKPY | 1593 |
| | . . . . . | |
| 1578 | YSDYSRFRLLVHHLCTSHYLDLFITGVIGLNVVTMAMEHYQQPQILDEAL | 1627 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1594 | YSDYSRFRLLVHHLCTSHYLDLFITGVIGLNVVTMAMEHYQQPQILDEAL | 1643 |
| | . . . . . | |
| 1628 | KICNYIFTVIFVLESVFKLVAFGFRRFFQDRWNQLDLAIVLLSIMGITLE | 1677 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1644 | KICNYIFTVIFVLESVFKLVAFGFRRFFQDRWNQLDLAIVLLSIMGITLE | 1693 |
| | . . . . . | |
| 1678 | EIEVNASLPINPTIIRIMRVLRIARVLKLLKMAVGMRALLDTVMQALPQV | 1727 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1694 | EIEVNASLPINPTIIRIMRVLRIARVLKLLKMAVGMRALLDTVMQALPQV | 1743 |
| | . . . . . | |
| 1728 | GNLGLLGMLLFFIFAALGVELFGDLECDETHPCEGLGRHATFRNFGMAFL | 1777 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1744 | GNLGLLGMLLFFIFAALGVELFGDLECDETHPCEGLGRHATFRNFGMAFL | 1793 |
| | . . . . . | |
| 1778 | TLFRVSTGDNWNGIMKDTLRDCDQESTCYNTVISPIYFVSFVLTAQFVLV | 1827 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1794 | TLFRVSTGDNWNGIMKDTLRDCDQESTCYNTVISPIYFVSFVLTAQFVLV | 1843 |
| | . . . . . | |
| 1828 | NVVIAVLMKHLEESNKEAKEEAELEAELELEMKTLSPQPHSPLGSPFLWP | 1877 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1844 | NVVIAVLMKHLEESNKEAKEEAELEAELELEMKTLSPQPHSPLGSPFLWP | 1893 |
| | . . . . . | |
| 1878 | GVEGPDSPDSPKPGALHPAAHARSASHFSLEHPT................ | 1911 |
| | \|\|\|\|\|\|\|\|\|\|\|\|\|\|\|\|\|\|\|\|\|\|\|\|\|\|\|\|\|\|\|\|\|\| | |
| 1894 | GVEGPDSPDSPKPGALHPAAHARSASHFSLEHPTDRQLFDTISLLIQGSL | 1943 |
| | . . . . . | |
| 1911 | .................................................. | 1911 |
| 1944 | EWELKLMDELAGPGGQPSAFPSAPSLGGSDPQIPLAEMEALSLTSEIVSE | 1993 |
| | . . . . . | |
| 1912 | ...........................MQPHPTELPGPDLLTVRKSGVSR | 1934 |
| | \|\|\|\|\|\|\|\|\|\|\|\|\|\|\|\|\|\|\|\|\|\|\| | |
| 1994 | PSCSLALTDDSLPDDMHTLLLSALESNMQPHPTELPGPDLLTVRKSGVSR | 2043 |
| | . . . . . | |
| 1935 | THSLPNDSYMCRHGSTAEGPLGHRGWGLPKAQSGSVLSVHSQPADTSYIL | 1984 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2044 | THSLPNDSYMCRHGSTAEGPLGHRGWGLPKAQSGSVLSVHSQPADTSYIL | 2093 |
| | . . . . . | |
| 1985 | QLPKDAPHLLQPHSAPTWGTIPKLPPPGRSPLAQRPLRRQAAIRTDSLDV | 2034 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2094 | QLPKDAPHLLQPHSAPTWGTIPKLPPPGRSPLAQRPLRRQAAIRTDSLDV | 2143 |
| | . . . . . | |
| 2035 | QGLGSREDLLAEVSGPSPPLARAYSFWGQSSTQAQQHSRSHSKISKMMTP | 2084 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2144 | QGLGSREDLLAEVSGPSPPLARAYSFWGQSSTQAQQHSRSHSKISKHMTP | 2193 |
| | . . . . . | |
| 2085 | PAPCPGPEPNWGKGPPETRSSLELDTELSWISGDLLPPGGQEEPPSPRDL | 2134 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2194 | PAPCPGPEPNWGKGPPETRSSLELDTELSWISGDLLPPGGQEEPPSPRDL | 2243 |
| | . . . . . | |
| 2135 | KKCYSVEAQSCQRRPTSWLDEQRRHSIAVSCLDSGSQPHLGTDPSNLGGQ | 2184 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2244 | KKCYSVEAQSCQRRPTSWLDEQRRHSIAVSCLDSGSQPHLGTDPSNLGGQ | 2293 |
| | . . . . . | |
| 2185 | PLGGPGSRPKKKLSPPSITIDPPESQGPRTPPSPGICLRRRAPSSDSKDP | 2234 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2294 | PLGGPGSRPKKKLSPPSITIDPPESQGPRTPPSPGICLRRRAPSSDSKDP | 2343 |
| | . . . | |
| 2235 | LASGPPDSMAASPSPKKDVLSLSGLSSDPADLDP | 2268 |
| | \|\|\|\|\|\|\|\|\|\|\|\|\|\|\|\|\|\|\|\|\|\|\|\|\|\|\|\|\|\|\|\|\|\| | |
| 2344 | LASGPPDSMAASPSPKKDVLSLSGLSSDPADLDP | 2377 |

### EXAMPLE 10

This Example relates to the variant R12947_P14 (SEQ ID NO:19; the nucleic acid sequence is given by SEQ ID NO:41), which is a variant alpha 1 subunit according to the present invention, and more specifically, is a splice variant of the known protein CCAG_HUMAN, which is encoded by the CACNA1G gene. This protein is the alpha 1G subunit and results in a voltage-dependent T-type calcium channel. The subunit is also known as alpha subunit Cav3.1 and is described in greater detail with regard to Example 9.

The structure of the splice variant according to the present invention features a a skipped exon and a unique tail, as compared to the known protein sequence. An alignment is provided at the end of this section, while the comparison between the two sequences is described below.

According to preferred embodiments of the present invention, there is provided an isolated chimeric polypeptide encoding for R12947_P14, comprising a first amino acid sequence being at least 90% homologous to (SEQ ID NO: 112) MDEEEDGAGAEESGQPRSFMRLNDLSGAGGRPGPGSAEKDPGSADSEAEGLPYPALAPVVFFYLSQDSRPRSWCLRTVCNPWFERISMLVILLNCVTLGMFRPCEDIACDSQRCRILQAFDDFIFAFFAVEMVVKMVALGIFGKKCYLGDTWNRLDFFIVIAGMLEYSLDLQNVSFSAVRTVRVLRPLRAINRVPSMRILVTLLLDTLPMLGNVLLLCFFVFFIFGIVGVQLWAGLLRNRCFLPENFSLPLSVDLERYYQTENEDESPFICSQPRENGMRSCRSVPTLRGDGGGGPPCGLDYEAYNSSSNTTCVNWNQYYTNCSAGEHNPFKGAINFDNIGYAWIAIFQVITLEGWVDIMYFVMDAHSFYNFIYFILLIIVGSFFMINLCLVVIATQFSETKQRESQLMREQRVRFLSNASTLASFSEPGSCYEELLKYLVYILRKAARRLAQVSRAAGVRVGLLSSPAPLGGQETQPSSSCSRSHRRLSVHHLVHHHHHHHHHYHLGNGTLRAPRASPEIQDRDANGSRRLMLPPPSTPALSGAPPGGAESVHSFYHADCHLEPVRCQAPPPRSPSEASGRTVGSGKVYPTVHTSPPPETLKEKALVEVAASSGPPTLTSLNIPPGPYSSMHKLLETQSTGACQSSCKISSPCLKADSGACGPDSCPYCARAGAGEVELADREMPDSDSEAVYEFTQDAQHSDLRDPHSRRQRSLGPDAEPSSVLAFWRLICDTFRKIVDSKYFGRGIMIAILVNTLSMGIEYHEQPEELTNALEISNIVFTSLFALEMLLKLLVYGPFGYIKNPYNIFDGVIVVISVWEIVGQQGGGLSVLRTFRLMRVLKLVRFLPALQRQLVVLMKTMDNVATFCMLLMLFIFIFSILGMHLFGCKFASERDGDTLPDRKNFDSLLWAIVTVFQILTQEDWNKVLYNGMASTSSWAALYFIALMTFGNYVLFNLLVAILVEGFQAE corresponding to amino acids 1-970 of CCAG_HUMAN, which also corresponds to amino acids 1-970 of R12947_P14, a second amino acid sequence being at least 90% homologous to (SEQ ID NO: 113) GDANKSESEPDFFSPSLDGDGDRKKCLALVSLGEHPELRKSLLPPLIIHTAATPMSLPKSTSTGLGEALGPASRRTSSSGSAEPGAAHEMKSPPSARSSPHSPWSAASSWTSRRSSRNSLGRAPSLKRRSPSGERRSLLSGEGQESQDEEESSEEERASPAGSDHRHRGSLEREAKSSFDLPDTLQVPGLHRTASGRGSASEHQDCNGKSASGRLARALRPDDPPLDGDDADDEGNLSKGERVRAWIRARLPACCLERDSWSAYIFPPQSRFRLLCHRIITHKMFDHVVLVIIFLNCITIAMERPKIDPHSAERIFLTLSNYIFTAVFLAEMTVKVVALGWCFGEQAYLRSSWNVLDGLLVLISVIDILVSMVSDSGTKILGMLRVLRLLRTLRPLRVISRAQGLKLVVETLMSSLKPIGNIVVICCAFFIIFGILGVQLFKGKFFVCQGEDTRNITNKSDCAEASYRWVRHKYNFDNLGQALMSLFVLASKDGWVDIMYDGLDAVGVDQQ corresponding to amino acids 994-1504 of CCAG_HUMAN, which also corresponds to amino acids 971-1481 of R12947_P14, and a third amino acid sequence being at least 70%, optionally at least 80%, preferably at least 85%, more preferably at least 90% and most preferably at least 95% homologous to a polypeptide having the sequence (SEQ ID NO: 114) VGLRWAGSICGLRSLSMGCFQDRKSKAGCKMP corresponding to amino acids 1482-1513 of R12947_P14, wherein said first amino acid sequence, second amino acid sequence and third amino acid sequence are contiguous and in a sequential order.

According to preferred embodiments of the present invention, there is provided an isolated chimeric polypeptide encoding for an edge portion of R12947_P14, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EG, having a structure as follows: a sequence starting from any of amino acid numbers 970−x to 970; and ending at any of amino acid numbers 971+((n−2)−x), in which x varies from 0 to n−2.

According to preferred embodiments of the present invention, there is provided an isolated polypeptide encoding for a tail of R12947_P14, comprising a polypeptide being at least 70%, optionally at least about 80%, preferably at least about 85%, more preferably at least about 90% and most preferably at least about 95% homologous to the sequence (SEQ ID NO: 115) VGLRWAGSICGLRSLSMGCFQDRKSKAGCKMP in R12947_P14.

Domains affected by alternative splicing include the extracellular loop between S1 and S2 transmembrane domains of domain II; the S2 transmembrane domain of domain II; the pore loop region between S5 and S6 transmembrane domains of domain III; the S6 transmembrane domain of domain III; the cytoplasmic loop between domain III and domain IV; the S1 ransmembrane domain of domain IV; the extracellular loop between S1 and S2 transmembrane domains of domain IV; the S2 transmembrane domain of domain IV; the extracellular loop between S2 and S3 transmembrane domains of domain IV; the S3 transmembrane domain of domain IV; the extracellular loop between S3 and S4 transmembrane domains of domain IV; the S4 transmembrane domain of domain IV; the extracellular loop between S4 and S5 transmembrane domains of domain IV; the S5 transmembrane domain of domain IV; the pore loop region between S5 and S6 transmembrane domains of domain IV; the S6 transmembrane domain of domain IV; and the cytoplasmic C-terminus region of the protein.

Alternatively spliced C-termini in T-type channels have been described for Cav3.3. They are produced by combinations of alternative splice acceptor sites in exons 33 and 34 which shorten these exons to different lengths. One of the variants produces a frame shift leading to a premature truncation of the C-terminus; however, interestingly it is not the truncation of the C-terminus which seems to cause the functional effects, but rather the presence of the shortened exon 33 in the transcript. These functional effects include slowed activation, accelerated inactivation, and slowed recovery from inactivation. These changes to calcium current kinetics may be expected to influence neuronal function in such a manner that the slowly inactivating variants would be liable to sustain firing patterns. Analogous splice variants are present in T-type Cav3.1-encoding genes affecting exons 34, 35 and 38 but their functional effect is not known; however, it may be assumed to be similar.

With regard to the effect on the cytoplasmic loop between domain III and domain IV, an explanation is provided with regard to Example 9 above.

From the above description of the effect of changes to the C-terminus domain of this variant, it is expected that the variant will influence neuronal function in such a manner that the slowly inactivating variants would be liable to sustain firing patterns.

From the above description of the effect of changes to the cytoplasmic loop between domain III and domain IV of this variant, it is expected that the variant will have a right shift of activation and inactivation and slowing of activation kinetics or a left shift of inactivation and accelerated activation kinetics.

Sequence name: CCAG_HUMAN

documentation:

of: R12947_P14 (residues 1-1481 of SEQ ID NO: 19)×CCAG_HUMAN (SEQ ID NO: 116) . . .

segment 1/1:

Quality: 14413.00

Escore: 0

Matching length: 1481 Total

length: 1504

Matching Percent Similarity: 100.00 Matching Percent

Identity: 100.00

Total Percent Similaritya: 98.47 Total Percent

Identity: 98.47

Gaps: 1

| | . . . . . | |
|---|---|---|
| 1 | MDEEEDGAGAEESGQPRSFMRLNDLSGAGGRPGPGSAEKDPGSADSEAEG | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MDEEEDGAGAEESGQPRSFMRLNDLSGAGGRPGPGSAEKDPGSADSEAEG | 50 |
| | . . . . . | |
| 51 | LPYPALAPVVFFYLSQDSRPRSWCLRTVCNPWFERISMLVILLNCVTLGM | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | LPYPALAPVVFFYLSQDSRPRSWCLRTVCNPWFERISMLVILLNCVTLGM | 100 |
| | . . . . . | |
| 101 | FRPCEDIACDSQRCRILQAFDDFIFAFFAVEMVVKMVALGIFGKKCYLGD | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | FRPCEDIACDSQRCRILQAFDDFIFAFFAVEMVVKMVALGIFGKKCYLGD | 150 |
| | . . . . . | |
| 151 | TWNRLDFFIVIAGMLEYSLDLQNVSFSAVRTVRVLRPLRAINRVPSMRIL | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | TWNRLDFFIVIAGMLEYSLDLQNVSFSAVRTVRVLRPLRAINRVPSMRIL | 200 |
| | . . . . . | |
| 201 | VTLLLDTLPMLGNVLLLCFFVFFIFGIVGVQLWAGLLRNRCFLPENFSLP | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | VTLLLDTLPMLGNVLLLCFFVFFIFGIVGVQLWAGLLRNRCFLPENFSLP | 250 |
| | . . . . . | |
| 251 | LSVDLERYYQTENEDESPFICSQPRENGMRSCRSVPTLRGDGGGGPPCGL | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | LSVDLERYYQTENEDESPFICSQPRENGMRSCRSVPTLRGDGGGGPPCGL | 300 |
| | . . . . . | |
| 301 | DYEAYNSSSNTTCVNWNQYYTNCSAGEHNPFKGAINFDNIGYAWIAIFQV | 350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | DYEAYNSSSNTTCVNWNQYYTNCSAGEHNPFKGAINFDNIGYAWIAIFQV | 350 |
| | . . . . . | |
| 351 | ITLEGWVDIMYFVMDAHSFYNFIYFILLIIVGSEFMINLCLVVIATQFSE | 400 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 351 | ITLEGWVDIMYFVMDAHSFYNFIYFILLIIVGSFFMINLCLVVIATQFSE | 400 |
| | . . . . . | |
| 401 | TKQRESQLMREQRVRFLSNASTLASFSEPGSCYEELLKYLVYILRKAARR | 450 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 401 | TKQRESQLMREQRVRFLSNASTLASFSEPGSCYEELLKYLVYILRKAARR | 450 |
| | . . . . . | |
| 451 | LAQVSRAAGVRVGLLSSPAPLGGQETQPSSSCSRSHRRLSVHHLVHHHHH | 500 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 451 | LAQVSRAAGVRVGLLSSPAPLGGQETQPSSSCSRSHRRLSVHHLVHHHHH | 500 |
| | . . . . . | |
| 501 | HHHHYHLGNGTLRAPRASPEIQDRDANGSRRLMLPPPSTPALSGAPPGGA | 550 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 501 | HHHHYHLGNGTLRAPRASPEIQDRDANGSRRLMLPPPSTPALSGAPPGGA | 550 |
| | . . . . . | |
| 551 | ESVHSFYHADCHLEPVRCQAPPPRSPSEASGRTVGSGKVYPTVHTSPPPE | 600 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 551 | ESVHSFYHADCHLEPVRCQAPPPRSPSEASGRTVGSGKVYPTVHTSPPPE | 600 |
| | . . . . . | |
| 601 | TLKEKALVEVAASSGPPTLTSLNIPPGPYSSMHKLLETQSTGACQSSCKI | 650 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 601 | TLKEKALVEVAASSGPPTLTSLNIPPGPYSSMHKLLETQSTGACQSSCKI | 650 |
| | . . . . . | |
| 651 | SSPCLKADSGACGPDSCPYCARAGAGEVELADREMPDSDSEAVYEFTQDA | 700 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 651 | SSPCLKADSGACGPDSCPYCARAGAGEVELADREMPDSDSEAVYEFTQDA | 700 |
| | . . . . . | |
| 701 | QHSDLRDPHSRRQRSLGPDAEPSSVLAFWRLICDTFRKIVDSKYFGRGIM | 750 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 701 | QHSDLRDPHSRRQRSLGPDAEPSSVLAFWRLICDTFRKIVDSKYFGRGIM | 750 |
| | . . . . . | |
| 751 | IAILVNTLSMGIEYHEQPEELTNALEISNIVFTSLFALEMLLKLLVYGPF | 800 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 751 | IAILVNTLSMGIEYHEQPEELTNALEISNIVFTSLFALEMLLKLLVYGPF | 800 |
| | . . . . . | |
| 801 | GYIKNPYNIFDGVIVVISVWEIVGQQGGGLSVLRTFRLMRVLKLVRFLPA | 850 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 801 | GYIKNPYNIFDGVIVVISVWEIVGQQGGGLSVLRTFRLMRVLKLVRFLPA | 850 |
| | . . . . . | |
| 851 | LQRQLVVLMKTMDNVATFCMLLMLFIFIFSILGMHLFGCKFASERDGDTL | 900 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 851 | LQRQLVVLMKTMDNVATFCMLLMLFIFIFSILGMHLFGCKFASERDGDTL | 900 |
| | . . . . . | |
| 901 | PDRKNFDSLLWAIVTVFQILTQEDWNKVLYNGMASTSSWAALYFIALMTF | 950 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 901 | PDRKNFDSLLWAIVTVFQILTQEDWNKVLYNGMASTSSWAALYFIALMTF | 950 |
| | . . . . . | |
| 951 | GNYVLFNLLVAILVEGFQAE.......................GDANKSE | 977 |
| | \|\|\|\|\|\|\|\|\|\|\|\|\|\|\|\|\|\|\|\| \|\|\|\|\|\|\| | |
| 951 | GNYVLFNLLVAILVEGFQAEEISKREDASGQLSCIQLPVDSQGGDANKSE | 1000 |
| | . . . . . | |
| 978 | SEPDFFSPSLDGDGDRKKCLALVSLGEHPELRKSLLPPLIIHTAATPMSL | 1027 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1001 | SEPDFFSPSLDGDGDRKKCLALVSLGEHPELRKSLLPPLIIHTAATPMSL | 1050 |
| | . . . . . | |
| 1028 | PKSTSTGLGEALGPASRRTSSSGSAEPGAAHEMKSPPSARSSPHSPWSAA | 1077 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1051 | PKSTSTGLGEALGPASRRTSSSGSAEPGAAHEMKSPPSARSSPHSPWSAA | 1100 |
| | . . . . . | |
| 1078 | SSWTSRRSSRNSLGRAPSLKRRSPSGERRSLLSGEGQESQDEEESSEEER | 1127 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1101 | SSWTSRRSSRNSLGRAPSLKRRSPSGERRSLLSGEGQESQDEEESSEEER | 1150 |
| | . . . . . | |
| 1128 | ASPAGSDHRHRGSLEREAKSSFDLPDTLQVPGLHRTASGRGSASEHQDCN | 1177 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1151 | ASPAGSDHRHRGSLEREAKSSFDLPDTLQVPGLHRTASGRGSASEHQDCN | 1200 |
| | . . . . . | |
| 1178 | GKSASGRLARALRPDDPPLDGDDADDEGNLSKGERVRAWIRARLPACCLE | 1227 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1201 | GKSASGRLARALRPDDPPLDGDDADDEGNLSKGERVRAWIRARLPACCLE | 1250 |
| | . . . . . | |
| 1228 | RDSWSAYIFPPQSRFRLLCHRIITHKMFDHVVLVIIFLNCITIAMERPKI | 1277 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1251 | RDSWSAYIFPPQSRFRLLCHRIITHKMFDHVVLVIIFLNCITIAMERPKI | 1300 |
| | . . . . . | |
| 1278 | DPHSAERIFLTLSNYIFTAVFLAEMTVKVVALGWCFGEQAYLRSSWNVLD | 1327 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1301 | DPHSAERIFLTLSNYIFTAVFLAEMTVKVVALGWCFGEQAYLRSSWNVLD | 1350 |
| | . . . . . | |
| 1328 | GLLVLISVIDILVSMVSDSGTKILGMLRVLRLLRTLRPLRVISRAQGLKL | 1377 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1351 | GLLVLISVIDILVSMVSDSGTKILGMLRVLRLLRTLRPLRVISRAQGLKL | 1400 |
| | . . . . . | |
| 1378 | VVETLMSSLKPIGNIVVICCAFFIIFGILGVQLFKGKFFVCQGEDTRNIT | 1427 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1401 | VVETLMSSLKP1TGNIWICCAFFIIFGILGVQLFKGKFFVCQGEDTRNIT | 1450 |
| | . . . . . | |
| 1428 | NKSDCAEASYRWVRHKYNFDNLGQALMSLFVLASKDGWVDIMYDGLDAVG | 1477 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1451 | NKSDCAEASYRWVRHKYNFDNLGQALMSLFVLASKDGWVDIMYDGLDAVG | 1500 |
| 1478 | VDQQ | 1481 |
| | \|\|\|\| | |
| 1501 | VDQQ | 1504 |

### EXAMPLE 11

This Example relates to the variant T80376_P2 (SEQ ID NO:1; the nucleic acid sequence is given by SEQ ID NO:23), which is a variant alpha 1 subunit according to the present invention, and more specifically, is a splice variant of the known protein CCAH_HUMAN, which is encoded by the CACNA1H gene. This alpha subunit is the alpha 1H subunit, which forms the voltage-dependent T-type calcium channel. It is also known as alpha subunit Cav3.2.

T-type calcium channels belong to the “low-voltage activated (LVA)” group and are strongly blocked by nickel and mibefradil as described above with regard to Example 10. T-type channels serve pacemaking functions in both central neurons and cardiac nodal cells and support calcium signaling in secretory cells and vascular smooth muscle. They may also be involved in the modulation of firing patterns of neurons. This alpha subunit is expressed in kidney, liver, heart, brain; at least one isoform (Isoform 2) seems to be testis-specific.

This alpha subunit has the following structure. Each of the four internal repeats contains five hydrophobic transmembrane segments (S1, S2, S3, S5, S6) and one positively charged transmembrane segment (S4). S4 segments probably represent the voltage-sensor and are characterized by a series of positively charged amino acids at every third position. In response to an increase in intracellular calcium, the T-type channels are activated by CaM-kinase II.

The structure of the splice variant according to the present invention features a skipped exon, as compared to the known protein sequence. An alignment is provided at the end of this section, while the comparison between the two sequences is described below.

According to preferred embodiments of the present invention, there is provided an isolated chimeric polypeptide encoding for T80376_P2, comprising a first amino acid sequence being at least 90% homologous to (SEQ ID NO: 117) MTEGARAADEVRVPLGAPPPGPAALVGASPESPGAPGREAERGSELGVSPSESPAAERGAELGADEEQRVPYPALAATVFFCLGQTTRPRSWCLRLVCNPWFEHVSMLVIMLNCVTLGMFRPCEDVECGSERCNILEAFDAFIFAFFAVEMVIKMVALGLFGQKCYLGDTWNRLDFFIVVAGMMEYSLDGHNVSLSAIRTVRVLRPLRAINRVPSMRILVTLLLDTLPMLGNVLLLCFFVFFIFGIVGVQLWAGLLRNRCFLDSAFVRNNNLTFLRPYYQTEEGEENPFICSSRRDNGMQKCSHIPGRRELRMPCTLGWEAYTQPQAEGVGAARNACINWNQYYNVCRSGDSNPHNGAINFDNIGYAWIAIFQVITLEGWVDIMYYVMDAHSFYNFIYFILLIIVGSFFMINLCLVVIATQFSETKQRESQLMREQRARHLSNDSTLASFSEPGSCYEELLKYVGHIFRKVKRRSLRLYARWQSRWRKKVDPSAVQGQGPGHRQRRAGRHTASVHHLVYHHHHHHHHHYHFSHGSPRRPGPEPGACDTRLVRAGAPPSPPSPGRGPPDAESVHSIYHADCHIEGPQERARVAHAAATAAASLRLATGLGTMNYPTILPSGVGSGKGSTSPGPKGKWAGGPPGTGGHGPLSLNSPDPYEKIPHVVGEHGLGQAPGHLSGLSVPCPLPSPPAGTLTCELKSCPYCTRALEDPEGELSGSESGDSDGRGVYEFTQDVRHGDRWDPTRPPRATDTPGPGPGSPQRRAQQRAAPGEPGWMGRLWVTFSGKLRRIVDSKYFSRGIMMAILVNTLSMGVEYHEQPEELTNALEISNIVFTSMFALEMLLKLLACGPLGYIRNPYNIFDGIIVVISVWEIVGQADGGLSVLRTFRLLRVLKLVRFLPALRRQLVVLVKTMDNVATFCTLLMLFIFIFSILGMHLFGCKFSLKTDTGDTVPDRKNFDSLLWAIVTVFQILTQEDWNVVLYNGMASTSSWAALYFVALMTFGNYVLFNLLVAILVEGFQAEGDANRSDTDEDKTSVHFEEDFHKLRELQTTELKMCSLAVTPNGHLEGRGSLSPPLIMCTAATPMPTPKSSPFLDAAPSLPDSRRGSSSSGDPPLGDQKPPASLRSSPCAPWGPSGAWSSRRSSWSSLGRAPSLKRRGQCGERESLLSGEGKGSTDDEAEDGRAAPGPRATPLRRAESLDPRPLRPAALPPTKCRDRDGQVVALPSDFFLRIDSHREDAAELDDDSEDSCCLRLHKVLEPYKPQWCRSREAWALYLFSPQNRFRVSCQKVITHKMFDHVVLVFIFLNCVTIALERPDIDPGSTERVFLSVSNYIFTAIFVAEMMVKVVALGLLSGEHAYLQSSWNLLDGLLVLVSLVDIVVAMASAGGAKILGVLRVLRLLRTLRPLRVISRAPGLKLVVETLISSLRPIGNIVLICCAFFIIFGILGVQLFKGKFYYCEGPDTRNISTKAQCRAAHYRWVRRKYNFDNLGQALMSLFVLSSKDGWVNIMYDGLDAVGVDQQPVQNHNPWMLLYFISFLLIVSFFVLNMFVGVVVENFHKCRQHQEAEEARRREEKRLRRLERRRR corresponding to amino acids 1-1586 of CCAH_HUMAN, which also corresponds to amino acids 1-1586 of T80376_P2, a second amino acid sequence bridging amino acid sequence comprising of K, and a third amino acid sequence being at least 90% homologous to (SEQ ID NO: 118) AQRRPYYADYSPTRRSIHSLCTSHYLDLFITFIICVNVITMSMEHYNQPKSLDEALKYCNYVFTIVFVFEAALKLVAFGFRRFFKDRWNQLDLAIVLLSLMGITLEEIEMSAALPINPTIIRIMRVLRIARVLKLLKMATGMRALLDTVVQALPQVGNLGLLFMLLFFIYAALGVELFGRLECSEDNPCEGLSRHATFSNFGMAFLTLFRVSTGDNWNGIMKDTLRECSREDKHCLSYLPALSPVYFVTFVLVAQFVLVNVVVAVLMKHLEESNKEAREDAELDAEIELEMAQGPGSARRVDADRPPLPQESPGARDAPNLVARKVSVSRMLSLPNDSYMFRPVVPASAPHPRPLQEVEMETYGAGTPLGSVASVHSPPAESCASLQIPLAVSSPARSGEPLHALSPRGTARSPSLSRLLCRQEAVHTDSLEGKIDSPRDTLDPAEPGEKTPVRPVTQGGSLQSPPRSPRPASVRTRKHTFGQRCVSSRPAAPGGEEAEASDPADEEVSHITSSACPWQPTAEPHGPEASPVAGGERDLRRLYSVDAQGFLDKPGRADEQWRPSAELGSGEPGEAKAWGPEAEPALGARRKKKMSPPCISVEPPAEDEGSARPSAAEGGSTTLRRRTPSCEATPHRDSLEPTEGSGAGGDPAAKGERWGQASCRAEHLTVPSFAFEPLDLGVPSGDPFLDGSHSVTPESRASSSGAIVPLEPPESEPPMPVGDPPEKRRGLYLTVPQCPLEKPGSPSATPAPGGGADDPV corresponding to amino acids 1594-2353 of CCAH_HUMAN, which also corresponds to amino acids 1588-2347 of T80376_P2, wherein said first amino acid sequence, second amino acid sequence and third amino acid sequence are contiguous and in a sequential order.

According to preferred embodiments of the present invention, there is provided an isolated polypeptide encoding for an edge portion of T80376_P2, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least three amino acids comprise RKA having a structure as follows (numbering according to T80376_P2): a sequence starting from any of amino acid numbers 1586−x to 1586; and ending at any of amino acid numbers 1588+((n−2)−x), in which x varies from 0 to n−2.

Domains affected by alternative splicing include the cytoplasmic loop between domain III and domain IV, and the cytoplasmic C-terminus region of the protein. An explanation of expected effects may be found in Examples 2 and 9.

From the above description of the effect of changes to the C-terminus domain of this variant, it is expected that the variant will influence neuronal function in such a manner that the slowly inactivating variants would be liable to sustain firing patterns.

From the above description of the effect of changes to the cytoplasmic loop between domain III and domain IV of this variant, it is expected that the variant will have a right shift of activation and inactivation and slowing of activation kinetics or a left shift of inactivation and accelerated activation kinetics.

Sequence name: CCAH_HUMAN

documentation:

of: T80376_P2 (SEQ ID NO: 1)×CCAH_HUMAN (SEQ ID NO: 119) . . .

segment 1/1:

Quality: 22970.00

Escore: 0

Matching length: 2347 Total

length: 2353

Matching Percent Similarity: 100.00 Matching Percent

Identity: 99.96

Total Percent Similarity: 99.75 Total Percent

Identity: 99.70

Gaps: 1

| | . . . . . | |
|---|---|---|
| 1 | MTEGARAADEVRVPLGAPPPGPAALVGASPESPGAPGREAERGSELGVSP | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MTEGARAADEVRVPLGAPPPGPAALVGASPESPGAPGREAERGSELGVSP | 50 |
| | . . . . . | |
| 51 | SESPAAERGAELGADEEQRVPYPALAATVFFCLGQTTRPRSWCLRLVCNP | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | SESPAAERGAELGADEEQRVPYPALAATVFFCLGQTTRPRSWCLRLVCNP | 100 |
| | . . . . . | |
| 101 | WFEHVSMLVIMLNCVTLGMFRPCEDVECGSERCNILEAFDAFIFAFFAVE | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | WFEHVSMLVIMLNCVTLGMFRPCEDVECGSERCNILEAFDAFIFAFFAVE | 150 |
| | . . . . . | |
| 151 | MVTKMVALGLFGQKCYLGDTWNRLDFFIVVAGMMEYSLDGHNVSLSAIRT | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | MVIKMVALGLFGQKCYLGDTWNRLDFFIVVAGMMEYSLDGHNVSLSAIRT | 200 |
| | . . . . . | |
| 201 | VRVLRPLRAINRVPSMRILVTLLLDTLPMLGNVLLLCFFVFFIFGIVGVQ | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | VRVLRPLRAINRVPSMRILVTLLLDTLPMLGNVLLLCFFVFFIFGIVGVQ | 250 |
| | . . . . . | |
| 251 | LWAGLLRNRCFLDSAFVRNNNLTFLRPYYQTEEGEENPFICSSRRDNGMQ | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | LWAGLLRNRCFLDSAFVRNNNLTFLRPYYQTEEGEENPFICSSRRDNGMQ | 300 |
| | . . . . . | |
| 301 | KCSHIPGRRELRMPCTLGWEAYTQPQAEGVGAARNACINWNQYYNVCRSG | 350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | KCSHIPGRRELRMPCTLGWEAYTQPQAEGVGAARNACINWNQYYNVCRSG | 350 |
| | . . . . . | |
| 351 | DSNPHNGAINFDNIGYAWIAIFQVITLEGWVDIMYYVMDAHSFYNFIYFI | 400 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 351 | DSNPHNGAINFDNIGYAWIAIFQVITLEGWVDIMYYVMDAHSFYNFIYFI | 400 |
| | . . . . . | |
| 401 | LLIIVGSFFMINLCLVVIATQFSETKQRESQLMREQRARHLSNDSTLASF | 450 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 401 | LLIIVGSFFMINLCLVVIATQFSETKQRESQLMREQRARHLSNDSTLASF | 450 |
| | . . . . . | |
| 451 | SEPGSCYEELLKYVGHIFRKVKRRSLRLYARWQSRWRKKVDPSAVQGQGP | 500 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 451 | SEPGSCYEELLKYVGHIFRKVKRRSLRLYARWQSRWRKKVDPSAVQGQGP | 500 |
| | . . . . . | |
| 501 | GHRQRRAGRHTASVHHLVYHHHHHHHHHYHFSHGSPRRPGPEPGACDTRL | 550 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 501 | GHRQRRAGRHTASVHHLVYHHHHHHHHHYHFSHGSPRRPGPEPGACDTRL | 550 |
| | . . . . . | |
| 551 | VRAGAPPSPPSPGRGPPDAESVHSIYHADCHIEGPQERARVAHAAATAAA | 600 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 551 | VRAGAPPSPPSPGRGPPDAESVHSIYHADCHIEGPQERARVAHAAATAAA | 600 |
| | . . . . . | |
| 601 | SLRLATGLGTMNYPTILPSGVGSGKGSTSPGPKGKWAGGPPGTGGHGPLS | 650 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 601 | SLRLATGLGTMNYPTILPSGVGSGKGSTSPGPKGKWAGGPPGTGGHGPLS | 650 |
| | . . . . . | |
| 651 | LNSPDPYEKIPHVVGEHGLGQAPGHLSGLSVPCPLPSPPAGTLTCELKSC | 700 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 651 | LNSPDPYEKIPHVVGEHGLGQAPGHLSGLSVPCPLPSPPAGTLTCELKSC | 700 |
| | . . . . . | |
| 701 | PYCTRALEDPEGELSGSESGDSDGRGVYEFTQDVRHGDRWDPTRPPRATD | 750 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 701 | PYCTRALEDPEGELSGSESGDSDGRGVYEFTQDVRHGDRWDPTRPPRATD | 750 |
| | . . . . . | |
| 751 | TPGPGPGSPQRRAQQRAAPGEPGWMGRLWVTFSGKLRRIVDSKYFSRGIM | 800 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 751 | TPGPGPGSPQRRAQQRAAPGEPGWMGRLWVTFSGKLRRIVDSKYFSRGIM | 800 |
| | . . . . . | |
| 801 | MAILVNTLSMGVEYHEQPEELTNALEISNIVFTSMFALEMLLKLLACGPL | 850 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 801 | MAILVNTLSMGVEYHEQPEELTNALEISNIVFTSMFALEMLLKLLACGPL | 850 |
| | . . . . . | |
| 851 | GYIRNPYNIFDGIIVVISVWEIVGQADGGLSVLRTFRLLRVLKLVRFLPA | 900 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 851 | GYIRNPYNIFDGIIVVISVWEIVGQADGGLSVLRTFRLLRVLKLVRFLPA | 900 |
| | . . . . . | |
| 901 | LRRQLVVLVKTMDNVATFCTLLMLFIFIFSILGMHLFGCKFSLKTDTGDT | 950 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 901 | LRRQLVVLVKTMDNVATFCTLLMLFIFIFSILGMHLFGCKFSLKTDTGDT | 950 |
| | . . . . . | |
| 951 | VPDRKNFDSLLWAIVTVFQILTQEDWNVVLYNGMASTSSWAALYFVALMT | 1000 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 951 | VPDRKNFDSLLWAIVTVFQILTQEDWNVVLYNGMASTSSWAALYFVALMT | 1000 |
| | . . . . . | |
| 1001 | FGNYVLFNLLVAILVEGFQAEGDANRSDTDEDKTSVHFEEDFHKLRELQT | 1050 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1001 | FGNYVLFNLLVAILVEGFQAEGDANRSDTDEDKTSVHFEEDFHKLRELQT | 1050 |
| | . . . . . | |
| 1051 | TELKMCSLAVTPNGHLEGRGSLSPPLIMCTAATPMPTPKSSPFLDAAPSL | 1100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1051 | TELKMCSLAVTPNGHLEGRGSLSPPLIMCTAATPMPTPKSSPFLDAAPSL | 1100 |
| | . . . . . | |
| 1101 | PDSRRGSSSSGDPPLGDQKPPASLRSSPCAPWGPSGAWSSRRSSWSSLGR | 1150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1101 | PDSRRGSSSSGDPPLGDQKPPASLRSSPCAPWGPSGAWSSRRSSWSSLGR | 1150 |
| | . . . . . | |
| 1151 | APSLKRRGQCGERESLLSGEGKGSTDDEAEDGRAAPGPRATPLRRAESLD | 1200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1151 | APSLKRRGQCGERESLLSGEGKGSTDDEAEDGRAAPGPRATPLRRAESLD | 1200 |
| | . . . . . | |
| 1201 | PRPLRPAALPPTKCRDRDGQVVALPSDFFLRIDSHREDAAELDDDSEDSC | 1250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1201 | PRPLRPAALPPTKCRDRDGQVVALPSDFFLRIDSHREDAAELDDDSEDSC | 1250 |
| | . . . . . | |
| 1251 | CLRLHKVLEPYKPQWCRSREAWALYLFSPQNRFRVSCQKVITHKMFDHVV | 1300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1251 | CLRLHKVLEPYKPQWCRSREAWALYLFSPQNRFRVSCQKVITHKMFDHVV | 1300 |
| | . . . . . | |
| 1301 | LVFIFLNCVTIALERPDIDPGSTERVFLSVSNYIFTAIFVAEMMVKVVAL | 1350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1301 | LVFLFLNCVTIALERPDIDPGSTERVFLSVSNYIFTAIFVAEMMVKVVAL | 1350 |
| | . . . . . | |
| 1351 | GLLSGEHAYLQSSWNLLDGLLVLVSLVDIVVAMASAGGAKILGVLRVLRL | 1400 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1351 | GLLSGEHAYLQSSWNLLDGLLVLVSLVDIVVAMASAGGAKILGVLRVLRL | 1400 |
| | . . . . . | |
| 1401 | LRTLRPLRVISRAPGLKLVVETLISSLRPIGNIVLICCAFFIIFGILGVQ | 1450 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1401 | LRTLRPLRVISRAPGLKLVVETLISSLRPIGNIVLICCAFFIIFGILGVQ | 1450 |
| | . . . . . | |
| 1451 | LFKGKFYYCEGPDTRNISTKAQCRAAHYRWVRRKYNFDNLGQALMSLFVL | 1500 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1451 | LFKGKFYYCEGPDTRNISTKAQCRAAHYRWVRRKYNFDNLGQALMSLFVL | 1500 |
| | . . . . . | |
| 1501 | SSKDGWVNIMYDGLDAVGVDQQPVQNHNPWMLLYFISFLLIVSFFVLNMF | 1550 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1501 | SSKDGWVNIMYDGLDAVGVDQQPVQNHNPWMLLYFISFLLIVSFFVLNMF | 1550 |
| | . . . . . | |
| 1551 | VGVVVENFHKCRQHQEAEEARRREEKRLRRLERRRR......KAQRRPYY | 1594 |
| | \|\|\|\|\|\|\|\|\|\|\|\|\|\|\|\|\|\|\|\|\|\|\|\|\|\|\|\|\|\|\|\|\|\|\|\| \|\|\|\|\|\|\|\| | |
| 1551 | VGVVVENFHKCRQHQEAEEARRREEKRLRRLERRRRSTFPSPEAQRRPYY | 1600 |
| | . . . . . | |
| 1595 | ADYSPTRRSIHSLCTSHYLDLFITFIICVNVITMSMEHYNQPKSLDEALK | 1644 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1601 | ADYSPTRRSIHSLCTSHYLDLFITFIICVNVITMSMEHYNQPKSLDEALK | 1650 |
| | . . . . . | |
| 1645 | YCNYVFTIVFVFEAALKLVAFGFRRFFKDRWNQLDLAIVLLSLMGITLEE | 1694 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1651 | YCNYVFTIVFVFEAALKLVAFGFRRFFKDRWNQLDLAIVLLSLMGITLEE | 1700 |
| | . . . . . | |
| 1695 | IEMSAALPINPTIIRIMRVLRIARVLKLLKMATGMRALLDTVVQALPQVG | 1744 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1701 | IEMSAALPINPTIIRIMRVLRIARVLKLLKMATGMRALLDTVVQALPQVG | 1750 |
| | . . . . . | |
| 1745 | NLGLLFMLLFFIYAALGVELFGRLECSEDNPCEGLSRHATFSNFGMAFLT | 1794 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1751 | NLGLLFMLLFFIYAALGVELFGRLECSEDNPCEGLSRHATFSNFGMAFLT | 1800 |
| | . . . . . | |
| 1795 | LFRVSTGDNWNGIMKDTLRECSREDKHCLSYLPALSPVYFVTFVLVAQFV | 1844 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1801 | LFRVSTGDNWNGIMKDTLRECSREDKHCLSYLPALSPVYFVTFVLVAQFV | 1850 |
| | . . . . . | |
| 1845 | LVNVVVAVLMKHLEESNKEAREDAELDAEIELEMAQGPGSARRVDADRPP | 1894 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1851 | LVNVVVAVLMKHLEESNKEAREDAELDAEIELEMAQGPGSARRVDADRPP | 1900 |
| | . . . . . | |
| 1895 | LPQESPGARDAPNLVARKVSVSRMLSLPNDSYMFRPVVPASAPHPRPLQE | 1944 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1901 | LPQESPGARDAPNLVARKVSVSRMLSLPNDSYMFRPVVPASAPHPRPLQE | 1950 |
| | . . . . . | |
| 1945 | VEMETYGAGTPLGSVASVHSPPAESCASLQIPLAVSSPARSGEPLHALSP | 1994 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1951 | VEMETYGAGTPLGSVASVHSPPAESCASLQIPLAVSSPARSGEPLHALSP | 2000 |
| | . . . . . | |
| 1995 | RGTARSPSLSRLLCRQEAVHTDSLEGKIDSPRDTLDPAEPGEKTPVRPVT | 2044 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2001 | RGTARSPSLSRLLCRQEAVHTDSLEGKIDSPRDTLDPAEPGEKTPVRPVT | 2050 |
| | . . . . . | |
| 2045 | QGGSLQSPPRSPRPASVRTRKHTFGQRCVSSRPAAPGGEEAEASDPADEE | 2094 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2051 | QGGSLQSPPRSPRPASVRTRKHTFGQRCVSSRPAAPGGEEAEASDPADEE | 2100 |
| | . . . . . | |
| 2095 | VSHITSSACPWQPTAEPHGPEASPVAGGERDLRRLYSVDAQGFLDKPGRA | 2144 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2101 | VSHITSSACPWQPTAEPHGPEASPVAGGERDLRRLYSVDAQGFLDKPGRA | 2150 |
| | . . . . . | |
| 2145 | DEQWRPSAELGSGEPGEAKAWGPEAEPALGARRKKKMSPPCISVEPPAED | 2194 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2151 | DEQWRPSAELGSGEPGEAKAWGPEAEPALGARRKKKMSPPCISVEPPAED | 2200 |
| | . . . . . | |
| 2195 | EGSARPSAAEGGSTTLRRRTPSCEATPHRDSLEPTEGSGAGGDPAAKGER | 2244 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2201 | EGSARPSAAEGGSTTLRRRTPSCEATPHRDSLEPTEGSGAGGDPAAKGER | 2250 |
| | . . . . . | |
| 2245 | WGQASCRAEHLTVPSFAFEPLDLGVPSGDPFLDGSHSVTPESRASSSGAI | 2294 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2251 | WGQASCRAEHLTVPSFAFEPLDLGVPSGDPFLDGSHSVTPESRASSSGAI | 2300 |
| | . . . . . | |
| 2295 | VPLEPPESEPPMPVGDPPEKRRGLYLTVPQCPLEKPGSPSATPAPGGGAD | 2344 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2301 | VPLEPPESEPPMPVGDPPEKRRGLYLTVPQCPLEKPGSPSATPAPGGGAD | 2350 |
| 2345 | DPV | 2347 |
| \|\|\| | | |
| 2351 | DPV | 2353 |

### EXAMPLE 12

This Example relates to the variant T80376_P3 (SEQ ID NO:2; the nucleic acid sequence is given by SEQ ID NO:24), which is a variant alpha 1 subunit according to the present invention, and more specifically, is a splice variant of the known protein CCAH_HUMAN, which is encoded by the CACNA1H gene. This alpha subunit is the alpha 1H subunit, which forms the voltage-dependent T-type calcium channel. It is also known as alpha subunit Cav3.2. The known protein is described with regard to Example 11 above.

The structure of the splice variant according to the present invention features a skipped exon, as compared to the known protein sequence. An alignment is provided at the end of this section, while the comparison between the two sequences is described below.

According to preferred embodiments of the present invention, there is provided an isolated chimeric polypeptide encoding for T80376_P3, comprising a first amino acid sequence being at least 90% homologous to (SEQ ID NO: 120) MHLFGCKFSLKTDTGDTVPDRKNFDSLLWAIVTVFQILTQEDWNVVLYNGMASTSSWAALYFVALMTFGNYVLFNLLVAILVEGFQAEGDANRSDTDEDKTSVHFEEDFHKLRELQTTELKMCSLAVTPNGHLEGRGSLSPPLIMCTAATPMPTPKSSPFLDAAPSLPDSRRGSSSSGDPPLGDQKPPASLRSSPCAPWGPSGAWSSRRSSWSSLGRAPSLKRRGQCGERESLLSGEGKGSTDDEAEDGRAAPGPRATPLRRAESLDPRPLRPAALPPTKCRDRDGQVVALPSDFFLRIDSHREDAAELDDDSEDSCCLRLHKVLEPYKPQWCRSREAWALYLFSPQNRFRVSCQKVITHKMFDHVVLVFIFLNCVTIALERPDIDPGSTERVFLSVSNYIFTAIFVAEMMVKVVALGLLSGEHAYLQSSWNLLDGLLVLVSLVDIVVAMASAGGAKILGVLRVLRLLRTLRPLRVISRAPGLKLVVETLISSLRPIGNIVLICCAFFIIFGILGVQLFKGKFYYCEGPDTRNISTKAQCRAAHYRWVRRKYNFDNLGQALMSLFVLSSKDGWVNIMYDGLDAVGVDQQPVQNHNPWMLLYFISFLLIVSFFVLNMFVGVVVENFHKCRQHQEAEEARRREEKRLRRLERRRR corresponding to amino acids 934-1586 of CCAH_HUMAN, which also corresponds to amino acids 1-653 of T80376_P3, a second amino acid sequence bridging amino acid sequence comprising of K, and a third amino acid sequence being at least 90% homologous to (SEQ ID NO: 121) AQRRPYYADYSPTRRSIHSLCTSHYLDLFITFIICVNVITMSMEHYNQPKSLDEALKYCNYVFTIVFVFEAALKLVAFGFRRFFKDRWNQLDLAIVLLSLMGITLEEIEMSAALPINPTIIRIMRVLRIARVLKLLKMATGMRALLDTVVQALPQVGNLGLLFMLLFFIYAALGVELFGRLECSEDNPCEGLSRHATFSNFGMAFLTLFRVSTGDNWNGIMKDTLRECSREDKHCLSYLPALSPVYFVTFVLVAQFVLVNVVVAVLMKHLEESNKEAREDAELDAEIELEMAQGPGSARRVDADRPPLPQESPGARDAPNLVARKVSVSRMLSLPNDSYMFRPVVPASAPHPRPLQEVEMETYGAGTPLGSVASVHSPPAESCASLQIPLAVSSPARSGEPLHALSPRGTARSPSLSRLLCRQEAVHTDSLEGKIDSPRDTLDPAEPGEKTPVRPVTQGGSLQSPPRSPRPASVRTRKHTFGQRCVSSRPAAPGGEEAEASDPADEEVSHITSSACPWQPTAEPHGPEASPVAGGERDLRRLYSVDAQGFLDKPGRADEQWRPSAELGSGEPGEAKAWGPEAEPALGARRKKKMSPPCISVEPPAEDEGSARPSAAEGGSTTLRRRTPSCEATPHRDSLEPTEGSGAGGDPAAKGERWGQASCRAEHLTVPSFAFEPLDLGVPSGDPFLDGSHSVTPESRASSSGAIVPLEPPESEPPMPVGDPPEKRRGLYLTVPQCPLEKPGSPSATPAPGGGADDPV corresponding to amino acids 1594-2353 of CCAH_HUMAN, which also corresponds to amino acids 655-1414 of T80376_P3, wherein said first amino acid sequence, second amino acid sequence and third amino acid sequence are contiguous and in a sequential order.

According to preferred embodiments of the present invention, there is provided an isolated polypeptide encoding for an edge portion of T80376_P3, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least three amino acids comprise RKA having a structure as follows (numbering according to T80376_P3): a sequence starting from any of amino acid numbers 653−x to 653; and ending at any of amino acid numbers 655+((n−2)−x), in which x varies from 0 to n−2.

Domains affected by alternative splicing include the cytoplasmic N-terminus region of the protein; the S1 transmembrane domain of domain I; the extracellular loop between S1 and S2 transmembrane domains of domain I; the S2 transmembrane domain of domain I; the extracellular loop between S2 and S3 transmembrane domains of domain I; the S3 transmembrane domain of domain I; the extracellular loop between S3 and S4 transmembrane domains of domain I; the S4 transmembrane domain of domain I; the extracellular loop between S4 and S5 transmembrane domains of domain I; the S5 transmembrane domain of domain I; the pore loop region between S5 and S6 transmembrane domains of domain I; the S6 transmembrane domain of domain I; the cytoplasmic loop between domain I and domain II; the S1 transmembrane domain of domain II; the extracellular loop between S1 and S2 transmembrane domains of domain II; the S2 transmembrane domain of domain II; the extracellular loop between S2 and S3 transmembrane domains of domain II; the S3 transmembrane domain of domain II; the extracellular loop between S3 and S4 transmembrane domains of domain II; the S4 transmembrane domain of domain II; the extracellular loop between S4 and S5 transmembrane domains of domain II; the S5 transmembrane domain of domain II; the cytoplasmic loop between domain III and domain IV.

Certain Effects of These Changes are Described with Regard to Example 9.

The domain I-II interlinker (cytoplasmic loop) is important for G-protein modulation, inactivation and possibly beta subunit interaction.

From the above description of the effect of changes to the cytoplasmic loop between domain III and domain IV of this variant, it is expected that the variant will have a right shift of activation and inactivation and slowing of activation kinetics or a left shift of inactivation and accelerated activation kinetics.

From the above description of the effect of changes to the cytoplasmic loop between domain I and domain II of this variant, it is expected that the variant will influence the G-protein modulation, inactivation and possibly beta subunit interaction.

| | . . . . . | |
|---|---|---|
| 1 | MHLFGCKFSLKTDTGDTVPDRKNFDSLLWAIVTVFQILTQEDWNVVLYNG | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 934 | MHLFGCKFSLKTDTGDTVPDRKNFDSLLWAIVTVFQILTQEDWNVVLYNG | 983 |
| | . . . . . | |
| 51 | MASTSSWAALYFVALMTFGNYVLFNLLVAILVEGFQAEGDANRSDTDEDK | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 984 | MASTSSWAALYFVALMTFGNYVLFNLLVAILVEGFQAEGDANRSDTDEDK | 1033 |
| | . . . . . | |
| 101 | TSVHFEEDFHKLRELQTTELKMCSLAVTPNGHLEGRGSLSPPLIMCTAAT | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1034 | TSVHFEEDFHKLRELQTTELKMCSLAVTPNGHLEGRGSLSPPLIMCTAAT | 1083 |
| | . . . . . | |
| 151 | PMPTPKSSPFLDAAPSLPDSRRGSSSSGDPPLGDQKPPASLRSSPCAPWG | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1084 | PMPTPKSSPFLDAAPSLPDSRRGSSSSGDPPLGDQKPPASLRSSPCAPWG | 1133 |
| | . . . . . | |
| 201 | PSGAWSSRRSSWSSLGRAPSLKRRGQCGERESLLSGEGKGSTDDEAEDGR | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1134 | PSGAWSSRRSSWSSLGRAPSLKRRGQCGERESLLSGEGKGSTDDEAEDGR | 1183 |
| | . . . . . | |
| 251 | AAPGPRATPLRRAESLDPRPLRPAALPPTKCRDRDGQVVALPSDFFLRID | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1184 | AAPGPRATPLRRAESLDPRPLRPAALPPTKCRDRDGQVVALPSDFFLRID | 1233 |
| | . . . . . | |
| 301 | SHREDAAELDDDSEDSCCLRLHKVLEPYKPQWCRSREAWALYLFSPQNRF | 350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1234 | SHREDAAELDDDSEDSCCLRLHKVLEPYKPQWCRSREAWALYLFSPQNRF | 1283 |
| | . . . . . | |
| 351 | RVSCQKVITHKMFDHVVLVFIFLNCVTIALERPDIDPGSTERVFLSVSNY | 400 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1284 | RVSCQKVITHKMFDHVVLVFIFLNCVTIALERPDIDPGSTERVFLSVSNY | 1333 |
| | . . . . . | |
| 401 | IFTAIFVAEMMVKVVALGLLSGEHAYLQSSWNLLDGLLVLVSLVDIVVAM | 450 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1334 | IFTAIFVAEMMVKVVALGLLSGEHAYLQSSWNLLDGLLVLVSLVDIVVAM | 1383 |
| | . . . . . | |
| 451 | ASAGGAKILGVLRVLRLLRTLRPLRVISRAPGLKLVVETLISSLRPIGNI | 500 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1384 | ASAGGAKILGVLRVLRLLRTLRPLRVISRAPGLKLVVETLISSLRPIGNI | 1433 |
| | . . . . . | |
| 501 | VLICCAFFIIFGILGVQLFKGKFYYCEGPDTRNISTKAQCRAAHYRWVRR | 550 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1434 | VLICCAFFIIFGILGVQLFKGKFYYCEGPDTRNISTKAQCRAAHYRWVRR | 1483 |
| | . . . . . | |
| 551 | KYNFDNLGQALMSLFVLSSKDGWVNIMYDGLDAVGVDQQPVQNHNPWMLL | 600 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1484 | KYNFDNLGQALMSLFVLSSKDGWVNIMYDGLDAVGVDQQPVQNHNPWMLL | 1533 |
| | . . . . . | |
| 601 | YFISFLLIVSFFVLNMFVGVVVENFHKCRQHQEAEEARRREEKRLRRLER | 650 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1534 | YFISFLLIVSFFVLNMFVGVVVENFHKCRQHQEAEEARRREEKRLRRLER | 1583 |
| | . . . . . | |
| 651 | RRR......KAQRRPYYADYSPTRRSIHSLCTSHYLDLFITFIICVNVIT | 694 |
| | \|\|\| : \|\|\|\|\|\|\|\|\|\|\|\|\|\|\|\|\|\|\|\|\|\|\|\|\|\|\|\|\|\|\|\|\|\|\|\|\|\|\|\| | |
| 1584 | RRRSTFPSPEAQRRPYYADYSPTRRSIHSLCTSHYLDLFITFIICVNVIT | 1633 |
| | . . . . . | |
| 695 | MSMEHYNQPKSLDEALKYCNYVFTIVFVFEAALKLVAFGFRRFFKDRWNQ | 744 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1634 | MSMEHYNQPKSLDEALKYCNYVFTIVFVFEAALKLVAFGFRRFFKDRWNQ | 1683 |
| | . . . . . | |
| 745 | LDLAIVLLSLMGITLEEIEMSAALPINPTIIRIMRVLRIARVLKLLKMAT | 794 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1684 | LDLAIVLLSLMGITLEEIEMSAALPINPTIIRIMRVLRIARVLKLLKMAT | 1733 |
| | . . . . . | |
| 795 | GMRALLDTVVQALPQVGNLGLLFMLLFFIYAALGVELFGRLECSEDNPCE | 844 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1734 | GMRALLDTVVQALPQVGNLGLLFMLLFFIYAALGVELFGRLECSEDNPCE | 1783 |
| | . . . . . | |
| 845 | GLSRHATFSNFGMAFLTLFRVSTGDNWNGIMKDTLRECSREDKHCLSYLP | 894 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1784 | GLSRHATFSNFGMAFLTLFRVSTGDNWNGIMKDTLRECSREDKHCLSYLP | 1833 |
| | . . . . . | |
| 895 | ALSPVYFVTFVLVAQFVLVNVVVAVLMKHLEESNKEAREDAELDAEIELE | 944 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1834 | ALSPVYFVTFVLVAQFVLVNVVVAVLMKHLEESNKEAREDAELDAEIELE | 1883 |
| | . . . . . | |
| 945 | MAQGPGSARRVDADRPPLPQESPGARDAPNLVARKVSVSRMLSLPNDSYM | 994 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1884 | MAQGPGSARRVDADRPPLPQESPGARDAPNLVARKVSVSRMLSLPNDSYM | 1933 |
| | . . . . . | |
| 995 | FRPVVPASAPHPRPLQEVEMETYGAGTPLGSVASVHSPPAESCASLQIPL | 1044 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1934 | FRPVVPASAPHPRPLQEVEMETYGAGTPLGSVASVHSPPAESCASLQIPL | 1983 |
| | . . . . . | |
| 1045 | AVSSPARSGEPLHALSPRGTARSPSLSRLLCRQEAVHTDSLEGKIDSPRD | 1094 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1984 | AVSSPARSGEPLHALSPRGTARSPSLSRLLCRQEAVHTDSLEGKIDSPRD | 2033 |
| | . . . . . | |
| 1095 | TLDPAEPGEKTPVRPVTQGGSLQSPPRSPRPASVRTRKHTFGQRCVSSRP | 1144 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2034 | TLDPAEPGEKTPVRPVTQGGSLQSPPRSPRPASVRTRKHTFGQRCVSSRP | 2083 |
| | . . . . . | |
| 1145 | AAPGGEEAEASDPADEEVSHITSSACPWQPTAEPHGPEASPVAGGERDLR | 1194 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2084 | AAPGGEEAEASDPADEEVSHITSSACPWQPTAEPHGPEASPVAGGERDLR | 2133 |
| | . . . . . | |
| 1195 | RLYSVDAQGFLDKPGRADEQWRPSAELGSGEPGEAKAWGPEAEPALGARR | 1244 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2134 | RLYSVDAQGFLDKPGRADEQWRPSAELGSGEPGEAKAWGPEAEPALGARR | 2183 |
| | . . . . . | |
| 1245 | KKKMSPPCISVEPPAEDEGSARPSAAEGGSTTLRRRTPSCEATPHRDSLE | 1294 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2184 | KKKMSPPCISVEPPAEDEGSARPSAAEGGSTTLRRRTPSCEATPHRDSLE | 2233 |
| | . . . . . | |
| 1295 | PTEGSGAGGSPAAKGERWGQASCRAEHLTVPSFAFEPLDLGVPSGDPFLD | 1344 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2234 | PTEGSGAGGDPAAKGERWGQASCRAEHLTVPSFAFEPLDLGVPSGDPFLD | 2283 |
| | . . . . . | |
| 1345 | GSHSVTPESRASSSGAIVPLEPPESEPPMPVGDPPEKRRGLYLTVPQCPL | 1394 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2284 | GSHSVTPESRASSSGAIVPLEPPESEPPMPVGDPPEKRRGLYLTVPQCPL | 2333 |
| | . . | |
| 1395 | EKPGSPSATPAPGGGADDPV | 1414 |
| | \|\|\|\|\|\|\|\|\|\|\|\|\|\|\|\|\|\|\|\| | |
| 2334 | EKPGSPSATPAPGGGADDPV | 2353 |

### EXAMPLE 13

This Example relates to the variant T80376_P4 (SEQ ID NO:3; the nucleic acid sequence is given by SEQ ID NO:25), which is a variant alpha 1 subunit according to the present invention, and more specifically, is a splice variant of the known protein CCAH_HUMAN, which is encoded by the CACNA1H gene. This alpha subunit is the alpha 1H subunit, which forms the voltage-dependent T-type calcium channel. It is also known as alpha subunit Cav3.2. The known protein is described with regard to Example 11 above.

The structure of the splice variant according to the present invention features a unique tail, as compared to the known protein sequence. An alignment is provided at the end of this section, while the comparison between the two sequences is described below.

According to preferred embodiments of the present invention, there is provided an isolated chimeric polypeptide encoding for T80376_P4, comprising a first amino acid sequence being at least 90% homologous to (SEQ ID NO: 123) MTEGARAADEVRVPLGAPPPGPAALVGASPESPGAPGREAERGSELGVSPSESPAAERGAELGADEEQRVPYPALAATVFFCLGQTTRPRSWCLRLVCNPWFEHVSMLVIMLNCVTLGMFRPCEDVECGSERCNILEAFDAFIFAFFAVEMVIKMVALGLFGQKCYLGDTWNRLDFFIVVAGMMEYSLDGHNVSLSAIRTVRVLRPLRAINRVPSMRILVTLLLDTLPMLGNVLLLCFFVFFIFGIVGVQLWAGLLRNRCFLDSAFVR corresponding to amino acids 1-268 of CCAH_HUMAN, which also corresponds to amino acids 1-268 of T80376_P4, and a second amino acid sequence being at least 70%, optionally at least 80%, preferably at least 85%, more preferably at least 90% and most preferably at least 95% homologous to a polypeptide having the sequence (SEQ ID NO: 124) CPGPTPVRPLPRWPCPPCR corresponding to amino acids 269-287 of T80376_P4, wherein said first amino acid sequence and second amino acid sequence are contiguous and in a sequential order.

According to preferred embodiments of the present invention, there is provided an isolated polypeptide encoding for a tail of T80376_P4, comprising a polypeptide being at least 70%, optionally at least about 80%, preferably at least about 85%, more preferably at least about 90% and most preferably at least about 95% homologous to the sequence (SEQ ID NO: 125) CPGPTPVRPLPRWPCPPCR in T80376_P4.

Domains affected by alternative splicing include the pore loop region between S5 and S6 transmembrane domains of domain I; the S6 transmembrane domain of domain I; the cytoplasmic loop between domain I and domain II; the S1 transmembrane domain of domain II; the extracellular loop between S1 and S2 transmembrane domains of domain II; the S2 transmembrane domain of domain II; the extracellular loop between S2 and S3 transmembrane domains of domain II; the S3 transmembrane domain of domain II; the extracellular loop between S3 and S4 transmembrane domains of domain II; the S4 transmembrane domain of domain II; the extracellular loop between S4 and S5 transmembrane domains of domain II; tThe S5 transmembrane domain of domain II; the pore loop region between S5 and S6 transmembrane domains of domain II; the S6 transmembrane domain of domain II; the cytoplasmic loop between domain II and domain III; the S1 transmembrane domain of domain III; the extracellular loop between S1 and S2 transmembrane domains of domain III; the S2 transmembrane domain of domain III; the extracellular loop between S2 and S3 transmembrane domains of domain III; the S3 transmembrane domain of domain III; the extracellular loop between S3 and S4 transmembrane domains of domain III; the S4 transmembrane domain of domain III; the extracellular loop between S4 and S5 transmembrane domains of domain III; the S5 transmembrane domain of domain III; the pore loop region between S5 and S6 transmembrane domains of domain III; the S6 transmembrane domain of domain III; the cytoplasmic loop between domain III and domain IV; the S1 transmembrane domain of domain IV; the extracellular loop between S1 and S2 transmembrane domains of domain IV; the S2 transmembrane domain of domain IV; the extracellular loop between S2 and S3 transmembrane domains of domain IV; the S3 transmembrane domain of domain IV; the extracellular loop between S3 and S4 transmembrane domains of domain IV; the S4 transmembrane domain of domain IV; the extracellular loop between S4 and S5 transmembrane domains of domain IV; the S5 transmembrane domain of domain IV; the pore loop region between S5 and S6 transmembrane domains of domain IV; the S6 transmembrane domain of domain IV; the cytoplasmic C-terminus region of the protein.

The effects of certain of these changes were explained in Examples 2, 9 and 12 above.

From the above description of the effect of changes to the cytoplasmic loop between domain III and domain IV of this variant, it is expected that the variant will have a right shift of activation and inactivation and slowing of activation kinetics or a left shift of inactivation and accelerated activation kinetics.

From the above description of the effect of changes to the cytoplasmic loop between domain I and domain II of this variant, it is expected that the variant will influence the G-protein modulation, inactivation and possibly beta subunit interaction.

From the above description of the effect of changes to the C-terminus domain of this variant, it is expected that the variant will influence neuronal function in such a manner that the slowly inactivating variants would be liable to sustain firing patterns.

Sequence name: CCAH_HUMAN

documentation:

of T80376_P4 (residues 1-277 of SEQ ID NO: 3)x CCAH_HUMAN (SEQ ID NO: 126) . . .

seqment 1/1:

Quality: 2617.00

Escore: 0

Matching length: 277 Total

length: 277

Matching Percent Similarity: 98.19 Matching Percent

Identity: 97.83

Total Percent Similarity: 98.19 Total Percent

Identity: 97.83

Gaps: 0

| | . . . . . | |
|---|---|---|
| 1 | MTEGARAADEVRVPLGAPPPGPAALVGASPESPGAPGREAERGSELGVSP | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MTEGARAADEVRVPLGAPPPGPAALVGASPESPGAPGREAERGSELGVSP | 50 |
| | . . . . . | |
| 51 | SESPAAERGAELGADEEQRVPYPALAATVFFCLGQTTRPRSWCLRLVCNP | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | SESPAAERGAELGADEEQRVPYPALAATVFFCLGQTTRPRSWCLRLVCNP | 100 |
| | . . . . . | |
| 101 | WFEHVSMLVIMLNCVTLGMFRPCEDVECGSERCNILEAFDAFIFAFFAVE | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | WFEHVSMLVIMLNCVTLGMFRPCEDVECGSERCNILEAFDAFIFAFFAVE | 150 |
| | . . . . . | |
| 151 | MVIKMVALGLFGQKCYLGDTWNRLDFFIVVAGMMEYSLDGHNVSLSAIRT | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | MVIKMVALGLFGQKCYLGDTWNRLDFFIVVAGMMEYSLDGHNVSLSAIRT | 200 |
| | . . . . . | |
| 201 | VRVLRPLRAINRVPSMRILVTLLLDTLPMLGNVLLLCFFVFFIFGIVGVQ | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | VRVLRPLRAINRVPSMRILVTLLLDTLPMLGNVLLLCFFVFFIFGIVGVQ | 250 |
| | . . | |
| 251 | LWAGLLRNRCFLDSAFVRCPGPTPVRP | 277 |
| | \|\|\|\|\|\|\|\|\|\|\|\|\|\|\|\|\|\| \| :\|\| | |
| 251 | LWAGLLRNRCFLDSAFVRNNNLTFLRP | 277 |

### EXAMPLE 14

This Example relates to the variant HUMLVDCCB_P19 (SEQ ID NO:4; the nucleic acid sequence is given by SEQ ID NO:26), which is a variant alpha 1 subunit according to the present invention, and more specifically, is a splice variant of the known protein CCAC_HUMAN, which is encoded by the CACNA1C gene. This alpha subunit is the alpha 1C subunit, which forms the voltage-dependent L-type calcium channel. This subunit is also called the alpha subunit Cav1.2. This subunit results in L-type calcium channels as described above. Calcium channels containing the alpha-1C subunit play an important role in excitation-contraction coupling in the heart. The various isoforms display marked differences in the sensitivity to DHP compounds.

This subunit is expressed in heart, ovary, pancreatic beta-cells and in the brain.

The structure is as follows. Each of the four internal repeats contains five hydrophobic transmembrane segments (S1, S2, S3, S5, S6) and one positively charged transmembrane segment (S4). S4 segments probably represent the voltage-sensor and are characterized by a series of positively charged amino acids at every third position.

It is predicted that phosphorylation by PKA activates the channel.

The structure of the splice variant according to the present invention features a unique tail, a unique insertion and a skipped exon, as compared to the known protein sequence. An alignment is provided at the end of this section, while the comparison between the two sequences is described below.

According to preferred embodiments of the present invention, there is provided an isolated chimeric polypeptide encoding for HUMLVDCCB_P19, comprising a first amino acid sequence being at least 90% homologous to (SEQ ID NO: 127) MVNENTRMYIPEENHQGSNYGSPRPAHANMNANAAAGLAPEHIPTPGAALSWQAAIDAARQAKLMGSAGNATISTVSSTQRKRQQYGKPKKQGSTTATRPPRALLCLTLKNPIRRACISIVEWKPFEIIILLTIFANCVALAIYIPFPEDDSNATNSNLERVEYLFLIIFTVEAFLKVIAYGLLFHPNAYLRNGWNLLDFIIVVVGLFSAILEQATKADGANALGGKGAGFDVKALRAFRVLRPLRLVSGVPSLQVVLNSIIKAMVPLLHIALLVLFVIIIYAIIGLELFMGKMHKTCYNQEGIADVPAEDDPSPCALETGHGRQCQNGTVCKPGWDGPKHGITNFDNFAFAMLTVFQCITMEGWTDVLYWVNDAVGRDWPWIYFVTLIIIGSFFVLNLVLGVLSGEFSKEREKAKARGDFQKLREKQQLEEDLKGYLDWITQAEDIDPENEDEGMDEEKPRN corresponding to amino acids 1-463 of CCAC_HUMAN, which also corresponds to amino acids 1-463 of HUMLVDCCB_P19, a second amino acid sequence being at least 70%, optionally at least 80%, preferably at least 85%, more preferably at least 90% and most preferably at least 95% homologous to a polypeptide having the sequence (SEQ ID NO: 128) RGTPAGMLDQKKGKFAWFSHSTETHV corresponding to amino acids 464-489 of HUMLVDCCB_P19, a third amino acid sequence being at least 90% homologous to (SEQ ID NO: 129) SMPTSETESVNTENVAGGDIEGENCGARLAHRISKSKFSRYWRRWNRFCRRKCRAAVKSNVFYWLVIFLVFLNTLTIASEHYNQPNWLTEVQDTANKALLALFTAEMLLKMYSLGLQAYFVSLFNRFDCFVVCGGILETILVETKIMSPLGISVLRCVRLLRIFKITRYWNSLSNLVASLLNSVRSIASLLLLLFLFIIIFSLLGMQLFGGKFNFDEMQTRRSTFDNFPQSLLTVFQILTGEDWNSVMYDGIMAYGGPSFPGMLVCIYFIILFICGNYILLNVFLAIAVDNLADAESLTSAQKEEEEEKERKKLARTASPEKKQELVEKPAVGESKEEKIELKSITADGESPPATKINMDDLQPNENEDKSPYPNPETTGEEDEEEPEMPVGPRPRPLSELHLKEKAVPMPEASAFFIFSSNNRFRLQCHRIVNDTIFTNLILFFILLSSISLAAEDPVQHTSFRNH corresponding to amino acids 465-931 of CCAC_HUMAN, which also corresponds to amino acids 490-956 of HUMLVDCCB_P19, and a fourth amino acid sequence being at least 70%, optionally at least 80%, preferably at least 85%, more preferably at least 90% and most preferably at least 95% homologous to a polypeptide having the sequence (SEQ ID NO: 130) VCIACVFCTPSPWGCARPTASVADIIVQSGAQF corresponding to amino acids 957-989 of HUMLVDCCB_P19, wherein said first amino acid sequence, second amino acid sequence, third amino acid sequence and fourth amino acid sequence are contiguous and in a sequential order.

According to preferred embodiments of the present invention, there is provided an isolated polypeptide encoding for a unique insertion of HUMLVDCCB_P19, comprising an amino acid sequence being at least 70%, optionally at least about 80%, preferably at least about 85%, more preferably at least about 90% and most preferably at least about 95% homologous to the sequence encoding for (SEQ ID NO: 131) RGTPAGMLDQKKGKFAWFSHSTETHV, corresponding to HUMLVDCCB_P19.

According to preferred embodiments of the present invention, there is provided a bridge portion of HUMLVDCCB_P19, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise NR, having a structure as follows (numbering according to HUMLVDCCB_P19): a sequence starting from any of amino acid numbers 463−x to 463; and ending at any of amino acid numbers 464+((n−2)−x), in which x varies from 0 to n−2.

According to preferred embodiments of the present invention, there is provided an isolated polypeptide encoding for a tail of HUMLVDCCB_P19, comprising a polypeptide being at least 70%, optionally at least about 80%, preferably at least about 85%, more preferably at least about 90% and most preferably at least about 95% homologous to the sequence (SEQ ID NO: 132) VCIACVFCTPSPWGCARPTASVADIIVQSGAQF in HUMLVDCCB_P19.

Domains affected by alternative splicing include the cytoplasmic loop between domain I and domain II; the extracellular loop between S1 and S2 transmembrane domains of domain III; the S2 transmembrane domain of domain III; the extracellular loop between S2 and S3 transmembrane domains of domain III; the S3 transmembrane domain of domain III; the extracellular loop between S3 and S4 transmembrane domains of domain III; the S4 transmembrane domain of domain III; the extracellular loop between S4 and S5 transmembrane domains of domain III.

The S5 transmembrane domain of domain III; the pore loop region between S5 and S6 transmembrane domains of domain III; the S6 transmembrane domain of domain III; the cytoplasmic loop between domain III and domain IV; the S1 transmembrane domain of domain IV; the extracellular loop between S1 and S2 transmembrane domains of domain IV; the S2 transmembrane domain of domain IV; the extracellular loop between S2 and S3 transmembrane domains of domain IV; the S3 transmembrane domain of domain IV; the extracellular loop between S3 and S4 transmembrane domains of domain IV; the S4 transmembrane domain of domain IV; the extracellular loop between S4 and S5 transmembrane domains of domain IV; the S5 transmembrane domain of domain IV; the pore loop region between S5 and S6 transmembrane domains of domain IV; the S6 transmembrane domain of domain IV; the cytoplasmic C-terminus region of the protein.

The effect on the cytoplasmic C-terminus region of the protein is expected to increase current amplitude and also calcium-dependent inactivation. For L-type channels, variants with shortened C-terminus in heart and skeletal muscle Cav1.2 and Cav1.1 have long been known but the truncation takes place at the protein level rather than being the result of RNA splicing. In the cardiac Cav1.2 channel, exons 40-43 show combinations of usage of an alternative splice donor site at the 3′ end of exon 40 or alternative splice acceptor sites at the 5′ ends of exons 42 and 43, inclusion of a supplementary cassette exon following exon 40 or skipping of exon 42.

The S2 transmembrane domain of domain III in cardiac L-type Cav1.2 channels is encoded by mutually exclusive exons 21 and 22, which differ by seven amino acids; this difference influences the voltage-dependent action of dihydropyridines.

The S3 transmembrane domain of domain IV in the same channels is encoded by mutually exclusive exons 31 and 32 which work as a developmentally regulated switch coinciding with major changes in excitation; and mutually exclusive exons 31a or 31b which influence dihydropyridine sensitivity.

From the above description of the effect of changes to the C-terminus domain of this variant, it is expected that the variant will have greater current amplitude from the known protein and a stronger calcium dependence of inactivation. It is also expected to modulate the Ca⁺²-dependent inactivation of the channel.

In addition, the changes in the S2 transmembrane domain of domain III might influence the voltage-dependent action of dihydropyridines, an important class of calcium channel blockers.

In addition, the changes in the S3 transmembrane domain of domain IV might influence dihydropyridine sensitivity and also may work as a developmentally regulated switch coinciding with major changes in excitation.

Sequence name: CCAC_HUMAN

documentation:

of: HUMLVDCCB_P19 (residues 1-957 of SEQ ID NO: 4)×CCAC_HUMAN (SEQ ID NO: 133) . . .

seqment 1/1:

Quality: 8982.00

Escore: 0

Matching length: 932 Total

length: 957

Matching Percent Similarity: 100.00 Matching Percent

Identity: 99.79

Total Percent Similarity: 97.39 Total Percent

Identity: 97.18

Gaps: 1

| | . . . . . | |
|---|---|---|
| 1 | MVNENTRMYIPEENHQGSNYGSPRPAHANMNANAAAGLAPEHIPTPGAAL | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MVNENTRMYIPEENHQGSNYGSPRPAHANMNANAAAGLAPEHIPTPGAAL | 50 |
| | . . . . . | |
| 51 | SWQAAIDAARQAKLMGSAGNATISTVSSTQRKRQQYGKPKKQGSTTATRP | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | SWQAAIDAARQAKLMGSAGNATISTVSSTQRKRQQYGKPKKQGSTTATRP | 100 |
| | . . . . . | |
| 101 | PRALLCLTLKNPIRRACISIVEWKPFEIIILLTIFANCVALAIYIPFPED | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | PRALLCLTLKNPIRRACISIVEWKPFEIIILLTIFANCVALAIYIPFPED | 150 |
| | . . . . . | |
| 151 | DSNATNSNLERVEYLFLIIFTVEAFLKVIAYGLLFHPNAYLRNGWNLLDF | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | DSNATNSNLERVEYLFLIIFTVEAFLKVIAYGLLFHPNAYLRNGWNLLDF | 200 |
| | . . . . . | |
| 201 | IIVVVGLFSAILEQATKADGANALGGKGAGFDVKALRAFRVLRPLRLVSG | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | IIVVVGLFSAILEQATKADGANALGGKGAGFDVKALRAFRVLRPLRLVSG | 250 |
| | . . . . . | |
| 251 | VPSLQVVLNSIIKAMVPLLHIALLVLFVIIIYAIIGLELFMGKMHKTCYN | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | VPSLQVVLNSIIKAMVPLLHIALLVLFVIIIYAIIGLELFMGKMHKTCYN | 300 |
| | . . . . . | |
| 301 | QEGIADVPAEDDPSPCALETGHGRQCQNGTVCKPGWDGPKHGITNFDNFA | 350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | QEGIADVPAEDDPSPCALETGHGRQCQNGTVCKPGWDGPKHGITNFDNFA | 350 |
| | . . . . . | |
| 351 | FAMLTVFQCITMEGWTDVLYWVNDAVGRDWPWIYFVTLIIIGSFFVLNLV | 400 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 351 | FAMLTVFQCITMEGWTDVLYWVNDAVGRDWPWIYFVTLIIIGSFFVLNLV | 400 |
| | . . . . . | |
| 401 | LGVLSGEFSKEREKAKARGDFQKLREKQQLEEDLKGYLDWITQAEDIDPE | 450 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 401 | LGVLSGEFSKEREKAKARGDFQKLREKQQLEEDLKGYLDWITQAEDIDPE | 450 |
| | . . . . . | |
| 451 | NEDEGMDEEKPRNRGTPAGMLDQKKGKFAWFSHSTETHVSMPTSETESVN | 500 |
| | \|\|\|\|\|\|\|\|\|\|\|\|\| :\|\|\|\|\|\|\|\|\|\|\| | |
| 451 | NEDEGMDEEKPRN.........................MSMPTSETESVN | 475 |
| | . . . . . | |
| 501 | TENVAGGDIEGENCGARLAHRISKSKFSRYWRRWNRFCRRKCRAAVKSNV | 550 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 476 | TENVAGGDIEGENCGARLAHRISKSKFSRYWRRWNRFCRRKCRAAVKSNV | 525 |
| | . . . . . | |
| 551 | FYWLVIFLVFLNTLTIASEHYNQPNWLTEVQDTANKALLALFTAEMLLKM | 600 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 526 | FYWLVIFLVFLNTLTIASEHYNQPNWLTEVQDTANKALLALFTAEMLLKM | 575 |
| | . . . . . | |
| 601 | YSLGLQAYFVSLFNRFDCFVVCGGILETILVETKIMSPLGISVLRCVRLL | 650 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 576 | YSLGLQAYFVSLFNRFDCFVVCGGILETILVETKIMSPLGISVLRCVRLL | 625 |
| | . . . . . | |
| 651 | RIFKITRYWNSLSNLVASLLNSVRSIASLLLLLFLFIIIFSLLGMQLFGG | 700 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 626 | RIFKITRYWNSLSNLVASLLNSVRSIASLLLLLFLFIIIFSLLGMQLFGG | 675 |
| | . . . . . | |
| 701 | KFNFDEMQTRRSTFDNFPQSLLTVFQILTGEDWNSVMYDGIMAYGGPSFP | 750 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 676 | KFNFDEMQTRRSTFDNFPQSLLTVFQILTGEDWNSVMYDGIMAYGGPSFP | 725 |
| | . . . . . | |
| 751 | GMLVCIYFIILFICGNYILLNVFLAIAVDNLADAESLTSAQKEEEEEKER | 800 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 726 | GMLVCIYFIILFICGNYILLNVFLAIAVDNLADAESLTSAQKEEEEEKER | 775 |
| | . . . . . | |
| 801 | KKLARTASPEKKQELVEKPAVGESKEEKIELKSITADGESPPATKINMDD | 850 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 776 | KKLARTASPEKKQELVEKPAVGESKEEKIELKSITADGESPPATKINMDD | 825 |
| | . . . . . | |
| 851 | LQPNENEDKSPYPNPETTGEEDEEEPEMPVGPRPRPLSELHLKEKAVPMP | 900 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 826 | LQPNENEDKSPYPNPETTGEEDEEEPEMPVGPRPRPLSELHLKEKAVPMP | 875 |
| | . . . . . | |
| 901 | EASAFFIFSSNNRFRLQCHRIVNDTIFTNLILFFILLSSISLAAEDPVQH | 950 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 876 | EASAFFIFSSNNRFRLQCHRIVNDTIFTNLILFFILLSSISLAAEDPVQH | 925 |
| 951 | TSFRNHV | 957 |
| | \|\|\|\|\|\|: | |
| 926 | TSFRNHI | 932 |

### EXAMPLE 15

This Example relates to the variant HUMLVDCCB_P26 (SEQ ID NO:5; the nucleic acid sequence is given by SEQ ID NO:27), which is a variant alpha 1 subunit according to the present invention, and more specifically, is a splice variant of the known protein CCAC_HUMAN, which is encoded by the CACNA1C gene. This alpha subunit is the alpha 1C subunit, which forms the voltage-dependent L-type calcium channel. This subunit is also called the alpha subunit Cav1.2. The known protein is described in greater detail above with regard to Example 14.

The structure of the splice variant according to the present invention features a unique insertion and a skipped exon, as compared to the known protein sequence. An alignment is provided at the end of this section, while the comparison between the two sequences is described below.

According to preferred embodiments of the present invention, there is provided an isolated chimeric polypeptide encoding for HUMLVDCCB_P26, comprising a first amino acid sequence being at least 90% homologous to (SEQ ID NO: 134) MNANAAAGLAPEHIPTPGAALSWQAAIDAARQAKLMGSAGNATISTVSSTQRKR corresponding to amino acids 30-83 of CCAC_HUMAN, which also corresponds to amino acids 1-54 of HUMLVDCCB_P26, a bridging amino acid R corresponding to amino acid 55 of HUMLVDCCB_P26, a second amino acid sequence being at least 90% homologous to (SEQ ID NO: 135) QYGKPKKQGSTTATRPPRALLCLTLKNPIRRACISIVEWKPFEIIILLTIFANCVALAIYIPFPEDDSNATNSNLERVEYLFLIIFTVEAFLKVIAYGLLFHPNAYLRNGWNLLDFIIVVVGLFSAILEQATKADGANALGGKGAGFDVKALRAFRVLRPLRLVSGVPSLQVVLNSIIKAMVPLLHIALLVLFVIIIYAIIGLELFMGKMHKTCYNQEGIADVPAEDDPSPCALETGHGRQCQNGTVCKPGWDGPKHGITNFDNFAFAMLTVFQCITMEGWTDVLYW corresponding to amino acids 85-371 of CCAC_HUMAN, which also corresponds to amino acids 56-342 of HUMLVDCCB_P26, a third amino acid sequence being at least 70%, optionally at least 80%, preferably at least 85%, more preferably at least 90% and most preferably at least 95% homologous to a polypeptide having the sequence (SEQ ID NO: 136) MQDAMGYELPWVYFVSLVIF corresponding to amino acids 343-362 of HUMLVDCCB_P26; a fourth amino acid sequence being at least 90% homologous to (SEQ ID NO: 137) GSFFVLNLVLGVLSGEFSKEREKAKARGDFQKLREKQQLEEDLKGYLDWITQAEDIDPENEDEGMDEEKPRNMSMPTSETESVNTENVAGGDIEGENCGARLAHRISKSKFSRYWRRWNRFCRRKCRAAVKSNVFYWLVIFLVFLNTLTIASEHYNQPNWLTEVQDTANKALLALFTAEMLLKMYSLGLQAYFVSLFNRFDCFVVCGGILETILVETKIMSPLGISVLRCVRLLRIFKITRYWNSLSNLVASLLNSVRSIASLLLLLFLFIIIFSLLGMQLFGGKFNFDEMQTRRSTFDNFPQSLLTVFQILTGEDWNSVMYDGIMAYGGPSFPGMLVCIYFIILFICGNYILLNVFLAIAVDNLADAESLTSAQKEEEEEKERKKLARTASPEKKQELVEKPAVGESKEEKIELKSITADGESPPATKINMDDLQPNENEDKSPYPNPETTGEEDEEEPEMPVGPRPRPLSELHLKEKAVPMPEASAFFIFSSNNRFRLQCHRIVNDTIFTNLILFFILLSSISLAAEDPVQHTSFR NHILFYFDIVFTTIFTIEIA corresponding to amino acids 392-949 of CCAC_HUMAN, which also corresponds to amino acids 363-920 of HUMLVDCCB_P26, a fifth amino acid sequence being at least 90% homologous to (SEQ ID NO: 138) LKMTAYGAFLHKGSFCRNYFNILDLLVVSVSLISFGIQSSAINVVKILRVLRVLRPLRAINRAKGLKHVVQCVFVAIRTIGNIVIVTTLLQFMFACIGVQLFKGKLYTCSDSSKQTEAECKGNYITYKDGEVDHPIIQPRSWENSKFDFDNVLAAMMALFTVSTFEGWPELLYRSIDSHTEDKGPIYNYRVEISIFFIIYIIIIAFFMMNIFVGFVIVTFQEQGEQEYKNCELDKNQRQCVEYALKARPLRRYIPKNQHQYKVWYVVNSTYFEYLMFVLILLNTICLAMQHYGQSCLFKIAMNILNMLFTGLFTVEMILKLIAFKPK corresponding to amino acids 970-1296 of CCAC_HUMAN, which also corresponds to amino acids 921-1247 of HUMLVDCCB_P26, a sixth amino acid sequence being at least 90% homologous to (SEQ ID NO: 139) HYFCDAWNTFDALIVVGSIVDIAITEVN corresponding to amino acids 1325-1352 of CCAC_HUMAN, which also corresponds to amino acids 1248-1275 of HUMLVDCCB_P26, a seventh amino acid sequence being at least 90% homologous to (SEQ ID NO: 140) NAEENSRISITFFRLFRVMRLVKLLSRGEGIRTLLWTFIKSFQALPYVALLIVMLFFIYAVIGMQVFGKIALNDTTEFQTFPQAVLLLFRCATGEAWQDIMLACMPGKKCAPESEPSNSTEGETPCGSSFAVFYFISFYMLCAFLIINLFVAVIMDNFDYLTRDWSILGPHHLDEFKRIWAEYDPEAKGRIKHLDVVTLLRRIQPPLGFGKLCPHRVACKRLVSMNMPLNSDGTVMFNATLFALVRTALRIKTEGNLEQANEELRAIIKKIWKRTSMKLLDQVVPPAGDDEVTVGKFYATFLIQEYFRKFKKRKEQGLVGKPSQRNALSLQAGLRTLHDIGPEIRRAISGDLTAEEELDKAMKEAVSAASEDDIFRRAGGLFGNHVSYYQSDGRSAFPQTFTTQRPLHINKAGSSQGDTESPSHEKLVDSTFTPSSYSSTGSNANINNANNTALGRLPRPAGYPSTVSTVEGHGPPLSPAIRVQEVAWKLSSNR corresponding to amino acids 1364-1863 of CCAC_HUMAN, which also corresponds to amino acids 1276-1775 of HUMLVDCCB_P26, an eighth amino acid sequence being at least 70%, optionally at least 80%, preferably at least 85%, more preferably at least 90% and most preferably at least 95% homologous to a polypeptide having the sequence (SEQ ID NO: 141) MHCCDMLDGGTFPPALGPRRAPPCLHQQLQGSLAGLREDTPCIVPGHASLCCSSRVGEWLPAGCTAPQHA corresponding to amino acids 1776-1845 of HUMLVDCCB_P26; a ninth amino acid sequence being at least 90% homologous to (SEQ ID NO: 142) RCHSRESQAAMAGQEETSQDETYEVKMNHDTEACSEPSLLSTEMLSYQDDENRQLTLPEEDKRDIRQSPKRGFLRSASLGRRASFHLECLKRQKDRGGDISQKTVLPLHLVHHQALAVAGLSPLLQRSHSPASFPRPFATPPATPGSRGWPPQPVPTLRLEGVESSEKLNSSFPSIHCGSWAETTPGGGGSSAARRVRPVSLMVPSQAGAPGRQFHGSASSLVEAVLISEGLGQFAQDPKFIEVTTQELADACDMTIEEMESAADNILSGGAPQSPNGALLPFVNCRDAGQDRAGGEEDAGCVRARG corresponding to amino acids 1898-2204 of CCAC_HUMAN, which also corresponds to amino acids 1846-2152 of HUMLVDCCB_P26, a bridging amino acid R corresponding to amino acid 2153 of HUMLVDCCB_P26, and a tenth amino acid sequence being at least 90% homologous to (SEQ ID NO: 143) PSEEELQDSRVYVSSL corresponding to amino acids 2206-2221 of CCAC_HUMAN, which also corresponds to amino acids 2154-2169 of HUMLVDCCB_P26, wherein said first amino acid sequence, bridging amino acid, second amino acid sequence, third amino acid sequence, fourth amino acid sequence, fifth amino acid sequence, sixth amino acid sequence, seventh amino acid sequence, eight amino acid sequence, ninth amino acid sequence, bridging amino acid, and tenth amino acid sequence are contiguous and in a sequential order.

According to preferred embodiments of the present invention, there is provided an isolated chimeric polypeptide encoding for an edge portion of HUMLVDCCB_P26, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise AL, having a structure as follows: a sequence starting from any of amino acid numbers 920−x to 921; and ending at any of amino acid numbers 921+((n−2)−x), in which x varies from 0 to n−2.

According to preferred embodiments of the present invention, there is provided an isolated chimeric polypeptide encoding for an edge portion of HUMLVDCCB_P26, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise KH, having a structure as follows: a sequence starting from any of amino acid numbers 1247−x to 1248; and ending at any of amino acid numbers 1248+((n−2)−x), in which x varies from 0 to n−2.

According to preferred embodiments of the present invention, there is provided an isolated chimeric polypeptide encoding for an edge portion of HUMLVDCCB_P26, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise NN, having a structure as follows: a sequence starting from any of amino acid numbers 1275−x to 1276; and ending at any of amino acid numbers 1276+((n−2)−x), in which x varies from 0 to n−2.

According to preferred embodiments of the present invention, there is provided an isolated chimeric polypeptide encoding for an edge portion of HUMLVDCCB_P26, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise CD, having a structure as follows: a sequence starting from any of amino acid numbers 1779−x to 1780; and ending at any of amino acid numbers 1780+((n−2)−x), in which x varies from 0 to n−2.

According to preferred embodiments of the present invention, there is provided an isolated polypeptide encoding for an edge portion of HUMLVDCCB_P26, comprising an amino acid sequence being at least 70%, optionally at least about 80%, preferably at least about 85%, more preferably at least about 90% and most preferably at least about 95% homologous to the sequence encoding for (SEQ ID NO: 144) CLHQQLQGSLAGLREDTPCIVPGHASLCCSSRVGEWLPAGCTAPQHA, corresponding to HUMLVDCCB_P26.

According to preferred embodiments of the present invention, there is provided a bridge portion of HUMLVDCCB_P26, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise P, having a structure as follows (numbering according to HUMLVDCCB_P26): a sequence starting from any of amino acid numbers 1896−x to 1896; and ending at any of amino acid numbers 1799+((n−2)−x), in which x varies from 0 to n−2.

According to preferred embodiments of the present invention, there is provided a unique insertion, comprising a polypeptide being at least 70%, optionally at least 80%, preferably at least 85%, more preferably at least 90% and most preferably at least 95% homologous to a polypeptide having the sequence (SEQ ID NO: 145) MQDAMGYELPWVYFVSLVIF of HUMLVDCCB_P26.

According to preferred embodiments of the present invention, there is provided a unique insertion, comprising a polypeptide being at least 70%, optionally at least 80%, preferably at least 85%, more preferably at least 90% and most preferably at least 95% homologous to a polypeptide having the sequence (SEQ ID NO: 146) MHCCDMLDGGTFPPALGPRRAPPCLHQQLQGSLAGLREDTPCIVPGHASLCCSSRVGEWLPAGCTAPQHA of HUMLVDCCB_P26.

Domains affected by alternative splicing include the S2 transmembrane domain of domain III; the extracellular loop between S2 and S3 transmembrane domains of domain III; the extracellular loop between S2 and S3 transmembrane domains of domain IV; the S3 transmembrane domain of domain IV; the extracellular loop between S3 and S4 transmembrane domains of domain IV; the cytoplasmic C-terminus region of the protein.

Certain changes in the variant are explained with regard to example 14.

From the above description of the effect of changes to the C-terminus domain of this variant, it is expected that the variant will have greater current as compared to the known protein and a stronger calcium dependence of inactivation. It is also expected to modulate the Ca⁺²⁻ dependent inactivation of the channel.

In addition, the changes in the S2 transmembrane domain of domain III might influence the voltage-dependent action of dihydropyridines.

Sequence name: CCAC_HUMAN

documentation:

of HUMLVDCCP_P26 (SEQ ID NO: 5)×CCAC_HUMAN (SEQ ID NO: 147) . . .

segment 1/1:

Alignment segment 1/1:

Quality: 1961.00

Escore: 0

Matching length: 2071 Total

length: 2290

Matching Percent Similarity: 100.00 Matching Percent

Identity: 100.00

Total Percent Similarity: 90.44 Total Percent

Identity: 90.44

Gaps: 11

Alignment:

| | . . . . . | |
|---|---|---|
| 1 | MNANAAAGLAPEHIPTPGAALSWQAAIDAARQAKLMGSAGNATISTVSST | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 30 | MNANAAAGLAPEHIPTPGAALSWQAAIDAARQAKLMGSAGNATISTVSST | 79 |
| | . . . . . | |
| 51 | QRKRRQYGKPKKQGSTTATRPPRALLCLTLKNPIRRACISIVEWKPFEI | 99 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 80 | QRKRQQYGKPKKQGSTTATRPPRALLCLTLKNPIRRACISIVEWKPFEI | 128 |
| | . . . . . | |
| 100 | IILLTIFANCVALAIYIPFPEDDSNATNSNLERVEYLFLIIFTVEAFLKV | 149 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 129 | IILLTIFANCVALAIYIPFPEDDSNATNSNLERVEYLFLIIFTVEAFLKV | 178 |
| | . . . . . | |
| 150 | IAYGLLFHPNAYLRNGWNLLDFIIVVVGLFSAILEQATKADGANALGGKG | 199 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 179 | IAYGLLFHPNAYLRNGWNLLDFIIVVVGLFSAILEQATKADGANALGGKG | 228 |
| | . . . . . | |
| 200 | AGFDVKALRAFRVLRPLRLVSGVPSLQVVLNSIIKAMVPLLHIALLVLFV | 249 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 229 | AGFDVKALRAFRVLRPLRLVSGVPSLQVVLNSIIKAMVPLLHIALLVLFV | 278 |
| | . . . . . | |
| 250 | IIIYAIIGLELFMGKMHKTCYNQEGIADVPAEDDPSPCALETGHGRQCQN | 299 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 279 | IIIYAIIGLELFMGKMHKTCYNQEGIADVPAEDDPSPCALETGHGRQCQN | 328 |
| | . . . . . | |
| 300 | GTVCKPGWDGPKHGITNFDNFAFAMLTVFQCITMEGWTDVLYWMQDAMGY | 349 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 329 | GTVCKPGWDGPKHGITNFDNFAFAMLTVFQCITMEGWTDVLYW....... | 371 |
| | . . . . . | |
| 350 | ELPWVYFVSLVIF....................GSFFVLNLVLGVL | 375 |
| | \|\|\|\|\|\|\|\|\|\|\|\|\| | |
| 372 | .............VNDAVGRDWPWIYFVTLIIIGSFFVLNLVLGVL | 404 |
| | . . . . . | |
| 376 | SGEFSKEREKAKARGDFQKLREKQQLEEDLKGYLDWITQAEDIDPENEDE | 425 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 405 | SGEFSKEREKAKARGDFQKLREKQQLEEDLKGYLDWITQAEDIDPENEDE | 454 |
| | . . . . . | |
| 426 | GMDEEKPRNMSMPTSETESVNTENVAGGDIEGENCGARLAHRISKSKFSR | 475 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 455 | GMDEEKPRNMSMPTSETESVNTENVAGGDIEGENCGARLAHRISKSKFSR | 504 |
| | . . . . . | |
| 476 | YWRRSNRFCRRKCRAAVKSNVFYWLVIFLVFLNTLTIASEHYNQPNWLTE | 525 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 505 | YWRRSNRFCRRKCRAAVKSNVFYWLVIFLVFLNTLTIASEHYNQPNWLTE | 554 |
| | . . . . . | |
| 526 | VQDTANKALLALFTAEMLLKMYSLGLQAYFVSLFNRFDCFVVCGGILETI | 575 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 555 | VQDTANKALLALFTAEMLLKMYSLGLQAYFVSLFNRFDCFVVCGGILETI | 604 |
| | . . . . . | |
| 576 | LVETKIMSPLGISVLRCVRLLRIFKITRYWNSLSNLVASLLNSVRSIASL | 625 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 605 | LVETKIMSPLGISVLRCVRLLRIFKITRYWNSLSNLVASLLNSVRSIASL | 654 |
| | . . . . . | |
| 626 | LLLLFLFIIIFSLLGMQLFGGKFNFDEMQTRRSTFDNFPQSLLTVFQILT | 675 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 655 | LLLLFLFIIIFSLLGMQLFGGKFNFDEMQTRRSTFDNFPQSLLTVFQILT | 704 |
| | . . . . . | |
| 676 | GEDWNSVMYDGIMAYGGPSFPGMLVDIYFIILFICGNYILLNVFLAIAVD | 725 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 705 | GEDWNSVMYDGIMAYGGPSFPGMLVDIYFIILFICGNYILLNVFLAIAVD | 754 |
| | . . . . . | |
| 726 | NLADAESLTSAQKEEEEEKERKKLARTASPEKKQELVEKPAVGESKEEKI | 775 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 755 | NLADAESLTSAQKEEEEEKERKKLARTASPEKKQELVEKPAVGESKEEKI | 804 |
| | . . . . . | |
| 776 | ELKSITADGESPPATKINMDDLQPNENEDKSPYPNPETTGEEDEEEPEMP | 825 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 805 | ELKSITADGESPPATKINMDDLQPNENEDKSPYPNPETTGEEDEEEPEMP | 854 |
| | . . . . . | |
| 826 | VGPRPRPLSELHLKEKAVPMPEASAFFIFSSNNRFRLQCHRIVNDTIFTN | 875 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 855 | VGPRPRPLSELHLKEKAVPMPEASAFFIFSSNNRFRLQCHRIVNDTIFTN | 904 |
| | . . . . . | |
| 876 | LILFFILLSSISLAAEDPVQHTSFRNHILFYFDIVFTTIFTIEIA..... | 920 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 905 | LILFFILLSSISLAAEDPVQHTSFRNHILFYFDIVFTTIFTIEIALKILG | 954 |
| | . . . . . | |
| 921 | ...............LKMTAYGAFLHKGSFCRNYFNILDLLVVSVSLISF | 955 |
| | \|\|\|\|\|\|\|\|\|\|\|\|\|\|\|\|\|\|\|\|\|\|\|\|\|\|\|\|\|\|\|\|\|\|\| | |
| 955 | NADYVFTSIFTLEIILKMTAYGAFLHKGSFCRNYFNILDLLVVSVSLISF | 1004 |
| | . . . . . | |
| 956 | GIQSSAINVVKILRVLRVLRPLRAINRAKGLKHVVQCVFVAIRTIGNIVI | 1005 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1005 | GIQSSAINVVKILRVLRVLRPLRAINRAKGLKHVVQCVFVAIRTIGNIVI | 1054 |
| | . . . . . | |
| 1006 | VTTLLQFMFACIGVQLFKGKLYTCSDSSKQTEAECKGNYITYKDGEVDHP | 1055 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1055 | VTTLLQFMFACIGVQLFKGKLYTCSDSSKQTEAECKGNYITYKDGEVDHP | 1104 |
| | . . . . . | |
| 1056 | IIQPRSWENSKFDFDNVLAAMMALFTVSTFEGWPELLYRSIDSHTEDKGP | 1105 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1105 | IIQPRSWENSKFDFDNVLAAMMALFTVSTFEGWPELLYRSIDSHTEDKGP | 1154 |
| | . . . . . | |
| 1106 | IYNYRVEISIFFIIYIIIIAFFMMNIFVGFVIVTFQEQGEQEYKNCELDK | 1155 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1155 | IYNYRVEISIFFIIYIIIIAFFMMNIFVGFVIVTFQEQGEQEYKNCELDK | 1204 |
| | . . . . . | |
| 1156 | NQRQCVEYALKARPLRRYIPKNQHQYKVWYVVNSTYFEYLMFVLILLNTI | 1205 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1205 | NQRQCVEYALKARPLRRYIPKNQHQYKVWYVVNSTYFEYLMFVLILLNTI | 1254 |
| | . . . . . | |
| 1206 | CLAMQHYGQSCLFKIAMNILNMLFTGLFTVEMILKLIAFKPK........ | 1247 |
| | \|\|\|\|\|\|\|\|\|\|\|\|\|\|\|\|\|\|\|\|\|\|\|\|\|\|\|\|\|\|\|\|\|\|\|\|\|\|\|\|\|\| | |
| 1255 | CLAMQHYGQSCLFKIAMNILNMLFTGLFTVEMILKLIAFKPKGYFSDPWN | 1304 |
| | . . . . . | |
| 1248 | ....................HYFCDAWNTFDALTVVGSIVDIAITEVN.. | 1275 |
| | \|\|\|\|\|\|\|\|\|\|\|\|\|\|\|\|\|\|\|\|\|\|\|\|\|\|\|\| | |
| 1305 | VFDFLIVIGSIIDVILSETNHYFCDAWNTFDALIVVGSIVDIAITEVNPA | 1354 |
| | . . . . . | |
| 1276 | .........NAEENSRISITFFRLFRVMRLVKLLSRGEGIRTLLWTFIKS | 1316 |
| | \|\|\|\|\|\|\|\|\|\|\|\|\|\|\|\|\|\|\|\|\|\|\|\|\|\|\|\|\|\|\|\|\|\|\|\|\|\|\|\|\| | |
| 1355 | EHTQCSPSMNAEENSRISITFFRLFRVMRLVKLLSRGEGIRTLLWTFIKS | 1404 |
| | . . . . . | |
| 1317 | FQALPYVALLIVMLFFIYAVIGMQVFGKIALNDTTEINRNNNFQTFPQAV | 1366 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1405 | FQALPYVALLIVMLFFIYAVIGMQVFGKIALNDTTEINRNNNFQTFPQAV | 1454 |
| | . . . . . | |
| 1367 | LLLFRCATGEAWQDIMLACMPGKKCAPESEPSNSTEGETPCGSSFAVFYF | 1416 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1455 | LLLFRCATGEAWQDIMLACMPGKKCAPESEPSNSTEGETPCGSSFAVFYF | 1504 |
| | . . . . . | |
| 1417 | ISFYMLCAFLIINLFVAVIMDNFDYLTRDWSILGPHHLDEFKRIWAEYDP | 1466 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1505 | ISFYMLCAFLIINLFVAVIMDNFDYLTRDWSILGPHHLDEFKRIWAEYDP | 1554 |
| | . . . . . | |
| 1467 | EAKGRIKHLDVVTLLRRIQPPLGFGKLCPHRVACKRLVSMNMPLNSDGTV | 1516 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1555 | EAKGRIKHLDVVTLLRRIQPPLGFGKLCPHRVACKRLVSMNMPLNSDGTV | 1604 |
| | . . . . . | |
| 1517 | MFNATLFALVRTALRIKTEGNLEQANEELRAIIKKIWKRTSMKLLDQVVP | 1566 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1605 | MFNATLFALVRTALRIKTEGNLEQANEELRAIIKKIWKRTSMKLLDQVVP | 1654 |
| | . . . . . | |
| 1567 | PAGDDEVTVGKFYATFLIQEYFRKFKKRKEQGLVGKPSQRNALSLQAGLR | 1616 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1655 | PAGDDEVTVGKFYATFLIQEYFRKFKKRKEQGLVGKPSQRNALSLQAGLR | 1704 |
| | . . . . . | |
| 1617 | TLHDIGPEIRRAISGDLTAEEELDKAMKEAVSAASEDDIFRRAGGLFGNH | 1666 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1705 | TLHDIGPEIRRAISGDLTAEEELDKAMKEAVSAASEDDIFRRAGGLFGNH | 1754 |
| | . . . . . | |
| 1667 | VSYYQSDGRSAFPQTFTTQRPLHINKAGSSQGDTESPSHEKLVDSTFTPS | 1716 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1755 | VSYYQSDGRSAFPQTFTTQRPLHINKAGSSQGDTESPSHEKLVDSTFTPS | 1804 |
| | . . . . . | |
| 1717 | SYSSTGSNANINNANNTALGRLPRPAGYPSTVSTVEGHGPPLSPAIRVQE | 1766 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1805 | SYSSTGSNANINNANNTALGRLPRPAGYPSTVSTVEGHGPPLSPAIRVQE | 1854 |
| | . . . . . | |
| 1767 | VAWKLSSNR..................................MHCCDM | 1781 |
| | \|\|\|\|\|\|\|\|\| | |
| 1855 | VAWKLSSNRERHVPVCEDLELRRDSGSAGTQAHCLLLRRANPS...... | 1897 |
| | . . . . . | |
| 1782 | LDGGTFPPALGPRRAPPCLHQQLQGSLAGLREDTPCIVPGHASLCCSSRV | 1831 |
| 1897 | .................................................. | 1897 |
| | . . . . . | |
| 1832 | GEWLPAGCTAPQHARCHSRESQAAMAGQEETSQDETYEVKMNHDTEACSE | 1881 |
| | \|\|\|\|\|\|\|\|\|\|\|\|\|\|\|\|\|\|\|\|\|\|\|\|\|\|\|\|\|\|\|\|\|\|\|\| | |
| 1898 | ..............RCHSRESQAAMAGQEETSQDETYEVKMNHDTEACSE | 1933 |
| | . . . . . | |
| 1882 | PSLLSTEMLSYQDDENRQLTLPEEDKRDIRQSPKRGFLRSASLGRRASFH | 1931 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1934 | PSLLSTEMLSYQDDENRQLTLPEEDKRDIRQSPKRGFLRSASLGRRASFH | 1983 |
| | . . . . . | |
| 1932 | LECLKRQKDRGGDISQKTVLPLHLVHHQALAVAGLSPLLQRSHSPASFPR | 1981 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1984 | LECLKRQKDRGGDISQKTVLPLHLVHHQALAVAGLSPLLQRSHSPASFPR | 2033 |
| | . . . . . | |
| 1982 | PFATPPATPGSRGWPPQPVPTLRLEGVESSEKLNSSFPSIHCGSWAETTP | 2031 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2034 | PFATPPATPGSRGWPPQPVPTLRLEGVESSEKLNSSFPSIHCGSWAETTP | 2083 |
| | . . . . . | |
| 2032 | GGGGSSAARRVRPVSLMVPSQAGAPGRQFHGSASSLVEAVLISEGLGQFA | 2081 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2084 | GGGGSSAARRVRPVSLMVPSQAGAPGRQFHGSASSLVEAVLISEGLGQFA | 2133 |
| | . . . . . | |
| 2082 | QDPKFIEVTTQELADACDMTIEEMESAADNILSGGAPQSPNGALLPFVNC | 2131 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2134 | QDPKFIEVTTQELADACDMTIEEMESAADNILSGGAPQSPNGALLPFVNC | 2183 |
| | . . . . | |
| 2132 | RDAGQDRAGGEEDAGCVRARGRPSEEELQDSRVYVSSL | 2169 |
| | \|\|\|\|\|\|\|\|\|\|\|\|\|\|\|\|\|\|\|\|\| \|\|\|\|\|\|\|\|\|\|\|\|\|\|\|\| | |
| 2184 | RDAGQDRAGGEEDAGCVRARGAPSEEELQDSRVYVSSL | 2221 |

### EXAMPLE 16

This Example relates to the variant HUMLVDCCB_P20 (SEQ ID NO:6; the nucleic acid sequence is given by SEQ ID NO:28), which is a variant alpha 1 subunit according to the present invention, and more specifically, is a splice variant of the known protein CCAC_HUMAN, which is encoded by the CACNA1C gene. This alpha subunit is the alpha 1C subunit, which forms the voltage-dependent L-type calcium channel. This subunit is also called the alpha subunit Cav1.2. The known protein is described in greater detail above with regard to Example 14.

The structure of the splice variant according to the present invention features truncation of the protein, as compared to the known protein sequence. An alignment is provided at the end of this section, while the comparison between the two sequences is described below.

According to preferred embodiments of the present invention, there is provided an isolated chimeric polypeptide encoding for HUMLVDCCB_P20, consisting essentially of an amino acid sequence being at least 90% homologous to (SEQ ID NO: 148) MVNENTRMYIPEENHQGSNYGSPRPAHANMNANAAAGLAPEHIPTPGAALSWQAAIDAARQAKLMGSAGNATISTVSSTQRKRQQYGKPKKQGSTTATRPPRALLCLTLKNPIRRACISIVEWKPFEIIILLTIFANCVALAIYIPFPEDDSNATNSNLERVEYLFLIIFTVEAFLKVIAYGLLFHPNAYLRNGWNLLDFIIVVVG corresponding to amino acids 1-206 of CCAC_HUMAN, which also corresponds to amino acids 1-206 of HUMLVDCCB_P20.

Domains affected by alternative splicing include the S3 transmembrane domain of domain I; the extracellular loop between S3 and S4 transmembrane domains of domain I; the S4 transmembrane domain of domain I; the extracellular loop between S4 and S5 transmembrane domains of domain I; the S5 transmembrane domain of domain I; the pore loop region between S5 and S6 transmembrane domains of domain I; the S6 transmembrane domain of domain I; the cytoplasmic loop between domain I and domain II; the S1 transmembrane domain of domain II; the extracellular loop between S1 and S2 transmembrane domains of domain II; the S2 transmembrane domain of domain II; the extracellular loop between S2 and S3 transmembrane domains of domain II; the S3 transmembrane domain of domain II; the extracellular loop between S3 and S4 transmembrane domains of domain II; the S4 transmembrane domain of domain II; the extracellular loop between S4 and S5 transmembrane domains of domain II; the S5 transmembrane domain of domain II; the pore loop region between S5 and S6 transmembrane domains of domain II; the S6 transmembrane domain of domain II; the cytoplasmic loop between domain II and domain III; the S1 transmembrane domain of domain III; the extracellular loop between S1 and S2 transmembrane domains of domain III; the S2 transmembrane domain of domain III; the extracellular loop between S2 and S3 transmembrane domains of domain III; the S3 transmembrane domain of domain III; the extracellular loop between S3 and S4 transmembrane domains of domain III; the S4 transmembrane domain of domain III; the extracellular loop between S4 and S5 transmembrane domains of domain III; the S5 transmembrane domain of domain III; the pore loop region between S5 and S6 transmembrane domains of domain III; the S6 transmembrane domain of domain III; the cytoplasmic loop between domain III and domain IV; the S1 transmembrane domain of domain IV; the extracellular loop between S1 and S2 transmembrane domains of domain IV; the S2 transmembrane domain of domain IV; the extracellular loop between S2 and S3 transmembrane domains of domain IV; the S3 transmembrane domain of domain IV; the extracellular loop between S3 and S4 transmembrane domains of domain IV; the S4 transmembrane domain of domain IV; the extracellular loop between S4 and S5 transmembrane domains of domain IV; the S5 transmembrane domain of domain IV; the pore loop region between S5 and S6 transmembrane domains of domain IV; the S6 transmembrane domain of domain IV; the cytoplasmic C-terminus region of the protein.

One domain/two-domain channels are known in the art. It was previously demonstrated for N-type Cav2.2 channels that a channel consisting of domains I and II is not functional when expressed alone but only when coexpressed with a construct forming domains III and IV. However, coexpression of these very short one or two domain isoforms with the full length channel protein markedly reduced protein quantity and current density of the full length channel. Similarly, a three-domain channel which lacked the first domain and a part of the second domain by using an alternative promotor does not produce a measurable calcium current but instead, inhibits the functional expression of the full-length form. Therefore, this type of splicing may be a mechanism to transiently down-regulate a specific calcium channel without influencing promotor regulation.

From the above description of the function of variants lacking several domains, it is expected that the variant will function as a down regulation mechanism of a known functional channel protein. This variant may optionally be used for diagnostic applications, because the presence, absence or level of such downregulation may have diagnostic implications, for example for detecting a disease and/or for selecting a therapy for the disease.

Sequence name: CCAC_HUMAN documentation:

of: HUMLVDCCB_P20 (SEQ ID NO: 6)×CCAC_HUMAN (SEQ ID NO: 149) . . .

segment 1/1:

Quality: 2006.00

Escore: 0

Matching length: 206 Total

length: 206

Matching Percent Similarity: 100.00 Matching Percent

Identity: 100.00

Total Percent Similarity: 100.00 Total Percent

Identity: 100.00

Gaps: 0

| | . . . . . | |
|---|---|---|
| 1 | MVNENTRMYIPEENHQGSNYGSPRPAHANMNANAAAGLAPEHIPTPGAAL | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MVNENTRMYIPEENHQGSNYGSPRPAHANMNANAAAGLAPEHIPTPGAAL | 50 |
| | . . . . . | |
| 51 | SWQAAIDAARQAKLMGSAGNATISTVSSTQRKRQQYGKPKKQGSTTATRP | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | SWQAAIDAARQAKLMGSAGNATISTVSSTQRKRQQYGKPKKQGSTTATRP | 100 |
| | . . . . . | |
| 101 | PRALLCLTLKNPIRRACISIVEWKPFEIIILLTIFANCVALAIYIPFPED | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | PRALLCLTLKNPIRRACISIVEWKPFEIIILLTIFANCVALAIYIPFPED | 150 |
| | . . . . . | |
| 151 | DSNATNSNLERVEYLFLIIFTVEAFLKVIAYGLLFHPNAYLRNGWNLLDF | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | DSNATNSNLERVEYLFLIIFTVEAFLKVIAYGLLFHPNAYLRNGWNLLDF | 200 |
| 201 | IIVVVG | 206 |
| | \|\|\|\|\|\| | |
| 201 | IIVVVG | 206 |

### EXAMPLE 17

This Example relates to the variant HUMLVDCCB_P21 (SEQ ID NO:7; the nucleic acid sequence is given by SEQ ID NO:29), which is a variant alpha 1 subunit according to the present invention, and more specifically, is a splice variant of the known protein CCAC_HUMAN, which is encoded by the CACNA1C gene. This alpha subunit is the alpha 1C subunit, which forms the voltage-dependent L-type calcium channel. This subunit is also called the alpha subunit Cav1.2. The known protein is described in greater detail above with regard to Example 14.

The structure of the splice variant according to the present invention features truncation of the protein, as compared to the known protein sequence. An alignment is provided at the end of this section, while the comparison between the two sequences is described below.

According to preferred embodiments of the present invention, there is provided an isolated chimeric polypeptide encoding for HUMLVDCCB_P21, comprising a first amino acid sequence being at least 90% homologous to (SEQ ID NO: 150) MVNENTRMYIPEENHQGSNYGSPRPAHANMNANAAAGLAPEHIPTPGAALSWQAAIDAARQAKLMGSAGNATISTVSSTQRKRQQYGKPKKQGSTTATRPPRALLCLTLKNPIRRACISIVEWKPFEIIILLTIFANCVALAIYIPFPEDDSNATNSNLERVEYLFLIIFTVEAFLKVIAYGLLFHPNAYLRNGWNLLDFIIVVVGLFSAILEQATKADGANALGGKGAGFDVKALRAFRVLRPLRLVSGVPSLQVVLNSIIKAMVPLLHIALLVLFVIIIYAIIGLELFMGKMHKTCYNQEGIADVPAEDDPSPCALETGHGRQCQNGTVCKPGWDGPKHGITNFDNFAFAMLTVFQCITMEGWTDVLYWVNDAVGRDWPWIYFVTLIIIGSFFVLNLVLGVLSG corresponding to amino acids 1-406 of CCAC_HUMAN, which also corresponds to amino acids 1-406 of HUMLVDCCB_P21.

Domains affected by alternative splicing include the S3 transmembrane domain of domain I; the extracellular loop between S3 and S4 transmembrane domains of domain I; the S4 transmembrane domain of domain I; the extracellular loop between S4 and S5 transmembrane domains of domain I; the S5 transmembrane domain of domain I; the pore loop region between S5 and S6 transmembrane domains of domain I; the S6 transmembrane domain of domain I; the cytoplasmic loop between domain I and domain II; the S1 transmembrane domain of domain II; the extracellular loop between S1 and S2 transmembrane domains of domain II; the S2 transmembrane domain of domain II; the extracellular loop between S2 and S3 transmembrane domains of domain II; the S3 transmembrane domain of domain II; the extracellular loop between S3 and S4 transmembrane domains of domain II; the S4 transmembrane domain of domain II; the extracellular loop between S4 and S5 transmembrane domains of domain II; the S5 transmembrane domain of domain II; the pore loop region between S5 and S6 transmembrane domains of domain II; the S6 transmembrane domain of domain II; the cytoplasmic loop between domain II and domain III; the S1 transmembrane domain of domain III; the extracellular loop between S1 and S2 transmembrane domains of domain III; the S2 transmembrane domain of domain III; the extracellular loop between S2 and S3 transmembrane domains of domain III; the S3 transmembrane domain of domain III; the extracellular loop between S3 and S4 transmembrane domains of domain III; the S4 transmembrane domain of domain III; the extracellular loop between S4 and S5 transmembrane domains of domain III; the S5 transmembrane domain of domain III; the pore loop region between S5 and S6 transmembrane domains of domain III; the S6 transmembrane domain of domain III; the cytoplasmic loop between domain III and domain IV; the S1 transmembrane domain of domain IV; the extracellular loop between S1 and S2 transmembrane domains of domain IV; the S2 transmembrane domain of domain IV; the extracellular loop between S2 and S3 transmembrane domains of domain IV; the S3 transmembrane domain of domain IV; the extracellular loop between S3 and S4 transmembrane domains of domain IV; the S4 transmembrane domain of domain IV; the extracellular loop between S4 and S5 transmembrane domains of domain IV; the S5 transmembrane domain of domain IV; the pore loop region between S5 and S6 transmembrane domains of domain IV; the S6 transmembrane domain of domain IV; the cytoplasmic C-terminus region of the protein.

One domain/two-domain channels are known in the art. It was previously demonstrated for N-type Cav2.2 channels that a channel consisting of domains I and II is not functional when expressed alone but only when coexpressed with a construct forming domains III and IV. However, coexpression of these very short one or two domain isoforms with the full length channel protein markedly reduced protein quantity and current density of the full length channel. Similarly, a three-domain channel which lacked the first domain and a part of the second domain by using an alternative promotor does not produce a measurable calcium current but instead, inhibits the functional expression of the full-length form. Therefore, this type of splicing may be a mechanism to transiently down-regulate a specific calcium channel without influencing promotor regulation.

From the above description of the function of variants lacking several domains, it is expected that the variant will function as a down regulation mechanism of a known functional channel protein. This variant may optionally be used for diagnostic applications, because the presence, absence or level of such downregulation may have diagnostic implications, for example for detecting a disease and/or for selecting a therapy for the disease.

Sequence name: CCAC_HUMAN

documentation:

of: HUMLVDCCB_P21 (SEQ ID NO: 7)×CCAC_HUMAN (SEQ ID NO; 151) . . .

segment 1/1:

Quality: 3947.00

Escore: 0

Matching length: 406 Total

length: 406

Matching Percent Similarity: 100.00 Matching Percent

Identity: 100.00

Total Percent Similarity: 100.00 Total Percent

Identity: 100.00

Gaps: 0

| | . . . . | |
|---|---|---|
| 1 | MVNENTRMYIPEENHQGSNYGSPRPAHANMNANAAAGLAPEHIPTPGAAL | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MVNENTRMYIPEENHQGSNYGSPRPAHANMNANAAAGLAPEHIPTPGAAL | 50 |
| | . . . . | |
| 51 | SWQAAIDAARQAKLMGSAGNATISTVSSTQRKRQQYGKPKKQGSTTATRP | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | SWQAAIDAARQAKLMGSAGNATISTVSSTQRKRQQYGKPKKQGSTTATRP | 100 |
| | . . . . | |
| 101 | PRALLCLTLKNPIRRACISIVEWKPFEIIILLTIFANCVALAIYIPFPED | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | PRALLCLTLKNPIRRACISIVEWKPFEIIILLTIFANCVALAIYIPFPED | 150 |
| | . . . . | |
| 151 | DSNATNSNLERVEYLFLIIFTVEAFLKVIAYGLLFHPNAYLRNGWNLLDF | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | DSNATNSNLERVEYLFLIIFTVEAFLKVIAYGLLFHPNAYLRNGWNLLDF | 200 |
| | . . . . | |
| 201 | IIVVVGLFSAILEQATKADGANALGGKGAGFDVKALRAFRVLRPLRLVSG | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | IIVVVGLFSAILEQATKADGANALGGKGAGFDVKALRAFRVLRPLRLVSG | 250 |
| | . . . . | |
| 251 | VPSLQVVLNSIIKAMVPLLHIALLVLFVIIIYAIIGLELFMGKMHKTCYN | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | VPSLQVVLNSIIKAMVPLLHIALLVLFVIIIYAIIGLELFMGKMHKTCYN | 300 |
| | . . . . | |
| 301 | QEGIADVPAEDDPSPCALETGHGRQCQNGTVCKPGWDGPKHGITNFDNFA | 350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | QEGIADVPAEDDPSPCALETGHGRQCQNGTVCKPGWDGPKHGITNFDNFA | 350 |
| | . . . . | |
| 351 | FAMLTVFQCITMEGWTDVLYWVNDAVGRDWPWIYFVTLIIIGSFFVLNLV | 400 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 351 | FAMLTVFQCITMEGWTDVLYWVNDAVGRDWPWIYFVTLIIIGSFFVLNLV | 400 |
| 401 | LGVLSG | 406 |
| | \|\|\|\|\|\| | |
| 401 | LGVLSG | 406 |

### EXAMPLE 18

This Example relates to the variant HUMLVDCCB_P13 (SEQ ID NO:8; the nucleic acid sequence is given by SEQ ID NO:30), which is a variant alpha 1 subunit according to the present invention, and more specifically, is a splice variant of the known protein CCAC_HUMAN, which is encoded by the CACNA1C gene. This alpha subunit is the alpha 1C subunit, which forms the voltage-dependent L-type calcium channel. This subunit is also called the alpha subunit Cav1.2. The known protein is described in greater detail above with regard to Example 14.

The structure of the splice variant according to the present invention features truncation of the protein, as compared to the known protein sequence. An alignment is provided at the end of this section, while the comparison between the two sequences is described below.

According to preferred embodiments of the present invention, there is provided an isolated chimeric polypeptide encoding for HUMLVDCCB_P13, comprising a first amino acid sequence being at least 90% homologous to (SEQ ID NO: 152) MVNENTRMYIPEENHQGSNYGSPRPAHANMNANAAAGLAPEHIPTPGAALSWQAAIDAARQAKLMGSAGNATISTVSSTQRKRQQYGKPKKQGSTTATRPPRALLCLTLKNPIRRACISIVEWKPFEIIILLTIFANCVALAIYIPFPEDDSNATNSNLERVEYLFLIIFTVEAFLKVIAYGLLFHPNAYLRNGWNLLDFIIVVVGLFSAILEQATKADGANALGGKGAGFDVKALRAFRVLRPLRLVSGVPSLQVVLNSIIKAMVPLLHIALLVLFVIIIYAIIGLELFMGKMHKTCYNQEGIADVPAEDDPSPCALETGHGRQCQNGTVCKPGWDGPKHGITNFDNFAFAMLTVFQCITMEGWTDVLYWVNDAVGRDWPWIYFVTLIIIGSFFVLNLVLGVLSGEFSKEREKAKARGDFQKLREKQQLEEDLKGYLDWITQAEDIDPENEDEGMDEEKPRN corresponding to amino acids 1-463 of CCAC_HUMAN, which also corresponds to amino acids 1-463 of HUMLVDCCB_P13, a second amino acid sequence being at least 70%, optionally at least 80%, preferably at least 85%, more preferably at least 90% and most preferably at least 95% homologous to a polypeptide having the sequence (SEQ ID NO: 153) RGTPAGMLDQKKGKFAWFSHSTETHV corresponding to amino acids 464-489 of HUMLVDCCB_P13, a third amino acid sequence being at least 90% homologous to (SEQ ID NO: 154) SMPTSETESVNTENVAGGDIEGENCGARLAHRISKSKFSRYWRRWNRFCRRKCRAAVKSNVFYWLVIFLVFLNTLTIASEHYNQPNWLTEVQDTANKALLALFTAEMLLKMYSLGLQAYFVSLFNRFDCFVVCGGILETILVETKIMSPLGISVLRCVRLLRIFKITRYWNSLSNLVASLLNSVRSIASLLLLLFLFIIIFSLLGMQLFGGKFNFDEMQTRRSTFDNFPQSLLTVFQILTGEDWNSVMYDGIMAYGGPSFPGMLVCIYFIILFICGNYILLNVFLAIAVDNLADAESLTSAQKEEEEEKERKKLARTASPEKKQELVEKPAVGESKEEKIELKSITADGESPPATKINMDDLQPNENEDKSPYPNPETTGEEDEEEPEMPVGPRPRPLSELHLKEKAVPMPEASAFFIFSSNNRFRLQCHRIVNDTIFTNLILFFILLSSISLAAEDPVQHTSFRNHILFYFDIVFTTIFTIEI A corresponding to amino acids 465-949 of CCAC_HUMAN, which also corresponds to amino acids 490-974 of HUMLVDCCB_P13, a fourth amino acid sequence being at least 90% homologous to (SEQ ID NO: 155) LKMTAYGAFLHKGSFCRNYFNILDLLVVSVSLISFGIQSSAINVVKILRVLRVLRPLRAINRAKGLKHVVQCVFVAIRTIGNIVIVTTLLQFMFACIGVQLFKGKLYTCSDSSKQTEAECKGNYITYKDGEVDHPIIQPRSWENSKFDFDNVLAAMMALFTVSTFEGWPELLYRSIDSHTEDKGPIYNYRVEISIFFIIYIIIIAFFMMNIFVGFVIVTFQEQGEQEYKNCELDKNQRQCVEYALKARPLRRYIPKNQHQYKVWYVVNSTYFEYLMFVLILLNTICLAMQHYGQSCLFKIAMNILNMLFTGLFTVEMILKLIAFKPK corresponding to amino acids 970-1296 of CCAC_HUMAN, which also corresponds to amino acids 975-1301 of HUMLVDCCB_P13, a fifth amino acid sequence being at least 90% homologous to (SEQ ID NO: 156) HYFCDAWNTFDALIVVGSIVDIAITEVNPAEHTQCSPSMNAEENSRISITFFRLFRVMRLVKLLSRGEGIRTLLWTFIKSFQALPYVALLIVMLFFIYAVIGMQVFGKIALNDTTEINRNNNFQTFPQAVLLLFRCATGEAWQDIMLACMPGKKCAPESEPSNSTEGETPCGSSFAVFYFISFYMLCAFLIINLFVAVIMDNFDYLTRDWSILGPHHLDEFKRIWAEYDPEAKGRIKHLDVVTLLRRIQPPLGFGKLCPHRVACKRLVSMNMPLNSDGTVMFNATLFALVRTALRIKTE corresponding to amino acids 1325-1623 of CCAC_HUMAN, which also corresponds to amino acids 1302-1600 of HUMLVDCCB_P13, a sixth amino acid sequence being at least 70%, optionally at least 80%, preferably at least 85%, more preferably at least 90% and most preferably at least 95% homologous to a polypeptide having the sequence (SEQ ID NO: 157) EGPSPSEAHQGAEDPFRPA corresponding to amino acids 1601-1619 of HUMLVDCCB_P113, a seventh amino acid sequence being at least 90% homologous to (SEQ ID NO: 158) GNLEQANEELRAIIKKIWKRTSMKLLDQVVPPAGDDEVTVGKFYATFLIQEYFRKFKKRKEQGLVGKPSQRNALSLQAGLRTLHDIGPEIRRAISGDLTAEEELDKAMKEAVSAASEDDIFRRAGGLFGNHVSYYQSDGRSAFPQTFTTQRPLHINKAGSSQGDTESPSHEKLVDSTFTPSSYSSTGSNANINNANNTALGRLPRPAGYPSTVSTVEGHGPPLSPAIRVQEVAWKLSSN corresponding to amino acids 1624-1862 of CCAC_HUMAN, which also corresponds to amino acids 1620-1858 of HUMLVDCCB_P13, a eight amino acid sequence being at least 90% homologous to (SEQ ID NO: 159) RCHSRESQAAMAGQEETSQDETYEVKMNHDTEACSEPSLLSTEMLSYQDDENRQLTLPEEDKRDIRQSPKRGFLRSASLGRRASFHLECLKRQKDRGGDISQKTVLPLHLVHHQALAVAGLSPLLQRSHSPASFPRPFATPPATPGSRGWPPQPVPTLRLEGVESSEKLNSSFPSIHCGSWAETTPGGGGSSAARRVRPVSLMVPSQAGAPGRQFHGSASSLVEAVLISEGLGQFAQDPKFIEVTTQELADACDMTIEEMESAADNILSGGAPQSPNGALLPFVNCRDAGQDRAGGEEDAGCVRARGAPSEEELQDSRVYVSSL corresponding to amino acids 1898-2221 of CCAC_HUMAN, which also corresponds to amino acids 1859-2182 of HUMLVDCCB_P13, wherein said first amino acid sequence, second amino acid sequence, third amino acid sequence, fourth amino acid sequence, fifth amino acid sequence, sixth amino acid sequence, seventh amino acid sequence and eight amino acid sequence are contiguous and in a sequential order.

According to preferred embodiments of the present invention, there is provided an isolated polypeptide encoding for an edge portion of HUMLVDCCB_P13, comprising an amino acid sequence being at least 70%, optionally at least about 80%, preferably at least about 85%, more preferably at least about 90% and most preferably at least about 95% homologous to the sequence encoding for (SEQ ID NO: 160) RGTPAGMLDQKKGKFAWFSHSTETHV, corresponding to HUMLVDCCB_P13.

According to preferred embodiments of the present invention, there is provided an bridge portion of HUMLVDCCB_P13, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise NR, having a structure as follows (numbering according to HUMLVDCCB_P13): a sequence starting from any of amino acid numbers 463−x to 463; and ending at any of amino acid numbers 464+((n−2)−x), in which x varies from 0 to n−2.

According to preferred embodiments of the present invention, there is provided an isolated chimeric polypeptide encoding for an edge portion of HUMLVDCCB_P13, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise AL, having a structure as follows: a sequence starting from any of amino acid numbers 974−x to 974; and ending at any of amino acid numbers 975+((n−2)−x), in which x varies from 0 to n−2.

According to preferred embodiments of the present invention, there is provided an isolated chimeric polypeptide encoding for an edge portion of HUMLVDCCB_P113, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise KH, having a structure as follows: a sequence starting from any of amino acid numbers 1301−x to 1301; and ending at any of amino acid numbers 1302+((n−2)−x), in which x varies from 0 to n−2.

According to preferred embodiments of the present invention, there is provided an isolated polypeptide encoding for an edge portion of HUMLVDCCB_P13, comprising an amino acid sequence being at least 70%, optionally at least about 80%, preferably at least about 85%, more preferably at least about 90% and most preferably at least about 95% homologous to the sequence encoding for (SEQ ID NO: 161) EGPSPSEAHQGAEDPFRPA, corresponding to HUMLVDCCB_P13.

According to preferred embodiments of the present invention, there is provided an isolated chimeric polypeptide encoding for an edge portion of HUMLVDCCB_P13, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise NR, having a structure as follows: a sequence starting from any of amino acid numbers 1858−x to 1858; and ending at any of amino acid numbers 1859+((n−2)−x), in which x varies from 0 to n−2.

1. Domains affected by alternative splicing include: the cytoplasmic loop between domain I and domain II; the S2 transmembrane domain of domain III; the extracellular loop between S2 and S3 transmembrane domains of domain III; the extracellular loop between S2 and S3 transmembrane domains of domain IV; the S3 transmembrane domain of domain IV; the extracellular loop between S3 and S4 transmembrane domains of domain IV the cytoplasmic C-terminus region of the protein.

The potential effect of these changes is described with regard to Example 14 above.

From the above description of the effect of changes to the C-terminus domain of this variant, it is expected that the variant will have greater current amplitude as compared to the known protein and a stronger calcium dependence of inactivation. It is also expected to modulate the Ca+2 dependent inactivation of the channel.

In addition, the changes in the S2 transmembrane domain of domain III might influence the voltage-dependent action of dihydropyridines.

Sequence name: CCAC_HUMAN

documentation:

of: HUMLVDCCB_P13 (SEQ ID NO: 8)×CCAC_HUMAN (SEQ ID NO: 162) . . . segment 1/1:

Quality: 20374.00

Escore: 0

Matching lenght: 2138 Total

length: 2265

Matching Percent Similarity: 100.00 Matching Percent

Identity: 99.95

Total Percent Similarity: 94.39 Total Percent

Identity: 94.35

Gaps:

| | . . . . | |
|---|---|---|
| 1 | MVNENTRMYIPEENHQGSNYGSPRPAHANMNANAAAGLAPEHIPTPGAAL | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MVNENTRMYIPEENHQGSNYGSPRPAHANMNANAAAGLAPEHIPTPGAAL | 50 |
| | . . . . | |
| 51 | SWQAAIDAARQAKLMGSAGNATISTVSSTQRKRQQYGKPKKQGSTTATRP | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | SWQAAIDAARQAKLMGSAGNATISTVSSTQRKRQQYGKPKKQGSTTATRP | 100 |
| | . . . . | |
| 101 | PRALLCLTLKNPIRRACISIVEWKPFEIIILLTIFANCVALAIYIPFPED | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | PRALLCLTLKNPIRRACISIVEWKPFEIIILLTIFANCVALAIYIPFPED | 150 |
| | . . . . | |
| 151 | DSNATNSNLERVEYLFLIIFTVEAFLKVIAYGLLFHPNAYLRNGWNLLDF | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | DSNATNSNLERVEYLFLIIFTVEAFLKVIAYGLLFHPNAYLRNGWNLLDF | 200 |
| | . . . . | |
| 201 | IIVVVGLFSAILEQATKADGANALGGKGAGFDVKALRAFRVLRPLRLVSG | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | IIVVVGLFSAILEQATKADGANALGGKGAGFDVKALRAFRVLRPLRLVSG | 250 |
| | . . . . | |
| 251 | VPSLQVVLNSIIKAMVPLLHIALLVLFVIIIYAIIGLELFMGKMHKTCYN | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | VPSLQVVLNSIIKAMVPLLHIALLVLFVIIIYAIIGLELFMGKMHKTCYN | 300 |
| | . . . . | |
| 301 | QEGIADVPAEDDPSPCALETGHGRQCQNGTVCKPGWDGPKHGITNFDNFA | 350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | QEGIADVPAEDDPSPCALETGHGRQCQNGTVCKPGWDGPKHGITNFDNFA | 350 |
| | . . . . | |
| 351 | FAMLTVFQCITMEGWTDVLYWVNDAVGRDWPWIYFVTLIIIGSFFVLNLV | 400 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 351 | FAMLTVFQCITMEGWTDVLYWVNDAVGRDWPWIYFVTLIIIGSFFVLNLV | 400 |
| | . . . . | |
| 401 | LGVLSGEFSKEREKAKARGDFQKLREKQQLEEDLKGYLDWITQAEDIDPE | 450 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 401 | LGVLSGEFSKEREKAKARGDFQKLREKQQLEEDLKGYLDWITQAEDIDPE | 450 |
| | . . . . | |
| 451 | NEDEGMDEEKPRNRGTPAGMLDQKKGKFAWFSHSTETHVSMPTSETESVN | 500 |
| | \|\|\|\|\|\|\|\|\|\|\|\|\| :\|\|\|\|\|\|\|\|\|\|\| | |
| 451 | NEDEGMDEEKPRN.........................MSMPTSETESVN | 475 |
| | . . . . | |
| 501 | TENVAGGDIEGENCGARLAHRISKSKFSRYWRRWNRFCRRKCRAAVKSNV | 550 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 476 | TENVAGGDIEGENCGARLAHRISKSKFSRYWRRWNRFCRRKCRAAVKSNV | 525 |
| | . . . . | |
| 551 | FYWLVIFLVFLNTLTIASEHYNQPNWLTEVQDTANKALLALFTAEMLLKM | 600 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 526 | FYWLVIFLVFLNTLTIASEHYNQPNWLTEVQDTANKALLALFTAEMLLKM | 575 |
| | . . . . | |
| 601 | YSLGLQAYFVSLFNRFDCFVVCGGILETILVETKIMSPLGISVLRCVRLL | 650 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 576 | YSLGLQAYFVSLFNRFDCFVVCGGILETILVETKIMSPLGISVLRCVRLL | 625 |
| | . . . . | |
| 651 | RIFKITRYWNSLSNLVASLLNSVRSIASLLLLLFLFIIIFSLLGMQLFGG | 700 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 626 | RIFKITRYWNSLSNLVASLLNSVRSIASLLLLLFLFIIIFSLLGMQLFGG | 675 |
| | . . . . | |
| 701 | KFNFDEMQTRRSTFDNFPQSLLTVFQILTGEDWNSVMYDGIMAYGGPSFP | 750 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 676 | KFNFDEMQTRRSTFDNFPQSLLTVFQILTGEDWNSVMYDGIMAYGGPSFP | 725 |
| | . . . . | |
| 751 | GMLVCIYFIILFICGNYILLNVFLAIAVDNLADAESLTSAQKEEEEEKER | 800 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 726 | GMLVCIYFIILFICGNYILLNVFLAIAVDNLADAESLTSAQKEEEEEKER | 775 |
| | . . . . | |
| 801 | KKLARTASPEKKQELVEKPAVGESKEEKIELKSITADGESPPATKINMDD | 850 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 776 | KKLARTASPEKKQELVEKPAVGESKEEKIELKSITADGESPPATKINMDD | 825 |
| | . . . . | |
| 851 | LQPNENEDKSPYPNPETTGEEDEEEPEMPVGPRPRPLSELHLKEKAVPMP | 900 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 826 | LQPNENEDKSPYPNPETTGEEDEEEPEMPVGPRPRPLSELHLKEKAVPMP | 875 |
| | . . . . | |
| 901 | EASAFFIFSSNNRFRLQCHRIVNDTIFTNLILFFILLSSISLAAEDPVQH | 950 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 876 | EASAFFIFSSNNRFRLQCHRIVNDTIFTNLILFFILLSSISLAAEDPVQH | 925 |
| | . . . . | |
| 951 | TSFRNHILFYFDIVFTTIFTIEIA....................LKMTAY | 980 |
| | \|\|\|\|\|\|\|\|\|\|\|\|\|\|\|\|\|\|\|\|\|\|\|\| \|\|\|\|\|\| | |
| 926 | TSFRNHILFYFDIVFTTIFTIEIALKILGNADYVFTSIFTLEIILKMTAY | 975 |
| | . . . . | |
| 981 | GAFLHKGSFCRNYFNILDLLVVSVSLISFGIQSSAINVVKILRVLRVLRP | 1030 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 976 | GAFLHKGSFCRNYFNILDLLVVSVSLISFGIQSSAINVVKILRVLRVLRP | 1025 |
| | . . . . | |
| 1031 | LRAINRAKGLKHVVQCVFVAIRTIGNIVIVTTLLQFMFACIGVQLFKGKL | 1080 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1026 | LRAINRAKGLKHVVQCVFVAIRTIGNIVIVTTLLQFMFACIGVQLFKGKL | 1075 |
| | . . . . | |
| 1081 | YTCSDSSKQTEAECKGNYITYKDGEVDHPIIQPRSWENSKFDFDNVLAAM | 1130 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1076 | YTCSDSSKQTEAECKGNYITYKDGEVDHPIIQPRSWENSKFDFDNVLAAM | 1125 |
| | . . . . | |
| 1131 | MALFTVSTFEGWPELLYRSIDSHTEDKGPIYNYRVEISIFFIIYIIIIAF | 1180 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1126 | MALFTVSTFEGWPELLYRSIDSHTEDKGPIYNYRVEISIFFIIYIIIIAF | 1175 |
| | . . . . | |
| 1181 | FMMNIFVGFVIVTFQEQGEQEYKNCELDKNQRQCVEYALKARPLRRYIPK | 1230 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1176 | FMMNIFVGFVIVTFQEQGEQEYKNCELDKNQRQCVEYALKARPLRRYIPK | 1225 |
| | . . . . | |
| 1231 | NQHQYKVWYVVNSTYFEYLMFVLILLNTICLAMQHYGQSCLFKIAMNILN | 1280 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1226 | NQHQYKVWYVVNSTYFEYLMFVLILLNTICLAMQHYGQSCLFKIAMNILN | 1275 |
| | . . . . | |
| 1281 | MLFTGLFTVEMILKLIAFKPK............................H | 1302 |
| | \|\|\|\|\|\|\|\|\|\|\|\|\|\|\|\|\|\|\|\|\| \| | |
| 1276 | MLFTGLFTVEMILKLIAFKPKGYFSDPWNVFDFLIVIGSIIDVILSETNH | 1325 |
| | . . . . | |
| 1303 | YFCDAWNTFDALIVVGSIVDIAITEVNPAEHTQCSPSMNAEENSRISITF | 1352 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1326 | YFCDAWNTFDALIVVGSIVDIAITEVNPAEHTQCSPSMNAEENSRISITF | 1375 |
| | . . . . | |
| 1353 | FRLFRVMRLVKLLSRGEGIRTLLWTFIKSFQALPYVALLIVMLFFIYAVI | 1402 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1376 | FRLFRVMRLVKLLSRGEGIRTLLWTFIKSFQALPYVALLIVMLFFIYAVI | 1425 |
| | . . . . | |
| 1403 | GMQVFGKIALNDTTEINRNNNFQTFPQAVLLLFRCATGEAWQDIMLACMP | 1452 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1426 | GMQVFGKIALNDTTEINRNNNFQTFPQAVLLLFRCATGEAWQDIMLACMP | 1475 |
| | . . . . | |
| 1453 | GKKCAPESEPSNSTEGETPCGSSFAVFYFISFYMLCAFLIINLFVAVIMD | 1502 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1476 | GKKCAPESEPSNSTEGETPCGSSFAVFYFISFYMLCAFLIINLFVAVIMD | 1525 |
| | . . . . | |
| 1503 | NFDYLTRDWSILGPHHLDEFKRIWAEYDPEAKGRIKHLDVVTLLRRIQPP | 1552 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1526 | NFDYLTRDWSILGPHHLDEFKRIWAEYDPEAKGRIKHLDVVTLLRRIQPP | 1575 |
| | . . . . | |
| 1553 | LGFGKLCPHRVACKRLVSMNMPLNSDGTVMFNATLFALVRTALRIKTEEG | 1602 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1576 | LGFGKLCPHRVACKRLVSMNMPLNSDGTVMFNATLFALVRTALRIKTE.. | 1623 |
| | . . . . | |
| 1603 | PSPSEAHQGAEDPFRPAGNLEQANEELRAIIKKIWKRTSMKLLDQVVPPA | 1652 |
| | \|\|\|\|\|\|\|\|\|\|\|\|\|\|\|\|\|\|\|\|\|\|\|\|\|\|\|\|\|\|\|\|\| | |
| 1624 | .................GNLEQANEELRAIIKKIWKRTSMKLLDQVVPPA | 1656 |
| | . . . . | |
| 1653 | GDDEVTVGKFYATFLIQEYFRKFKKRKEQGLVGKPSQRNALSLQAGLRTL | 1702 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1657 | GDDEVTVGKFYATFLIQEYFRKFKKRKEQGLVGKPSQRNALSLQAGLRTL | 1706 |
| | . . . . | |
| 1703 | HDIGPEIRRAISGDLTAEEELDKAMKEAVSAASEDDIFRRAGGLFGNHVS | 1752 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1707 | HDIGPEIRRAISGDLTAEEELDKAMKEAVSAASEDDIFRRAGGLFGNHVS | 1756 |
| | . . . . | |
| 1753 | YYQSDGRSAFPQTFTTQRPLHINKAGSSQGDTESPSHEKLVDSTFTPSSY | 1802 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1757 | YYQSDGRSAFPQTFTTQRPLHINKAGSSQGDTESPSHEKLVDSTFTPSSY | 1806 |
| | . . . . | |
| 1803 | SSTGSNANINNANNTALGRLPRPAGYPSTVSTVEGHGPPLSPAIRVQEVA | 1852 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1807 | SSTGSNANINNANNTALGRLPRPAGYPSTVSTVEGHGPPLSPAIRVQEVA | 1856 |
| | . . . . | |
| 1853 | WKLSSN...................................RCHSRESQA | 1867 |
| | \|\|\|\|\|\| \|\|\|\|\|\|\|\|\| | |
| 1857 | WKLSSNRERHVPVCEDLELRRDSGSAGTQAHCLLLRRANPSRCHSRESQA | 1906 |
| | . . . . | |
| 1868 | AMAGQEETSQDETYEVKMNHDTEACSEPSLLSTEMLSYQDDENRQLTLPE | 1917 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1907 | AMAGQEETSQDETYEVKMNHDTEACSEPSLLSTEMLSYQDDENRQLTLPE | 1956 |
| | . . . . | |
| 1918 | EDKRDIRQSPKRGFLRSASLGRRASFHLECLKRQKDRGGDISQKTVLPLH | 1967 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1957 | EDKRDIRQSPKRGFLRSASLGRRASFHLECLKRQKDRGGDISQKTVLPLH | 2006 |
| | . . . . | |
| 1968 | LVHHQALAVAGLSPLLQRSHSPASFPRPFATPPATPGSRGWPPQPVPTLR | 2017 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2007 | LVHHQALAVAGLSPLLQRSHSPASFPRPFATPPATPGSRGWPPQPVPTLR | 2056 |
| | . . . . | |
| 2018 | LEGVESSEKLNSSFPSIHCGSWAETTPGGGGSSAARRVRPVSLMVPSQAG | 2067 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2057 | LEGVESSEKLNSSFPSIHCGSWAETTPGGGGSSAARRVRPVSLMVPSQAG | 2106 |
| | . . . . | |
| 2068 | APGRQFHGSASSLVEAVLISEGLGQFAQDPKFIEVTTQELADACDMTIEE | 2117 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2107 | APGRQFHGSASSLVEAVLISEGLGQFAQDPKFIEVTTQELADACDMTIEE | 2156 |
| | . . . . | |
| 2118 | MESAADNILSGGAPQSPNGALLPFVNCRDAGQDRAGGEEDAGCVRARGAP | 2167 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2157 | MESAADNILSGGAPQSPNGALLPFVNCRDAGQDRAGGEEDAGCVRARGAP | 2206 |
| | . | |
| 2168 | SEEELQDSRVYVSSL | 2182 |
| | \|\|\|\|\|\|\|\|\|\|\|\|\|\|\| | |
| 2207 | SEEELQDSRVYVSSL | 2221 |

### EXAMPLE 19

This Example relates to the variant HUMCACH1A_P6 (SEQ ID NO:21; the nucleic acid sequence is given by SEQ ID NO:43), which is a variant alpha 1 subunit according to the present invention, and more specifically, is a splice variant of the known protein CCAD_HUMAN, which is encoded by the CACNA1D gene. This alpha subunit is the alpha 1D subunit, which forms the voltage-dependent L-type calcium channel. It is also known as alpha subunit Cav1.3.

This alpha subunit is expressed in pancreatic islets and in brain, where it has been seen in hippocampus, basal ganglia, habenula and thalamus. The protein structure is as follows. Each of the four internal repeats contains five hydrophobic transmembrane segments (S1, S2, S3, S5, S6) and one positively charged transmembrane segment (S4). S4 segments probably represent the voltage-sensor and are characterized by a series of positively charged amino acids at every third position.

The structure of the splice variant according to the present invention features a unique insertion, as compared to the known protein sequence. An alignment is provided at the end of this section, while the comparison between the two sequences is described below.

According to preferred embodiments of the present invention, there is provided an isolated chimeric polypeptide encoding for HUMCACH1A_P6, comprising a first amino acid sequence being at least 90% homologous to (SEQ ID NO: 163) MMMMMMMKKMQHQRQQQADHANEANYARGTRLPLSGEGPTSQPNSSKQTVLSWQAAIDAARQAKAAQTMSTSAPPPVGSLSQRKRQQYAKSKKQGNSSNSRPARALFCLSLNNPIRRACISIVEWKPFDIFILLAIFANCVALAIYIPFPEDDSNSTNHNLEKVEYAFLIIFTVETFLKIIAYGLLLHPNAYVRNGWNLLDFVIVIVGLFSVILEQLTKETEGGNHSSGKSGGFDVKALRAFRVLRPLRLVSGVPSLQVVLNSIIKAMVPLLHIALLVLFVIIIYAIIGLELFIGKMHKTCFFADSDIVAEEDPAPCAFSGNGRQCTANGTECRSGWVGPNGGITNFDNFAFAMLTVFQCITMEGWTDVLYW corresponding to amino acids 1-372 of CCAD_HUMAN, which also corresponds to amino acids 1-372 of HUMCACH1A_P6, a second amino acid sequence comprising a polypeptide being at least 70%, optionally at least about 80%, preferably at least about 85%, more preferably at least about 90% and most preferably at least about 95% homologous to the sequence encoding for (SEQ ID NO: 164) VNDAIGWEWPWVYFVSLIIL corresponding to amino acids 373-392 of HUMCACH1A_P6 and a third amino acid sequence being at least 90% homologous to (SEQ ID NO: 165) GSFFVLNLVLGVLSG corresponding to amino acids 393-407 of CCAD_HUMAN, which also corresponds to amino acids 393-407 of HUMCACH1A_P6, wherein said first amino acid sequence, second amino acid sequence and third amino acid sequence are contiguous and in a sequential order.

According to preferred embodiments of the present invention, there is provided an isolated polypeptide encoding for a unique insertion of HUMCACH1A_P6, comprising an amino acid sequence being at least 70%, optionally at least about 80%, preferably at least about 85%, more preferably at least about 90% and most preferably at least about 95% homologous to the sequence encoding for (SEQ ID NO: 166) VNDAIGWEWPWVYFVSLIIL, corresponding to HUMCACH1A_P6.

1. Domains affected by alternative splicing include the cytoplasmic loop between domain I and domain II; the S1 transmembrane domain of domain II; the extracellular loop between S1 and S2 transmembrane domains of domain II; the S2 transmembrane domain of domain II; the extracellular loop between S2 and S3 transmembrane domains of domain II; the S3 transmembrane domain of domain II; the extracellular loop between S3 and S4 transmembrane domains of domain II; the S4 transmembrane domain of domain II; the extracellular loop between S4 and S5 transmembrane domains of domain II; the S5 transmembrane domain of domain II; the pore loop region between S5 and S6 transmembrane domains of domain II; the S6 transmembrane domain of domain II; the cytoplasmic loop between domain II and domain III; the S1 transmembrane domain of domain III; the extracellular loop between S1 and S2 transmembrane domains of domain III; the S2 transmembrane domain of domain III; the extracellular loop between S2 and S3 transmembrane domains of domain III; the S3 transmembrane domain of domain III; the extracellular loop between S3 and S4 transmembrane domains of domain III; the S4 transmembrane domain of domain III; the extracellular loop between S4 and S5 transmembrane domains of domain III; the S5 transmembrane domain of domain III; the pore loop region between S5 and S6 transmembrane domains of domain III; the S6 transmembrane domain of domain III; the cytoplasmic loop between domain III and domain IV; the S1 transmembrane domain of domain IV; the extracellular loop between S1 and S2 transmembrane domains of domain IV; the S2 transmembrane domain of domain IV; the extracellular loop between S2 and S3 transmembrane domains of domain IV; the S3 transmembrane domain of domain IV; the extracellular loop between S3 and S4 transmembrane domains of domain IV; the S4 transmembrane domain of domain IV; the extracellular loop between S4 and S5 transmembrane domains of domain IV; the S5 transmembrane domain of domain IV; the pore loop region between S5 and S6 transmembrane domains of domain IV; the S6 transmembrane domain of domain IV; the cytoplasmic C-terminus region of the protein.

One domain/two-domain channels are known in the art. It was previously demonstrated for N-type Cav2.2 channels that a channel consisting of domains I and II is not functional when expressed alone but only when coexpressed with a construct forming domains III and IV. However, coexpression of these very short one or two domain isoforms with the full length channel protein markedly reduced protein quantity and current density of the full length channel. Similarly, a three-domain channel which lacked the first domain and a part of the second domain by using an alternative promotor does not produce a measurable calcium current but instead, inhibits the functional expression of the full-length form. Therefore, this type of splicing may be a mechanism to transiently down-regulate a specific calcium channel without influencing promotor regulation.

From the above description of the function of variants lacking several domains, it is expected that the variant will function as a down regulation mechanism of a known functional channel protein. This variant may optionally be used for diagnostic applications, because the presence, absence or level of such downregulation may have diagnostic implications, for example for detecting a disease and/or for selecting a therapy for the disease.

Sequence name: CCAD_HUMAN

documentaiton:

of: HUMCACH1A_P6 (SEQ ID NO: 21)×CCAD_HUMAN (SEQ ID NO: 167) . . .

segment 1/1:

Quality: 365.00

Escore: 0

Matching length: 385 Total

length: 429

Matching Percent Similarity: 100.00 Matching Percent

Identity: 100.00

Total Percent Similarity: 89.74 Total Percent

Identity: 89.74

Gaps: 2

Alignment:

| | . . . . . | |
|---|---|---|
| 1 | MMMMMMMKKMQHQRQQQADHANEANYARGTRLPLSGEGPTSQPNSSKQTV | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MMMMNMMKKMQHQRQQQADHANEANYARGTRLPLSGEGPTSQPNSSKQTV | 50 |
| | . . . . . | |
| 51 | LSWQAAIDAARQAKAAQTMSTSAPPPVGSLSQRKRQQYAKSKKQGNSSNS | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | LSWQAAIDAARQAKAAQTMSTSAPPPVGSLSQRKRQQYAKSKKQGNSSNS | 100 |
| | . . . . . | |
| 101 | RPARALFCLSLNNPIRRACISIVEWKPFDIFILLAIFANCVALAIYIPFP | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | RPARALFCLSLNNPIRRACISIVEWKPFDIFILLAIFANCVALAIYIPFP | 150 |
| | . . . . . | |
| 151 | EDDSNSTNHNLEKVEYAFLIIFTVETFLKIIAYGLLLHPNAYVRNGWNLL | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | EDDSNSTNHNLEKVEYAFLIIFTVETFLKIIAYGLLLHPNAYVRNGWNLL | 200 |
| | . . . . . | |
| 201 | DFVIVIVGLFSVILEQLTKETEGGNHSSGKSGGFDVKALRAFRVLRPLRL | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | DFVIVIVGLFSVILEQLTKETEGGNHSSGKSGGFDVKALRAFRVLRPLRL | 250 |
| | . . . . . | |
| 251 | VSGVPSLQVVLNSIIKAMVPLLHIALLVLFVIIIYAIIGLELFIGKMHKT | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | VSGVPSLQVVLNSIIKAMVPLLHIALLVLFVIIIYAIIGLELFIGKMHKT | 300 |
| | . . . . . | |
| 301 | CFFADSDIVAEEDPAPCAFSGNGRQCTANGTECRSGWVGPNGGITNFDNF | 350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | CFFADSDIVAEEDPAPCAFSGNGRQCTANGTECRSGWVGPNGGITNFDNF | 350 |
| | . . . . . | |
| 351 | AFAMLTVFQCITMEGWTDVLYWVNDAIGWEWPWVYFVSLIIL...... | 392 |
| | \|\|\|\|\|\|\|\|\|\|\|\|\|\|\|\|\|\|\|\|\|\| | |
| 351 | AFAMLTVFQCITMEGWTDVLYW....................MNDAMG | 378 |
| | . . | |
| 393 | ..............GSFFVLNLVLGVLSG | 407 |
| | \|\|\|\|\|\|\|\|\|\|\|\|\|\|\| | |
| 379 | FELPWVYFVSLVIFGSFFVLNLVLGVLSG | 407 |

### EXAMPLE 20

This Example relates to the variant HUMLVDCCB_P16 (SEQ ID NO:9; the nucleic acid sequence is given by SEQ ID NO:31), which is a variant alpha 1 subunit according to the present invention, and more specifically, is a splice variant of the known protein CCAC_HUMAN, which is encoded by the CACNA1C gene. This alpha subunit is the alpha 1C subunit, which forms the voltage-dependent L-type calcium channel. This subunit is also called the alpha subunit Cav1.2. The known protein is described in greater detail above with regard to Example 14.

The structure of the splice variant according to the present invention features unique insertions, skipped exon and unique tail, as compared to the known protein sequence. An alignment is provided at the end of this section, while the comparison between the two sequences is described below.

According to preferred embodiments of the present invention, there is provided an isolated chimeric polypeptide encoding for HUMLVDCCB_P16, comprising a first amino acid sequence being at least 90% homologous to (SEQ ID NO: 168) MVNENTRMYIPEENHQGSNYGSPRPAHANMNANAAAGLAPEHIPTPGAALSWQAAIDAARQAKLMGSAGNATISTVSSTQRKRQQYGKPKKQGSTTATRPPRALLCLTLKNPIRRACISIVEWKPFEIIILLTIFANCVALAIYIPFPEDDSNATNSNLERVEYLFLIIFTVEAFLKVIAYGLLFHPNAYLRNGWNLLDFIIVVVGLFSAILEQATKADGANALGGKGAGFDVKALRAFRVLRPLRLVSGVPSLQVVLNSIIKAMVPLLHIALLVLFVIIIYAIIGLELFMGKMHKTCYNQEGIADVPAEDDPSPCALETGHGRQCQNGTVCKPGWDGPKHGITNFDNFAFAMLTVFQCITMEGWTDVLYWVNDAVGRDWPWIYFVTLIIIGSFFVLNLVLGVLSGEFSKEREKAKARGDFQKLREKQQLEEDLKGYLDWITQAEDIDPENEDEGMDEEKPRN corresponding to amino acids 1-463 of CCAC_HUMAN, which also corresponds to amino acids 1-463 of HUMLVDCCB_P16, a second amino acid sequence being at least 70%, optionally at least 80%, preferably at least 85%, more preferably at least 90% and most preferably at least 95% homologous to a polypeptide having the sequence (SEQ ID NO: 169) RGTPAGMLDQKKGKFAWFSHSTETHV corresponding to amino acids 464-489 of HUMLVDCCB_P16, a third amino acid sequence being at least 90% homologous to (SEQ ID NO: 170) SMPTSETESVNTENVAGGDIEGENCGARLAHRISKSKFSRYWRRWNRFCRRKCRAAVKSNVFYWLVIFLVFLNTLTIASEHYNQPNWLTEVQDTANKALLALFTAEMLLKMYSLGLQAYFVSLFNRFDCFVVCGGILETILVETKIMSPLGISVLRCVRLLRIFKITRYWNSLSNLVASLLNSVRSIASLLLLLFLFIIIFSLLGMQLFGGKFNFDEMQTRRSTFDNFPQSLLTVFQILTGEDWNSVMYDGIMAYGGPSFPGMLVCIYFIILFICGNYILLNVFLAIAVDNLADAESLTSAQKEEEEEKERKKLARTASPEKKQELVEKPAVGESKEEKIELKSITADGESPPATKINMDDLQPNENEDKSPYPNPETTGEEDEEEPEMPVGPRPRPLSELHLKEKAVPMPEASAFFIFSSNNRFRLQCHRIVNDTIFTNLILFFILLSSISLAAEDPVQHTSFRNHILFYFDIVFTTIFTIEIA corresponding to amino acids 465-949 of CCAC_HUMAN, which also corresponds to amino acids 490-974 of HUMLVDCCB_P16, a fourth amino acid sequence being at least 90% homologous to (SEQ ID NO: 171) LKMTAYGAFLHKGSFCRNYFNILDLLVVSVSLISFGIQSSAINVVKILRVLRVLRPLRAINRAKGLKHVVQCVFVAIRTIGNIVIVTTLLQFMFACIGVQLFKGKLYTCSDSSKQTEAECKGNYITYKDGEVDHPIIQPRSWENSKFDFDNVLAAMMALFTVSTFEGWPELLYRSIDSHTEDKGPIYNYRVEISIFFIIYIIIIAFFMMNIFVGFVIVTFQEQGEQEYKNCELDKNQRQCVEYALKARPLRRYIPKNQHQYKVWYVVNSTYFEYLMFVLILLNTICLAMQHYGQSCLFKIAMNILNMLFTGLFTVEMILKLIAFKPK corresponding to amino acids 970-1296 of CCAC_HUMAN, which also corresponds to amino acids 975-1301 of HUMLVDCCB_P16, a fifth amino acid sequence being at least 90% homologous to (SEQ ID NO: 172) HYFCDAWNTFDALIVVGSIVDIAITEVNPAEHTQCSPSMNAEENSRISITFFRLFRVMRLVKLLSRGEGIRTLLWTFIKSFQALPYVALLIVMLFFIYAVIGMQVFGKIALNDTTEINRNNNFQTFPQAVLLLFRCATGEAWQDIMLACMPGKKCAPESEPSNSTEGETPCGSSFAVFYFISFYMLCAFLIINLFVAVIMDNFDYLTRDWSILGPHHLDEFKRIWAEYDPEAKGRIKHLDVVTLLRRIQPPLGFGKLCPHRVACKRLVSMNMPLNSDGTVMFNATLFALVRTALRIKTE corresponding to amino acids 1325-1623 of CCAC_HUMAN, which also corresponds to amino acids 1302-1600 of HUMLVDCCB_P16, a sixth amino acid sequence being at least 70%, optionally at least 80%, preferably at least 85%, more preferably at least 90% and most preferably at least 95% homologous to a polypeptide having the sequence (SEQ ID NO: 173) EGPSPSEAHQGAEDPFRPA corresponding to amino acids 1601-1619 of HUMLVDCCB_P16, a seventh amino acid sequence being at least 90% homologous to (SEQ ID NO: 174) GNLEQANEELRAIIKKIWKRTSMKLLDQVVPPAGDDEVTVGKFYATFLIQEYFRKFKKRKEQGLVGKPSQRNALSLQAGLRTLHDIGPEIRRAISGDLTAEEELDKAMKEAVSAASEDDIFRRAGGLFGNHVSYYQSDGRSAFPQTFTTQRPLHINKAGSSQGDTESPSHEKLVDSTFTPSSYSSTGSNANINNANNTALGRLPRPAGYPSTVSTVEGHGPPLSPAIRVQEVAWKLSSN corresponding to amino acids 1624-1862 of CCAC_HUMAN, which also corresponds to amino acids 1620-1858 of HUMLVDCCB_P16, a eight amino acid sequence being at least 90% homologous to (SEQ ID NO: 175) RCHSRESQAAMAGQEETSQDETYEVKMNHDTEACSEPSLLSTEMLSYQDDENRQLTLPEEDKRDIRQSPKRGFLRSASLGRRASFHLECLKRQKDRGGDISQKTVLPLHLVHHQALAVAGLSPLLQRSHSPASFPRPFATPPATPGSRGWPPQPVPTLRLEGVESSEKLNSSFPSIHCGSWAETTPGGGGSSAARRVRPVSLMVPSQAGAPGRQFHGSASSLVEAV corresponding to amino acids 1898-2123 of CCAC_HUMAN, which also corresponds to amino acids 1859-2084 of HUMLVDCCB_P16, a ninth amino acid sequence being at least 70%, optionally at least 80%, preferably at least 85%, more preferably at least 90% and most preferably at least 95% homologous to a polypeptide having the sequence (SEQ ID NO: 176) GDSQMGRGERPRATRGLGMRG corresponding to amino acids 2085-2105 of HUMLVDCCB_P16, wherein said first amino acid sequence, second amino acid sequence, third amino acid sequence, fourth amino acid sequence, fifth amino acid sequence, sixth amino acid sequence, seventh amino acid sequence, eight amino acid sequence and ninth amino acid sequence are contiguous and in a sequential order.

According to preferred embodiments of the present invention, there is provided an isolated polypeptide encoding for an edge portion of HUMLVDCCB_P16, comprising an amino acid sequence being at least 70%, optionally at least about 80%, preferably at least about 85%, more preferably at least about 90% and most preferably at least about 95% homologous to the sequence encoding for (SEQ ID NO: 177) RGTPAGMLDQKKGKFAWFSHSTETHV, corresponding to HUMLVDCCB_P16.

According to preferred embodiments of the present invention, there is provided a bridge portion of HUMLVDCCB_P16, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise N, having a structure as follows (numbering according to HUMLVDCCB_P16): a sequence starting from any of amino acid numbers 463−x to 463; and ending at any of amino acid numbers 464+((n−2)−x), in which x varies from 0 to n−2.

According to preferred embodiments of the present invention, there is provided an isolated chimeric polypeptide encoding for an edge portion of HUMLVDCCB_P16, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise AL, having a structure as follows: a sequence starting from any of amino acid numbers 974−x to 974; and ending at any of amino acid numbers 975+((n−2)−x), in which x varies from 0 to n−2.

According to preferred embodiments of the present invention, there is provided an isolated chimeric polypeptide encoding for an edge portion of HUMLVDCCB_P16, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise KH, having a structure as follows: a sequence starting from any of amino acid numbers 1301−x to 1301; and ending at any of amino acid numbers 1302+((n−2)−x), in which x varies from 0 to n−2.

According to preferred embodiments of the present invention, there is provided an isolated polypeptide encoding for an edge portion of HUMLVDCCB_P116, comprising an amino acid sequence being at least 70%, optionally at least about 80%, preferably at least about 85%, more preferably at least about 90% and most preferably at least about 95% homologous to the sequence encoding for (SEQ ID NO: 178) EGPSPSEAHQGAEDPFRPA, corresponding to HUMLVDCCB_P16.

According to preferred embodiments of the present invention, there is provided an isolated chimeric polypeptide encoding for an edge portion of HUMLVDCCB_P16, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise NR, having a structure as follows: a sequence starting from any of amino acid numbers 1858−x to 1858; and ending at any of amino acid numbers 1859+((n−2)−x), in which x varies from 0 to n−2.

According to preferred embodiments of the present invention, there is provided an isolated polypeptide encoding for a tail of HUMLVDCCB_P16, comprising a polypeptide being at least 70%, optionally at least about 80%, preferably at least about 85%, more preferably at least about 90% and most preferably at least about 95% homologous to the sequence (SEQ ID NO: 179) GDSQMGRGERPRATRGLGMRG in HUMLVDCCB_P16.

1. Domains affected by alternative splicing include: the cytoplasmic loop between domain I and domain II; the S2 transmembrane domain of domain III; the extracellular loop between S2 and S3 transmembrane domains of domain III; the extracellular loop between S2 and S3 transmembrane domains of domain IV; the S3 transmembrane domain of domain IV; the extracellular loop between S3 and S4 transmembrane domains of domain IV the cytoplasmic C-terminus region of the protein.

Potential effects of changes in this variant are described in Example 14.

From the above description of the effect of changes to the C-terminus domain of this variant, it is expected that the variant will have greater current amplitude as compared to the known protein and a stronger calcium dependence of inactivation. It is also expected to modulate the Ca⁺²-dependent inactivation of the channel.

In addition, the changes in the S2 transmembrane domain of domain III might influence the voltage-dependent action of dihydropyridines.

Sequence name: CCAC_HUMAN

documentation:

of: HUMLVDCCB_P16 (residues 1-2084 of SEQ ID NO: 9)×CCAC_(SEQ ID NO: 180) . . .

segment 1/1:

Quality: 19433.00

Escore: 0

Matching length: 2040 Total

length: 2167

Matching Percent Similarity: 100.00 Matching Percent

Identity: 99.95

Total Percent Similarity: 94.14 Total Percent

Identity: 94.09

Gaps: 5

| | . . . . . | |
|---|---|---|
| 1 | MVNENTRMYIPEENHQGSNYGSPRPAHANMNANAAAGLAPEHIPTPGAAL | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MVNENTRMYIPEENHQGSNYGSPRPAHANMNANAAAGLAPEHIPTPGAAL | 50 |
| | . . . . . | |
| 51 | SWQAAIDAARQAKLMGSAGNATISTVSSTQRKRQQYGKPKKQGSTTATRP | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | SWQAAIDAARQAKLMGSAGNATISTVSSTQRKRQQYGKPKKQGSTTATRP | 100 |
| | . . . . . | |
| 101 | PRALLCLTLKNPIRRACISIVEWKPFEIIILLTIFANCVALAIYIPFPED | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | PRALLCLTLKNPIRRACISIVEWKPFEIIILLTIFANCVALAIYIPFPED | 150 |
| | . . . . . | |
| 151 | DSNATNSNLERVEYLFLIIFTVEAFLKVIAYGLLFHPNAYLRNGWNLLDF | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | DSNATNSNLERVEYLFLIIFTVEAFLKVIAYGLLFHPNAYLRNGWNLLDF | 200 |
| | . . . . . | |
| 201 | IIVVVGLFSAILEQATKADGANALGGKGAGFDVKALRAFRVLRPLRLVSG | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | IIVVVGLFSAILEQATKADGANALGGKGAGFDVKALRAFRVLRPLRLVSG | 250 |
| | . . . . . | |
| 251 | VPSLQVVLNSIIKAMVPLLHIALLVLFVIIIYAIIGLELFMGKMHKTCYN | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | VPSLQVVLNSIIKAMVPLLHIALLVLFVIIIYAIIGLELFMGKMHKTCYN | 300 |
| | . . . . . | |
| 301 | QEGIADVPAEDDPSPCALETGHGRQCQNGTVCKPGWDGPKHGITNFDNFA | 350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | QEGIADVPAEDDPSPCALETGHGRQCQNGTVCKPGWDGPKHGITNFDNFA | 350 |
| | . . . . . | |
| 351 | FAMLTVFQCITMEGWTDVLYWVNDAVGRDWPWIYFVTLIIIGSFFVLNLV | 400 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 351 | FAMLTVFQCITMEGWTDVLYWVNDAVGRDWPWIYFVTLIIIGSFFVLNLV | 400 |
| | . . . . . | |
| 401 | LGVLSGEFSKEREKAKARGDFQKLREKQQLEEDLKGYLDWITQAEDIDPE | 450 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 401 | LGVLSGEFSKEREKAKARGDFQKLREKQQLEEDLKGYLDWITQAEDIDPE | 450 |
| | . . . . . | |
| 451 | NEDEGMDEEKPRNRGTPAGMLDQKKGKFAWFSHSTETHVSMPTSETESVN | 500 |
| | \|\|\|\|\|\|\|\|\|\|\|\|\| :\|\|\|\|\|\|\|\|\|\|\| | |
| 451 | NEDEGMDEEKPRN.........................MSMPTSETESVN | 475 |
| | . . . . . | |
| 501 | TENVAGGDIEGENCGARLAHRISKSKFSRYWRRWNRFCRRKCRAAVKSNV | 550 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 476 | TENVAGGDIEGENCGARLAHRISKSKFSRYWRRWNRFCRRKCRAAVKSNV | 525 |
| | . . . . . | |
| 551 | FYWLVIFLVFLNTLTIASEHYNQPNWLTEVQDTANKALLALFTAEMLLKM | 600 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 526 | FYWLVIFLVFLNTLTIASEHYNQPNWLTEVQDTANKALLALFTAEMLLKM | 575 |
| | . . . . . | |
| 601 | YSLGLQAYFVSLFNRFDCFVVCGGILETILVETKIMSPLGISVLRCVRLL | 650 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 576 | YSLGLQAYFVSLFNRFDCFVVCGGILETILVETKIMSPLGISVLRCVRLL | 625 |
| | . . . . . | |
| 651 | RIFKITRYWNSLSNLVASLLNSVRSIASLLLLLFLFIIIFSLLGMQLFGG | 700 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 626 | RIFKITRYWNSLSNLVASLLNSVRSIASLLLLLFLFIIIFSLLGMQLFGG | 675 |
| | . . . . . | |
| 701 | KFNFDEMQTRRSTFDNFPQSLLTVFQILTGEDWNSVMYDGIMAYGGPSFP | 750 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 676 | KFNFDEMQTRRSTFDNFPQSLLTVFQILTGEDWNSVMYDGIMAYGGPSFP | 725 |
| | . . . . . | |
| 751 | GMLVCIYFIILFICGNYILLNVFLAIAVDNLADAESLTSAQKEEEEEKER | 800 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 726 | GMLVCIYFIILFICGNYILLNVFLAIAVDNLADAESLTSAQKEEEEEKER | 775 |
| | . . . . . | |
| 801 | KKLARTASPEKKQELVEKPAVGESKEEKIELKSITADGESPPATKINMDD | 850 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 776 | KKLARTASPEKKQELVEKPAVGESKEEKIELKSITADGESPPATKINMDD | 825 |
| | . . . . . | |
| 851 | LQPNENEDKSPYPNPETTGEEDEEEPEMPVGPRPRPLSELHLKEKAVPMP | 900 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 826 | LQPNENEDKSPYPNPETTGEEDEEEPEMPVGPRPRPLSELHLKEKAVPMP | 875 |
| | . . . . . | |
| 901 | EASAFFIFSSNNRFRLQCHRIVNDTIFTNLILFFILLSSISLAAEDPVQH | 950 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 876 | EASAFFIFSSNNRFRLQCHRIVNDTIFTNLILFFILLSSISLAAEDPVQH | 925 |
| | . . . . . | |
| 951 | TSFRNHILFYFDIVFTTIFTIEIA....................LKMTAY | 980 |
| | \|\|\|\|\|\|\|\|\|\|\|\|\|\|\|\|\|\|\|\|\|\|\|\| \|\|\|\|\|\| | |
| 926 | TSFRNHILFYFDIVFTTIFTIEIALKILGNADYVETSIFTLEIILKMTAY | 975 |
| | . . . . . | |
| 981 | GAFLHKGSFCRNYFNILDLLVVSVSLISFGIQSSAINVVKILRVLRVLRP | 1030 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 976 | GAFLHKGSFCRNYFNILDLLVVSVSLISFGIQSSAINVVKILRVLRVLRP | 1025 |
| | . . . . . | |
| 1031 | LRAINRAKGLKHVVQCVFVAIRTIGNIVIVTTLLQFMFACIGVQLFKGKL | 1080 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1026 | LRAINRAKGLKHVVQCVFVAIRTIGNIVIVTTLLQFMFACIGVQLFKGKL | 1075 |
| | . . . . . | |
| 1081 | YTCSDSSKQTEAKCKGNYITYKDGEVDHPIIQPRSWENSKFDFDNVLAAM | 1130 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1076 | YTCSDSSKQTEAKCKGNYITYKDGEVDHPIIQPRSWENSKFDFDNVLAAM | 1125 |
| | . . . . . | |
| 1131 | MALFTVSTFEGWPELLYRSIDSHTEDKGPIYNYRVEISIFFIIYIIIIAF | 1180 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1126 | MALFTVSTFEGWPELLYRSIDSHTEDKGPIYNYRVEISIFFIIYIIIIAF | 1175 |
| | . . . . . | |
| 1181 | FMMNIFVGFVIVTFQEQGEQEYKNCELDKNQRQCVEYALKARPLRRYIPK | 1230 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1176 | FMMNIFVGFVIVTFQEQGEQEYKNCELDKNQRQCVEYALKARPLRRYIPK | 1225 |
| | . . . . . | |
| 1231 | NQHQYKVWYVVNSTYFEYLMFVLILLNTICLAMQHYGQSCLFKIAMNILN | 1280 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1226 | NQHQYKVWYVVNSTYFEYLMFVLILLNTICLAMQHYGQSCLFKIAMNILN | 1275 |
| | . . . . . | |
| 1281 | MLFTGLFTVEMILKLIAFKPK............................H | 1302 |
| | \|\|\|\|\|\|\|\|\|\|\|\|\|\|\|\|\|\|\|\|\| \| | |
| 1276 | MLFTGLFTVEMILKLIAFKPKGYFSDPWNVFDFLIVIGSIIDVILSETNH | 1325 |
| | . . . . . | |
| 1303 | YFCDAWNTFDALIVVGSIVDIAITEVNPAEHTQCSPSMNAEENSRISITF | 1352 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1326 | YFCDAWNTFDALIVVGSIVDIAITEVNPAEHTQCSPSMNAEENSRISITF | 1375 |
| | . . . . . | |
| 1353 | FRLFRVMRLVKLLSRGEGIRTLLWTFIKSFQALPYVALLIVMLFFIYAVI | 1402 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1376 | FRLFRVMRLVKLLSRGEGIRTLLWTFIKSFQALPYVALLIVMLFFIYAVI | 1425 |
| | . . . . . | |
| 1403 | GMQVFGKIALNDTTEINRNNNFQTFPQAVLLLFRCATGEAWQDIMLACMP | 1452 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1426 | GMQVFGKIALNDTTEINRNNNFQTFPQAVLLLFRCATGEAWQDIMLACMP | 1475 |
| | . . . . . | |
| 1453 | GKKCAPESEPSNSTEGETPCGSSFAVFYFISFYMLCAFLIINLFVAVIMD | 1502 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1476 | GKKCAPESEPSNSTEGETPCGSSFAVFYFISFYMLCAFLITNLFVAVIMD | 1525 |
| | . . . . . | |
| 1503 | NFDYLTRDWSILGPHHLDEFKRIWAEYDPEAKGRIKHLDVVTLLRRIQPP | 1552 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1526 | NFDYLTRDWSILGPHHLDEFKRIWAEYDPEAKGRIKHLDVVTLLRRIQPP | 1575 |
| | . . . . . | |
| 1553 | LGFGKLCPHRVACKRLVSMNMPLNSDGTVMFNATLFALVRTALRIKTEEG | 1602 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1576 | LGFGKLCPHRVACKRLVSMNMPLNSDGTVMFNATLFALVRTALRIKTE.. | 1623 |
| | . . . . . | |
| 1603 | PSPSEAHQGAEDPFRPAGNLEQANEELRAIIKKIWKRTSMKLLDQVVPPA | 1652 |
| | \|\|\|\|\|\|\|\|\|\|\|\|\|\|\|\|\|\|\|\|\|\|\|\|\|\|\|\|\|\|\|\|\| | |
| 1624 | .................GNLEQANEELRAIIKKIWKRTSMKLLDQVVPPA | 1656 |
| | . . . . . | |
| 1653 | GDDEVTVGKFYATFLIQEYFRKFKKRKEQGLVGKPSQRNALSLQAGLRTL | 1702 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1657 | GDDEVTVGKFYATFLIQEYFRKFKKRKEQGLVGKPSQRNALSLQAGLRTL | 1706 |
| | . . . . . | |
| 1703 | HDIGPEIRRAISGDLTAEEELDKAMKEAVSAASEDDIFRRAGGLFGNHVS | 1752 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1707 | HDIGPEIRRAISGDLTAEEELDKAMKEAVSAASEDDIFRRAGGLFGNHVS | 1756 |
| | . . . . . | |
| 1753 | YYQSDGRSAFPQTFTTQRPLHINKAGSSQGDTESPSHEKLVDSTFTPSSY | 1802 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1757 | YYQSDGRSAFPQTFTTQRPLHINKAGSSQGDTESPSHEKLVDSTFTPSSY | 1806 |
| | . . . . . | |
| 1803 | SSTGSNANINNANNTALGRLPRPAGYPSTVSTVEGHGPPLSPAIRVQEVA | 1852 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1807 | SSTGSNANINNANNTALGRLPRPAGYPSTVSTVEGHGPPLSPAIRVQEVA | 1856 |
| | . . . . . | |
| 1853 | WKLSSN...................................RCHSRESQA | 1867 |
| | \|\|\|\|\|\| \|\|\|\|\|\|\|\|\| | |
| 1857 | WKLSSNRERHVPVCEDLELRRDSGSAGTQAHCLLLRRANPSRCHSRESQA | 1906 |
| | . . . . . | |
| 1868 | AMAGQEETSQDETYEVKMNHDTEACSEPSLLSTEMLSYQDDENRQLTLPE | 1917 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1907 | AMAGQEETSQDETYEVKMNHDTEACSEPSLLSTEMLSYQDDENRQLTLPE | 1956 |
| | . . . . . | |
| 1918 | EDKRDIRQSPKRGFLRSASLGRRASFHLECLKRQKDRGGDISQKTVLPLH | 1967 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1957 | EDKRDIRQSPKRGFLRSASLGRRASFHLECLKRQKDRGGDISQKTVLPLH | 2006 |
| | . . . . . | |
| 1968 | LVHHQALAVAGLSPLLQRSHSPASFPRPFATPPATPGSRGWPPQPVPTLR | 2017 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2007 | LVHHQALAVAGLSPLLQRSHSPASFPRPFATPPATPGSRGWPPQPVPTLR | 2056 |
| | . . . . . | |
| 2018 | LEGVESSEKLNSSFPSIHCGSWAETTPGGGGSSAARRVRPVSLMVPSQAG | 2067 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2057 | LEGVESSEKLNSSFPSIHCGSWAETTPGGGGSSAARRVRPVSLMVPSQAG | 2106 |
| | . | |
| 2068 | APGRQEHGSASSLVEAV | 2084 |
| | \|\|\|\|\|\|\|\|\|\|\|\|\|\|\|\|\| | |
| 2107 | APGRQFHGSASSLVEAV | 2123 |

### EXAMPLE 21

This Example relates to the variant HUMCACH1A_P4 (SEQ ID NO:20; the nucleic acid sequence is given by SEQ ID NO:42), which is a variant alpha 1 subunit according to the present invention, and more specifically, is a splice variant of the known protein CCAD_HUMAN, which is encoded by the CACNA1D gene. This alpha subunit is the alpha 1D subunit, which forms the voltage-dependent L-type calcium channel. It is also known as alpha subunit Cav1.3. The known protein is described in Example 19.

The structure of the splice variant according to the present invention features a unique tail, as compared to the known protein sequence. An alignment is provided at the end of this section, while the comparison between the two sequences is described below.

According to preferred embodiments of the present invention, there is provided an isolated chimeric polypeptide encoding for HUMCACH1A_P4, comprising a first amino acid sequence being at least 90% homologous to (SEQ ID NO: 181) MMMMMMMKKMQHQRQQQADHANEANYARGTRLPLSGEGPTSQPNSSKQTVLSWQAAIDAARQAKAAQTMSTSAPPPVGSLSQRKRQQYAKSKKQGNSSNSRPARALFCLSLNNPIRRACISIVEWKPFDIFILLAIFANCVALAIYIPFPEDDSNSTNHNLEKVEYAFLIIFTVETFLKIIAYGLLLHPNAYVRNGWNLLDFVIVIVGLFSVILEQLTKETEGGNHSSGKSGGFDVKALRAFRVLRPLRLVSGVPSLQVVLNSIIKAMVPLLHIALLVLFVIIIYAIIGLELFIGKMHKTCFFADSDIVAEEDPAPCAFSGNGRQCTANGTECRSGWVGPNGGITNFDNFAFAMLTVFQCITMEGWTDVLYWMNDAMGFELPWVYFVSLVIFGSFFVLNLVLGVLSG corresponding to amino acids 1-407 of CCAD_HUMAN, which also corresponds to amino acids 1-407 of HUMCACH1A_P4, and a second amino acid sequence being at least 70%, optionally at least 80%, preferably at least 85%, more preferably at least 90% and most preferably at least 95% homologous to a polypeptide having the sequence HSGSRL corresponding to amino acids 408-413 of HUMCACH1A_P4, wherein said first amino acid sequence and second amino acid sequence are contiguous and in a sequential order.

According to preferred embodiments of the present invention, there is provided an isolated polypeptide encoding for a tail of HUMCACH1A_P4, comprising a polypeptide being at least 70%, optionally at least about 80%, preferably at least about 85%, more preferably at least about 90% and most preferably at least about 95% homologous to the sequence HSGSRL in HUMCACH1A_P4.

Domains affected by alternative splicing include: the cytoplasmic loop between domain I and domain II; the S1 transmembrane domain of domain II; the extracellular loop between S1 and S2 transmembrane domains of domain II; the S2 transmembrane domain of domain II; the extracellular loop between S2 and S3 transmembrane domains of domain II; the S3 transmembrane domain of domain II; the extracellular loop between S3 and S4 transmembrane domains of domain II; the S4 transmembrane domain of domain II; the extracellular loop between S4 and S5 transmembrane domains of domain II; the S5 transmembrane domain of domain II; the pore loop region between S5 and S6 transmembrane domains of domain II; the S6 transmembrane domain of domain II; the cytoplasmic loop between domain II and domain III; the S1 transmembrane domain of domain III; the extracellular loop between S1 and S2 transmembrane domains of domain III; the S2 transmembrane domain of domain III; the extracellular loop between S2 and S3 transmembrane domains of domain III; the S3 transmembrane domain of domain III; the extracellular loop between S3 and S4 transmembrane domains of domain III; the S4 transmembrane domain of domain III; the extracellular loop between S4 and S5 transmembrane domains of domain III; the S5 transmembrane domain of domain III; the pore loop region between S5 and S6 transmembrane domains of domain III; the S6 transmembrane domain of domain III; the cytoplasmic loop between domain III and domain IV; the S1 transmembrane domain of domain IV; the extracellular loop between S1 and S2 transmembrane domains of domain IV; the S2 transmembrane domain of domain IV; the extracellular loop between S2 and S3 transmembrane domains of domain IV; the S3 transmembrane domain of domain IV; the extracellular loop between S3 and S4 transmembrane domains of domain IV; the S4 transmembrane domain of domain IV; the extracellular loop between S4 and S5 transmembrane domains of domain IV; the S5 transmembrane domain of domain IV; the pore loop region between S5 and S6 transmembrane domains of domain IV; the S6 transmembrane domain of domain IV the cytoplasmic C-terminus region of the protein.

The potential effect of such very short (one or two domain) variants is described above with regard to Example 19. From the above description of the function of variants lacking several domains, it is expected that the variant will function as a down regulation of a known functional channel protein (in particular of this alpha subunit type). Sequence name: CCAD_HUMAN

documentation:

of: HUMCACH1A_P4 (residues 1-407 of SEQ ID NO: 20)×CCAD_HUMAN (SEQ ID NO: 182). . .

segment 1/1:

Quality: 3976.00

Escore: 0

Matching length: 407 Total

length: 407

Matching Percent Similarity: 100.00 Matching Percent

Identity: 100.00

Total Percent Similarity: 100.00 Total Percent

Identity: 100.00

Gaps: 0

| | . . . . . | |
|---|---|---|
| 1 | MMMMMMMKKMQHQRQQQADHANEANYARGTRLPLSGEGPTSQPNSSKQTV | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MMMMMMMKKMQHQRQQQADHANEANYARGTRLPLSGEGPTSQPNSSKQTV | 50 |
| | . . . . . | |
| 51 | LSWQAAIDAARQAKAAQTMSTSAPPPVGSLSQRKRQQYAKSKKQGNSSNS | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | LSWQAAIDAARQAKAAQTMSTSAPPPVGSLSQRKRQQYAKSKKQGNSSNS | 100 |
| | . . . . . | |
| 101 | RPARALFCLSLNNPIRRACISIVEWKPFDIFILLAIFANCVALAIYIPFP | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | RPARALFCLSLNNPIRRACISIVEWKPFDIFILLAIFANCVALAIYIPFP | 150 |
| | . . . . . | |
| 151 | EDDSNSTNHNLEKVEYAFLIIFTVETFLKIIAYGLLLHPNAYVRNGWNLL | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | EDDSNSTNHNLEKVEYAFLIIFTVETFLKIIAYGLLLHPNAYVRNGWNLL | 200 |
| | . . . . . | |
| 201 | DFVIVIVGLFSVILEQLTKETEGGNHSSGKSGGFDVKALRAFRVLRPLRL | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | DFVIVIVGLFSVILEQLTKETEGGNHSSGKSGGFDVKALRAFRVLRPLRL | 250 |
| | . . . . . | |
| 251 | VSGVPSLQVVLNSIIKAMVPLLHIALLVLFVIIIYAIIGLELFIGKMHKT | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | VSGVPSLQVVLNSIIKAMVPLLHIALLVLFVIIIYAIIGLELFIGKMHKT | 300 |
| | . . . . . | |
| 301 | CFFADSDIVAEEDPAPCAFSGNGRQCTQNGTECRSGWVGPNGGITNFDNF | 350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | CFFADSDIVAEEDPAPCAFSGNGRQCTQNGTECRSGWVGPNGGITNFDNF | 350 |
| | . . . . . | |
| 351 | AFAMLTVFQCITMEGWTDVLYWMNDAMGFELPWVYFVSLVIFGSFFVLNL | 400 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 351 | AFAMLTVFQCITMEGWTDVLYWMNDAMGFELPWVYFVSLVIFGSFFVLNL | 400 |
| 401 | VLGVLSG | 407 |
| | \|\|\|\|\|\|\| | |
| 401 | VLGVLSG | 407 |

### EXAMPLE 22

This Example relates to the variant HUMCACH1A_P3 (SEQ ID NO:22; the nucleic acid sequence is given by SEQ ID NO:44), which is a variant alpha 1 subunit according to the present invention, and more specifically, is a splice variant of the known protein CCAD_HUMAN, which is encoded by the CACNA1D gene. This alpha subunit is the alpha 1D subunit, which forms the voltage-dependent L-type calcium channel. It is also known as alpha subunit Cav1.3. The known protein is described in Example 19.

The structure of the splice variant according to the present invention features a skipped exon, as compared to the known protein sequence. An alignment is provided at the end of this section, while the comparison between the two sequences is described below.

According to preferred embodiments of the present invention, there is provided an isolated chimeric polypeptide encoding for HUMCACH1A_P3, comprising a first amino acid sequence being at least 90% homologous to (SEQ ID NO: 183) MMMMMMMKKMQHQRQQQADHANEANYARGTRLPLSGEGPTSQPNSSKQTVLSWQAAIDAARQAKAAQTMSTSAPPPVGSLSQRKRQQYAKSKKQGNSSNSRPARALFCLSLNNPIRRACISIVEWKPFDIFILLAIFANCVALAIYIPFPEDDSNSTNHNLEKVEYAFLIIFTVETFLKIIAYGLLLHPNAYVRNGWNLLDFVIVIVGLFSVILEQLTKETEGGNHSSGKSGGFDVKALRAFRVLRPLRLVSGVPSLQVVLNSIIKAMVPLLHIALLVLFVIIIYAIIGLELFIGKMHKTCFFADSDIVAEEDPAPCAFSGNGRQCTANGTECRSGWVGPNGGITNFDNFAFAMLTVFQCITMEGWTDVLYWMNDAMGFELPWVYFVSLVIFGSFFVLNLVLGVLSGEFSKEREKAKARGDFQKLREKQQLEEDLKGYLDWITQAEDIDPENEEEGGEEGKRNTSMPTSETESVNTENVSGEGENRGCCGSLCQAISKSKLSRRWRRWNRFNRRRCRAAVKSVTFYWLVIVLVFLNTLTISSEHYNQPDWLTQIQDIANKVLLALFTCEMLVKMYSLGLQAYFVSLFNRFDCFVVCGGITETILVELEIMSPLGISVFRCVRLLRIFKVTRHWTSL corresponding to amino acids 1-636 of CCAD_HUMAN, which also corresponds to amino acids 1-636 of HUMCACH1A_P3, a bridging amino acid S corresponding to amino acid 637 of HUMCACH1A_P3, a second amino acid sequence being at least 90% homologous to (SEQ ID NO: 191) NLVASLLNSMKS corresponding to amino acids 638-649 of CCAD_HUMAN, which also corresponds to amino acids 638-649 of HUMCACH1A_P3, a bridging amino acid I corresponding to amino acid 650 of HUMCACH1A_P3, a third amino acid sequence being at least 90% homologous to (SEQ ID NO: 184) ASLLLLLFLFIIIFSLLGMQLFGGKFNFDETQTKRSTFDNFPQALLTVFQILTGEDWNAVMYDGIMAYGGPSSSGMIVCIYFIILFICGNYILLNVFLAIAVDNLADAESLNTAQKEEAEEKERKKIARKESLENKKNNKPEVNQIANSDNKVTIDDYREEDEDKDPYPPCDVPVGEEEEEEEEDEPEVPAGPRPRRISELNMKEKIAPIPEGSAFFILSKTNPIRVGCHKLINHHIFTNLILVFIMLSSAALAAEDPIRSHSFRNTILGYFDYAFTAIFTVEILLKMTTFGAFLHKGAFCRNYFNLLDMLVVGVSLVSFGIQSSAISVVKILRVLRVLRPLRAINRAKGLKHVVQCVFVAIRTIGNIMIVTTLLQFMFACIGVQLFKGKFYRCTDEAKSNPEECRGLFILYKDGDVDSPVVRERIWQNSDFNFDNVLSAMMALFTVSTFEGWPALLYKAIDSNGENIGPIYNHRVEISIFFIIYIIIVAFFMMNIFVGFVIVTFQEQGEKEYKNCELDKNQRQCVEYALKARPLRRYIPKNPYQYKFWYVVNSSPFEYMMFVLIMLNTLCLAMQHYEQSKMFNDAMDILNMVFTGVFTVEMVLKVIAFKPKGYFSDAWNTFDSLIVIGSIIDVALSEADPTESENVPVPTATPGNSEESNRISITFFRLFRVMRLVKLLSRGEGIRTLLWTFIK corresponding to amino acids 651-1345 of CCAD_HUMAN, which also corresponds to amino acids 651-1345 of HUMCACH1A_P3, a bridging amino acid S corresponding to amino acid 1346 of HUMCACH1A_P3, a fourth amino acid sequence being at least 90% homologous to (SEQ ID NO: 185) FQALPYVALLIAMLFFIYAVIGMQMFGKVAMRDNNQNNFQTFPQAVLLLFRCATGEAWQEIMLACLPGKLCDPESDYNPGEE corresponding to amino acids 1347-1432 of CCAD_HUMAN, which also corresponds to amino acids 1347-1432 of HUMCACH1A_P3, a bridging amino acid Y corresponding to amino acid 1433 of HUMCACH1A_P3, a fifth amino acid sequence being at least 90% homologous to (SEQ ID NO: 186) TCGSNFAIVYFISFYMLCAFLIINLFVAVIMDNFDYLTRDWSILGPHHLDEFKRIWSEYDPAKGRIKHLDVVTLLRRIQPPLGFGKLCPHRVACKRLVAMNMPLNSDGTVMFNATLFALVRTALKIKTEGNLEQANEELRAVIKKIWKKTSMKLLDQVVPPAGDDEVTVGKFYATFLIQDYFRKFKKRKEQGLVGKYPAKNTTIALQAGLRTLHDIGPEIRRAISCDLQDDEPEETKREEEDDVFKRNGALLGNHVNHVNSDRRDSLQQTNTTHRPLHVQRPSIPPASDTEKPLFPPAGNSVCHNHHNHNSIGKQVPTSTNANLNNANMSKAAHGKRPSIGNLEHVSENGHHSSHKHDREPQRRSSVK corresponding to amino acids 1434-1802 of CCAD_HUMAN, which also corresponds to amino acids 1434-1802 of HUMCACH1A_P3, a sixth amino acid sequence being at least 90% homologous to (SEQ ID NO: 187) RSDSGDEQLPTICREDPEIHGYFRDPHCLGEQEYFSSEECYEDDSSPTWSRQNYGYYSRYPGRNIDSERPRGYHHPQGFLEDDDSPVCYDSRRSPRRRLLPPTPASHRRSSFNFECLRRQSSQEEVPSSPIFPHRTALPLHLMQQQIMAVAGLDSSKAQKYSPSHSTRSWATPPATPPYRDWTPCYTPLIQVEQSEALDQVNGSLPSLHRSSWYTDEPDISYRTFTPASLTVPSSFRNKNSDKQRSADSLVEAVLISEGLGRYARDPKFVSATKHEIADACDLTIDEMESAASTLLNGNVRPRANGDVGPLSHRQDYELQDFGPGYSDEEPDPGRDEEDLADEMICITTL corresponding to amino acids 1812-2161 of CCAD_HUMAN, which also corresponds to amino acids 1803-2152 of HUMCACH1A_P3, wherein said first amino acid sequence, bridging amino acid, second amino acid sequence, bridging amino acid, third amino acid sequence, bridging amino acid, fourth amino acid sequence, bridging amino acid, fifth amino acid sequence and sixth amino acid sequence are contiguous and in a sequential order.

According to preferred embodiments of the present invention, there is provided an isolated chimeric polypeptide encoding for an edge portion of HUMCACH1A_P3, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise KR, having a structure as follows: a sequence starting from any of amino acid numbers 1802−x to 1802; and ending at any of amino acid numbers 1803+((n−2)−x), in which x varies from 0 to n−2. Domains affected by alternative splicing include the cytoplasmic C-terminus region of the protein.

As the C-terminus becomes shorter, the current amplitude increases, as does the calcium-dependent inactivation. In neuronal Cav1.3 channels, replacement of exon 41 by a mutually exclusive exon 41a leads to an early stop codon, truncating over 500 amino acid residues encoded by exons 42-49, which results in a twofold increase of current amplitude without change of voltage dependence of gating.

From the above description of the effect of changes to the C-terminus domain of this variant, it is expected that the variant will have an increase of current amplitude without change of voltage dependence of gating. It is also expected to modulate the Ca+2-dependent inactivation of the channel.

Sequence name: CCAD_HUMAN

documentation:

of: HUMCACH1A_P3 (SEQ ID NO: 22)×CCAD_HUMAN (SEQ ID NO: 188) . . .

segment 1/1:

Quality: 21052.00

Escore: 0

Matching length: 2152 Total

length: 2161

Matching Percent Similarity: 9986 Matching Percent

Identity: 99.81

Total Percent Similarity: 99.44 Total Percent

Identity: 99.40

Gaps: 1

| | . . . . . | |
|---|---|---|
| 1 | MMMMMMMKKMQHQRQQQADHANEANYARGTRLPLSGEGPTSQPNSSKQTV | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MMMMMMMKKMQHQRQQQADHANEANYARGTRLPLSGEGPTSQPNSSKQTV | 50 |
| | . . . . . | |
| 51 | LSWQAAIDAARQAKAAQTMSTSAPPPVGSLSQRKRQQYAKSKKQGNSSNS | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | LSWQAAIDAARQAKAAQTMSTSAPPPVGSLSQRKRQQYAKSKKQGNSSNS | 100 |
| | . . . . . | |
| 101 | RPARALFCLSLNNPIRRACISIVEWKPFDIFILLAIFANCVALAIYIPFP | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | RPARALFCLSLNNPIRRACISIVEWKPFDIFILLAIFANCVALAIYIPFP | 150 |
| | . . . . . | |
| 151 | EDDSNSTNHNLEKVEYAFLIIFTVETFLKIIAYGLLLHPNAYVRNGWNLL | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | EDDSNSTNHNLEKVEYAFLIIFTVETFLKIIAYGLLLHPNAYVRNGWNLL | 200 |
| | . . . . . | |
| 201 | DFVIVIVGLFSVILEQLTKETEGGNHSSGKSGGFDVKALRAFRVLRPLRL | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | DFVIVIVGLFSVILEQLTKETEGGNHSSGKSGGFDVKALRAFRVLRPLRL | 250 |
| | . . . . . | |
| 251 | VSGVPSLQVVLNSIIKAMVPLLHIALLVLFVIIIYAIIGLELFIGKMHKT | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | VSGVPSLQVVLNSIIKAMVPLLHIALLVLFVIIIYAIIGLELFIGKMHKT | 300 |
| | . . . . . | |
| 301 | CFFADSDIVAEEDPAPCAFSGNGRQCTANGTECRSGWVGPNGGITNFDNF | 350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | CFFADSDIVAEEDPAPCAFSGNGRQCTANGTECRSGWVGPNGGITNFDNF | 350 |
| | . . . . . | |
| 351 | AFAMLTVFQCITMEGWTDVLYWMNDAMGFELPWVYFVSLVIFGSFFVLNL | 400 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 351 | AFAMLTVFQCITMEGWTDVLYWMNDAMGFELPWVYFVSLVIFGSFFVLNL | 400 |
| | . . . . . | |
| 401 | VLGVLSGEFSKEREKAKARGDFQKLREKQQLEEDLKGYLDWITQAEDIDP | 450 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 401 | VLGVLSGEFSKEREKAKARGDFQKLREKQQLEEDLKGYLDWITQAEDIDP | 450 |
| | . . . . . | |
| 451 | ENEEEGGEEGKRNTSMPTSETESVNTENVSGEGENRGCCGSLCQAISKSK | 500 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 451 | ENEEEGGEEGKRNTSMPTSETESVNTENVSGEGENRGCCGSLCQAISKSK | 500 |
| | . . . . . | |
| 501 | LSRRWRRWNRFNRRRCRAAVKSVTFYWLVIVLVFLNTLTISSEHYNQPDW | 550 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 501 | LSRRWRRWNRFNRRRCRAAVKSVTFYWLVIVLVFLNTLTISSEHYNQPDW | 550 |
| | . . . . . | |
| 551 | LTQIQDIANKVLLALFTCEMLVKMYSLGLQAYFVSLFNRFDCFVVCGGIT | 600 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 551 | LTQIQDIANKVLLALFTCEMLVKMYSLGLQAYFVSLFNRFDCFVVCGGIT | 600 |
| | . . . . . | |
| 601 | ETILVELEIMSPLGISVFRCVRLLRIFKVTRHWTSLSNLVASLLNSMKSI | 650 |
| | \|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\| | |
| 601 | ETILVELEIMSPLGISVFRCVRLLRIFKVTRHWTSLCNLVASLLNSMKSS | 650 |
| | . . . . . | |
| 651 | ASLLLLLFLFIIIFSLLGMQLFGGKFNFDETQTKRSTFDNFPQALLTVFQ | 700 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 651 | ASLLLLLFLFIIIFSLLGMQLFGGKFNFDETQTKRSTFDNFPQALLTVFQ | 700 |
| | . . . . . | |
| 701 | ILTGEDWNAVMYDGIMAYGGPSSSGMIVCIYFIILFICGNYILLNVFLAI | 750 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 701 | ILTGEDWNAVMYDGIMAYGGPSSSGMIVCIYFIILFICGNYILLNVFLAI | 750 |
| | . . . . . | |
| 751 | AVDNLADAESLNTAQKEEAEEKERKKIARKESLENKKNNKPEVNQIANSD | 800 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 751 | AVDNLADAESLNTAQKEEAEEKERKKIARKESLENKKNNKPEVNQIANSD | 800 |
| | . . . . . | |
| 801 | NKVTIDDYREEDEDKDPYPPCDVPVGEEEEEEEEDEPEVPAGPRPRRISE | 850 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 801 | NKVTIDDYREEDEDKDPYPPCDVPVGEEEEEEEEDEPEVPAGPRPRRISE | 850 |
| | . . . . . | |
| 851 | LNMKEKIAPIPEGSAFFILSKTNPIRVGCHKLINHHIFTNLILVFIMLSS | 900 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 851 | LNMKEKIAPIPEGSAFFILSKTNPIRVGCHKLINHHIFTNLILVFIMLSS | 900 |
| | . . . . . | |
| 901 | AALAAEDPIRSHSFRNTILGYFDYAFTAIFTVEILLKMTTFGAFLHKGAF | 950 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 901 | AALAAEDPIRSHSFRNTILGYFDYAFTAIFTVEILLKMTTFGAFLHKGAF | 950 |
| | . . . . . | |
| 951 | CRNYFNLLDMLVVGVSLVSFGIQSSAISVVKILRVLRVLRPLRAINRAKG | 1000 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 951 | CRNYFNLLDMLVVGVSLVSFGIQSSAISVVKILRVLRVLRPLRAINRAKG | 1000 |
| | . . . . . | |
| 1001 | LKHVVQCVFVAIRTIGNIMIVTTLLQFMFACIGVQLFKGKFYRCTDEAKS | 1050 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1001 | LKHVVQCVFVAIRTIGNIMIVTTLLQFMFACIGVQLFKGKFYRCTDEAKS | 1050 |
| | . . . . . | |
| 1051 | NPEECRGLFILYKDGDVDSPVVRERIWQNSDFNFDNVLSAMMALFTVSTF | 1100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1051 | NPEECRGLFILYKDGDVDSPVVRERIWQNSDFNFDNVLSAMMALFTVSTF | 1100 |
| | . . . . . | |
| 1101 | EGWPALLYKAIDSNGENIGPIYNHRVEISIFFIIYIIIVAFFMMNIFVGF | 1150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1101 | EGWPALLYKAIDSNGENIGPIYNHRVEISIFFIIYIIIVAFFMMNIFVGF | 1150 |
| | . . . . . | |
| 1151 | VIVTFQEQGEKEYKNCELDKNQRQCVEYALKARPLRRYIPKNPYQYKFWY | 1200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1151 | VIVTFQEQGEKEYKNCELDKNQRQCVEYALKARPLRRYIPKNPYQYKFWY | 1200 |
| | . . . . . | |
| 1201 | VVNSSPFEYMMFVLIMLNTLCLAMQHYEQSKMFNDAMDILNMVFTGVFTV | 1250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1201 | VVNSSPFEYMMFVLIMLNTLCLAMQHYEQSKMFNDAMDILNMVFTGVFTV | 1250 |
| | . . . . . | |
| 1251 | EMVLKVIAFKPKGYFSDAWNTFDSLIVIGSIIDVALSEADPTESENVPVP | 1300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1251 | EMVLKVIAFKPKGYFSDAWNTFDSLIVIGSIIDVALSEADPTESENVPVP | 1300 |
| | . . . . . | |
| 1301 | TATPGNSEESNRISITFFRLFRVMRLVKLLSRGEGIRTLLWTFIKSFQAL | 1350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1301 | TATPGNSEESNRISITFFRLFRVMRLVKLLSRGEGIRTLLWTFIKSFQAL | 1350 |
| | . . . . . | |
| 1351 | PYVALLIAMLFFIYAVIGMQMFGKVAMRDNNQINRNNNFQTFPQAVLLLF | 1400 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1351 | PYVALLIAMLFFIYAVIGMQMFGKVAMRDNNQINRNNNFQTFPQAVLLLF | 1400 |
| | . . . . . | |
| 1401 | RCATGEAWQEIMLACLPGKLCDPESDYNPGEEYTCGSNFAIVYFISFYML | 1450 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1401 | RCATGEAWQEIMLACLPGKLCDPESDYNPGEEYTCGSNFAIVYFISFYML | 1450 |
| | . . . . . | |
| 1451 | CAFLIINLFVAVIMDNFDYLTRDWSILGPHHLDEFKRIWSEYDPEAKGRI | 1500 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1451 | CAFLIINLFVAVIMDNFDYLTRDWSILGPHHLDEFKRIWSEYDPEAKGRI | 1500 |
| | . . . . . | |
| 1501 | KHLDVVTLLRRIQPPLGFGKLCPHRVACKRLVAMNMPLNSDGTVMFNATL | 1550 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1501 | KHLDVVTLLRRIQPPLGFGKLCPHRVACKRLVAMNMPLNSDGTVMFNATL | 1550 |
| | . . . . . | |
| 1551 | FALVRTALKIKTEGNLEQANEELRAVIKKIWKKTSMKLLDQVVPPAGDDE | 1600 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1551 | FALVRTALKIKTEGNLEQANEELRAVIKKIWKKTSMKLLDQVVPPAGDDE | 1600 |
| | . . . . . | |
| 1601 | VTVGKFYATFLIQDYFRKFKKRKEQGLVGKYPAKNTTIALQAGLRTLHDI | 1650 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1601 | VTVGKFYATFLIQDYFRKFKKRKEQGLVGKYPAKNTTIALQAGLRTLHDI | 1650 |
| | . . . . . | |
| 1651 | GPEIRRAISCDLQDDEPEETKREEEDDVFKRNGALLGNHVNHVNSDRRDS | 1700 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1651 | GPEIRRAISCDLQDDEPEETKREEEDDVFKRNGALLGNHVNHVNSDRRDS | 1700 |
| | . . . . . | |
| 1701 | LQQTNTTHRPLHVQRPSIPPASDTEKPLFPPAGNSVCHNHHNHNSIGKQV | 1750 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1701 | LQQTNTTHRPLHVQRPSIPPASDTEKPLFPPAGNSVCHNHHNHNSIGKQV | 1750 |
| | . . . . . | |
| 1751 | PTSTNANLNNANMSKAAHGKRPSIGNLEHVSENGHHSSHKHDREPQRRSS | 1800 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1751 | PTSTNANLNNANMSKAAHGKRPSIGNLEHVSENGHHSSHKHDREPQRRSS | 1800 |
| | . . . . . | |
| 1801 | VK.........RSDSGDEQLPTICREDPEIHGYFRDPHCLGEQEYFSSEE | 1841 |
| | \|\| \|\|\|\|\|\|\|\|\|\|\|\|\|\|\|\|\|\|\|\|\|\|\|\|\|\|\|\|\|\|\|\|\|\|\|\|\|\|\| | |
| 1801 | VKRTRYYETYIRSDSGDEQLPTICREDPEIHGYFRDPHCLGEQEYFSSEE | 1850 |
| | . . . . . | |
| 1842 | CYEDDSSPTWSRQNYGYYSRYPGRNIDSERPRGYHHPQGFLEDDDSPVCY | 1891 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1851 | CYEDDSSPTWSRQNYGYYSRYPGRNIDSERPRGYHHPQGFLEDDDSPVCY | 1900 |
| | . . . . . | |
| 1892 | DSRRSPRRRLLPPTPASHRRSSFNFECLRRQSSQEEVPSSPIFPHRTALP | 1941 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1901 | DSRRSPRRRLLPPTPASHRRSSFNFECLRRQSSQEEVPSSPIFPHRTALP | 1950 |
| | . . . . . | |
| 1942 | LHLMQQQIMAVAGLDSSKAQKYSPSHSTRSWATPPATPPYRDWTPCYTPL | 1991 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1951 | LHLMQQQIMAVAGLDSSKAQKYSPSHSTRSWATPPATPPYRDWTPCYTPL | 2000 |
| | . . . . . | |
| 1992 | IQVEQSEALDQVNGSLPSLHRSSWYTDEPDISYRTFTPASLTVPSSFRNK | 2041 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2001 | IQVEQSEALDQVNGSLPSLHRSSWYTDEPDISYRTFTPASLTVPSSFRNK | 2050 |
| | . . . . . | |
| 2042 | NSDKQRSADSLVEAVLISEGLGRYARDPKFVSATKHEIADACDLTIDEME | 2091 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2051 | NSDKQRSADSLVEAVLISEGLGRYARDPKFVSATKHEIADACDLTIDEME | 2100 |
| | . . . . . | |
| 2092 | SAASTLLNGNVRPRANGDVGPLSHRQDYELQDFGPGYSDEEPDPGRDEED | 2141 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2101 | SAASTLLNGNVRPRANGDVGPLSHRQDYELQDFGPGYSDEEPDPGRDEED | 2150 |
| | . | |
| 2142 | LADEMICITTL | 2152 |
| | \|\|\|\|\|\|\|\|\|\|\| | |
| 2151 | LADEMICITTL | 2161 |

### EXAMPLE 23

The calcium channel alpha subunit splice variants according to the present invention are expected to have diagnostic and/or therapeutic utility. With regard to therapeutic utility, these variants are preferably targets for a drug or drugs, which may optionally be a small molecule for example.

A “variant-treatable” disease refers to any disease that is treatable through targeting a splice variant of any of the calcium channel alpha 1 subunits according to the present invention, and/or by targeting a calcium channel having such an alpha 1 subunit splice variant. “Treatment” also encompasses prevention, amelioration, elimination and control of the disease and/or pathological condition. The diseases for which such variants may be useful targets depends upon the functionality of the known protein (for example, cardiac vs. neuronal, T-type vs. L-type and so forth). The variants themselves are described by “cluster” or by gene, as these variants are splice variants of known proteins. Therefore, a “cluster-related disease” or a “protein-related disease” refers to a disease that may be treated by a particular protein, with regard to the description of such diseases below a therapeutic protein variant according to the present invention.

The term “biologically active”, as used herein, refers to a protein being suitable as a target for a drug or drugs. Likewise, “immunologically active” refers to the capability of the natural, recombinant, or synthetic ligand, or any oligopeptide thereof, to induce a specific immune response in appropriate animals or cells and to bind with specific antibodies.

The term “modulate”, as used herein, refers to a change in the activity of at least one calcium channel activity. For example, modulation may cause an increase or a decrease in protein activity, binding characteristics, or any other biological, functional or immunological properties of a calcium channel.

Methods of Treatment

As mentioned hereinabove the novel therapeutic protein variants of the present invention can be used as targets for drug or drugs to treat cluster or protein-related diseases, disorders or conditions.

Thus, according to an additional aspect of the present invention there is provided a method of treating cluster or protein-related disease, disorder or condition in a subject.

The subject according to the present invention is a mammal, preferably a human which is diagnosed with one of the disease, disorder or conditions described hereinabove, or alternatively is predisposed to at least one type of the cluster or protein-related disease, disorder or conditions described hereinabove.

As used herein the term “treating” refers to preventing, curing, reversing, attenuating, alleviating, minimizing, suppressing or halting the deleterious effects of the above-described diseases, disorders or conditions.

Treating, according to the present invention, can be effected by specifically upregulating or alternatively downregulating the expression of at least one of the polypeptides of the present invention in the subject.

Optionally, upregulation may be effected by administering to the subject at least one of the polypeptides of the present invention (e.g., recombinant or synthetic) or an active portion thereof, as described herein. However, since the bioavailability of large polypeptides may potentially be relatively small due to high degradation rate and low penetration rate, administration of polypeptides is preferably confined to small peptide fragments (e.g., about 100 amino acids). The polypeptide or peptide may optionally be administered in as part of a pharmaceutical composition, described in more detail below.

It will be appreciated that treatment of the above-described diseases according to the present invention may be combined with other treatment methods known in the art (i.e., combination therapy). Thus, treatment of malignancies using the agents of the present invention may be combined with, for example, radiation therapy, antibody therapy and/or chemotherapy.

Alternatively or additionally, an upregulating method may optionally be effected by specifically upregulating the amount (optionally expression) in the subject of at least one of the polypeptides of the present invention or active portions thereof.

As is mentioned hereinabove and in the Examples section which follows, the biomolecular sequences of this aspect of the present invention may be used as valuable therapeutic tools in the treatment of diseases, disorders or conditions in which altered activity or expression of the wild-type (known) gene product is known to contribute to disease, disorder or condition onset or progression.

It will be appreciated that the polypeptides of the present invention may also have agonistic properties as targets, such that increasing their rate or level of function may be desired. As such, the biomolecular sequences of this aspect of the present invention may be used to treat conditions or diseases in which the wild-type gene product plays a favorable role, for example, increasing angiogenesis in cases of diabetes or ischemia.

Upregulating expression of the therapeutic protein or polypeptide variants of the present invention may be effected via the administration of at least one of the exogenous polynucleotide sequences of the present invention, ligated into a nucleic acid expression construct (as described in greater detail hereinabove) designed for expression of coding sequences in eukaryotic cells (e.g., mammalian cells), as described above. Accordingly, the exogenous polynucleotide sequence may be a DNA or RNA sequence encoding the variants of the present invention or active portions thereof.

It will be appreciated that the nucleic acid construct can be administered to the individual employing any suitable mode of administration including in vivo gene therapy (e.g., using viral transformation as described hereinabove). Alternatively, the nucleic acid construct is introduced into a suitable cell via an appropriate gene delivery vehicle/method (transfection, transduction, homologous recombination, etc.) and an expression system as needed and then the modified cells are expanded in culture and returned to the individual (i.e., ex-vivo gene therapy).

Such cells (i.e., which are transfected with the nucleic acid construct of the present invention) can be any suitable cells, such as kidney, bone marrow, keratinocyte, lymphocyte, adult stem cells, cord blood cells, embryonic stem cells which are derived from the individual and are transfected ex vivo with an expression vector containing the polynucleotide designed to express the polypeptide of the present inevntion as described hereinabove.

Administration of the ex vivo transfected cells of the present invention can be effected using any suitable route such as intravenous, intra peritoneal, intra kidney, intra gastrointestinal track, subcutaneous, transcutaneous, intramuscular, intracutaneous, intrathecal, epidural and rectal. According to presently preferred embodiments, the ex vivo transfected cells of the present invention are introduced to the individual using intravenous, intra kidney, intra gastrointestinal track and/or intra peritoneal administrations.

The ex vivo transfected cells of the present invention can be derived from either autologous sources such as self bone marrow cells or from allogeneic sources such as bone marrow or other cells derived from non-autologous sources. Since non-autologous cells are likely to induce an immune reaction when administered to the body several approaches have been developed to reduce the likelihood of rejection of non-autologous cells. These include either suppressing the recipient immune system or encapsulating the non-autologous cells or tissues in immunoisolating, semipermeable membranes before transplantation.

Encapsulation techniques are generally classified as microencapsulation, involving small spherical vehicles and macroencapsulation, involving larger flat-sheet and hollow-fiber membranes (Uludag, H. et al. Technology of mammalian cell encapsulation. Adv Drug Deliv Rev. 2000; 42: 29-64).

Methods of preparing microcapsules are known in the arts and include for example those disclosed by Lu M Z, et al., Cell encapsulation with alginate and alpha-phenoxycinnamnylideneacetylated poly(allylamine). Biotechnol Bioeng. 2000, 70: 479-83, Chang T M and Prakash S. Procedures for microencapsulation of enzymes, cells and genetically engineered microorganisms. Mol Biotechnol. 2001, 17: 249-60, and Lu M Z, et al., A novel cell encapsulation method using photosensitive poly(allylamine alpha-cyanocinnamylideneacetate). J Microencapsul. 2000, 17: 245-51.

For example, microcapsules are prepared by complexing modified collagen with a ter-polymer shell of 2-hydroxyethyl methylacrylate (HEMA), methacrylic acid (MAA) and methyl methacrylate (MMA), resulting in a capsule thickness of 2-5 μm. Such microcapsules can be further encapsulated with additional 2-5 μm ter-polymer shells in order to impart a negatively charged smooth surface and to minimize plasma protein absorption (Chia, S. M. et al. Multi-layered microcapsules for cell encapsulation Biomaterials. 2002 23: 849-56).

Other microcapsules are based on alginate, a marine polysaccharide (Sambanis, A. Encapsulated islets in diabetes treatment. Diabetes Thechnol. Ther. 2003, 5: 665-8) or its derivatives. For example, microcapsules can be prepared by the polyelectrolyte complexation between the polyanions sodium alginate and sodium cellulose sulphate with the polycation poly(methylene-co-guanidine) hydrochloride in the presence of calcium chloride.

It will be appreciated that cell encapsulation is improved when smaller capsules are used. Thus, the quality control, mechanical stability, diffusion properties, and in vitro activities of encapsulated cells improved when the capsule size was reduced from 1 mm to 400 μm (Canaple L. et al., Improving cell encapsulation through size control. J Biomater Sci Polym Ed. 2002; 13: 783-96). Moreover, nanoporous biocapsules with well-controlled pore size as small as 7 nm, tailored surface chemistries and precise microarchitectures were found to successfully immunoisolate microenvironments for cells (Williams D. Small is beautiful: microparticle and nanoparticle technology in medical devices. Med Device Technol. 1999, 10: 6-9; Desai, T. A. Microfabrication technology for pancreatic cell encapsulation. Expert Opin Biol Ther. 2002, 2: 633-46).

It will be appreciated that the present methodology may also be effected by specifically upregulating the expression of the variants of the present invention endogenously in the subject. Agents for upregulating endogenous expression of specific splice variants of a given gene include antisense oligonucleotides, which are directed at splice sites of interest, thereby altering the splicing pattern of the gene. This approach has been successfully used for shifting the balance of expression of the two isoforms of Bcl-x [Taylor (1999) Nat. Biotechnol. 17:1097-1100; and Mercatante (2001) J. Biol. Chem. 276:16411-16417]; IL-5R [Karras (2000) Mol. Pharmacol. 58:380-387]; and c-myc [Giles (1999) Antisense Acid Drug Dev. 9:213-220].

For example, interleukin 5 and its receptor play a critical role as regulators of hematopoiesis and as mediators in some inflammatory diseases such as allergy and asthma. Two alternatively spliced isoforms are generated from the IL-5R gene, which include (i.e., long form) or exclude (i.e., short form) exon 9. The long form encodes for the intact membrane-bound receptor, while the shorter form encodes for a secreted soluble non-functional receptor. Using 2′-O-MOE-oligonucleotides specific to regions of exon 9, Karras and co-workers (supra) were able to significantly decrease the expression of the wild type receptor and increase the expression of the shorter isoforms. Design and synthesis of oligonucleotides which can be used according to the present invention are described hereinbelow and by Sazani and Kole (2003) Progress in Moleclular and Subcellular Biology 31:217-239.

Treatment can preferably effected by agents which are capable of specifically downregulating expression (or activity) of at least one of the polypeptide variants of the present invention.

Down regulating the expression of the therapeutic protein variants of the present invention may be achieved using oligonucleotide agents such as those described in greater detail below.

SiRNA molecules—Small interfering RNA (siRNA) molecules can be used to down-regulate expression of the therapeutic protein variants of the present invention. RNA interference is a two-step process. The first step, which is termed as the initiation step, input dsRNA is digested into 21-23 nucleotide (nt) small interfering RNAs (siRNA), probably by the action of Dicer, a member of the RNase III family of dsRNA-specific ribonucleases, which processes (cleaves) dsRNA (introduced directly or via a transgene or a virus) in an ATP-dependent manner. Successive cleavage events degrade the RNA to 19-21 bp duplexes (siRNA), each with 2-nucleotide 3′ overhangs [Hutvagner and Zamore Curr. Opin. Genetics and Development 12:225-232 (2002); and Bernstein Nature 409:363-366 (2001)].

In the effector step, the siRNA duplexes bind to a nuclease complex to from the RNA-induced silencing complex (RISC). An ATP-dependent unwinding of the siRNA duplex is required for activation of the RISC. The active RISC then targets the homologous transcript by base pairing interactions and cleaves the mRNA into 12 nucleotide fragments from the 3′ terminus of the siRNA [Hutvagner and Zamore Curr. Opin. Genetics and Development 12:225-232 (2002); Hammond et al. (2001) Nat. Rev. Gen. 2:110-119 (2001); and Sharp Genes. Dev. 15:485-90 (2001)]. Although the mechanism of cleavage is still to be elucidated, research indicates that each RISC contains a single siRNA and an RNase [Hutvagner and Zamore Curr. Opin. Genetics and Development 12:225-232 (2002)].

Because of the remarkable potency of RNAi, an amplification step within the RNAi pathway has been suggested. Amplification could occur by copying of the input dsRNAs which would generate more siRNAs, or by replication of the siRNAs formed. Alternatively or additionally, amplification could be effected by multiple turnover events of the RISC [Hammond et al. Nat. Rev. Gen. 2:110-119 (2001), Sharp Genes. Dev. 15:485-90 (2001); Hutvagner and Zamore Curr. Opin. Genetics and Development 12:225-232 (2002)]. For more information on RNAi see the following reviews Tuschl ChemBiochem. 2:239-245 (2001); Cullen Nat. Immunol. 3:597-599 (2002); and Brantl Biochem. Biophys. Act. 1575:15-25 (2002).

Synthesis of RNAi molecules suitable for use with the present invention can be effected as follows. First, the mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www.ambion.com/techlib/tn/91/912.html).

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. Target sites are selected from the unique nucleotide sequences of each of the polynucleotides of the present invention, such that each polynucleotide is specifically down regulated. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

DNAzyme molecules—Another agent capable of downregulating expression of the polypeptides of the present invention is a DNAzyme molecule capable of specifically cleaving an mRNA transcript or DNA sequence of the polynucleotides of the present invention. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Target sites for DNAzymes are selected from the unique nucleotide sequences of each of the polynucleotides of the present invention, such that each polynucleotide is specifically down regulated.

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al, 20002, Abstract 409, Ann Meeting Am Soc Gen Ther www.asgt.org). In another application, DNAzymes complementary to bcr-ab1 oncogenes were successful in inhibiting the oncogenes expression in leukemia cells, and lessening relapse rates in autologous bone marrow transplant in cases of CML and ALL.

Antisense molecules—Downregulation of the polynucleotides of the present invention can also be effected by using an antisense polynucleotide capable of specifically hybridizing with an mRNA transcript encoding the polypeptide variants of the present invention. The term “antisense”, as used herein, refers to any composition containing nucleotide sequences, which are complementary to a specific DNA or RNA sequence.

The term “antisense strand” is used in reference to a nucleic acid strand that is complementary to the “sense” strand. Antisense molecules also include peptide nucleic acids and may be produced by any method including synthesis or transcription. Once introduced into a cell, the complementary nucleotides combine with natural sequences produced by the cell to form duplexes and block either transcription or translation. The designation “negative” is sometimes used in reference to the antisense strand, and “positive” is sometimes used in reference to the sense strand. Antisense oligonucleotides are also used for modulation of alternative splicing in vivo and for diagnostics in vivo and in vitro (Khelifi C. et al., 2002, Current Pharmaceutical Design 8:451-1466; Sazani, P., and Kole. R. Progress in Molecular and Cellular Biology, 2003, 31:217-239).

Design of antisense molecules which can be used to efficiently downregulate expression of the polypeptides of the present invention must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Luft J Mol Med 76: 75-6 (1998); Kronenwett et al. Blood 91: 852-62 (1998); Rajur et al. Bioconjug Chem 8: 935-40 (1997); Lavigne et al. Biochem Biophys Res Commun 237: 566-71 (1997) and Aoki et al. (1997) Biochem Biophys Res Commun 231: 540-5 (1997)].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al., Nature Biotechnology 16: 1374-1375 (1998)].

Several clinical trials have demonstrated safety, feasibility and activity of antisense oligonucleotides. For example, antisense oligonucleotides suitable for the treatment of cancer have been successfully used [Holmund et al., Curr Opin Mol Ther 1:372-85 (1999)], while treatment of hematological malignancies via antisense oligonucleotides targeting c-myb gene, p53 and Bcl-2 had entered clinical trials and had been shown to be tolerated by patients [Gerwitz Curr Opin Mol Ther 1:297-306 (1999)].

More recently, antisense-mediated suppression of human heparanase gene expression has been reported to inhibit pleural dissemination of human cancer cells in a mouse model [Uno et al., Cancer Res 61:7855-60 (2001)].

Thus, the current consensus is that recent developments in the field of antisense technology which, as described above, have led to the generation of highly accurate antisense design algorithms and a wide variety of oligonucleotide delivery systems, enable an ordinarily skilled artisan to design and implement antisense approaches suitable for down-regulating expression of known sequences without having to resort to undue trial and error experimentation.

Target sites for antisense molecules are selected from the unique nucleotide sequences of each of the polynucleotides of the present invention, such that each polynucleotide is specifically down regulated.

Ribozymes—Another agent capable of downregulating expression of the polypeptides of the present invention is a ribozyme molecule capable of specifically cleaving an mRNA transcript encoding the polypeptide variants of the present invention. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

An additional method of regulating the expression of a spcific gene in cells is via triplex forming oligonuclotides (TFOs). Recent studies have shown that TFOs can be designed which can recognize and bind to polypurine/polypirimidine regions in double-stranded helical DNA in a sequence-specific manner. These recognition rules are outlined by Maher III, L. J., et al., Science, 1989; 245:725-730; Moser, H. E., et al., Science, 1987; 238:645-630; Beal, P. A., et al, Science, 1992; 251:1360-1363; Cooney, M., et al., Science, 1988; 241:456-459; and Hogan, M. E., et al., EP Publication 375408. Modification of the oligonuclotides, such as the introduction of intercalators and backbone substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer, J Clin Invest 2003; 112:487-94).

In general, the triplex-forming oligonucleotide has the sequence correspondence:

| | |
|---|---|
| oligo | 3′--A G G T |
| duplex | 5′--A G C T |
| duplex | 3′--T C G A |

However, it has been shown that the A-AT and G-GC triplets have the greatest triple helical stability (Reither and Jeltsch, BMC Biochem, 2002, Sep. 12, Epub). The same authors have demonstrated that TFOs designed according to the A-AT and G-GC rule do not form non-specific triplexes, indicating that the triplex formation is indeed sequence specific.

Thus for any given sequence in the gene regulatory region a triplex forming sequence may be devised. Triplex-forming oligonucleotides preferably are at least 15, more preferably 25, still more preferably 30 or more nucleotides in length, up to 50 or 100 bp.

Transfection of cells (for example, via cationic liposomes) with TFOs, and formation of the triple helical structure with the target DNA induces steric and functional changes, blocking transcription initiation and elongation, allowing the introduction of desired sequence changes in the endogenous DNA and resulting in the specific downregulation of gene expression. Examples of such suppression of gene expression in cells treated with TFOs include knockout of episomal supFG1 and endogenous HPRT genes in mammalian cells (Vasquez et al., Nucl Acids Res. 1999; 27:1176-81, and Puri, et al, J Biol Chem, 2001; 276:28991-98), and the sequence- and target specific downregulation of expression of the Ets2 transcription factor, important in prostate cancer etiology (Carbone, et al, Nucl Acid Res. 2003; 31:833-43), and the pro-inflammatory ICAM-1 gene (Besch et al, J Biol Chem, 2002; 277:32473-79). In addition, Vuyisich and Beal have recently shown that sequence specific TFOs can bind to dsRNA, inhibiting activity of dsRNA-dependent enzymes such as RNA-dependent kinases (Vuyisich and Beal, Nuc. Acids Res 2000; 28:2369-74).

Additionally, TFOs designed according to the abovementioned principles can induce directed mutagenesis capable of effecting DNA repair, thus providing both downregulation and upregulation of expression of endogenous genes (Seidman and Glazer, J Clin Invest 2003; 12:487-94). Detailed description of the design, synthesis and administration of effective TFOs can be found in U.S. Patent Application Nos. 2003 017068 and 2003 0096980 to Froehler et al, and 2002 0128218 and 2002 0123476 to Emanuele et al, and U.S. Pat. No. 5,721,138 to Lawn.

Alternatively, down regulation of the polypeptide variants of the present invention may be achieved at the polypeptide level using downregulating agents such as antibodies or antibody fragments capabale of specifically binding the polypeptides of the present invention and inhibiting the activity thereof (i.e., neutralizing antibodies). Such antibodies can be directed for example, to the heterodimerizing domain on the variant, or to a putative ligand binding domain. Further description of antibodies and methods of generating same is provided below.

Alternatively, down regulation of the polypeptide variants of the present invention may be achieved using small, unique peptide sequences (e.g., of about 50-100 amino acids) which are capable of specifically binding to their target molecules (e.g., a receptor subunit) and thus prevent endogenous subunit assembly or association and therefore antagonize the receptor activity. Such peptides can be natural or synthetic peptides which are derived from the polypeptide of the present invention.

Pharmaceutical Compositions and Delivery Thereof

The present invention features a pharmaceutical composition comprising a therapeutically effective amount of a therapeutic agent according to the present invention, which preferably binds to and/or affects a calcium channel splice variant as described herein. Optionally and alternatively, the therapeutic agent could be an antibody or an oligonucleotide that specifically recognizes and binds to the therapeutic protein variant, but not to the corresponding known protein.

The pharmaceutical composition according to the present invention is preferably used for the treatment of cluster or protein-related disease, disorder or condition.

“Treatment” refers to both therapeutic treatment and prophylactic or preventative measures. Those in need of treatment include those already with the disorder as well as those in which the disorder is to be prevented. Hence, the mammal to be treated herein may have been diagnosed as having the disorder or may be predisposed or susceptible to the disorder. “Mammal” for purposes of treatment refers to any animal classified as a mammal, including humans, domestic and farm animals, and zoo, sports, or pet animals, such as dogs, horses, cats, cows, etc. Preferably, the mammal is human.

A “disorder” is any condition that would benefit from treatment with the agent according to the present invention. This includes chronic and acute disorders or diseases including those pathological conditions which predispose the mammal to the disorder in question. Non-limiting examples of disorders to be treated herein are described with regard to specific examples given herein.

The term “therapeutically effective amount” refers to an amount of agent according to the present invention that is effective to treat a disease or disorder in a mammal. In the case of cancer, the therapeutically effective amount of the agent may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the agent may prevent growth and/or kill existing cancer cells, it may be cytostatic and/or cytotoxic. For cancer therapy, efficacy can, for example, be measured by assessing the time to disease progression (TTP) and/or determining the response rate (RR).

The therapeutic agents of the present invention can be provided to the subject per se, or as part of a pharmaceutical composition where they are mixed with a pharmaceutically acceptable carrier.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the preparation accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases. One of the ingredients included in the pharmaceutically acceptable carrier can be for example polyethylene glycol (PEG), a biocompatible polymer with a wide range of solubility in both organic and aqueous media (Mutter et al. (1979).

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, intraperitoneal, intranasal, or intraocular injections. Alternately, one may administer a preparation in a local rather than systemic manner, for example, via injection of the preparation directly into a specific region of a patient's body.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The preparation of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated.

Determination of a therapeutically effective amount is well within the capability of those skilled in the art.

For any preparation used in the methods of the invention, the therapeutically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions including the preparation of the present invention formulated in a compatible pharmaceutical carrier may also be prepared, placed in an appropriate container, and labeled for treatment of an indicated condition.

Pharmaceutical compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

Immunogenic Compositions

A therapeutic agent according to the present invention may optionally be a molecule, which promotes a specific immunogenic response against at least one of the polypeptides of the present invention in the subject. The molecule can be polypeptide variants of the present invention, a fragment derived therefrom or a nucleic acid sequence encoding thereof. Although such a molecule can be provided to the subject per se, the agent is preferably administered with an immunostimulant in an immunogenic composiiton. An immunostimulant may be any substance that enhances or potentiates an immune response (antibody and/or cell-mediated) to an exogenous antigen. Examples of immunostimulants include adjuvants, biodegradable microspheres (e.g., polylactic galactide) and liposomes into which the compound is incorporated (see e.g., U.S. Pat. No. 4,235,877). Vaccine preparation is generally described in, for example, M. F. Powell and M. J. Newman, eds., “Vaccine Design (the subunit and adjuvant approach),” Plenum Press (NY, 1995).

Illustrative immunogenic compositions may contain DNA encoding one or more of the polypeptides as described above, such that the polypeptide is generated in situ. The DNA may be present within any of a variety of delivery systems known to those of ordinary skill in the art, including nucleic acid expression systems (see below), bacteria and viral expression systems. Numerous gene delivery techniques are well known in the art, such as those described by Rolland, Crit. Rev. Therap. Drug Carrier Systems 15:143-198, 1998, and references cited therein. Appropriate nucleic acid expression systems contain the necessary DNA sequences for expression in the subject (such as a suitable promoter and terminating signal). Bacterial delivery systems involve the administration of a bacterium (such as Bacillus-Calmette-Guerrin) that expresses an immunogenic portion of the polypeptide on its cell surface or secretes such an epitope. In a preferred embodiment, the DNA may be introduced using a viral expression system (e.g., vaccinia or other pox virus, retrovirus, or adenovirus), which may involve the use of a non-pathogenic (defective), replication competent virus. Suitable systems are disclosed, for example, in Fisher-Hoch et al., Proc. Natl. Acad. Sci. USA 86:317-321, 1989; Flexner et al., Ann. N.Y. Acad. Sci. 569:86-103, 1989; Flexner et al., Vaccine 8:17-21, 1990; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6:616-627, 1988; Rosenfeld et al., Science 252:431-434, 1991; Kolls et al., Proc. Natl. Acad. Sci. USA 91:215-219, 1994; Kass-Eisler et al., Proc. Natl. Acad. Sci. USA 90:11498-11502, 1993; Guzman et al., Circulation 88:2838-2848, 1993; and Guzman et al., Cir. Res. 73:1202-1207, 1993. Techniques for incorporating DNA into such expression systems are well known to those of ordinary skill in the art. The DNA may also be “naked,” as described, for example, in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

It will be appreciated that an immunogenic composition may comprise both a polynucleotide and a polypeptide component. Such immunogenic compositions may provide for an enhanced immune response.

Any of a variety of immunostimulants may be employed in the immunogenic compositions of this invention. For example, an adjuvant may be included. Most adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, may also be used as adjuvants.

The adjuvant composition may be designed to induce an immune response predominantly of the Th1 type. High levels of Th1-type cytokines (e.g., IFN-.gamma., TNF.alpha., IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of an immunogenic composition as provided herein, the subject will support an immune response that includes Th1- and Th2-type responses. The levels of these cytokines may be readily assessed using standard assays. For a review of the families of cytokines, see Mosmann and Coffinan, Ann. Rev. Immunol. 7:145-173, 1989.

Preferred adjuvants for use in eliciting a predominantly Th1-type response include, for example, a combination of monophosphoryl lipid A, preferably 3-de-O-acylated monophosphoryl lipid A (3D-MPL), together with an aluminum salt. MPL adjuvants are available from Corixa Corporation (Seattle, Wash.; see U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996. Another preferred adjuvant is a saponin, preferably QS21 (Aquila Biopharmaceuticals Inc., Framingham, Mass.), which may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other preferred formulations comprise an oil-in-water emulsion and tocopherol. A particularly potent adjuvant formulation involving QS21, 3D-MPL and tocopherol in an oil-in-water emulsion is described in WO 95/17210.

Other preferred adjuvants include Montanide ISA 720 (Seppic, France), SAF (Chiron, Calif., United States), ISCOMS (CSL), MF-59 (Chiron), the SBAS series of adjuvants (e.g., SBAS-2 or SBAS-4, available from SmithKline Beecham, Rixensart, Belgium), Detox (Corixa, Hamilton, Mont.), RC-529 (Corixa, Hamilton, Mont.) and other aminoalkyl glucosaminide 4-phosphates (AGPs), such as those described in pending U.S. patent application Ser. Nos. 08/853,826 and 09/074,720.

A delivery vehicle may be employed within the immunogenic composition of the present invention to facilitate production of an antigen-specific immune response that targets tumor cells. Delivery vehicles include antigen presenting cells (APCs), such as dendritic cells, macrophages, B cells, monocytes and other cells that may be engineered to be efficient APCs. Such cells may be genetically modified to increase the capacity for presenting the antigen, to improve activation and/or maintenance of the T cell response, to have anti-tumor effects per se and/or to be immunologically compatible with the receiver (i.e., matched HLA haplotype). APCs may generally be isolated from any of a variety of biological fluids and organs, including tumor and peritumoral tissues, and may be autologous, allogeneic, syngeneic or xenogeneic cells.

Dendritic cells are highly potent APCs (Banchereau and Steinman, Nature 392:245-251, 1998) and have been shown to be effective as a physiological adjuvant for eliciting prophylactic or therapeutic antitumor immunity (see Timmeman and Levy, Ann. Rev. Med. 50:507-529, 1999). In general, dendritic cells may be identified based on their typical shape (stellate in situ, with marked cytoplasmic processes (dendrites) visible in vitro), their ability to take up, process and present antigens with high efficiency and their ability to activate naive T cell responses. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within an immunogenic composition (see Zitvogel et al., Nature Med. 4:594-600, 1998).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNF.alpha. to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNF.alpha., CD40 ligand, LPS, flt3 ligand and/or other compound(s) that induce differentiation, maturation and proliferation of dendritic cells.

Dendritic cells are categorized as “immature” and “mature” cells, which allows a simple way to discriminate between two well characterized phenotypes. Immature dendritic cells are characterized as APC with a high capacity for antigen uptake and processing, which correlates with the high expression of Fcy receptor and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1BB).

APCs may generally be transfected with at least one polynucleotide encoding a polypeptide of the present invention, such that variant II, or an immunogenic portion thereof, is expressed on the cell surface. Such transfection may take place ex vivo, and a composition comprising such transfected cells may then be used for therapeutic purposes, as described herein. Alternatively, a gene delivery vehicle that targets a dendritic or other antigen presenting cell may be administered to the subject, resulting in transfection that occurs in vivo. In vivo and ex vivo transfection of dendritic cells, for example, may generally be performed using any methods known in the art, such as those described in WO 97/24447, or the gene gun approach described by Mahvi et al., Immunology and cell Biology 75:456-460, 1997. Antigen loading of dendritic cells may be achieved by incubating dendritic cells or progenitor cells with a polypeptide of the present inventio, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule) such as described above. Alternatively, a dendritic cell may be pulsed with a non-conjugated immunological partner, separately or in the presence of the polypeptide.

Preferred embodiments of the present invention encompass novel naturally occurring secreted (i.e., extracellular) and non-secreted (i.e., intracellular or membranal) variants of genes and gene products, which, as is described in the Examples section which follows, play pivotal roles in disease onset and progression. As such these variants can be used for a wide range of diagnostic and/or therapeutic uses.

Diagnostic Methods

The term “marker” in the context of the present invention refers to a nucleic acid fragment, a peptide, or a polypeptide, which is differentially present in a sample taken from patients having or predisposed to a cluster or protein-related disease, disorder or condition as compared to a comparable sample taken from subjects who do not have a such a disease, disorder or condition. For example, optionally the presence or absence or level of a calcium splice variant according to the present invention may optionally be measured as a diagnostic for a disease and/or disorder. More preferably, at least one therapy is selected according to such a diagnosis.

The methods for detecting these markers have many applications. For example, one marker or combination of markers can be measured to differentiate between various types of cluster or protein-related disease, disorder or condition, and thus are useful as an aid in the accurate diagnosis of cluster or protein-related disease, disorder or condition in a patient. For example, one marker or combination of markers can be measured to differentiate between various types of lung cancers, such as small cell or non-small cell lung cancer, and further between non-small cell lung cancer types, such as adenocarcinomas, squamous cell and large cell carcinomas, and thus are useful as an aid in the accurate diagnosis of lung cancer in a patient. In another example, the present methods for detecting these markers can be applied to in vitro cluster or protein-related cancers cells or in vivo animal models for cluster or protein-related cancers to assay for and identify compounds that modulate expression of these markers.

The phrase “differentially present” refers to differences in the quantity of a marker present in a sample taken from patients having cluster or protein-related disease, disorder or condition as compared to a comparable sample taken from patients who do not have such disease, disorder or condition. For example, a nucleic acid fragment may optionally be differentially present between the two samples if the amount of the nucleic acid fragment in one sample is significantly different from the amount of the nucleic acid fragment in the other sample, for example as measured by hybridization and/or NAT-based assays. A polypeptide is differentially present between the two samples if the amount of the polypeptide in one sample is significantly different from the amount of the polypeptide in the other sample. It should be noted that if the marker is detectable in one sample and not detectable in the other, then such a marker can be considered to be differentially present. One of ordinary skill in the art could easily determine such relative levels of the markers; further guidance is provided below.

As used herein the phrase “diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The phrase “predisposition” used herein refers to the susceptibility to develop a disorder. A subject with a predisposition to develop a disorder is more likely to develop the disorder than a non-predisposed subject.

As used herein the phrase “diagnosing” refers to classifying a disease or a symptom, determining a severity of the disease, monitoring disease progression, forecasting an outcome of a disease and/or prospects of recovery. The term “detecting” may also optionally encompass any of the above.

Diagnosis of a disease according to the present invention can be effected by determining a level of a polynucleotide or a polypeptide of the present invention in a biological sample obtained from the subject, wherein the level determined can be correlated with predisposition to, or presence or absence of the disease.

As used herein “a biological sample” refers to a sample of tissue or fluid isolated from a subject, including but not limited to, for example, plasma, serum, spinal fluid, lymph fluid, the external sections of the skin, respiratory, intestinal, and genitourinary tracts, tears, saliva, sputum, milk, blood cells, tumors, neuronal tissue, organs, and also samples of in vivo cell culture constituents. It should be noted that a “biological sample obtained from the subject” may also optionally comprise a sample that has not been physically removed from the subject, as described in greater detail below.

As used herein, the term “level” refers to expression levels of RNA and/or protein or to DNA copy number of a marker of the present invention.

Typically the level of the marker in a biological sample obtained from the subject is different (i.e., increased or decreased) from the level of the same variant in a similar sample obtained from a healthy individual.

Numerous well known tissue or fluid collection methods can be utilized to collect the biological sample from the subject in order to determine the level of DNA, RNA and/or polypeptide of the variant of interest in the subject.

Examples include, but are not limited to, fine needle biopsy, needle biopsy, core needle biopsy and surgical biopsy (e.g., brain biopsy), and lavage. Regardless of the procedure employed, once a biopsy/sample is obtained the level of the variant can be determined and a diagnosis can thus be made.

Determining the level of the same variant in normal tissues of the same origin is preferably effected along-side to detect an elevated expression and/or amplification and/or a decreased expression, of the variant as opposed to the normal tissues.

A “test amount” of a marker refers to an amount of a marker in a subject's sample that is consistent with a diagnosis of a cluster or protein-related disease, disorder or condition related cancer or other UbcH10 related disease. A test amount can be either in absolute amount (e.g., microgram/ml) or a relative amount (e.g., relative intensity of signals).

A “control amount” of a marker can be any amount or a range of amounts to be compared against a test amount of a marker. For example, a control amount of a marker can be the amount of a marker in a patient which does not have the cluster or protein-related disease, disorder or condition. A control amount can be either in absolute amount (e.g., microgram/ml) or a relative amount (e.g., relative intensity of signals).

“Detect” refers to identifying the presence, absence or amount of the object to be detected.

A “label” includes any moiety or item detectable by spectroscopic, photo chemical, biochemical, immunochemical, or chemical means. For example, useful labels include ³²P, ³⁵S, fluorescent dyes, electron-dense reagents, enzymes (e.g., as commonly used in an ELISA), biotin-streptavidin, digoxigenin, haptens and proteins for which antisera or monoclonal antibodies are available, or nucleic acid molecules with a sequence complementary to a target. The label often generates a measurable signal, such as a radioactive, chromogenic, or fluorescent signal, that can be used to quantify the amount of bound label in a sample. The label can be incorporated in or attached to a primer or probe either covalently, or through ionic, van der Waals or hydrogen bonds, e.g., incorporation of radioactive nucleotides, or biotinylated nucleotides that are recognized by streptavidin. The label may be directly or indirectly detectable. Indirect detection can involve the binding of a second label to the first label, directly or indirectly. For example, the label can be the ligand of a binding partner, such as biotin, which is a binding partner for streptavidin, or a nucleotide sequence, which is the binding partner for a complementary sequence, to which it can specifically hybridize. The binding partner may itself be directly detectable, for example, an antibody may be itself labeled with a fluorescent molecule. The binding partner also may be indirectly detectable, for example, a nucleic acid having a complementary nucleotide sequence can be a part of a branched DNA molecule that is in turn detectable through hybridization with other labeled nucleic acid molecules (see, e.g., P. D. Fahrlander and A. Klausner, Bio/Technology 6:1165 (1988)). Quantitation of the signal is achieved by, e.g., scintillation counting, densitometry, or flow cytometry.

Exemplary detectable labels, optionally and preferably for use with immunoassays, include but are not limited to magnetic beads, fluorescent dyes, radiolabels, enzymes (e.g., horse radish peroxide, alkaline phosphatase and others commonly used in an ELISA), and calorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

“Immunoassay” is an assay that uses an antibody to specifically bind an antigen. The immunoassay is characterized by the use of specific binding properties of a particular antibody to isolate, target, and/or quantify the antigen.

The phrase “specifically (or selectively) binds” to an antibody or “specifically (or selectively) immunoreactive with” when referring to a protein or peptide (or other epitope), refers to a binding reaction that is determinative of the presence of the protein in a heterogeneous population of proteins and other biologics. Thus, under designated immunoassay conditions, the specified antibodies bind to a particular protein at least two times greater than the background (non-specific signal) and do not substantially bind in a significant amount to other proteins present in the sample. Specific binding to an antibody under such conditions may require an antibody that is selected for its specificity for a particular protein. For example, polyclonal antibodies raised to seminal basic protein from specific species such as rat, mouse, or human can be selected to obtain only those polyclonal antibodies that are specifically immunoreactive with seminal basic protein and not with other proteins, except for polymorphic variants and alleles of seminal basic protein. This selection may be achieved by subtracting out antibodies that cross-react with seminal basic protein molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

In another embodiment, this invention provides antibodies specifically recognizing the splice variants and polypeptide fragments thereof of this invention. Preferably such antibodies differentially recognize splice variants of the present invention but do not recognize a corresponding known protein (such known proteins are discussed with regard to their splice variants in the Examples below).

In another embodiment, this invention provides a method for detecting a splice variant according to the present invention in a biological sample, comprising: contacting a biological sample with an antibody specifically recognizing a splice variant according to the present invention under conditions whereby the antibody specifically interacts with the splice variant in the biological sample but do not recognize known corresponding proteins (wherein the known protein is discussed with regard to its splice variant(s) in the Examples below), and detecting the interaction; wherein the presence of an interaction correlates with the presence of a splice variant in the biological sample.

In another embodiment, this invention provides a method for detecting a splice variant nucleic acid sequences in a biological sample, comprising: hybridizing the isolated nucleic acid molecules or oligonucleotide fragments of at least about a minimum length to a nucleic acid material of a biological sample and detecting a hybridization complex; wherein the presence of a hybridization complex correlates with the presence of a splice variant nucleic acid sequence in the biological sample.

According to another embodiment of the present invention the detection of the splice variant nucleic acid sequences in the biological sample is effected by detecting at least one nucleic acid change within a nucleic acid material derived from the biological sample; wherein the presence of the at least one nucleic acid change correlates with the presence of a splice variant nucleic acid sequence in the biological sample.

According to the present invention, the splice variants described herein are non-limiting examples of markers for diagnosing the cluster or protein-related disease, disorder or condition. Each splice variant marker of the present invention can be used alone or in combination, for various uses, including but not limited to, prognosis, prediction, screening, early diagnosis, determination of progression, therapy selection and treatment monitoring of such a cancer, disease or pathology.

According to optional but preferred embodiments of the present invention, any marker according to the present invention may optionally be used alone or combination. Such a combination may optionally comprise a plurality of markers described herein, optionally including any subcombination of markers, and/or a combination featuring at least one other marker, for example a known marker. Furthermore, such a combination may optionally and preferably be used as described above with regard to determining a ratio between a quantitative or semi-quantitative measurement of any marker described herein to any other marker described herein, and/or any other known marker, and/or any other marker. With regard to such a ratio between any marker described herein (or a combination thereof) and a known marker, more preferably the known marker comprises the “known protein” as described in greater detail below with regard to each cluster or gene.

According to other preferred embodiments of the present invention, a splice variant protein or a fragment thereof, or a splice variant nucleic acid sequence or a fragment thereof, may be featured as a biomarker for detecting the cluster or protein-related disease, disorder or condiiton, such that a biomarker may optionally comprise any of the above.

Non-limiting examples of methods or assays are described below.

The present invention also relates to kits based upon such diagnostic methods or assays.

NAT Assays

Detection of a nucleic acid of interest in a biological sample may also optionally be effected by NAT-based assays, which involve nucleic acid amplification technology, such as PCR, or variations thereof (e.g., real-time PCR, RT-PCR and in situ RT-PCR).

As used herein, a “primer” defines an oligonucleotide which is capable of annealing to (hybridizing with) a target sequence, thereby creating a double stranded region which can serve as an initiation point for DNA synthesis under suitable conditions.

Amplification of a selected, or target, nucleic acid sequence may be carried out by a number of suitable methods. See generally Kwoh et al., 1990, Am. Biotechnol. Lab. 8:14. Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the q3 replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra).

The terminology “amplification pair” (or “primer pair”) refers herein to a pair of oligonucleotides (oligos) of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

In one particular embodiment, amplification of a nucleic acid sample from a patient is amplified under conditions which favor the amplification of the most abundant differentially expressed nucleic acid. In one preferred embodiment, RT-PCR is carried out on an mRNA sample from a patient under conditions which favor the amplification of the most abundant mRNA. In another preferred embodiment, the amplification of the differentially expressed nucleic acids is carried out simultaneously. It will be realized by a person skilled in the art that such methods could be adapted for the detection of differentially expressed proteins instead of differentially expressed nucleic acid sequences.

The nucleic acid (i.e. DNA or RNA) for practicing the present invention may be obtained according to well known methods.

Oligonucleotide primers of the present invention may be of any suitable length, depending on the particular assay format and the particular needs and targeted genomes employed. Optionally, the oligonucleotide primers are at least 12 nucleotides in length, preferably between 15 and 24 molecules, and they may be adapted to be especially suited to a chosen nucleic acid amplification system. As commonly known in the art, the oligonucleotide primers can be designed by taking into consideration the melting point of hybridization thereof with its targeted sequence (Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

It will be appreciated that antisense oligonucleotides may be employed to quantify expression of a splice isoform of interest. Such detection is effected at the pre-mRNA level. Essentially the ability to quantitate transcription from a splice site of interest can be effected based on splice site accessibility. Oligonucleotides may compete with splicing factors for the splice site sequences. Thus, low activity of the antisense oligonucleotide is indicative of splicing activity.

The polymerase chain reaction and other nucleic acid amplification reactions are well known in the art (various non-limiting examples of these reactions are described in greater detail below). The pair of oligonucleotides according to this aspect of the present invention are preferably selected to have compatible melting temperatures (Tm), e.g., melting temperatures which differ by less than that 7° C., preferably less than 5° C., more preferably less than 4° C., most preferably less than 3° C., ideally between 3° C. and 0° C.

Polymerase Chain Reaction (PCR): The polymerase chain reaction (PCR), as described in U.S. Pat. Nos. 4,683,195 and 4,683,202 to Mullis and Mullis et al., is a method of increasing the concentration of a segment of target sequence in a mixture of genomic DNA without cloning or purification. This technology provides one approach to the problems of low target sequence concentration. PCR can be used to directly increase the concentration of the target to an easily detectable level. This process for amplifying the target sequence involves the introduction of a molar excess of two oligonucleotide primers which are complementary to their respective strands of the double-stranded target sequence to the DNA mixture containing the desired target sequence. The mixture is denatured and then allowed to hybridize. Following hybridization, the primers are extended with polymerase so as to form complementary strands. The steps of denaturation, hybridization (annealing), and polymerase extension (elongation) can be repeated as often as needed, in order to obtain relatively high concentrations of a segment of the desired target sequence.

The length of the segment of the desired target sequence is determined by the relative positions of the primers with respect to each other, and, therefore, this length is a controllable parameter. Because the desired segments of the target sequence become the dominant sequences (in terms of concentration) in the mixture, they are the to be “PCR-amplified.”

Ligase Chain Reaction (LCR or LAR): The ligase chain reaction [LCR; sometimes referred to as “Ligase Amplification Reaction” (LAR)] has developed into a well-recognized alternative method of amplifying nucleic acids. In LCR, four oligonucleotides, two adjacent oligonucleotides which uniquely hybridize to one strand of target DNA, and a complementary set of adjacent oligonucleotides, which hybridize to the opposite strand are mixed and DNA ligase is added to the mixture. Provided that there is complete complementarity at the junction, ligase will covalently link each set of hybridized molecules. Importantly, in LCR, two probes are ligated together only when they base-pair with sequences in the target sample, without gaps or mismatches. Repeated cycles of denaturation, and ligation amplify a short segment of DNA. LCR has also been used in combination with PCR to achieve enhanced detection of single-base changes: see for example Segev, PCT Publication No. W09001069 A1 (1990). However, because the four oligonucleotides used in this assay can pair to form two short ligatable fragments, there is the potential for the generation of target-independent background signal. The use of LCR for mutant screening is limited to the examination of specific nucleic acid positions.

Self-Sustained Synthetic Reaction (3SR/NASBA): The self-sustained sequence replication reaction (3SR) is a transcription-based in vitro amplification system that can exponentially amplify RNA sequences at a uniform temperature. The amplified RNA can then be utilized for mutation detection. In this method, an oligonucleotide primer is used to add a phage RNA polymerase promoter to the 5′ end of the sequence of interest. In a cocktail of enzymes and substrates that includes a second primer, reverse transcriptase, RNase H, RNA polymerase and ribo-and deoxyribonucleoside triphosphates, the target sequence undergoes repeated rounds of transcription, cDNA synthesis and second-strand synthesis to amplify the area of interest. The use of 3SR to detect mutations is kinetically limited to screening small segments of DNA (e.g., 200-300 base pairs).

Q-Beta (Qβ) Replicase: In this method, a probe which recognizes the sequence of interest is attached to the replicatable RNA template for Qβ replicase. A previously identified major problem with false positives resulting from the replication of unhybridized probes has been addressed through use of a sequence-specific ligation step. However, available thermostable DNA ligases are not effective on this RNA substrate, so the ligation must be performed by T4 DNA ligase at low temperatures (37 degrees C.). This prevents the use of high temperature as a means of achieving specificity as in the LCR, the ligation event can be used to detect a mutation at the junction site, but not elsewhere.

A successful diagnostic method must be very specific. A straight-forward method of controlling the specificity of nucleic acid hybridization is by controlling the temperature of the reaction. While the 3SR/NASBA, and Qβ systems are all able to generate a large quantity of signal, one or more of the enzymes involved in each cannot be used at high temperature (i.e., >55 degrees C.). Therefore the reaction temperatures cannot be raised to prevent non-specific hybridization of the probes. If probes are shortened in order to make them melt more easily at low temperatures, the likelihood of having more than one perfect match in a complex genome increases. For these reasons, PCR and LCR currently dominate the research field in detection technologies.

The basis of the amplification procedure in the PCR and LCR is the fact that the products of one cycle become usable templates in all subsequent cycles, consequently doubling the population with each cycle. The final yield of any such doubling system can be expressed as: (1+X)n=y, where “X” is the mean efficiency (percent copied in each cycle), “n” is the number of cycles, and “y” is the overall efficiency, or yield of the reaction. If every copy of a target DNA is utilized as a template in every cycle of a polymerase chain reaction, then the mean efficiency is 100%. If 20 cycles of PCR are performed, then the yield will be 220, or 1,048,576 copies of the starting material. If the reaction conditions reduce the mean efficiency to 85%, then the yield in those 20 cycles will be only 1.8520, or 220,513 copies of the starting material. In other words, a PCR running at 85% efficiency will yield only 21% as much final product, compared to a reaction running at 100% efficiency. A reaction that is reduced to 50% mean efficiency will yield less than 1% of the possible product.

In practice, routine polymerase chain reactions rarely achieve the theoretical maximum yield, and PCRs are usually run for more than 20 cycles to compensate for the lower yield. At 50% mean efficiency, it would take 34 cycles to achieve the million-fold amplification theoretically possible in 20, and at lower efficiencies, the number of cycles required becomes prohibitive. In addition, any background products that amplify with a better mean efficiency than the intended target will become the dominant products.

Also, many variables can influence the mean efficiency of PCR, including target DNA length and secondary structure, primer length and design, primer and dNTP concentrations, and buffer composition, to name but a few. Contamination of the reaction with exogenous DNA (e.g., DNA spilled onto lab surfaces) or cross-contamination is also a major consideration. Reaction conditions must be carefully optimized for each different primer pair and target sequence, and the process can take days, even for an experienced investigator. The laboriousness of this process, including numerous technical considerations and other factors, presents a significant drawback to using PCR in the clinical setting. Indeed, PCR has yet to penetrate the clinical market in a significant way. The same concerns arise with LCR, as LCR must also be optimized to use different oligonucleotide sequences for each target sequence. In addition, both methods require expensive equipment, capable of precise temperature cycling.

Many applications of nucleic acid detection technologies, such as in studies of allelic variation, involve not only detection of a specific sequence in a complex background, but also the discrimination between sequences with few, or single, nucleotide differences. One method of the detection of allele-specific variants by PCR is based upon the fact that it is difficult for Taq polymerase to synthesize a DNA strand when there is a mismatch between the template strand and the 3′ end of the primer. An allele-specific variant may be detected by the use of a primer that is perfectly matched with only one of the possible alleles; the mismatch to the other allele acts to prevent the extension of the primer, thereby preventing the amplification of that sequence. This method has a substantial limitation in that the base composition of the mismatch influences the ability to prevent extension across the mismatch, and certain mismatches do not prevent extension or have only a minimal effect.

A similar 3′-mismatch strategy is used with greater effect to prevent ligation in the LCR. Any mismatch effectively blocks the action of the thermostable ligase, but LCR still has the drawback of target-independent background ligation products initiating the amplification. Moreover, the combination of PCR with subsequent LCR to identify the nucleotides at individual positions is also a clearly cumbersome proposition for the clinical laboratory.

The direct detection method according to various preferred embodiments of the present invention may be, for example a cycling probe reaction (CPR) or a branched DNA analysis.

When a sufficient amount of a nucleic acid to be detected is available, there are advantages to detecting that sequence directly, instead of making more copies of that target, (e.g., as in PCR and LCR). Most notably, a method that does not amplify the signal exponentially is more amenable to quantitative analysis. Even if the signal is enhanced by attaching multiple dyes to a single oligonucleotide, the correlation between the final signal intensity and amount of target is direct. Such a system has an additional advantage that the products of the reaction will not themselves promote further reaction, so contamination of lab surfaces by the products is not as much of a concern. Recently devised techniques have sought to eliminate the use of radioactivity and/or improve the sensitivity in automatable formats. Two examples are the “Cycling Probe Reaction” (CPR), and “Branched DNA” (bDNA).

Cycling probe reaction (CPR): The cycling probe reaction (CPR), uses a long chimeric oligonucleotide in which a central portion is made of RNA while the two termini are made of DNA. Hybridization of the probe to a target DNA and exposure to a thermostable RNase H causes the RNA portion to be digested. This destabilizes the remaining DNA portions of the duplex, releasing the remainder of the probe from the target DNA and allowing another probe molecule to repeat the process. The signal, in the form of cleaved probe molecules, accumulates at a linear rate. While the repeating process increases the signal, the RNA portion of the oligonucleotide is vulnerable to RNases that may carried through sample preparation.

Branched DNA: Branched DNA (bDNA), involves oligonucleotides with branched structures that allow each individual oligonucleotide to carry 35 to 40 labels (e.g., alkaline phosphatase enzymes). While this enhances the signal from a hybridization event, signal from non-specific binding is similarly increased.

The NAT assays of the present invention also include methods of detecting at least one nucleic acid change [e.g., a single nucleotide polymorphism (SNP] in the biological sample of the present invention.

The demand for tests which allow the detection of specific nucleic acid sequences and sequence changes is growing rapidly in clinical diagnostics. As nucleic acid sequence data for genes from humans and pathogenic organisms accumulates, the demand for fast, cost-effective, and easy-to-use tests for as yet mutations within specific sequences is rapidly increasing.

A handful of methods have been devised to scan nucleic acid segments for mutations or nucleic acid changes. One option is to determine the entire gene sequence of each test sample (e.g., a bacterial isolate). For sequences under approximately 600 nucleotides, this may be accomplished using amplified material (e.g., PCR reaction products). This avoids the time and expense associated with cloning the segment of interest. However, specialized equipment and highly trained personnel are required, and the method is too labor-intense and expensive to be practical and effective in the clinical setting.

In view of the difficulties associated with sequencing, a given segment of nucleic acid may be characterized on several other levels. At the lowest resolution, the size of the molecule can be determined by electrophoresis by comparison to a known standard run on the same gel. A more detailed picture of the molecule may be achieved by cleavage with combinations of restriction enzymes prior to electrophoresis, to allow construction of an ordered map. The presence of specific sequences within the fragment can be detected by hybridization of a labeled probe, or the precise nucleotide sequence can be determined by partial chemical degradation or by primer extension in the presence of chain-terminating nucleotide analogs.

Restriction fragment length polymorphism (RFLP): For detection of single-base differences between like sequences, the requirements of the analysis are often at the highest level of resolution. For cases in which the position of the nucleotide in question is known in advance, several methods have been developed for examining single base changes without direct sequencing. For example, if a mutation of interest happens to fall within a restriction recognition sequence, a change in the pattern of digestion can be used as a diagnostic tool (e.g., restriction fragment length polymorphism [RFLP] analysis).

Single point mutations have been also detected by the creation or destruction of RFLPs. Mutations are detected and localized by the presence and size of the RNA fragments generated by cleavage at the mismatches. Single nucleotide mismatches in DNA heteroduplexes are also recognized and cleaved by some chemicals, providing an alternative strategy to detect single base substitutions, generically named the “Mismatch Chemical Cleavage” (MCC). However, this method requires the use of osmium tetroxide and piperidine, two highly noxious chemicals which are not suited for use in a clinical laboratory.

RFLP analysis suffers from low sensitivity and requires a large amount of sample. When RFLP analysis is used for the detection of point mutations, it is, by its nature, limited to the detection of only those single base changes which fall within a restriction sequence of a known restriction endonuclease. Moreover, the majority of the available enzymes have 4 to 6 base-pair recognition sequences, and cleave too frequently for many large-scale DNA manipulations. Thus, it is applicable only in a small fraction of cases, as most mutations do not fall within such sites.

A handful of rare-cutting restriction enzymes with 8 base-pair specificities have been isolated and these are widely used in genetic mapping, but these enzymes are few in number, are limited to the recognition of G+C-rich sequences, and cleave at sites that tend to be highly clustered. Recently, endonucleases encoded by group I introns have been discovered that might have greater than 12 base-pair specificity, but again, these are few in number.

Allele specific oligonucleotide (ASO): If the change is not in a recognition sequence, then allele-specific oligonucleotides (ASOs), can be designed to hybridize in proximity to the mutated nucleotide, such that a primer extension or ligation event can bused as the indicator of a match or a mis-match. Hybridization with radioactively labeled allelic specific oligonucleotides (ASO) also has been applied to the detection of specific point mutations. The method is based on the differences in the melting temperature of short DNA fragments differing by a single nucleotide. Stringent hybridization and washing conditions can differentiate between mutant and wild-type alleles. The ASO approach applied to PCR products also has been extensively utilized by various researchers to detect and characterize point mutations in ras genes and gsp/gip oncogenes. Because of the presence of various nucleotide changes in multiple positions, the ASO method requires the use of many oligonucleotides to cover all possible oncogenic mutations.

With either of the techniques described above (i.e., RFLP and ASO), the precise location of the suspected mutation must be known in advance of the test. That is to say, they are inapplicable when one needs to detect the presence of a mutation within a gene or sequence of interest.

Denaturing/Temperature Gradient Gel Electrophoresis (DGGE/TGGE): Two other methods rely on detecting changes in electrophoretic mobility in response to minor sequence changes. One of these methods, termed “Denaturing Gradient Gel Electrophoresis” (DGGE) is based on the observation that slightly different sequences will display different patterns of local melting when electrophoretically resolved on a gradient gel. In this manner, variants can be distinguished, as differences in melting properties of homoduplexes versus heteroduplexes differing in a single nucleotide can detect the presence of mutations in the target sequences because of the corresponding changes in their electrophoretic mobilities. The fragments to be analyzed, usually PCR products, are “clamped” at one end by a long stretch of G−C base pairs (30-80) to allow complete denaturation of the sequence of interest without complete dissociation of the strands. The attachment of a GC “clamp” to the DNA fragments increases the fraction of mutations that can be recognized by DGGE. Attaching a GC clamp to one primer is critical to ensure that the amplified sequence has a low dissociation temperature. Modifications of the technique have been developed, using temperature gradients, and the method can be also applied to RNA:RNA duplexes.

Limitations on the utility of DGGE include the requirement that the denaturing conditions must be optimized for each type of DNA to be tested. Furthermore, the method requires specialized equipment to prepare the gels and maintain the needed high temperatures during electrophoresis. The expense associated with the synthesis of the clamping tail on one oligonucleotide for each sequence to be tested is also a major consideration. In addition, long running times are required for DGGE. The long running time of DGGE was shortened in a modification of DGGE called constant denaturant gel electrophoresis (CDGE). CDGE requires that gels be performed under different denaturant conditions in order to reach high efficiency for the detection of mutations.

A technique analogous to DGGE, termed temperature gradient gel electrophoresis (TGGE), uses a thermal gradient rather than a chemical denaturant gradient. TGGE requires the use of specialized equipment which can generate a temperature gradient perpendicularly oriented relative to the electrical field. TGGE can detect mutations in relatively small fragments of DNA therefore scanning of large gene segments requires the use of multiple PCR products prior to running the gel.

Single-Strand Conformation Polymorphism (SSCP): Another common method, called “Single-Strand Conformation Polymorphism” (SSCP) was developed by Hayashi, Sekya and colleagues and is based on the observation that single strands of nucleic acid can take on characteristic conformations in non-denaturing conditions, and these conformations influence electrophoretic mobility. The complementary strands assume sufficiently different structures that one strand may be resolved from the other. Changes in sequences within the fragment will also change the conformation, consequently altering the mobility and allowing this to be used as an assay for sequence variations.

The SSCP process involves denaturing a DNA segment (e.g., a PCR product) that is labeled on both strands, followed by slow electrophoretic separation on a non-denaturing polyacrylamide gel, so that intra-molecular interactions can form and not be disturbed during the run. This technique is extremely sensitive to variations in gel composition and temperature. A serious limitation of this method is the relative difficulty encountered in comparing data generated in different laboratories, under apparently similar conditions.

Dideoxy fingerprinting (ddF): The dideoxy fingerprinting (ddF) is another technique developed to scan genes for the presence of mutations. The ddF technique combines components of Sanger dideoxy sequencing with SSCP. A dideoxy sequencing reaction is performed using one dideoxy terminator and then the reaction products are electrophoresed on nondenaturing polyacrylamide gels to detect alterations in mobility of the termination segments as in SSCP analysis. While ddF is an improvement over SSCP in terms of increased sensitivity, ddF requires the use of expensive dideoxynucleotides and this technique is still limited to the analysis of fragments of the size suitable for SSCP (i.e., fragments of 200-300 bases for optimal detection of mutations).

In addition to the above limitations, all of these methods are limited as to the size of the nucleic acid fragment that can be analyzed. For the direct sequencing approach, sequences of greater than 600 base pairs require cloning, with the consequent delays and expense of either deletion sub-cloning or primer walking, in order to cover the entire fragment. SSCP and DGGE have even more severe size limitations. Because of reduced sensitivity to sequence changes, these methods are not considered suitable for larger fragments. Although SSCP is reportedly able to detect 90% of single-base substitutions within a 200 base-pair fragment, the detection drops to less than 50% for 400 base pair fragments. Similarly, the sensitivity of DGGE decreases as the length of the fragment reaches 500 base-pairs. The ddF technique, as a combination of direct sequencing and SSCP, is also limited by the relatively small size of the DNA that can be screened.

Reverse dot blot: This technique uses labeled sequence specific oligonucleotide probes and unlabeled nucleic acid samples. Activated primary amine-conjugated oligonucleotides are covalently attached to carboxylated nylon membranes. After hybridization and washing, the labeled probe, or a labeled fragment of the probe, can be released using oligomer restriction, i.e., the digestion of the duplex hybrid with a restriction enzyme. Circular spots or lines are visualized calorimetrically after hybridization through the use of streptavidin horseradish peroxidase incubation followed by development using tetramethylbenzidine and hydrogen peroxide, or via chemiluminescence after incubation with avidin alkaline phosphatase conjugate and a luminous substrate susceptible to enzyme activation, such as CSPD, followed by exposure to x-ray film.

It will be appreciated that advances in the field of SNP detection have provided additional accurate, easy, and inexpensive large-scale SNP genotyping techniques, such as Pyrosequencing™, Acycloprime™, dynamic allele-specific hybridization (DASH, Howell, W. M. et al., 1999. Dynamic allele-specific hybridization (DASH). Nat. Biotechnol. 17: 87-8), microplate array diagonal gel electrophoresis [MADGE, Day, I. N. et al., 1995. High-throughput genotyping using horizontal polyacrylamide gels with wells arranged for microplate array diagonal gel electrophoresis (MADGE). Biotechniques. 19: 830-5], the TaqMan system (Holland, P. M. et al., 1991. Detection of specific polymerase chain reaction product by utilizing the 5′→3′ exonuclease activity of Thermus aquaticus DNA polymerase. Proc Natl Acad Sci U S A. 88: 7276-80), as well as various DNA “chip” technologies such as the GeneChip microarrays (e.g., Affymetrix SNP chips) which are disclosed in U.S. Pat. No. 6,300,063 to Lipshutz, et al. 2001, which is fully incorporated herein by reference, Genetic Bit Analysis (GBA™) which is described by Goelet, P. et al. (PCT Appl. No. 92/15712), peptide nucleic acid (PNA, Ren B, et al., 2004. Nucleic Acids Res. 32: e42) and locked nucleic acids (LNA, Latorra D, et al., 2003. Hum. Mutat. 22: 79-85) probes, Molecular Beacons (Abravaya K, et al., 2003. Clin Chem Lab Med. 41: 468-74), intercalating dye [Germer, S. and Higuchi, R. Single-tube genotyping without oligonucleotide probes. Genome Res. 9:72-78 (1999)], FRET primers (Solinas A et al., 2001. Nucleic Acids Res. 29: E96), AlphaScreen (Beaudet L, et al., Genome Res. 2001, 11(4): 600-8), SNPstream (Bell P A, et al., 2002. Biotechniques. Suppl.: 70-2, 74, 76-7), Multiplex minisequencing (Curcio M, et al., 2002. Electrophoresis. 23: 1467-72), SnaPshot (Turner D, et al., 2002. Hum Immunol. 63: 508-13), MassEXTEND (Cashman J R, et al., 2001. Drug Metab Dispos. 29: 1629-37), GOOD assay (Sauer S, and Gut I G. 2003. Rapid Commun. Mass. Spectrom. 17: 1265-72), Microarray minisequencing (Liljedahl U, et al., 2003. Pharmacogenetics. 13: 7-17), arrayed primer extension (APEX) (Tonisson N, et al., 2000. Clin. Chem. Lab. Med. 38: 165-70), Microarray primer extension (O'Meara D, et al., 2002. Nucleic Acids Res. 30: e75), Tag arrays (Fan J B, et al., 2000. Genome Res. 10: 853-60), Template-directed incorporation (TDI) (Akula N, et al., 2002. Biotechniques. 32: 1072-8), fluorescence polarization (Hsu T M, et al., 2001. Biotechniques. 31: 560, 562, 564-8), Colorimetric oligonucleotide ligation assay (OLA, Nickerson D A, et al., 1990. Proc. Natl. Acad. Sci. USA. 87: 8923-7), Sequence-coded OLA (Gasparini P, et al., 1999. J. Med. Screen. 6: 67-9), Microarray ligation, Ligase chain reaction, Padlock probes, Rolling circle amplification, Invader assay (reviewed in Shi M M. 2001. Enabling large-scale pharmacogenetic studies by high-throughput mutation detection and genotyping technologies. Clin Chem. 47: 164-72), coded microspheres (Rao K V et al., 2003. Nucleic Acids Res. 31: e66) and MassArray (Leushner J, Chiu N H, 2000. Mol Diagn. 5: 341-80).

According to a presently preferred embodiment of the present invention the step of searching for any of the nucleic acid sequences described here, in tumor cells or in cells derived from a cancer patient is effected by any suitable technique, including, but not limited to, nucleic acid sequencing, polymerase chain reaction, ligase chain reaction, self-sustained synthetic reaction, Qβ-Replicase, cycling probe reaction, branched DNA, restriction fragment length polymorphism analysis, mismatch chemical cleavage, heteroduplex analysis, allele-specific oligonucleotides, denaturing gradient gel electrophoresis, constant denaturant gel electrophoresis, temperature gradient gel electrophoresis, dideoxy fingerprinting, Pyrosequencing™, Acycloprime™, and reverse dot blot.

Detection may also optionally be performed with a chip or other such device. The nucleic acid sample which includes the candidate region to be analyzed is preferably isolated, amplified and labeled with a reporter group. This reporter group can be a fluorescent group such as phycoerythrin. The labeled nucleic acid is then incubated with the probes immobilized on the chip using a fluidics station. For example, Manz et al. (1993) Adv in Chromatogr 1993; 33:1-66 describe the fabrication of fluidics devices and particularly microcapillary devices, in silicon and glass substrates.

Once the reaction is completed, the chip is inserted into a scanner and patterns of hybridization are detected. The hybridization data is collected, as a signal emitted from the reporter groups already incorporated into the nucleic acid, which is now bound to the probes attached to the chip. Since the sequence and position of each probe immobilized on the chip is known, the identity of the nucleic acid hybridized to a given probe can be determined.

Preferably, the detection of at least one nucleic acid change and/or the splice variant sequence of the present invention is effected in a biological sample containing RNA molecules using, for example, RT-PCR or in situ RT-PCR.

RT-PCR analysis: This method uses PCR amplification of relatively rare RNAs molecules. First, RNA molecules are purified from the cells and converted into complementary DNA (cDNA) using a reverse transcriptase enzyme (such as an MMLV-RT) and primers such as, oligo dT, random hexamers or gene specific primers. Then by applying gene specific primers and Taq DNA polymerase, a PCR amplification reaction is carried out in a PCR machine. Those of skills in the art are capable of selecting the length and sequence of the gene specific primers and the PCR conditions (i.e., annealing temperatures, number of cycles and the like) which are suitable for detecting specific RNA molecules. It will be appreciated that a semi-quantitative RT-PCR reaction can be employed by adjusting the number of PCR cycles and comparing the amplification product to known controls.

In situ RT-PCR stain: This method is described in Nuovo G J, et al. [Intracellular localization of polymerase chain reaction (PCR)-amplified hepatitis C cDNA. Am J Surg Pathol. 1993, 17: 683-90] and Komminoth P, et al. [Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract. 1994, 190: 1017-25]. Briefly, the RT-PCR reaction is performed on fixed cells by incorporating labeled nucleotides to the PCR reaction. The reaction is carried on using a specific in situ RT-PCR apparatus such as the laser-capture microdissection PixCell I LCM system available from Arcturus Engineering (Mountainview, Calif.).

It will be appreciated that when utilized along with automated equipment, the above described detection methods can be used to screen multiple samples for a disease and/or pathological condition both rapidly and easily.

Immunoassays

In another embodiment of the present invention, an immunoassay can be used to qualitatively or quantitatively detect and analyze markers in a sample. This method comprises: providing an antibody that specifically binds to a marker; contacting a sample with the antibody; and detecting the presence of a complex of the antibody bound to the marker in the sample.

To prepare an antibody that specifically binds to a marker, purified protein markers can be used. Antibodies that specifically bind to a protein marker can be prepared using any suitable methods known in the art.

After the antibody is provided, a marker can be detected and/or quantified using any of a number of well recognized immunological binding assays. Useful assays include, for example, an enzyme immune assay (EIA) such as enzyme-linked immunosorbent assay (ELISA), a radioimmune assay (RIA), a Western blot assay, or a slot blot assay see, e.g., U.S. Pat. Nos. 4,366,241; 4,376,110; 4,517,288; and 4,837,168). Generally, a sample obtained from a subject can be contacted with the antibody that specifically binds the marker.

Optionally, the antibody can be fixed to a solid support to facilitate washing and subsequent isolation of the complex, prior to contacting the antibody with a sample. Examples of solid supports include but are not limited to glass or plastic in the form of, e.g., a microtiter plate, a stick, a bead, or a microbead. Antibodies can also be attached to a solid support.

After incubating the sample with antibodies, the mixture is washed and the antibody-marker complex formed can be detected. This can be accomplished by incubating the washed mixture with a detection reagent. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays will be carried out at ambient temperature, although they can be conducted over a range of temperatures, such as 10° C. to 40° C.

The immunoassay can be used to determine a test amount of a marker in a sample from a subject. First, a test amount of a marker in a sample can be detected using the immunoassay methods described above. If a marker is present in the sample, it will form an antibody-marker complex with an antibody that specifically binds the marker under suitable incubation conditions described above. The amount of an antibody-marker complex can optionally be determined by comparing to a standard. As noted above, the test amount of marker need not be measured in absolute units, as long as the unit of measurement can be compared to a control amount and/or signal.

Preferably used are antibodies which specifically interact with the polypeptides of the present invention and not with wild type proteins or other isoforms thereof, for example. Such antibodies are directed, for example, to the unique sequence portions of the polypeptide variants of the present invention, including but not limited to bridges, heads, tails and insertions described in greater detail below. Preferred embodiments of antibodies according to the present invention are described in greater detail with regard to the section entitled “Antibodies”.

Radio-immunoassay (RIA): In one version, this method involves precipitation of the desired substrate and in the methods detailed hereinbelow, with a specific antibody and radiolabelled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabelled or enzyme linked as described hereinabove. Detection may be by autoradiography, calorimetric reaction or chemiluminescence. This method allows both quantitation of an amount of substrate and determination of its identity by a relative position on the membrane which is indicative of a migration distance in the acrylamide gel during electrophoresis.

Immunohistochemical analysis: This method involves detection of a substrate in situ in fixed cells by substrate specific antibodies. The substrate specific antibodies may be enzyme linked or linked to fluorophores. Detection is by microscopy and subjective evaluation. If enzyme linked antibodies are employed, a colorimetric reaction may be required.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Radio-Imaging Methods

These methods include but are not limited to, positron emission tomography (PET) single photon emission computed tomography (SPECT). Both of these techniques are non-invasive, and can be used to detect and/or measure a wide variety of tissue events and/or functions, such as detecting cancerous cells for example. Unlike PET, SPECT can optionally be used with two labels simultaneously. SPECT has some other advantages as well, for example with regard to cost and the types of labels that can be used. For example, U.S. Pat. No. 6,696,686 describes the use of SPECT for detection of breast cancer, and is hereby incorporated by reference as if fully set forth herein.

### EXAMPLE 24

The methodology undertaken to uncover the biomolecular sequences of the present invention includes the following as non-limiting examples only.

Human ESTs and cDNAs were obtained from GenBank versions 136 (Jun. 15, 2003 ftp.ncbi.nih.gov/genbank/release.notes/gb136.release.notes); NCBI genome assembly of April 2003; RefSeq sequences from June 2003; Genbank version 139 (December 2003); Human Genome from NCBI (Build 34) (from October 2003); and RefSeq sequences from December 2003; and from Incyte Corporation (Wilmington, Del., USA). With regard to GenBank sequences, the human EST sequences from the EST (GBEST) section and the human mRNA sequences from the primate (GBPRI) section were used; also the human nucleotide RefSeq mRNA sequences were used (see for example www.ncbi.nlm.nih.gov/Genbank/GenbankOverview.html and for a reference to the EST section, see www.ncbi.nlm.nih.gov/dbEST/; a general reference to dbEST, the EST database in GenBank, may be found in Boguski et al, Nat Genet. 1993 August; 4(4):332-3; all of which are hereby incorporated by reference as if fully set forth herein).

Novel splice variants were predicted using the LEADS clustering and assembly system as described in Sorek, R., Ast, G. & Graur, D. Alu-containing exons are alternatively spliced. Genome Res 12, 1060-7 (2002); U.S. Pat. No. 6,625,545; and U.S. patent application Ser. No. 10/426,002, published as US20040101876 on May 27, 2004; all of which are hereby incorporated by reference as if fully set forth herein. Briefly, the software cleans the expressed sequences from repeats, vectors and immunoglobulins. It then aligns the expressed sequences to the genome taking alternatively splicing into account and clusters overlapping expressed sequences into “clusters” that represent genes or partial genes.

These were annotated using the GeneCarta (Compugen, Tel-Aviv, Israel) platform. The GeneCarta platform includes a rich pool of annotations, sequence information (particularly of spliced sequences), chromosomal information, alignments, and additional information such as SNPs, gene ontology terms, expression profiles, functional analyses, detailed domain structures, known and predicted proteins and detailed homology reports.

EST libraries which included above-average levels of contamination, such as DNA contamination for example, were eliminated. The presence of such contamination was determined as follows. For each library, the number of unspliced ESTs that are not fully contained within other spliced sequences was counted. If the percentage of such sequences (as compared to all other sequences) was at least 4 standard deviations above the average for all libraries being analyzed, this library was tagged as being contaminated and was eliminated from further consideration in the below analysis (see also Sorek, R. & Safer, H. M. A novel algorithm for computational identification of contaminated EST libraries. Nucleic Acids Res 31, 1067-74 (2003) for further details).

### EXAMPLE 25

Various binding assays may optionally and preferably be used in order to determine the effect of the splice variants according to the present invention on calcium channel function and/or the effect of a therapeutic agent (such as a drug or drugs) on calcium channels having a splice variant according to the present invention. Preferably, these assays are performed with a known compound or compounds first, in order to establish the effect of the splice variant of the present invention. Such compounds are well known in the art and could easily be selected by one of ordinary skill in the art.

Methods for screening these compounds for their effects on calcium channel activity have also been disclosed (see for example, U.S. Pat. No. 6,096,514). In addition, some organic calcium channel blocking compounds have been described as being useful to treat stroke, cerebral ischemia, head trauma, or epilepsy involving calcium channel activity (see U.S. Pat. Nos. 6,294,533, 6,423,689). Therefore, a person skilled in the art can use methods known in the art, such as described in the references cited above, to screen for compounds that bind to, and in some cases functionally alter, a splice variant protein according to the present invention, or polypeptide fragments thereof.

Splice variants according to the present invention or fragments thereof can be used in binding studies to identify compounds binding to or interacting with splice variants or fragments thereof. In one embodiment, splice variants or fragments thereof can be used in binding studies, to identify compounds that: bind to or interact with splice variants as described herein; and bind to or interact with one or more other alpha 1 subunit isoforms and not with a splice variant according to the present invention. Such binding studies can be performed using different formats including competitive and non-competitive formats. Further competition studies can be carried out using additional compounds determined to bind to splice variants according to the present invention or other alpha 1 subunit isoforms.

The particular splice variant portions (amino acid sequences) involved in ligand binding can be identified by using labeled compounds that bind to the protein and different protein fragments. Different strategies can be employed to select fragments to be tested to narrow down the binding region. Examples of such strategies include testing consecutive fragments about 15 amino acids in length starting at the N-terminus, and testing longer length fragments. If longer length fragments are tested, a fragment binding to a compound can be subdivided to further locate the binding region. Fragments used for binding studies can be generated using recombinant nucleic acid techniques.

Binding assays can be performed using recombinantly produced splice variants polypeptides present in different environments. Such an environment may optionally be derived from cell portions or fragments, or other “natural” sources. Another exemplary environment is an artificial lipid bilayer into which the splice variant-containing calcium channel is inserted. Such technology is available for example from Nimbus Biotechnologie, Germany, which provides an assay for determining membrane affinity and pore forming properties of proteins/peptides by using solid supported lipid bilayers. This technology enables an ion channel to correct assemble and form a pore in a lipid bilayer which mimics the external membrane of a cell. This assay uses their TRANSIL® technology in a micro well plate (see for example Loidl-Stahlhofen et al., Solid-Supported Biomolecules on Modified Silica Surfaces: A Tool for Fast Physiocochemical Characterization and High-Throughput Screening. 2001, Adv. Mater. 13, 1829-1834). In this assay a calcium channel may optionally be incubated with a defined single lipid bilayer on a solid support. Unbound protein may optionally be removed by washing, for example. Pore formation is preferably measured by using ion sensitive fluorescence dyes on one side of the lipid bilayer; if the pore is successfully formed then migration of fluorescence is optionally and preferably measured by using a fluorescence spectrometer.

Alternatively, other functional assays may optionally be used to measure the activity of a calcium channel by methods known to those of skill in the art, including the electrophysiological methods (e.g., Williams et al., 1992 Science 257, 389-395; see also, for example, U.S. Pat. Nos. 6,353,091; 6,156,726; and 6,096,514).

For any of these assays, various compounds (such as DHP and/or toxins, as described above) may optionally be used in order to assess function of a calcium channel containing an alpha 1 subunit splice variant according to the present invention. Preferably, the effect of such compound(s) is compared to the effect on the known, WT protein.

### EXAMPLE 26

According to another embodiment of the present invention, there is provided a method to computationally identify novel mutually exclusive exons. According to this method, a group of known mutually exclusive events was collected and analyzed as described below. Preliminary results are also provided.

Materials and Methods

Creating a Dataset of Known Mutually Exclusive Exons Using the Leads Software.

For the classification of mutually exclusive exons the LEADS output obtained from NCBI GenBank version 136 (June 2003) was parsed. To define mutually exclusive events two distinct sequences (EST/RNA) that contain the same flanking upstream and downstream exons and two different exons between them were located. To find conserved events in the mouse sequence a LEADS-generated file of mouse ESTs that are mapped to the human chromosome was used. A conserved mouse exon should be mapped to the human chromosome in the same position as its parallel human exon. Thus, two mouse sequences that contain the same flanking exons and different exon between them with the same positions as the human mutually exclusive exons were located. Specific characteristics were found to be common to mutually exclusive exons. These features include a short intervening intron, certain conservation patterns, size identity between the two exons, and sequence identity between the exons (see “Preliminary Results”).

Finding potential novel exons. The input of this stage will be a set of 110,932 conserved internal human exons. Preliminary results show that mutually exclusive exons usually have the same length, or lengths that differ from each other by a multiple of 3, with a short intron between them. Therefore, optionally the method featuring taking 2000 nt from the intronic regions upstream and downstream to the common exon, and looking for canonical splice site boundaries that define a sequence with the same length as the common exon, or with a length that differs by a multiple of three bases. Matching sequences will be considered putative mutually exclusive partners. In addition, my preliminary results and other studies show that in some mutually exclusive events, the two exons have sequence similarity. Similarity is optionally examined by running the tBLASTx program to compare each putative novel exon against its common exon.

To find the protein translation frame it is possible to make an alignment between the sequence of the common exon and the sequence of the RNA that expresses it and calculate the translation frame according to the positions of the exon relative to the RNA and to the positions of the coding sequence (CDS). Then, the translation frame was used to search for stop codons in the putative novel exon and exons that cause protein truncation are preferably removed (see preliminary results).

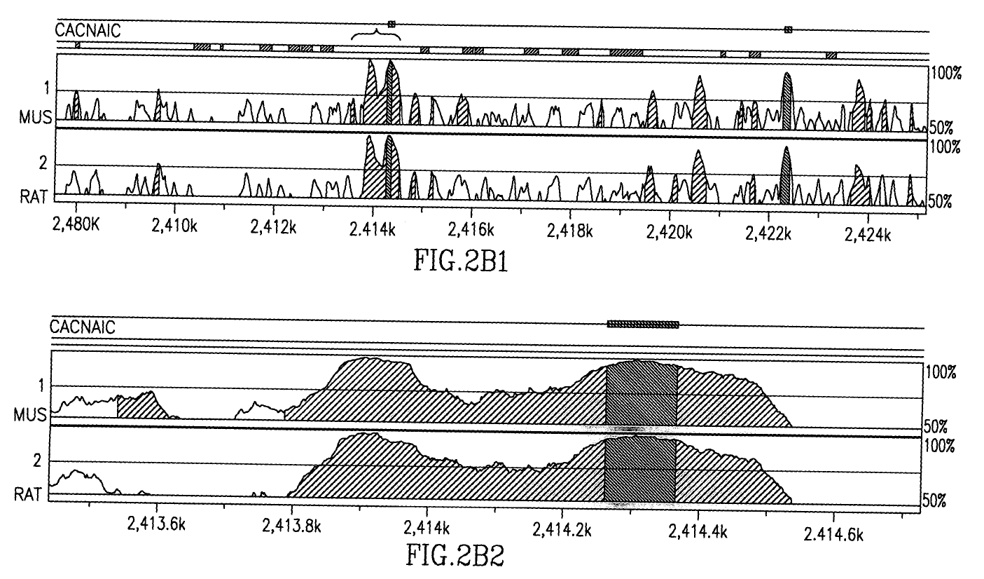

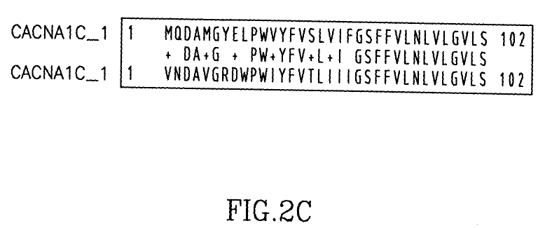

FIG. 2 illustrates some of these points with regard to mutually exclusive exons in the CACNA1C gene. (a) Part of the CACNA1C gene (from UCSC genome browser, http://genome.ucsc.edu). Introns marked as thin lines and exons marked as boxes. Two distinct mRNAs present each of the mutually exclusive exons (marked as red arrows). The intron between the two exons is relatively small (b) Human-Mouse and Human-Rat alignment of the CACNA1C gene corresponding to RefSeq NM_{—}000719 (from vista browser, pipeline.lbl.gov). The X-axis presents the nucleotide coordinates on human chromosome 12, according to the assembly version of the human genome from July 2003. The Y-axis presents the level of conservation between the human genome and the corresponding mouse/rat genome. Blue bars above the conservation area correspond to annotated exons. Blue areas within the conservation graph mark exons; orange areas mark conserved non-exonic sequences. [1] The area marked with a bracket presents the mutually exclusive exons; the other one is constitutively spliced exon. [2] Expanded view of the mutually exclusive exons. The left “hill” presents one of the mutually exclusive exons and the right “hill” presents the other. Note the high conservation rate in the exonic and the flanking intronic regions. (c) Alignment of the two mutually exclusive exons using the tBLASTx program (Altschul, S. F., Madden, T. L., Schaffer, A. A., Zhang, J., Zhang, Z., Miller, W., and Lipman D. J., Gapped BLAST and PSI-BLAST: a new generation of protein database search programs. Nucleic Acids Res 1997; 25:3389-3402), showing sequence similarity.

Searching for the ‘putative novel exon’ in the mouse sequence. In this stage it is determined whether the putative mutually exclusive events are conserved in mouse. The input will be mouse exons, which are orthologs to the common human exons that have putative novel exons. Using BLASTn (31) the putative novel human exons in the introns flanking the common mouse exons are searched. A putative novel exon will be defined as ‘conserved’ only if it will pass a certain alignment threshold, and only if it will be flanked by canonical splice sites in the mouse genome.

RT-PCR. Experimental validation is carried out by RT-PCR on RNAs from a panel of tissues. For each putative mutually exclusive event two distinct reactions are used. For the first reaction oligonucleotide primers will be designed from the common exon and from a flanking exon. For the second reaction oligonucleotide primers will be designed from the novel exon and from the same flanking exon as in the first reaction.

Preliminary Results and Discussion

Mutually exclusive exon characteristics. The search for known mutually exclusive events resulted in 845 human mutually exclusive cases supported by ESTs. Of them, 22 were conserved in mouse. In this set the exons themselves were conserved, as well as their pattern of alternative splicing. As human EST data can be noisy, the mutually exclusive exons were characterized according to the 22 conserved events. I found that there were unique features that characterize these exons:
1. The size of the two exons was frequently identical (12 out of 22 cases); If not identical, the difference was a multiple of 3 (8 out of 22).
2. Mutually exclusive exons were shorter than constitutively spliced exons (average 90 nt, compared to average 129 nt, respectively).
3. The intron that separates the two exons was smaller than usual (average 1500 with median 820, compared to average 3000 and median 2000 of regular introns).
4. No stop codon was found in frame of both exons.
5. Sequences of the two exons were frequently similar by their protein sequence (11/22 cases, using tBLASTx). 3/22 of the cases showed significant similarity in their nucleotide sequence.
6. Both exons were more conserved in mouse than constitutively spliced exons (average 96% and 89% (16) sequence identity, respectively).
7. In addition to the exonic conservation, both exons were flanked by conserved intronic sequences (averaging 100 bases from each side of each exon). This occurred in 19/22 cases.

The differences found probably stem from the special behavior of mutually exclusive exons. The short intron might be needed to cause steric interference that underlies the mutually exclusive splicing mechanism. In addition, since these exons are never incorporated into the same transcript, but they are switched with each other, the difference between their sizes should be a multiple of 3 in order to preserve the frame and prevent protein truncation.

Sequence similarity between mutually exclusive exons was demonstrated in the literature for some mutually exclusive events, and is attributed to ‘tandem exon duplication’ evolutionary events that give rise to mutually exclusive alternative splicing.

Exonic and intronic high conservation patterns were observed also for non-mutually exclusive alternative exons, and are attributed to sequences that regulate alternative splicing.

6.2 Potential novel exon dataset. Out of the 110,932 conserved common exons, 36,724 were found to have putative sequences that are flanked by canonical splice sites and are of a length that is equal to their common exon, or are different by a multiple of 3. On average, there were 100 putative sequences for each common exon, and the total number of putative sequences was 3,619,887. This set was the initial set of low-reliability novel exons, which was further screened for higher reliability candidates.

The first filter was human-mouse conservation filter. In order to localize a putative novel human exon on the mouse intronic region, the BLASTn program was used. 2245 (out of 36,724) common exons were found to have putative novel exons that are conserved in mouse with average identity level of 91.24%. Since exons are defined by canonical splice sites, all putative novel exons for which the mouse ortholog exon lacked such sites were excluded. After this selection, 1209 (out of 2245) common exons having putative novel exons with higher conservation identity level of 92.23% remained. Since the average conservation level of mutually exclusive exons in the training set was 96% (comparing to 89% in constitutive exons), the increasing conservation rate indicated enrichment in true novel exons.

As the next stage of the screening, human-mouse conservation in the upstream and/or downstream flanking intronic regions of both the common and the novel exons was required as a filter. It was found that 654 (out of 1209) common exons were conserved in their flanking regions and their novel exons were conserved in their flanking intronic regions too. These novel exons were conserved in mouse to a level of 93.77%, (indicating again on increasing enrichment of true novel exons).

None of the exons in the known conserved mutually exclusive set created a stop codon in the protein sequence. According to that criterion, novel exons that contained an in-frame stop codon were excluded. This resulted in a set of 119 common exons that have putative novel exon (conservation level of 94.25%)

In cases where the borders of the novel exon were not clear (several options were present), correct borders were selected according to various criteria such as: (i) there is minimal difference between the novel human exon size and the size of the novel mouse exon. (ii) the novel exon had maximal identity percentage when aligned to the mouse orthologous sequence. (iii) novel exons with the minimal difference between the common exon size and the size of the novel exon.

As written above, one of the features that distinguished mutually exclusive exons was sequence similarity between the two exons. To identify sequence similarity between common and novel exons the tBLASTx program was run with expectation value of 0.02. Only 917 out of 36,724 (2.5%) common exons were found to be similar by their protein sequence to their putative novel exons.

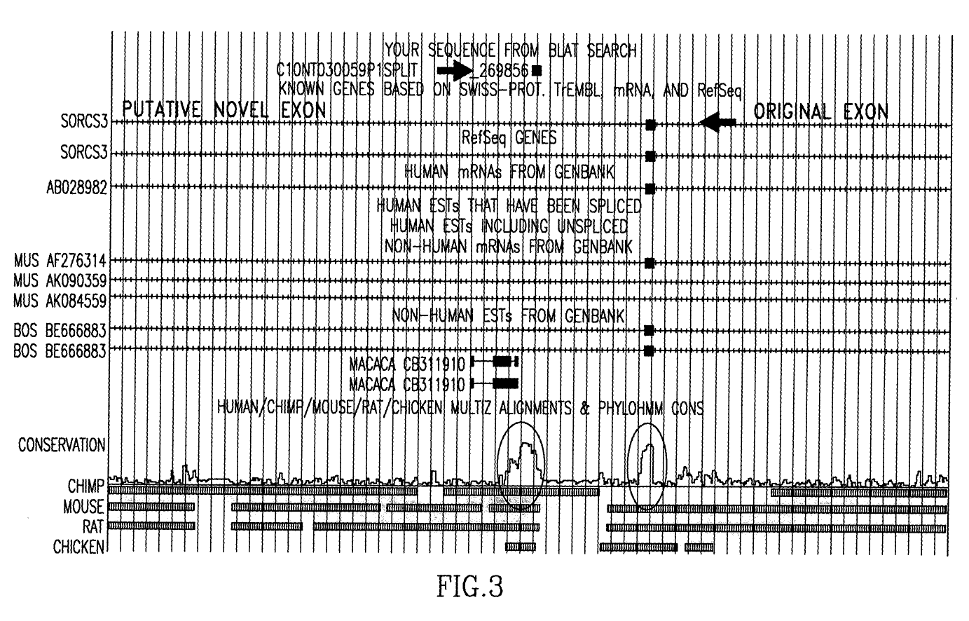

Final set characteristics. The final set contains 119 common and novel exons (FIG. 3). The average intron length of this set is 559.6. This is a relatively short intron, and that strengthens the possibility of mutually exclusive manner. The novel exons are conserved in mouse to a level of 94.25%, slightly less than the 96% average identity level measured in the training set. In addition, about ˜70 nt in the upstream and/or downstream intronic regions adjacent to the novel exon are conserved in mouse with average identity level of 78%. These results indicate that many of the exons in this set might be truly novel human mutually exclusive exons.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. An isolated polynucleotide comprising a nucleic acid sequence according to any of SEQ ID NO 23-44, or the complement thereof.

2. The isolated nucleic acid of claim 1, wherein said nucleic acid comprises a polynucleotide encoding for the amino acid sequence of any of SEQ ID NOs 1-22.

3. The isolated nucleic acid of claim 1, wherein said nucleotide sequence encodes for a polypeptide consisting of the amino acid sequence of any of SEQ ID NOs 1-22.

4. An isolated polypeptide comprising a polypeptide according to any of SEQ ID NOs 1-22.

5. The polypeptide of claim 4, wherein said polypeptide consists of the amino acid sequence according to any of SEQ ID NOs 1-22.

6. An expression vector comprising a nucleotide sequence encoding for any of SEQ ID NOs 1-22, wherein said nucleotide sequence is transcriptionally coupled to an exogenous promoter.

7. The expression vector of claim 6, wherein said nucleotide sequence encodes for a polypeptide consisting of the amino acid sequence according to any of SEQ ID NOs 1-22.

8. The expression vector of claim 6, wherein said nucleotide sequence comprises any of SEQ ID NO 23-44.

9. The expression vector of claim 6, wherein said nucleotide sequence consists of the sequence of any of SEQ ID NO 23-44.

10. A recombinant cell comprising the expression vector of claim 6, wherein said cell comprises an RNA polymerase recognized by said promoter.

11. A recombinant cell made by a process comprising the step of introducing the expression vector of claim 6 into said cell.

12. A method of preparing a splice variant polypeptide comprising growing the recombinant cell of claim 10 under conditions wherein said polypeptide is expressed from said expression vector.

13. A method of screening for compounds able to bind selectively to a splice variant according to the present invention comprising the steps of: (a) providing a splice variant polypeptide according to claim 4; (b) providing a WT protein polypeptide that is not said splice variant polypeptide, (c) contacting said splice variant polypeptide and said WT polypeptide with a test preparation comprising one or more compounds; and (d) determining the binding of said test preparation to said splice variant polypeptide and said WT polypeptide, wherein a test preparation which binds said splice variant polypeptide but does not bind said WT polypeptide contains a compound that selectively binds said splice variant polypeptide.

14. The method of claim 13, wherein said splice variant polypeptide is obtained by expression of said polypeptide from an expression vector comprising a polynucleotide encoding the amino acid sequence according to any of SEQ ID NOs 1-22.

15. The method of claim 14, wherein said polypeptide consists of the amino acid sequence according to any of SEQ ID NOs 1-22.

16. A method of screening for a compound able to bind to or interact with a splice variant protein or a fragment thereof comprising the steps of: (a) expressing a polypeptide comprising the amino acid sequence according to claim 4 or fragment thereof from a recombinant nucleic acid; (b) providing to said polypeptide a labeled ligand that specifically binds to said polypeptide and a test preparation comprising one or more compounds; and (c) measuring the effect of said test preparation on binding of said labeled ligand to said polypeptide, wherein a test preparation that alters the binding of said labeled ligand to said polypeptide contains a compound that binds to or interacts with said polypeptide.

17. The method of claim 16, wherein said steps (b) and (c) are performed in vitro.

18. The method of claim 16, wherein said steps (a), (b) and (c) are preformed using a whole cell.

19. The method of claim 16, wherein said polypeptide is expressed from an expression vector.

20. The method of claim 16, wherein said ligand is a calcium channel-binder.

21. The method of claim 20, wherein said polypeptide consists of an amino acid sequence according to any of SEQ ID NOs 1-22 or a fragment thereof.

22. The method of claim 20, wherein said test preparation contains one compound.
